(19) United States
(12) Patent Application Publication
(10) Pub. No.: US 2026/0137773 A1
(43) Pub. Date: May 21, 2026

(54) ONCOLYTIC VACCINIA VIRUSES AND RECOMBINANT VIRUSES AND METHODS OF USE THEREOF

(71) Applicant: ViroMissile, Inc., La Jolla, CA (US)

(72) Inventor: Nanhai George CHEN, San Diego, CA (US)

(73) Assignee: ViroMissile, Inc., La Jolla, CA (US)

(21) Appl. No.: 19/414,517

(22) Filed: Dec. 10, 2025

Related U.S. Application Data
(62) Division of application No. 18/349,083, filed on Jul. 7, 2023.
(60) Provisional application No. 63/368,029, filed on Jul. 8, 2022.

(51) Int. Cl. A61K 39/285; A61K 9/00; A61K 31/451; A61K 39/00; A61K 45/06; A61P 35/00

(52) U.S. Cl. CPC A61K 39/285; A61K 9/0019; A61K 31/451; A61K 45/06; A61P 35/00; A61K 2039/5252; A61K 2039/54

(57) ABSTRACT

Provided herein are clonal strains of a vaccinia virus that exhibits enhanced anti-tumor properties and/or reduced immunogenicity, and recombinant vaccinia virus derived from the same. Also provided herein are recombinant oncolytic virus strains that include an inactivating mutation in one or more viral genes, and/or one of more heterologous nucleic acids each encoding one or more heterologous gene products. The viruses, e.g., vaccinia viruses, provided herein, including recombinant vaccinia viruses, can be used as an oncolytic virus therapy, e.g., an oncolytic vaccinia virus therapy, for treating cancer. Also provided herein are pharmaceutical compositions and methods and uses of the viruses, e.g., vaccinia viruses, for treating cancer, as well as nucleic acids encoding the viruses.

### CROSS-REFERENCE TO RELATED APPLICATION

This application is a divisional of U.S. application Ser. No. 18/349,083, filed on Jul. 7, 2023, which claims priority to U.S. Provisional Application No. 63/368,029, filed Jul. 8, 2022, entitled “ONCOLYTIC VACCINIA VIRUSES AND RECOMBINANT VIRUSES AND METHODS OF USE THEREOF,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 773192000110SeqList.xml, created Dec. 9, 2025, which is 6,262,588 bytes in size. The information in the electronic format of the Sequence Listing is incorporated by reference in its entirety.

### FIELD

The present disclosure provides clonal strains of a vaccinia virus that exhibits enhanced anti-tumor properties and/or reduced immunogenicity, and recombinant vaccinia virus derived from the same. The vaccinia viruses of the disclosure, including recombinant vaccinia viruses, can be used as an oncolytic vaccinia virus therapy for treating cancer. The present disclosure also provides pharmaceutical compositions and methods and uses of the vaccinia viruses for treating cancer.

### BACKGROUND

Vaccinia is an oncolytic virus and accumulates in tumors. In some cases, oncolytic viruses (OVs) are viruses that replicate selectively or more efficiently in cancer cells than in non-cancer cells. Oncolytic vaccinia viruses include recombinant viruses that are engineered from a native virus by gene disruptions or gene additions so as to improve its anti-tumor properties, such as tumor selectivity or preferential replication in tumor cells, host tropism, surface attachment, lysis, and spread. Among such recombinant vaccinia viruses are attenuated viruses that are modified in one or more viral genes that results in loss or reduced expression of a viral gene or inactivation of a viral protein. However, the effectiveness of oncolytic viruses is hindered by the strong immune response induced by the virus. Immune factors such as antibodies neutralize the virus by binding to it directly and preventing a successful infection of the cells or by marking it for destruction either by complement or by other immune cells. Thus, there still exists a need for improved oncolytic vaccinia viruses that have reduced ability to induce antiviral defenses and have enhanced anti-tumor activities.

### SUMMARY

Provided herein is a recombinant oncolytic vaccinia virus, comprising: an inactivating mutation of B2R; a heterologous nucleic acid encoding interferon regulatory factor 3 (IRF3); and at least one heterologous nucleic acid encoding one or more cytokine and/or chemokine.

In some embodiments, the at least one heterologous nucleic acid encoding one or more cytokine and/or chemokine comprises a heterologous nucleic acid encoding chemokine ligand 9 (CXCL9) and/or IL-12. In some of any such embodiments, the at least one heterologous nucleic acid encoding one or more cytokine and/or chemokine is a heterologous nucleic acid encoding chemokine ligand 9 (CXCL9). In some of any such embodiments, the at least one heterologous nucleic acid encoding one or more cytokine and/or chemokine is a heterologous nucleic acid encoding IL-12. In some of any embodiments, the at least one heterologous nucleic acid encoding one or more cytokine and/or chemokine is a heterologous nucleic acid encoding CXCL9 and IL-12. In some embodiments, the at least one heterologous nucleic acid encoding one or more cytokine and/or chemokine is a heterologous nucleic acid encoding CXCL9 and a heterologous nucleic acid encoding IL-12.

In some of any embodiments: the CXCL9 is human CXCL9. In some embodiments, the CXCL9 comprises the amino acid sequence set forth in SEQ ID NO: 99, or an amino acid sequence that has at least 95%, 96%, 97%, 98%, or 99% sequence identity to the amino acid sequence of SEQ ID NO: 99. In some embodiments, the amino acid sequence of CXCL9 is set forth in SEQ ID NO: 99. In some of any embodiments, the CXCL9 is mouse CXCL9. In some embodiments, the CXCL9 comprises the amino acid sequence set forth in SEQ ID NO: 106, or an amino acid sequence that has at least 95%, 96%, 97%, 98%, or 99% sequence identity to the amino acid sequence of SEQ ID NO: 106. In some embodiments, the amino acid sequence of CXCL9 is set forth in SEQ ID NO: 106.

In some of any embodiments: the IL-12 is a human single-chain IL-12. In some embodiments, the single-chain IL-12 is composed of human IL-12A (p35) and human IL-12B (p40) subunits, optionally separated by a linker. In some embodiments, the single-chain IL-12 comprises the amino acid sequence set forth in SEQ ID NO: 103, or an amino acid sequence that has at least 95%, 96%, 97%, 98%, or 99% sequence identity to the amino acid sequence of SEQ ID NO: 103. In some embodiments, the amino acid sequence the single-chain IL-12 is set forth in SEQ ID NO: 103. In some of any embodiments, the IL-12 is a mouse single-chain IL-12. In some embodiments, the single-chain IL-12 is composed of mouse IL-12A (p35) and mouse IL-12B (p40) subunits, optionally separated by a linker. In some embodiments, the single-chain IL-12 comprises the amino acid sequence set forth in SEQ ID NO: 102, or an amino acid sequence that has at least 95%, 96%, 97%, 98%, or 99% sequence identity to the amino acid sequence of SEQ ID NO: 102. In some embodiments, the amino acid sequence the single-chain IL-12 is set forth in SEQ ID NO: 102.

In some of any embodiments, the at least one heterologous nucleic acid encoding one or more cytokine and/or chemokine comprises a heterologous nucleic acid encoding IL-2. In some embodiments, the IL-2 comprises an amino acid sequence set forth in any one of SEQ ID NOs: 98, 100, 101, 104, and 105, or an amino acid sequence that has at least 95%, 96%, 97%, 98%, or 99% sequence identity to the amino acid sequence of any one of SEQ ID NOs: 98, 100, 101, 104, and 105. In some embodiments, the IL-2 is set forth in SEQ ID NO: 105. In some embodiments, the IL-2 is a superkine of the sequence set forth in SEQ ID NO: 105 or a sequence that has at least 95%, 96%, 97%, 98%, or 99% sequence identity to SEQ ID NO: 105.

In some of any embodiments, the IL-2 is an IL-2 superkine. In some embodiments, the IL-2 superkine is H9, H9T, MDNA11, or MDNA11T. In some of any embodiments, the H9 IL-2 superkine comprises the amino acid sequence of SEQ ID NO: 100, or comprises an amino acid sequence that has at least 95%, 96%, 97%, 98%, or 99% sequence identity to the amino acid sequence of SEQ ID NO: 100. In some of any embodiments, the H9T IL-2 superkine comprises the amino acid sequence of SEQ ID NO: 104, or comprises an amino acid sequence that has at least 95%, 96%, 97%, 98%, or 99% sequence identity to the amino acid sequence of SEQ ID NO: 104. In some of any embodiments, the MDNA11 IL-2 superkine comprises the amino acid sequence of SEQ ID NO: 101, or comprises an amino acid sequence that has at least 95%, 96%, 97%, 98%, or 99% sequence identity to the amino acid sequence of SEQ ID NO: 101. In some of any embodiments, the MDNA11T IL-2 superkine comprises the amino acid sequence of SEQ ID NO: 98, or comprises an amino acid sequence that has at least 95%, 96%, 97%, 98%, or 99% sequence identity to the amino acid sequence of SEQ ID NO: 98. In some of any embodiments, the IL-2 superkine is MDNA11T, and the MDNA11T comprises the amino acid sequence set forth in SEQ ID NO: 98, or an amino acid sequence that has at least 95%, 96%, 97%, 98%, or 99% sequence identity to the amino acid sequence set forth in SEQ ID NO: 98.

In some of any embodiments, the recombinant oncolytic virus further comprises one or more heterologous gene product selected from the group consisting of a complement inhibitor, a T cell or NK cell evader, an immune stimulating protein, an anti-angiogenic protein, an interferon regulatory factor, an apoptosis inducible protein or a combination of any of the foregoing.

In some of any embodiments, the inactivating mutation of B2R is a deletion of all or a portion of the B2R gene loci. In some of any embodiments, said deletion is sufficient to render the encoded B2R gene product non-functional. In some embodiments, the inactivating mutation of B2R is one or more amino acid substitutions in the encoded gene product. In some of any embodiments, the inactivating mutation of B2R is characterized by insertion of the heterologous nucleic acid into the B2R gene loci, such as in place of the deletion of all or a portion of the B2R gene loci. In some embodiments, the heterologous nucleic acid encodes IRF3 or the cytokine and/or chemokine. In some of any embodiments, the inactivating mutation of B2R is by insertion of the heterologous nucleic acid encoding IRF3 and/or by insertion of at least one of the at least one heterologous nucleic acid encoding one or more cytokine and/or chemokine into the B2R gene loci. In some of any embodiments, the inactivating mutation of B2R is characterized by insertion of the heterologous nucleic acid encoding chemokine ligand 9 (CXCL9) and/or IL-12 into the B2R gene loci.

In some of any embodiments, the heterologous nucleic acid encoding IRF3 is inserted into the hemagglutinin (HA), J2R (thymidine kinase), F14.5L, A56R (hemagglutinin), vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus. In some of any embodiments, the at least one of the at least one heterologous nucleic acid encoding one or more cytokine and/or chemokine is inserted into the HA, J2R, F14.5L, A56R, vaccinia growth factor, A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus. In some of any such embodiments, the insertion is in place of a deletion of all or a portion of the respective gene loci.

In some of any embodiments, the nucleic acid genome of the recombinant oncolytic vaccinia virus is modified from a parental vaccinia virus that has a nucleic acid genome that has at least 99% sequence identity to the nucleic acid sequence set forth in SEQ ID NO: 1. In some embodiments, the nucleic acid genome of the recombinant oncolytic vaccinia virus is modified from a parental vaccinia virus that has the nucleic acid genome set forth in SEQ ID NO: 1.

In some of any embodiments, the nucleic acid genome of the parental vaccinia virus is characterized by one or more of: (i) a variant 017 open reading frame (ORF) encoding an amino acid sequence that has at least 95% sequence identity to SEQ ID NO: 57 and comprises a polar uncharged amino acid at position 66, optionally a threonine (T) at position 66; (ii) a variant 038 (K5L) ORF comprising a nucleotide insertion to effect a frameshift mutation, wherein the 038 (K5L) gene product is altered; (iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419; (iv) a variant 104 (H4L) ORF encoding an amino acid sequence having at least 95% sequence identity to SEQ ID NO: 61 and comprises a negatively charged amino acid at position 591, optionally aspartic acid (D) at position 591; and (v) a variant 182 (A56R) ORF comprising deletion of two nucleotides to effect a frameshift mutation, wherein the 182 (A56R) ORF gene product is altered.

In some of any embodiments, the nucleic acid genome of the parental virus is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine (C) at the position corresponding to position 92969 of SEQ ID NO: 1; (vi) the nucleic acid sequence CACTTATATAT at the positions corresponding to positions 106870 to 106880 of SEQ ID NO: 1; (vii) the nucleic acid sequence GTTTTCATTA at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC at the positions corresponding to positions 165844 to 185853 of SEQ ID NO: 1; and (x) a C at the position corresponding to position 187805 of SEQ ID NO: 1.

In some of any embodiments, the nucleic acid genome of the recombinant oncolytic vaccinia virus has at least 85%, 86%, 87%, 88%, 89%, 90%, 91%, 92%, 93%, 94%, 95%, 96%, 97%, 98%, or 99% sequence identity to the nucleic acid sequence set forth in SEQ ID NO: 1. In some of anyembodiments, the nucleic acid genome of the recombinant oncolytic vaccinia virus has at least 95%, 96%, 97%, 98%, or 99% sequence identity to the nucleic acid sequence set forth in SEQ ID NO: 1. In some of any embodiments, the nucleic acid genome of the recombinant oncolytic vaccinia virus has at least 95% sequence identity to the nucleic acid sequence set forth in SEQ ID NO: 1. In some of any embodiments, the nucleic acid genome of the recombinant oncolytic vaccinia virus has at least 96% sequence identity to the nucleic acid sequence set forth in SEQ ID NO: 1. In some of any embodiments, the nucleic acid genome of the recombinant oncolytic vaccinia virus has at least 97% sequence identity to the nucleic acid sequence set forth in SEQ ID NO: 1. In some of any embodiments, the nucleic acid genome of the recombinant oncolytic vaccinia virus has at least 98% sequence identity to the nucleic acid sequence set forth in SEQ ID NO: 1. In some of any embodiments, the nucleic acid genome of the recombinant oncolytic vaccinia virus has at least 99% sequence identity to the nucleic acid sequence set forth in SEQ ID NO: 1.

In some of any embodiments: the heterologous nucleic acid encoding IRF3 is inserted into the J2R (thymidine kinase) gene locus in the genome of the virus; and the at least one heterologous nucleic acid encoding one or more cytokine and/or chemokine comprises a heterologous nucleic acid encoding CXCL9 and IL-12, wherein the heterologous nucleic acid encoding CXCL9 and IL-12 is inserted into the A56R gene locus in the genome of the virus.

In some of any embodiments, the nucleic acid genome of the recombinant oncolytic vaccinia virus comprises the nucleic acid sequence of SEQ ID NO: 85, or a nucleic acid sequence that has at least 95%, 96%, 97%, 98%, or 99% sequence identity to the nucleic acid sequence set forth in SEQ ID NO: 85. In some embodiments, the nucleic acid genome of the recombinant oncolytic vaccinia virus is set forth in SEQ ID NO: 85.

In some of any embodiments, the heterologous nucleic acid encoding IRF3 is inserted into the B2R (viral cGAMP-specific nuclease) gene locus in the genome of the virus; and the at least one heterologous nucleic acid encoding one or more cytokine and/or chemokine comprises a heterologous nucleic acid encoding CXCL9 and IL-12, wherein the heterologous nucleic acid encoding CXCL9 and IL-12 is inserted into the A56R gene locus in the genome of the virus.

In some of any embodiments, the recombinant oncolytic vaccinia virus further comprises a heterologous nucleic acid encoding an apoptosis-inducible protein. In some of embodiments, the apoptosis-inducible protein is an inducible death effector domain (iDED). In some of any embodiments, the iDED comprises the amino acid sequence set forth in SEQ ID NO: 27 or a sequence of amino acids that has at least 85%, 90%, 91%, 92%, 93%, 94%, 95%, 96%, 97%, 98%, or 99% sequence identity to the amino acid sequence set forth in SEQ ID NO: 27. In some embodiments, the iDED is set forth in SEQ ID NO: 27. In some of any embodiments, the heterologous nucleic acid encoding an iDED is inserted into or in place of the J2R gene locus in the genome of the virus.

In some of any embodiments, the nucleic acid genome of the recombinant oncolytic vaccinia virus comprises the nucleic acid sequence of SEQ ID NO: 86, or a nucleic acid sequence that has at least 95%, 96%, 97%, 98%, or 99% sequence identity to the nucleic acid sequence set forth in SEQ ID NO: 86. In some embodiments, the nucleic acid genome of the recombinant oncolytic vaccinia virus is set forth in SEQ ID NO: 86.

In some of any embodiments, the recombinant oncolytic vaccinia virus further comprises a heterologous nucleic acid encoding one or more T cell or NK cell evader proteins. In some embodiments, the one or more T cell or NK cell evader proteins comprises a set of proteins encoded by Cowpox virus ORFs 012, 203 and 018 (CPXV012-203-018). In some embodiments, the set of proteins encoded by CPXV012-203-018 comprises: (i) the amino acid sequence set forth in SEQ ID NO: 20 (CPXV012) or an amino acid sequence that has at least 85%, 90%, 91%, 92%, 93%, 94%, 95%, 96%, 97%, 98%, or 99% sequence identity to the amino acid sequence set forth in SEQ ID NO: 20, (ii) the amino acid sequence set forth in SEQ ID NO: 21 (CPXV0203) or an amino acid sequence that has at least 85%, 90%, 91%, 92%, 93%, 94%, 95%, 96%, 97%, 98%, or 99% sequence identity to the amino acid sequence set forth in SEQ ID NO: 21, and (iii) the amino acid sequence set forth in SEQ ID NO: 22 (CPXV018) or an amino acid sequence that has at least 85%, 90%, 91%, 92%, 93%, 94%, 95%, 96%, 97%, 98%, or 99% sequence identity to the amino acid sequence set forth in SEQ ID NO: 22.

In some of any embodiments, the recombinant oncolytic vaccinia virus further comprises a heterologous nucleic acid encoding a complement inhibitor. In some embodiments, the complement inhibitor is Borrelia burgdorferi complement regulatory-acquiring surface protein-2 (CRASP-2). In some embodiments, the heterologous nucleic acid encoding CRASP-2 is fused with a viral membrane gene, optionally F14.5L, to produce a fusion gene encoding a fusion protein. In some of any embodiments, the fusion protein comprises the CRASP-2 fused to a viral membrane protein encoded by the viral membrane gene. In some of any embodiments, the viral membrane protein is F14.5L. In some embodiments, the fusion is at the C-terminus of F14.5L.

In some of any embodiments, the nucleic acid genome of the recombinant oncolytic vaccinia virus comprises the nucleic acid sequence of SEQ ID NO: 90, or a nucleic acid sequence that has at least 95%, 96%, 97%, 98%, or 99% sequence identity to the nucleic acid sequence set forth in SEQ ID NO: 90. In some embodiments, the nucleic acid genome of the recombinant oncolytic vaccinia virus is set forth in SEQ ID NO: 90.

In some of any embodiments, the heterologous nucleic acid encoding IRF3 is inserted into or in place of the B2R (viral cGAMP-specific nuclease) gene locus in the genome of the virus; and the at least one heterologous nucleic acid encoding one or more cytokine and/or chemokine comprises a heterologous nucleic acid encoding IL-2, wherein the IL-2 is an IL-2 superkine that is MDNA11T.

In some of any embodiments, the recombinant oncolytic vaccinia virus further comprises a heterologous nucleic acid encoding an immune stimulating protein, and/or a heterologous nucleic acid encoding one or more anti-angiogenic protein. In some embodiments, the immune stimulating protein is recombinant LIGHT. In some embodiments, the recombinant LIGHT comprises the amino acid sequence set forth in SEQ ID NO: 30, or an amino acid sequence that has at least 85%, 90%, 91%, 92%, 93%, 94%, 95%, 96%, 97%, 98%, or 99% sequence identity to the amino acid sequence set forth in SEQ ID NO: 30. In some embodiments, the recombinant LIGHT has the sequence set forth in SEQ ID NO: 30.

In some of any embodiments, the one or more anti-angiogenic protein comprises a VEGF inhibitor, an angiopoietin inhibitor, versikine, or a fusion protein of any two or more of the foregoing. In some of any embodiments, the one or more anti-angiogenic protein comprises an anti-VEGF antibody and/or an anti-Ang2 antibody. In some of any embodiments, the one or more anti-angiogenic protein is a bispecific anti-VEGF/anti-Ang2 antibody. In some embodiments, the bispecific anti-VEGF/anti-Ang2 antibody comprises the amino acid sequence set forth in SEQ ID NO: 23, or an amino acid sequence that has at least 85%, 90%, 91%, 92%, 93%, 94%, 95%, 96%, 97%, 98%, or 99% sequence identity to the sequence set forth in SEQ ID NO: 23. In some embodiments, the bispecific anti-VEGF/anti-Ang2 antibody has the sequence set forth in SEQ ID NO: 23.

In some of any embodiments, the nucleic acid genome of the recombinant oncolytic vaccinia virus comprises the nucleic acid sequence of SEQ ID NO: 88, or a nucleic acid sequence that has at least 95%, 96%, 97%, 98%, or 99% sequence identity to the nucleic acid sequence set forth in SEQ ID NO: 88. In some embodiments, the nucleic acid genome of the recombinant oncolytic vaccinia virus is set forth in SEQ ID NO: 88.

In some of any embodiments, one or more of the heterologous nucleic acid encoding any of the above heterologous gene products (e.g., IRF3, cytokine, chemokine or other heterologous gene product) is operably linked to a promoter. In some embodiments each of the one or more heterologous nucleic acid encoding a heterologous gene product is operably linked to a promoter. In some embodiments, the promoter is selected from the group consisting of 7.5E, 7.5E/L, SSE, 11KL, SSL, SSEL, mH5, and LEO. In some of any embodiments, each heterologous nucleic acid encoding a heterologous gene product is independently operably linked to a promoter, optionally wherein each heterologous nucleic acid encoding a heterologous gene product is independently operably linked to a promoter selected from the group consisting of 7.5E, 7.5E/L, SSE, 11KL, SSL, SSEL, mH5, and LEO. In some of any embodiments, the promoter is a poxviral promoter or is a variant or derivative thereof. In some of any embodiments, the promoter is a vaccinia virus promoter. In some of any embodiments, the promoter is selected from the group consisting of 7.5E, 7.5E/L, SSE, 11KL, SSL, SSEL, mH5, and LEO. In some of any embodiments, the promoter has the sequence of amino acids set forth in any one of SEQ ID NOS: 29, 53, 55, 68, 69, 70, 71, or 72. In some of any embodiments, the promoter is synthetic strong early promoter (SSE). In some of any embodiments, the promoter comprises the sequence set forth in SEQ ID NO: 29. In some of any embodiments, the promoter is a strong early/late promoter (SEL). In some of any embodiments, the promoter comprises the sequence set forth in SEQ ID NO: 55. In some of any embodiments, the promoter is mH5. In some of any embodiments, the mH5 promoter comprises the sequence set forth in SEQ ID NO: 53.

Also provided herein is a recombinant oncolytic virus, comprising: an inactivating mutation of at least one viral gene; and at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing.

In some of any embodiments, the oncolytic virus is a vaccinia virus, a herpes simplex virus, vesicular stomatitis virus (VSV), a Maraba virus (MARAV), a measles virus (MV), adenovirus, myxoma virus, orf virus, parvovirus, raccoonpox virus, coxsackievirus, reovirus, Newcastle disease virus, Seneca valley virus, Semliki Forest virus, mumps virus, influenza virus, echovirus, and a poliovirus (PV).

In some of any embodiments, the oncolytic virus is a vaccinia virus. In some of any embodiments, the nucleic acid genome of the recombinant oncolytic vaccinia virus is modified from a parental vaccinia virus that has a nucleic acid genome that has at least 99% sequence identity to the nucleic acid sequence set forth in SEQ ID NO: 1. In some of any embodiments, the nucleic acid genome of the recombinant oncolytic vaccinia virus is modified from a parental vaccinia virus that has a nucleic acid genome that has the nucleic acid genome set forth in SEQ ID NO: 1.

Also provided herein is a recombinant oncolytic virus, comprising at least one heterologous nucleic acid encoding one or more heterologous gene product, wherein the one or more heterologous gene product is or comprises a complement inhibitor, a T cell or NK cell evader, an immune modulating protein, an anti-angiogenic protein, an interferon regulatory factor, an apoptosis inducible protein, or a combination of any of the foregoing.

Also provided herein is a recombinant oncolytic virus, comprising: a nucleic acid genome that is modified from a parental vaccinia virus genome that has at least 99% sequence identity with the nucleic acid sequence set forth in SEQ ID NO: 1; and comprises at least one heterologous nucleic acid encoding one or more heterologous gene product inserted in the genome. Also provided herein is a recombinant oncolytic virus, comprising: a nucleic acid genome that is modified from a parental vaccinia virus genome that has the nucleic acid sequence set forth in SEQ ID NO: 1; and comprises at least one heterologous nucleic acid encoding one or more heterologous gene product inserted in the genome.

In some of any embodiments, the nucleic acid genome of the parental vaccinia virus is characterized by one or more of: (i) a variant 017 open reading frame (ORF) encoding an amino acid sequence that has at least 95% sequence identity to SEQ ID NO: 57 and comprises a polar uncharged amino acid at position 66, optionally a threonine (T) at position 66; (ii) a variant 038 (K5L) ORF comprising a nucleotide insertion to effect a frameshift mutation, wherein the 038 (K5L) gene product is altered; (iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419; (iv) a variant 104 (H4L) ORF encoding an amino acid sequence having at least 95% sequence identity to SEQ ID NO: 61 and comprises a negatively charged amino acid at position 591, optionally aspartic acid (D) at position 591; and (v) a variant 182 (A56R) ORF comprising deletion of two nucleotides to effect a frameshift mutation, wherein the 182 (A56R) ORF gene product is altered.

In some of any embodiments, the parental vaccinia virus genome is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine @ at the position corresponding to position 92969 of SEQ ID NO: 1; (vi) the nucleic acid sequence CACTTATATAT at the positions corresponding to positions 106870 to 106880 of SEQ ID NO: 1; (vii) the nucleic acid sequence GTTTTCATTA at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC at the positions corresponding to positions 165844 to 185853 of SEQ ID NO: 1; and (x) a C at the position corresponding to position 187805 of SEQ ID NO: 1.

In some of any embodiments, the nucleic acid genome of the recombinant oncolytic vaccinia virus has at least 85%, 86%, 87%, 88%, 89%, 90%, 91%, 92%, 93%, 94%, 95%, 96%, 97%, 98%, or 99% sequence identity to the nucleic acid sequence set forth in SEQ ID NO: 1. In some of any embodiments, the nucleic acid genome of the recombinant oncolytic vaccinia virus has at least 95%, 96%, 97%, 98%, or 99% sequence identity to the nucleic acid sequence set forth in SEQ ID NO: 1. In some of any of embodiments, the nucleic acid genome of the recombinant oncolytic vaccinia virus has at least 95% sequence identity to the nucleic acid sequence set forth in SEQ ID NO: 1. In some of any embodiments, the nucleic acid genome of the recombinant oncolytic vaccinia virus has at least 96% sequence identity to the nucleic acid sequence set forth in SEQ ID NO: 1. In some of any embodiments, the nucleic acid genome of the recombinant oncolytic vaccinia virus has at least 97% sequence identity to the nucleic acid sequence set forth in SEQ ID NO: 1. In some of any embodiments, the nucleic acid genome of the recombinant oncolytic vaccinia virus has at least 98% sequence identity to the nucleic acid sequence set forth in SEQ ID NO: 1. In some of any embodiments, the nucleic acid genome of the recombinant oncolytic vaccinia virus has at least 99% sequence identity to the nucleic acid sequence set forth in SEQ ID NO: 1.

In some of any embodiments, the recombinant oncolytic virus is a recombinant oncolytic vaccinia virus, and wherein the nucleic acid genome of the recombinant oncolytic vaccinia virus is characterized by one or more of: (i) a variant 017 open reading frame (ORF) encoding an amino acid sequence that has at least 95% sequence identity to SEQ ID NO: 57 and comprises a polar uncharged amino acid at position 66, optionally a threonine (T) at position 66; (ii) a variant 038 (K5L) ORF comprising a nucleotide insertion to effect a frameshift mutation, wherein the 038 (K5L) gene product is altered; (iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419; (iv) a variant 104 (H4L) ORF encoding an amino acid sequence having at least 95% sequence identity to SEQ ID NO: 61 and comprises a negatively charged amino acid at position 591, optionally aspartic acid (D) at position 591; and (v) a variant 182 (A56R) ORF comprising deletion of two nucleotides to effect a frameshift mutation, wherein the 182 (A56R) ORF gene product is altered.

In some of any embodiments, the recombinant oncolytic virus is a recombinant oncolytic vaccinia virus, and wherein the nucleic acid genome of the recombinant oncolytic vaccinia virus is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine (C) at the position corresponding to position 92969 of SEQ ID NO: 1; (vi) the nucleic acid sequence CACTTATATAT at the positions corresponding to positions 106870 to 106880 of SEQ ID NO: 1; (vii) the nucleic acid sequence GTTTTCATTA at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC at the positions corresponding to positions 165844 to 185853 of SEQ ID NO: 1; and (x) a C at the position corresponding to position 187805 of SEQ ID NO: 1.

In some of any embodiments, at least one of the at least one heterologous nucleic acid encoding the one or more heterologous gene product is inserted into a non-essential gene or region in the genome of the virus. In some of any such embodiments, the insertion is in place of a deletion of all or a portion of the gene or region.

In some of any embodiments, at least one of the at least one heterologous nucleic acid encoding the one or more heterologous gene product is inserted into the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus. In some of any embodiments, each of the at least one heterologous nucleic acid encoding the one or more heterologous gene product that is inserted into a non-essential gene or region in the genome of the virus is each independently inserted into the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus. In some of any embodiments, the at least one viral gene comprises one or more viral genes selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, and I4L, and any combination thereof. In some of any such embodiments, the insertion is in place of a deletion of all or a portion of the respective gene loci.

In some of any embodiments, the at least one viral gene loci into which at least one of the at least one heterologous nucleic acid is inserted is or comprises: (i) B2R; (ii) A35R; (iii) A35R and J2R; (iv) J2R; (v) B2R and J2R; (vi) A35R, B2R, and J2R; (vii) B2R, J2R, and A56R; or (viii) A35R, B2R, J2R, and A56R.

In some of any embodiments, the inactivating mutation of one or more of the at least one viral gene is, independently, by: insertion of at least one of the at least one heterologous nucleic acid encoding one or more heterologous gene product; a deletion of all or a portion of the at least one viral gene; and/or one or more nucleic acid substitutions in the at least one viral gene. In some of any embodiments, the inactivating mutation of one or more of the at least one viral gene is by insertion of at least one of the at least one heterologous nucleic acid encoding one or more heterologous gene product and a deletion of all or a portion of the at least one viral gene, in which the insertion is in place of the deletion of all or a portion of the viral gene.

In some of any embodiments, the inactivating mutation is a deletion of all or a portion of the at least one viral gene. In some of any embodiments: the deletion of the at least one viral gene is deletion of the entire gene ORF of a viral gene. In some of any embodiments: the deletion is sufficient to render the encoded viral gene product non-functional. In some of any embodiments, the inactivating mutation of one or more of the at least one viral gene is characterized by insertion of at least one of the at least one heterologous nucleic acid encoding one or more heterologous gene product into the viral gene loci. In some of any embodiments, the at least one viral gene comprises B2R. In some of any embodiments, the at least one viral gene comprises J2R. In some of any embodiments, the at least one viral gene comprises A35R. In some of any embodiments, the at least one viral gene comprises A56R. In some of any embodiments, the at least one viral gene comprises B2R, J2R, and A35R. In some of any embodiments, the at least one viral gene comprises B2R, J2R, A35R, and A56R. In some of any embodiments, the at least one viral gene comprises B2R, J2R, and A56R.

In some of any embodiments: at least one heterologous nucleic acid encoding the one or more heterologous gene product is inserted into or in place of F14.5L; and/or at least one heterologous nucleic acid encoding the one or more heterologous gene product is inserted into or in place of A35R; and/or at least one heterologous nucleic acid encoding the one or more heterologous gene product is inserted into or in place of J2R.

In some of any embodiments, the at least one heterologous nucleic acid encoding one or more heterologous gene product comprises one or more heterologous nucleic acid each encoding one or more immune modulating proteins. In some of any embodiments, the inactivating mutation of one or more of the at least one viral gene is by insertion of one or more heterologous nucleic acid each encoding one or more immune modulating proteins. In some of any embodiments, the one or more immune modulating proteins comprises one or more immune stimulating proteins. In some of any embodiments, the one or more immune modulating proteins comprises one or more cytokines and/or chemokines. In some of any embodiments, the one or more immune modulating proteins comprises one or more interferon regulatory factors. In some embodiments, the interferon regulatory factor is IRF3. In some of any embodiments, the one or more interferon regulatory factors is or comprises interferon regulatory factor 3 (IRF3). In some of any embodiments, the one or more immune modulating proteins comprises interferon regulatory factor 3 (IRF3) and one or more cytokines and/or chemokines.

In some of any embodiments, the one or more immune modulating proteins comprises one or more immune modulating proteins selected from the group consisting of LIGHT, IRF3, IL-2, IL-12, and CXCL9. In some of any embodiments: the CXCL9 is human CXCL9. In some of any embodiments: the CXCL9 is human CXCL9 and comprises the amino acid sequence set forth in SEQ ID NO: 99, or an amino acid sequence that has at least 95%, 96%, 97%, 98%, or 99% sequence identity to the amino acid sequence of SEQ ID NO: 99. In some of any embodiments, the CXCL9 is mouse CXCL9. In some of any embodiments, the CXCL9 is mouse CXCL9 and comprises the amino acid sequence set forth in SEQ ID NO: 106, or an amino acid sequence that has at least 95%, 96%, 97%, 98%, or 99% sequence identity to the amino acid sequence of SEQ ID NO: 106.

In some of any embodiments: the IL-12 is a human single-chain IL-12. In some of any embodiments: the IL-12 is a human single-chain IL-12 and comprises the amino acid sequence set forth in SEQ ID NO: 103, or an amino acid sequence that has at least 95%, 96%, 97%, 98%, or 99% sequence identity to the amino acid sequence of SEQ ID NO: 103. In some of any embodiments, the IL-12 is a mouse single-chain IL-12. In some of any embodiments, the IL-12 is a mouse single-chain IL-12 and comprises the amino acid sequence set forth in SEQ ID NO: 102, or an amino acid sequence that has at least 95%, 96%, 97%, 98%, or 99% sequence identity to the amino acid sequence of SEQ ID NO: 102.

In some of any embodiments, the one or more immune modulating proteins comprises IRF3. In some of any embodiments, the IRF3 is a human IRF3 (hIRF3). In some of any embodiments, the hIRF3 comprises the amino acid sequence set forth in SEQ ID NO: 51, or an amino acid sequence that has at least 95%, 96%, 97%, 98%, or 99% sequence identity to the amino acid sequence set forth in SEQ ID NO: 51. In some of any embodiments, the IRF3 is a mouse IRF3 (mIRF3). In some of any embodiments, the mIRF3 comprises the amino acid sequence set forth in SEQ ID NO: 52, or an amino acid sequence that has at least 95%, 96%, 97%, 98%, or 99% sequence identity to the amino acid sequence set forth in SEQ ID NO: 52.

In some of any embodiments, the nucleic acid genome of the recombinant oncolytic virus comprises the nucleic acid sequence set forth in any one of SEQ ID NOs: 49, 50, 80, 82, and 84-93, or a nucleic acid sequence that has at least 95%, 96%, 97%, 98%, or 99% sequence identity to the nucleic acid sequence set forth in any one of SEQ ID NOs: 49, 50, 80, 82, and 84-93.

In some of any embodiments, the one or more immune modulating proteins comprises IRF3 and one or more immune modulating proteins selected from the group consisting of LIGHT, IL-2, IL-12, and CXCL9. In some of any embodiments, the one or more immune modulating proteins comprises IL-2. In some of any embodiments, the one or more immune modulating proteins comprises IL-12. In some of any embodiments, the one or more immune modulating proteins comprises LIGHT. In some of any embodiments, the one or more immune modulating proteins comprises CXCL9. In some of any embodiments, the one or more immune modulating proteins is or comprises: (i) IRF3; (ii) LIGHT; (iii) IRF3 and LIGHT; (iv) IRF3 and IL-2; (v) IRF3, CXCL9, and IL-12; (vi) IRF3, LIGHT, and IL-2; (vii) IRF3 and CXCL9; or (viii) IRF3, CXCL9, and IL-2.

In some of any embodiments, the IL-2 is a human IL-2. In some of any embodiments, the IL-2 is an IL-2 superkine. In some of any embodiments, the IL-2 superkine is H9, H9T, MDNA11, or MDNA11T. In some of any embodiments: the H9 IL-2 superkine comprises the amino acid sequence of SEQ ID NO: 100, or comprises an amino acid sequence that has at least 95%, 96%, 97%, 98%, or 99% sequence identity to the amino acid sequence of SEQ ID NO: 100. In some of any embodiments, the H9T IL-2 superkine comprises the amino acid sequence of SEQ ID NO: 104, or comprises an amino acid sequence that has at least 95%, 96%, 97%, 98%, or 99% sequence identity to the amino acid sequence of SEQ ID NO: 104. In some of any embodiments, the MDNA11 IL-2 superkine comprises the amino acid sequence of SEQ ID NO: 101, or comprises an amino acid sequence that has at least 95%, 96%, 97%, 98%, or 99% sequence identity to the amino acid sequence of SEQ ID NO: 101. In some of any embodiments, the MDNA11T IL-2 superkine comprises the amino acid sequence of SEQ ID NO: 98, or comprises an amino acid sequence that has at least 95%, 96%, 97%, 98%, or 99% sequence identity to the amino acid sequence of SEQ ID NO: 98. In some of any embodiments, the IL-2 superkine is MDNA11 or MDNA11T. In some of any embodiments, the IL-2 superkine is MDNA11T, and the MDNA11T comprises the amino acid sequence set forth in SEQ ID NO: 98, or an amino acid sequence that has at least 95%, 96%, 97%, 98%, or 99% sequence identity to the amino acid sequence set forth in SEQ ID NO: 98.

In some of any embodiments, the LIGHT is recombinant LIGHT. In some embodiments, the recombinant LIGHT is a human LIGHT protein or is a mutant thereof. In some of any embodiments, the recombinant LIGHT comprises an amino acid sequence that has at least 95%, 96%, 97%, 98%, or 99% sequence identity to the amino acid sequence set forth in SEQ ID NO: 30. In some of any embodiments, the recombinant LIGHT is human LIGHT mutant (hmLIGHT) that is a human LIGHT mutant that binds human and mouse LTBR and HVEM. In some of any embodiments, the recombinant LIGHT comprises one or more mutations selected from the group consisting of a threonine at position 138, a glycine at position 160, a glycine at position 221, and a lysine at position 222. In some of any embodiments, the recombinant LIGHT comprises the amino acid sequence set forth in SEQ ID NO: 25, or an amino acid sequence that has at least 95%, 96%, 97%, 98%, or 99% sequence identity to the amino acid sequence set forth in SEQ ID NO: 25. In some of any embodiments, the recombinant LIGHT comprises the sequence set forth in SEQ ID NO: 25.

In some of any embodiments, the nucleic acid genome of the recombinant oncolytic virus comprises the nucleic acid sequence set forth in any one of SEQ ID NOs: 11, 82, 87, and 88, or a nucleic acid sequence that has at least 95%, 96%, 97%, 98%, or 99% sequence identity to the nucleic acid sequence set forth in any one of SEQ ID NOs: 11, 82, 87, and 88.

In some of any embodiments, the IL-12 is a human IL-12. In some embodiments, the human IL-12 is a human single chain IL-12 (hscIL-12). In some embodiments, the hscIL-12 comprises the amino acid sequence set forth in SEQ ID NO: 103, or an amino acid sequence that has at least 95%, 96%, 97%, 98%, or 99% sequence identity to the amino acid sequence set forth in SEQ ID NO: 103.

In some of any embodiments, the CXCL9 is a human CXCL9. In some embodiments, the human CXCL9 comprises the amino acid sequence set forth in SEQ ID NO: 99, or an amino acid sequence that has at least 95%, 96%, 97%, 98%, or 99% sequence identity to the amino acid sequence set forth in SEQ ID NO: 99.

In some of any embodiments, the at least one heterologous nucleic acid encoding one or more heterologous gene product comprises one or more heterologous nucleic acid each encoding an apoptosis-inducible protein. In some of any embodiments, the inactivating mutation of one or more of the at least one viral gene is by insertion of one or more heterologous nucleic acid each encoding an apoptosis-inducible protein. In some of any embodiments, the apoptosis-inducible protein comprises a proapoptotic molecule fused with an FKBP variant that is able to bind a chemical inducer of dimerization (CID). In some of any embodiments, the FKBP variant is FKBP-F36V. In some embodiments, the FKBP-F36V comprises the amino acid sequence set forth in SEQ ID NO: 56 or an amino acid sequence that has at least 95%, 96%, 97%, 98%, or 99% sequence identity to the amino acid sequence set forth in SEQ ID NO: 56.

In some of any embodiments, the chemical inducer of dimerization is AP1903 (Rimiducid). In some embodiments, the proapoptotic molecule is or comprises Fas, the death effector domain (DED) of the Fas-associated death domain-containing protein (FADD), or a caspase, optionally wherein the caspase is caspase 9. In some of any embodiments, the apoptosis-inducible protein is an inducible DED (iDED). In some of any embodiments, the iDED comprises the amino acid sequence set forth in SEQ ID NO: 27 or an amino acid sequence that has at least 95%, 96%, 97%, 98%, or 99% sequence identity to the amino acid sequence set forth in SEQ ID NO: 27. In some of any embodiments, the nucleic acid genome of the recombinant oncolytic virus comprises the nucleic acid sequence set forth in SEQ ID NO: 8 or 86, or a nucleic acid sequence that has at least 95%, 96%, 97%, 98%, or 99% sequence identity to the nucleic acid sequence set forth in SEQ ID NO: 8 or 86.

In some of any embodiments, the apoptosis-inducible protein is an inducible Fas (iFas). In some of any embodiments, the iFas comprises the amino acid sequence set forth in SEQ ID NO: 28 or an amino acid sequence that has at least 95%, 96%, 97%, 98%, or 99% sequence identity to the amino acid sequence set forth in SEQ ID NO: 28. In some of any embodiments, the nucleic acid genome of the recombinant oncolytic virus comprises the nucleic acid sequence set forth in SEQ ID NO: 9, or a nucleic acid sequence that has at least 95%, 96%, 97%, 98%, or 99% sequence identity to the nucleic acid sequence set forth in SEQ ID NO: 9.

In some of any embodiments, the apoptosis-inducible protein is an inducible caspase 9 (iCas9). In some of any embodiments, the iCas9 comprises the amino acid sequence set forth in SEQ ID NO: 26 or an amino acid sequence that has at least 95%, 96%, 97%, 98%, or 99% sequence identity to the amino acid sequence set forth in SEQ ID NO: 26. In some of any embodiments, the nucleic acid genome of the recombinant oncolytic virus comprises the nucleic acid sequence set forth in SEQ ID NO: 7, or a nucleic acid sequence that has at least 95%, 96%, 97%, 98%, or 99% sequence identity to the nucleic acid sequence set forth in SEQ ID NO: 7.

In some of any embodiments, the at least one heterologous nucleic acid encoding one or more heterologous gene product comprises one or more heterologous nucleic acid each encoding one or more T cell or NK cell evader proteins. In some of any embodiments, the inactivating mutation of one or more of the at least one viral gene is by insertion of one or more heterologous nucleic acid each encoding one or more T cell or NK cell evader proteins.

In some of any embodiments, the one or more T cell or NK cell evader proteins comprises a set of proteins encoded by Cowpox virus ORFs 012, 203 and 018 (CPXV012-203-018). In some of any embodiments, the one or more T cell or NK cell evader proteins comprises a set of proteins that is or comprises the CPXV012, CPXV203, and CPXV018 proteins. In some of any embodiments, the set of proteins encoded by CPXV012-203-018 comprises: (i) the amino acid sequence set forth in SEQ ID NO: 20 (CPXV012) or an amino acid sequence that has at least 85%, 90%, 91%, 92%, 93%, 94%, 95%, 96%, 97%, 98%, or 99% sequence identity to the amino acid sequence set forth in SEQ ID NO: 20, (ii) the amino acid sequence set forth in SEQ ID NO: 21 (CPXV0203) or an amino acid sequence that has at least 85%, 90%, 91%, 92%, 93%, 94%, 95%, 96%, 97%, 98%, or 99% sequence identity to the amino acid sequence set forth in SEQ ID NO: 21, and (iii) the amino acid sequence set forth in SEQ ID NO: 22 (CPXV018) or an amino acid sequence that has at least 85%, 90%, 91%, 92%, 93%, 94%, 95%, 96%, 97%, 98%, or 99% sequence identity to the amino acid sequence set forth in SEQ ID NO: 22. In some of any embodiments, the set of proteins encoded by CPXV012-203-018 comprises the amino acid sequences set forth in SEQ ID NO: 20, SEQ ID NO: 21, and SEQ ID NO: 22. In some of any embodiments, the nucleic acid genome of the recombinant oncolytic virus comprises the nucleic acid sequence set forth in any one of SEQ ID NOs: 10, 89, and 90, or a nucleic acid sequence that has at least 95%, 96%, 97%, 98%, or 99% sequence identity to the nucleic acid sequence set forth in any one of SEQ ID NOs: 10, 89, and 90.

In some of any embodiments, the at least one heterologous nucleic acid encoding one or more heterologous gene product comprises one or more heterologous nucleic acid each encoding one or more complement inhibitor. In some of any embodiments, the inactivating mutation of one or more of the at least one viral gene is by insertion of one or more heterologous nucleic acid each encoding one or more complement inhibitor.

In some of any embodiments, the one or more complement inhibitor is Borrelia burgdorferi complement regulatory-acquiring surface protein-2 (CRASP-2) and/or minimized complement regulator factor H (miniFH). In some of any embodiments, the one or more complement inhibitor is or comprises CRASP-2. In some of any embodiments, the CRASP-2 comprises the amino acid sequence set forth in SEQ ID NO: 18 or has an amino acid sequence that has at least 95%, 96%, 97%, 98%, or 99% sequence identity to the amino acid sequence set forth in SEQ ID NO: 18. In some of any embodiments, the one or more complement inhibitor is or comprises miniFH. In some of any embodiments, the miniFH comprises the amino acid sequence set forth in SEQ ID NO: 19 or has an amino acid sequence that has at least 95%, 96%, 97%, 98%, or 99% sequence identity to the amino acid sequence set forth in SEQ ID NO: 19.

In some of any embodiments, the one or more heterologous nucleic acid encoding the one or more complement inhibitor is introduced into a viral membrane gene, optionally F14.5L, to produce a fusion gene encoding a fusion protein. In some embodiments, the fusion protein comprises the complement inhibitor fused to a viral membrane protein encoded by the viral membrane gene. In some of any embodiments, the viral membrane gene is F14.5L, optionally wherein the fusion is at the C-terminus of the F14.5L protein. In some of any embodiments, the fusion protein is incorporated into the outer membrane of the intracellular mature virus (IMV). In some of any embodiments, the nucleic acid genome of the recombinant oncolytic virus comprises the nucleic acid sequence set forth in SEQ ID NO: 5, or a nucleic acid sequence that has at least 95%, 96%, 97%, 98%, or 99% sequence identity to the nucleic acid sequence set forth in SEQ ID NO: 5. In some of any embodiments, the nucleic acid genome of the recombinant oncolytic virus comprises the nucleic acid genome of the recombinant oncolytic virus comprises the nucleic acid sequence set forth in SEQ ID NO: 6, or a nucleic acid sequence that has at least 95%, 96%, 97%, 98%, or 99% sequence identity to the nucleic acid sequence set forth in SEQ ID NO: 6. In some of any embodiments, the nucleic acid genome of the recombinant oncolytic virus comprises the nucleic acid genome of the recombinant oncolytic virus comprises the nucleic acid sequence set forth in SEQ ID NO: 89, or a nucleic acid sequence that has at least 95%, 96%, 97%, 98%, or 99% sequence identity to the nucleic acid sequence set forth in SEQ ID NO: 89. In some of any embodiments, the nucleic acid genome of the recombinant oncolytic virus comprises the nucleic acid genome of the recombinant oncolytic virus comprises the nucleic acid sequence set forth in SEQ ID NO: 90, or a nucleic acid sequence that has at least 95%, 96%, 97%, 98%, or 99% sequence identity to the nucleic acid sequence set forth in SEQ ID NO: 90.

In some of any embodiments, the at least one heterologous nucleic acid encoding one or more heterologous gene product comprises one or more heterologous nucleic acid each encoding one or more anti-angiogenic protein. In some of any embodiments, the inactivating mutation of one or more of the at least one viral gene is by insertion of one or more heterologous nucleic acid each encoding one or more anti-angiogenic protein. In some of any embodiments, the one or more anti-angiogenic protein is a VEGF inhibitor, an angiopoietin inhibitor, versikine, or a fusion protein of any two or more of the foregoing. In some of any embodiments, the one or more anti-angiogenic protein comprises a VEGF inhibitor and/or an angiopoietin inhibitor, optionally an inhibitor of Ang2. In some of any embodiments, the one or more anti-angiogenic protein comprises an anti-VEGF antibody and/or an anti-Ang2 antibody. In some of any embodiments, the VEGF inhibitor is an anti-VEGF antibody, optionally an anti-VEGF-single chain antibody (scAb). In some of any embodiments, the angiopoietin inhibitor is an anti-Angriopoietin-2 (Ang2) antibody, optionally an anti-Ang2 single chain antibody (scAb). In some of any embodiments, the one or more anti-angiogenic protein is a bispecific anti-VEGF/anti-Ang2 antibody. In some of any embodiments, the bispecific anti-VEGF/anti-Ang2 antibody comprises the amino acid sequence set forth in SEQ ID NO: 23, or an amino acid sequence that has at least 85%, 90%, 91%, 92%, 93%, 94%, 95%, 96%, 97%, 98%, or 99% sequence identity to the sequence set forth in SEQ ID NO: 23. In some of any embodiments, the one or more anti-angiogenic protein comprises versikine. In some of any embodiments, the versikine comprises the amino acid sequence set forth in SEQ ID NO: 24, or comprises an amino acid sequence that has at least 85%, 90%, 91%, 92%, 93%, 94%, 95%, 96%, 97%, 98%, or 99% sequence identity to the sequence set forth in SEQ ID NO: 24. In some of any embodiments, the nucleic acid genome of the recombinant oncolytic virus comprises the nucleic acid sequence set forth in any one of SEQ ID NOs: 13, 47, 82, 87, and 88, or a nucleic acid sequence that has at least 95%, 96%, 97%, 98%, or 99% sequence identity to the nucleic acid sequence set forth in any one of SEQ ID NOs: 13, 47, 82, 87, and 88.

In some of any embodiments, the at least one heterologous nucleic acid encoding one or more heterologous gene product comprises one or more heterologous nucleic acid each encoding one or more therapeutic agent or diagnostic agent. In some of any embodiments, the inactivating mutation of one or more of the at least one viral gene is by insertion of one or more heterologous nucleic acid each encoding one or more one or more therapeutic agent or diagnostic agent.

In some of any embodiments, the one or more therapeutic agent or diagnostic agent are selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging. In some of any embodiments, the one or more therapeutic agent or diagnostic agent comprises a therapeutic agent selected from among a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor.

In some of any embodiments: the at least one viral gene is or comprises A35R, optionally wherein the nucleic acid genome of the recombinant oncolytic virus comprises the nucleic acid sequence of SEQ ID NO: 3, or a nucleic acid sequence that has at least 95%, 96%, 97%, 98%, or 99% sequence identity to the nucleic acid sequence set forth in SEQ ID NO: 3.

In some of any embodiments: the at least one viral gene is or comprises A35R and J2R, optionally wherein the nucleic acid genome of the recombinant oncolytic virus comprises the nucleic acid sequence of SEQ ID NO: 12, or a nucleic acid sequence that has at least 95%, 96%, 97%, 98%, or 99% sequence identity to the nucleic acid sequence set forth in SEQ ID NO: 12.

In some of any embodiments: the at least one viral gene is or comprises J2R, and the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018), and wherein the at least one heterologous nucleic acid encoding one or more heterologous gene product comprises one or more heterologous nucleic acid each encoding one or more complement inhibitor that is introduced into a viral membrane gene to produce a fusion gene encoding a fusion protein. In some embodiments, the viral membrane gene is F14.5L. In some embodiments, the fusion is at the C-terminus of the F14.5L protein. In some embodiments, the nucleic acid genome of the recombinant oncolytic virus comprises the nucleic acid sequence of SEQ ID NO: 10, or a nucleic acid sequence that has at least 95%, 96%, 97%, 98%, or 99% sequence identity to the nucleic acid sequence set forth in SEQ ID NO: 10.

In some of any embodiments: the at least one viral gene is or comprises J2R. In some embodiments, the nucleic acid genome of the recombinant oncolytic virus comprises the nucleic acid sequence of SEQ ID NO: 4, or a nucleic acid sequence that has at least 95%, 96%, 97%, 98%, or 99% sequence identity to the nucleic acid sequence set forth in SEQ ID NO: 4.

In some of any embodiments: the at least one viral gene is or comprises J2R and A35R, and the inactivating mutation of A35R is by insertion of one or more heterologous nucleic acid each encoding one or more immune modulating proteins. In some embodiments, the one or more immune modulating proteins is selected from the group consisting of LIGHT, IRF3, IL-2, IL-12, and CXCL9. In some embodiments, the one or more immune modulating proteins is LIGHT. In some embodiments, the nucleic acid genome of the recombinant oncolytic virus comprises the nucleic acid sequence of SEQ ID NO: 11, or a nucleic acid sequence that has at least 95%, 96%, 97%, 98%, or 99% sequence identity to the nucleic acid sequence set forth in SEQ ID NO: 11.

In some of any embodiments: the at least one viral gene is or comprises J2R and A35R, and the inactivating mutation of J2R is by insertion of the one or more heterologous nucleic acid encoding one or more anti-angiogenic protein. In some embodiments, the one or more anti-angiogenic protein comprises an inhibitor or VEGF and/or an inhibitor of Ang2. In some embodiments, the one or more anti-angiogenic protein is a bispecific anti-VEGF/anti-Ang2 antibody. In some embodiments, the nucleic acid genome of the recombinant oncolytic virus comprises the nucleic acid sequence of SEQ ID NO: 13, or a nucleic acid sequence that has at least 95%, 96%, 97%, 98%, or 99% sequence identity to the nucleic acid sequence set forth in SEQ ID NO: 13.

In some of any embodiments: the at least one viral gene is or comprises J2R and A35R, and the inactivating mutation of A35R is by insertion of the one or more heterologous nucleic acid encoding one or more immune modulating proteins and the inactivating mutation of J2R is by insertion of the one or more heterologous nucleic acid encoding one or more anti-angiogenic protein. In some embodiments, the one or more immune modulating proteins is selected from the group consisting of LIGHT, IRF3, IL-2, IL-12, and CXCL9. In some embodiments, the one or more immune modulating proteins is LIGHT. In some embodiments, the one or more anti-angiogenic protein comprises an inhibitor or VEGF and/or an inhibitor of Ang2. In some embodiments, the one or more anti-angiogenic protein is a bispecific anti-VEGF/anti-Ang2 antibody. In some embodiments, the nucleic acid genome of the recombinant oncolytic virus comprises the nucleic acid sequence of SEQ ID NO: 47, or a nucleic acid sequence that has at least 95%, 96%, 97%, 98%, or 99% sequence identity to the nucleic acid sequence set forth in SEQ ID NO: 47.

In some of any embodiments: the at least one viral gene is or comprises J2R, and the inactivating mutation of J2R is by insertion of one or more heterologous nucleic acid each encoding an apoptosis-inducible protein. In some embodiments, the apoptosis-inducible protein is an inducible DED (iDED), an inducible Fas (iFas), or an inducible Cas9 (iCas9). In some embodiments, the nucleic acid genome of the recombinant oncolytic virus comprises the nucleic acid sequence of SEQ ID NO: 7, 8, or 9, or a nucleic acid sequence that has at least 95%, 96%, 97%, 98%, or 99% sequence identity to the nucleic acid sequence set forth in SEQ ID NO: 7, 8, or 9. In some embodiments, the nucleic acid genome of the recombinant oncolytic virus comprises the nucleic acid sequence of SEQ ID NO: 7. In some embodiments, the nucleic acid genome of the recombinant oncolytic virus comprises the nucleic acid sequence of SEQ ID NO: 8. In some embodiments, the nucleic acid genome of the recombinant oncolytic virus comprises the nucleic acid sequence of SEQ ID NO: 9.

In some of any embodiments: the at least one viral gene is or comprises J2R, and the inactivating mutation of J2R is by insertion of one or more heterologous nucleic acid each encoding one or more immune modulating proteins. In some embodiments, the one or more immune modulating proteins is selected from the group consisting of LIGHT, IRF3, IL-2, IL-12, and CXCL9. In some embodiments, the one or more immune modulating proteins is IRF3. In some embodiments, the nucleic acid genome of the recombinant oncolytic virus comprises the nucleic acid sequence of SEQ ID NO: 49, 50, or 93, or a nucleic acid sequence that has at least 95%, 96%, 97%, 98%, or 99% sequence identity to the nucleic acid sequence set forth in SEQ ID NO: 49, 50, or 93.

In some of any embodiments: the at least one viral gene is or comprises J2R and B2R. In some embodiments, the nucleic acid genome of the recombinant oncolytic virus comprises the nucleic acid sequence of SEQ ID NO: 48, or a nucleic acid sequence that has at least 95%, 96%, 97%, 98%, or 99% sequence identity to the nucleic acid sequence set forth in SEQ ID NO: 48.

In some of any embodiments, the at least one viral gene is or comprises J2R and B2R.

In some of any embodiments: the at least one viral gene is or comprises J2R and B2R, and the inactivating mutation of J2R is by insertion of one or more heterologous nucleic acid each encoding one or more immune modulating proteins. In some embodiments, the one or more immune modulating proteins is selected from the group consisting of LIGHT, IRF3, IL-2, IL-12, and CXCL9. In some embodiments, the one or more immune modulating proteins is IRF3. In some embodiments, the nucleic acid genome of the recombinant oncolytic virus comprises the nucleic acid sequence of SEQ ID NO: 80, or a nucleic acid sequence that has at least 95%, 96%, 97%, 98%, or 99% sequence identity to the nucleic acid sequence set forth in SEQ ID NO: 80.

In some of any embodiments: the at least one viral gene is or comprises J2R, B2R, and A35R; wherein: the inactivating mutation of J2R is by insertion of the one or more heterologous nucleic acid encoding one or more anti-angiogenic protein, the inactivating mutation of B2R is by insertion of one or more heterologous nucleic acid each encoding one or more immune modulating proteins, and the inactivating mutation of A35R is by insertion of one or more heterologous nucleic acid encoding one or more immune modulating proteins. In some embodiments, the one or more anti-angiogenic protein comprises an inhibitor or VEGF and/or an inhibitor of Ang2. In some embodiments, the one or more anti-angiogenic protein is a bispecific anti-VEGF/anti-Ang2 antibody. In some embodiments, the inactivating mutation of B2R is by insertion of one or more heterologous nucleic acid each encoding one or more immune modulating proteins selected from the group consisting of LIGHT, IRF3, IL-2, IL-12, and CXCL9. In some embodiments, the one or more immune modulating proteins is IRF3. In some embodiments, the inactivating mutation of A35R is by insertion of one or more heterologous nucleic acid encoding one or more immune modulating proteins selected from the group consisting of LIGHT, IRF3, IL-2, IL-12, and CXCL9. In some embodiments, the one or more immune modulating proteins is LIGHT. In some embodiments, the nucleic acid genome of the recombinant oncolytic virus comprises the nucleic acid sequence of SEQ ID NO: 82, or a nucleic acid sequence that has at least 95%, 96%, 97%, 98%, or 99% sequence identity to the nucleic acid sequence set forth in SEQ ID NO: 82.

In some of any embodiments: the at least one viral gene is or comprises J2R, B2R, and A56R; wherein: the inactivating mutation of J2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins is IL-2. In some embodiments, the IL-2 is an IL-2 superkine. In some embodiments, the IL-2 superkine is MDNA11. In some embodiments, the nucleic acid genome of the recombinant oncolytic virus comprises the nucleic acid sequence of SEQ ID NO: 84, or a nucleic acid sequence that has at least 95%, 96%, 97%, 98%, or 99% sequence identity to the nucleic acid sequence set forth in SEQ ID NO: 84.

In some of any embodiments: the at least one viral gene is or comprises J2R, B2R, and A56R; wherein: the inactivating mutation of J2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9. In some embodiments, the two or more immune modulating proteins comprises IL-12 and CXCL9. In some embodiments, the nucleic acid genome of the recombinant oncolytic virus comprises the nucleic acid sequence of SEQ ID NO: 85, or a nucleic acid sequence that has at least 95%, 96%, 97%, 98%, or 99% sequence identity to the nucleic acid sequence set forth in SEQ ID NO: 85.

In some of any embodiments: the at least one viral gene is or comprises J2R, B2R, and A56R; wherein: the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, and the inactivating mutation of J2R is by insertion of one or more heterologous nucleic acid each encoding an apoptosis-inducible protein. In some embodiments, the two or more immune modulating proteins comprises IL-12 and CXCL9. In some embodiments, the apoptosis-inducible protein is an inducible DED (iDED). In some embodiments, the nucleic acid genome of the recombinant oncolytic virus comprises the nucleic acid sequence of SEQ ID NO: 86, or a nucleic acid sequence that has at least 95%, 96%, 97%, 98%, or 99% sequence identity to the nucleic acid sequence set forth in SEQ ID NO: 86.

In some of any embodiments: the at least one viral gene is or comprises J2R, B2R, A35R, and A56R; wherein: the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; the inactivating mutation of B2R is by insertion of one or more heterologous nucleic acid encoding one or more immune modulating proteins, optionally wherein the one or more immune modulating proteins is IRF3; the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; the inactivating mutation of A56R is by insertion of one or more heterologous nucleic acid encoding one or more immune modulating proteins, wherein the one or more immune modulating proteins is IL-2 superkine MDNA11. In some embodiments, the nucleic acid genome of the recombinant oncolytic virus comprises the nucleic acid sequence of SEQ ID NO: 87, or a nucleic acid sequence that has at least 95%, 96%, 97%, 98%, or 99% sequence identity to the nucleic acid sequence set forth in SEQ ID NO: 87.

In some of any embodiments: the at least one viral gene is or comprises J2R, B2R, A35R, and A56R; wherein: the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; the inactivating mutation of B2R is by insertion of one or more heterologous nucleic acid encoding one or more immune modulating proteins, optionally wherein the one or more immune modulating proteins is IRF3; the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; the inactivating mutation of A56R is by insertion of one or more heterologous nucleic acid encoding one or more immune modulating proteins, wherein the one or more immune modulating proteins is IL-2 superkine MDNA11T. In some embodiments, the MDNA11T comprises the amino acid sequence set forth in SEQ ID NO: 98. In some embodiments, the nucleic acid genome of the recombinant oncolytic virus comprises the nucleic acid sequence of SEQ ID NO: 88, or a nucleic acid sequence that has at least 95%, 96%, 97%, 98%, or 99% sequence identity to the nucleic acid sequence set forth in SEQ ID NO: 88.

In some of any embodiments: the at least one viral gene is or comprises J2R, B2R, and A56R; wherein: the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018); the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is an IL-2 superkine, optionally MDNA11 or MDNA11T; the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein. In some embodiments, the fusion is at the C-terminus of the F14.5L protein. In some embodiments, the nucleic acid genome of the recombinant oncolytic virus comprises the nucleic acid sequence of SEQ ID NO: 89, or a nucleic acid sequence that has at least 95%, 96%, 97%, 98%, or 99% sequence identity to the nucleic acid sequence set forth in SEQ ID NO: 89.

In some of any embodiments: the at least one viral gene is or comprises J2R, B2R, and A56R; wherein: the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018); the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein. In some embodiments, the fusion is at the C-terminus of the F14.5L protein. In some embodiments, the nucleic acid genome of the recombinant oncolytic virus comprises the nucleic acid sequence of SEQ ID NO: 90, or a nucleic acid sequence that has at least 95%, 96%, 97%, 98%, or 99% sequence identity to the nucleic acid sequence set forth in SEQ ID NO: 90.

In some of any embodiments: the at least one viral gene is or comprises B2R and J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3. In some embodiments, the nucleic acid genome of the recombinant oncolytic virus comprises the nucleic acid sequence of SEQ ID NO: 91, or a nucleic acid sequence that has at least 95%, 96%, 97%, 98%, or 99% sequence identity to the nucleic acid sequence set forth in SEQ ID NO: 91.

In some of any embodiments: the at least one viral gene is or comprises B2R, J2R, and A56R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9. In some embodiments, the nucleic acid genome of the recombinant oncolytic virus comprises the nucleic acid sequence of SEQ ID NO: 92, or a nucleic acid sequence that has at least 95%, 96%, 97%, 98%, or 99% sequence identity to the nucleic acid sequence set forth in SEQ ID NO: 92.

In some of any embodiments: the at least one viral gene is or comprises J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3. In some embodiments, the nucleic acid genome of the recombinant oncolytic virus comprises the nucleic acid sequence of SEQ ID NO: 93, or a nucleic acid sequence that has at least 95%, 96%, 97%, 98%, or 99% sequence identity to the nucleic acid sequence set forth in SEQ ID NO: 93.

In some of any embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93. In some of any embodiments, the nucleic acid genome of the recombinant oncolytic virus comprises a nucleic acid sequence set forth in any one of SEQ ID NOs: 85, 86, 88, and 90, or a nucleic acid sequence that has at least 95%, 96%, 97%, 98%, or 99% sequence identity to a nucleic acid sequence set forth in any one of SEQ ID NOs 85, 86, 88, and 90. In some of any embodiments, the nucleic acid genome of the recombinant oncolytic virus comprises a nucleic acid sequence set forth in SEQ ID NO: 85, or a nucleic acid sequence that has at least 95%, 96%, 97%, 98%, or 99% sequence identity to a nucleic acid sequence set forth in SEQ ID NO 85.

In some of any embodiments, one or more of the heterologous nucleic acid encoding a heterologous gene product is operably linked to a promoter.

In some of any embodiments, each of the one or more heterologous nucleic acid encoding a heterologous gene product that is operably linked to a promoter is selected from the group consisting of 7.5E, 7.5E/L, SSE, 11KL, SSL, SSEL, mH5, and LEO. In some of any embodiments, each heterologous nucleic acid encoding a heterologous gene product is independently operably linked to a promoter, optionally wherein each heterologous nucleic acid encoding a heterologous gene product is independently operably linked to a promoter selected from the group consisting of 7.5E, 7.5E/L, SSE, 11KL, SSL, SSEL, mH5, and LEO. In some of any embodiments, the promoter is a poxviral promoter or is a variant or derivative thereof. In some of any embodiments, the promoter is a vaccinia virus promoter. In some of any embodiments, the promoter is selected from the group consisting of 7.5E, 7.5E/L, SSE, 11KL, SSL, SSEL, mH5, and LEO. In some of any embodiments, the promoter has the sequence of amino acids set forth in any one of SEQ ID NOS: 29, 53, 55, 68, 69, 70, 71, or 72. In some of any embodiments, the promoter is synthetic strong early promoter (SSE). In some of any embodiments, the SSE promoter comprises the sequence set forth in SEQ ID NO: 29. In some of any embodiments, the promoter is a strong early/late promoter (SEL). In some of any embodiments, the SEL promoter comprises the sequence set forth in SEQ ID NO: 55. In some of any embodiments, the promoter is mH5. In some of any embodiments, the mH5 promoter comprises the sequence set forth in SEQ ID NO: 53.

Also provided herein is an isolated clonal vaccinia virus (VACV) strain comprising a nucleic acid genome that has at least 95% sequence identity with the nucleic acid sequence set forth in SEQ ID NO: 1 and wherein the nucleic acid genome is characterized by one or more of: (i) a variant 017 open reading frame (ORF) encoding an amino acid sequence that has at least 95% sequence identity to SEQ ID NO: 57 and comprises a polar uncharged amino acid at position 66, optionally a threonine (T) at position 66; (ii) a variant 038 (K5L) ORF comprising a nucleotide insertion to effect a frameshift mutation, wherein the 038 (K5L) gene product is altered; (iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419; (iv) a variant 104 (H4L) ORF encoding an amino acid sequence having at least 95% sequence identity to SEQ ID NO: 61 and comprises a negatively charged amino acid at position 591, optionally aspartic acid (D) at position 591; and (v) a variant 182 (A56R) ORF comprising deletion of two nucleotides to effect a frameshift mutation, wherein the 182 (A56R) ORF gene product is altered.

In some of any embodiments of any recombinant oncolytic virus or any isolated clonal VACV strain, the nucleic acid genome is characterized by (i) and the variant 017 ORF encodes an amino acid sequence that has at least 96%, at least 97%, at least 98%, at least 99% or 100% sequence identity to SEQ ID NO: 57. In some of any embodiments of any recombinant oncolytic virus or any isolated clonal VACV strain, the nucleic acid genome is characterized by (i) and the variant 017 ORF encodes the amino acid sequence set forth in SEQ ID NO: 57. In some of any embodiments of any recombinant oncolytic virus or any isolated clonal VACV strain, the nucleic acid genome is characterized by (ii) and the nucleotide insertion is guanine (G) corresponding to insertion after nucleotide position 32135 of SEQ ID NO: 1, optionally wherein the variant 038 (K5L) ORF is set forth in SEQ ID NO: 58. In some of any embodiments of any recombinant oncolytic virus or any isolated clonal VACV strain, the nucleic acid genome is characterized by (ii) and the 038 (K5L) gene product is set forth in SEQ ID NO: 59. In some of any embodiments of any recombinant oncolytic virus or any isolated clonal VACV strain, the nucleic acid genome is characterized by (iii) and the variant 059 (E2L) ORF encodes an amino acid sequence that has at least 96%, at least 97%, at least 98%, at least 99% or 100% sequence identity to SEQ ID NO: 60. In some of any embodiments of any recombinant oncolytic virus or any isolated clonal VACV strain, the nucleic acid genome is characterized by (iii) and the variant 059 (E2L) ORF encodes the amino acid sequence set forth in SEQ ID NO: 60. In some of any embodiments of any recombinant oncolytic virus or any isolated clonal VACV strain, the nucleic acid genome is characterized by (iv) and the 104 (H4L) ORF encodes an amino acid sequence that has at least 96%, at least 97%, at least 98%, at least 99% or 100% sequence identity to SEQ ID NO: 61. In some of any embodiments of any recombinant oncolytic virus or any isolated clonal VACV strain, the nucleic acid genome is characterized by (iv) and wherein the variant 104 (H4L) ORF encodes the amino acid sequence set forth in SEQ ID NO: 61. In some of any embodiments of any recombinant oncolytic virus or any isolated clonal VACV strain, the nucleic acid genome is characterized by (v) and the deletion of two nucleotides is deletion of two contiguous nucleotides corresponding to nucleotides after nucleotide position 165972 of SEQ ID NO: 2, optionally wherein the variant 182 (A56R) is set forth in SEQ ID NO: 62. In some of any embodiments of any recombinant oncolytic virus or any isolated clonal VACV strain, the nucleic acid genome is characterized by (v) and the VACV protein is set forth in SEQ ID NO: 63.

In some of any embodiments of any recombinant oncolytic virus or any isolated clonal VACV strain, the nucleic acid genome is characterized by any two of (i)-(v). In some of any embodiments of any recombinant oncolytic virus or any isolated clonal VACV strain, the nucleic acid genome is characterized by any three of (i)-(v). In some of any embodiments of any recombinant oncolytic virus or any isolated clonal VACV strain, the nucleic acid genome is characterized by any four of (i)-(v). In some of any embodiments of any recombinant oncolytic virus or any isolated clonal VACV strain, the nucleic acid genome is characterized by each of (i)-(v).

Also provided herein is an isolated clonal vaccinia virus (VACV) strain comprising a nucleic acid genome that has at least 95% sequence identity with the nucleic acid sequence set forth in SEQ ID NO: 1, and wherein the nucleic acid genome is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine (C) at the position corresponding to position 92969 of SEQ ID NO: 1; (vi) the nucleic acid sequence CACTTATATAT at the positions corresponding to positions 106870 to 106880 of SEQ ID NO: 1; (vii) the nucleic acid sequence GTTTTCATTA at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC at the positions corresponding to positions 165844 to 185853 of SEQ ID NO: 1; and (x) a C at the position corresponding to position 187805 of SEQ ID NO: 1.

In some of any embodiments of any recombinant oncolytic virus or any isolated clonal VACV strain, the nucleic acid genome is characterized by any two of (i)-(x). In some of any embodiments of any recombinant oncolytic virus or any isolated clonal VACV strain, the nucleic acid genome is characterized by any three of (i)-(x). In some of any embodiments of any recombinant oncolytic virus or any isolated clonal VACV strain, the nucleic acid genome is characterized by any four of (i)-(x). In some of any embodiments of any recombinant oncolytic virus or any isolated clonal VACV strain, the nucleic acid genome is characterized by any five of (i)-(x). In some of any embodiments of any recombinant oncolytic virus or any isolated clonal VACV strain, the nucleic acid genome is characterized by any six of (i)-(x). In some of any embodiments of any recombinant oncolytic virus or any isolated clonal VACV strain, the nucleic acid genome is characterized by any seven of (i)-(x). In some of any embodiments of any recombinant oncolytic virus or any isolated clonal VACV strain, the nucleic acid genome is characterized by any eight of (i)-(x). In some of any embodiments of any recombinant oncolytic virus or any isolated clonal VACV strain, the nucleic acid genome is characterized by any nine of (i)-(x). In some of any embodiments of any recombinant oncolytic virus or any isolated clonal VACV strain, the nucleic acid genome is characterized by each of (i)-(x).

In some of any embodiments of any recombinant oncolytic virus or any isolated clonal VACV strain, the nucleic acid genome has at least 96% sequence identity with the sequence of nucleotides set forth in SEQ ID NO: 1. In some of any embodiments of any recombinant oncolytic virus or any isolated clonal VACV strain, the nucleic acid genome has at least 97% sequence identity with the sequence of nucleotides set forth in SEQ ID NO: 1. In some of any embodiments of any recombinant oncolytic virus or any isolated clonal VACV strain, the nucleic acid genome has at least 98% sequence identity with the sequence of nucleotides set forth in SEQ ID NO: 1. In some of any embodiments of any recombinant oncolytic virus or any isolated clonal VACV strain, the nucleic acid genome has at a least 99% sequence identity with the sequence of nucleotides set forth in SEQ ID NO: 1.

Also provided herein is an isolated clonal vaccinia virus (VACV) strain comprising a nucleic acid genome that has at least 99% sequence identity with the sequence of nucleotides set forth in SEQ ID NO: 1.

In some of any embodiments of any recombinant oncolytic virus or any isolated clonal VACV strain, the nucleic acid genome has at least 99.5% sequence identity with the sequence of nucleotides set forth in SEQ ID NO: 1. In some of any embodiments of any recombinant oncolytic virus or any isolated clonal VACV strain, the nucleic acid genome has at least 99.9% sequence identity with the sequence of nucleotides set forth in SEQ ID NO: 1. In some of any embodiments of any recombinant oncolytic virus or any isolated clonal VACV strain, the nucleic acid genome has at least 99.95% sequence identity with the sequence of nucleotides set forth in SEQ ID NO: 1. In some of any embodiments of any recombinant oncolytic virus or any isolated clonal VACV strain, the nucleic acid genome does not comprise the sequence of nucleotides set forth in SEQ ID NO: 2. In some of any embodiments of any recombinant oncolytic virus or any isolated clonal VACV strain, the nucleic acid genome is not modified to contain non-viral heterologous nucleic acid containing an open reading frame encoding a non-viral heterologous protein. In some of any embodiments of any recombinant oncolytic virus or any isolated clonal VACV strain, the nucleic acid genome is set forth in SEQ ID NO: 1.

In some of any embodiments of any recombinant oncolytic virus or any isolated clonal VACV strain, the clonal VACV strain exhibits enhanced production of extracellular enveloped virions (EEV) after cell infection, optionally as determined by percentage of EEV, wherein the percentage of EEV is determined by the formula: viral titer in supernatant/(viral titer in supernatant+viral titer in cell lysate)*100. In some of any embodiments of any recombinant oncolytic virus or any isolated clonal VACV strain, greater than 5% of infectious particles after cell infection are EEV. In some of any embodiments of any recombinant oncolytic virus or any isolated clonal VACV strain, greater than 10% of infectious particles after cell infection are EEV. In some of any embodiments of any recombinant oncolytic virus or any isolated clonal VACV strain, greater than 15% of infectious particles after cell infection are EEV. In some of any embodiments, the recombinant oncolytic virus or the clonal VACV strain exhibits enhanced production of extracellular enveloped virions (EEV) after cell infection, as determined by having a percentage of at least 5%, 10%, or 15% of infectious particles being EEV.

In some of any embodiments of any recombinant oncolytic virus or any isolated clonal VACV strain, the virus exhibits oncolytic activity to kill tumor cells.

Also provided herein is a VACV preparation comprising the isolated clonal VACV strain of any of isolated clonal VACV strain provided herein.

Also provided herein is a VACV preparation comprising any recombinant oncolytic vaccinia virus provided herein.

Also provided herein is a recombinant oncolytic virus preparation comprising any of the recombinant oncolytic viruses provided herein, wherein at least 70%, 80%, 90%, 95%, or 98% of the virus particles in the preparation have the genomic sequence of the clonal recombinant oncolytic virus.

In some of any embodiments, the VACV preparation is substantially homogenous wherein a plurality of the virus particles in the preparation has the genomic sequence of the clonal VACV strain.

In some of any embodiments, at least 70% of the virus particles in the preparation have the genomic sequence of the clonal VACV strain. In some of any embodiments, at least 80% of the virus particles in the preparation have the genomic sequence of the clonal VACV strain. In some of any embodiments, at least 90% of the virus particles in the preparation have the genomic sequence of the clonal VACV strain. In some of any embodiments, at least 95% of the virus particles in the preparation have the genomic sequence of the clonal VACV strain. In some of any embodiments, at least 98% of the virus particles in the preparation have the genomic sequence of the clonal VACV strain.

Also provided herein is a pharmaceutical composition comprising any if the isolated VACV clonal strains provided herein.

Also provided herein is a pharmaceutical composition comprising any of the VACV provided herein.

Also provided herein is a pharmaceutical composition comprising any of the recombinant oncolytic vaccinia viruses provided herein.

Also provided herein is a recombinant vaccinia virus (VACV) strain comprising a nucleic acid genome of any of the VACV clonal strains provided herein that comprises an inactivating mutation in at least one viral gene.

In some of any embodiments, the viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L and I4L. In some of any embodiments, the inactivating mutation is a deletion of all or a portion of the at least one viral gene. In some of any embodiments, the deletion of the at least one viral gene is deletion of the entire gene ORF of a viral gene. In some of any embodiments, the deletion of the at least one viral gene is a deletion of a portion of the ORF of a viral gene, and wherein said deletion is sufficient to render the encoded gene product non-functional.

In some of any embodiments, the at least one viral gene is or comprises A35R.

In some of any embodiments, the nucleic acid genome of the recombinant VACV strain comprises the nucleic acid sequence set forth in SEQ ID NO: 3, or a nucleic acid sequence that has at least 95%, at least 96%, at least 97%, at least 98% or at least 99% sequence identity to the sequence set forth in SEQ ID NO: 3.

In some of any embodiments, the at least one viral gene is or comprises J2R.

In some of any embodiments, the nucleic acid genome of the recombinant VACV strain comprises the nucleic acid sequence set forth in SEQ ID NO: 4, or a nucleic acid sequence that has at least 95%, at least 96%, at least 97%, at least 98% or at least 99% sequence identity to the sequence set forth in SEQ ID NO: 4.

In some of any embodiments, the at least one viral gene is or comprises B2R.

In some of any embodiments, the at least one viral gene is or comprises A35R and J2R.

In some of any embodiments, the nucleic acid genome of the recombinant VACV strain comprises the nucleic acid sequence set forth in SEQ ID NO: 12, or a nucleic acid sequence that has at least 95%, at least 96%, at least 97%, at least 98% or at least 99% sequence identity to the sequence set forth in SEQ ID NO: 12.

In some of any embodiments, the at least one viral gene is or comprises B2R and J2R.

In some of any embodiments, the nucleic acid genome of the recombinant VACV strain comprises the nucleic acid sequence set forth in SEQ ID NO: 48, or a nucleic acid sequence that has at least 95%, at least 96%, at least 97%, at least 98% or at least 99% sequence identity to the sequence set forth in SEQ ID NO: 48.

In some of any embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93. In some of any embodiments, the nucleic acid genome of the recombinant oncolytic virus comprises a nucleic acid sequence set forth in any one of SEQ ID NOs: 85, 86, 88, and 90, or a nucleic acid sequence that has at least 95%, 96%, 97%, 98%, or 99% sequence identity to a nucleic acid sequence set forth in any one of SEQ ID NOs 85, 86, 88, and 90. In some of any embodiments, the nucleic acid genome of the recombinant oncolytic virus comprises a nucleic acid sequence set forth in SEQ ID NO: 85, or a nucleic acid sequence that has at least 95%, 96%, 97%, 98%, or 99% sequence identity to a nucleic acid sequence set forth in SEQ ID NO 85.

Also provided herein is a nucleic acid comprising a genome of any of the recombinant oncolytic viruses provided herein or any of the isolated VACV clonal strains provided herein.

Also provided herein is a recombinant oncolytic virus comprising the nucleic acid of any of the recombinant oncolytic viruses provided herein.

In some of any embodiments, the recombinant oncolytic virus is a recombinant oncolytic vaccinia virus. In some of any embodiments, the recombinant oncolytic virus is a recombinant oncolytic vaccinia virus.

Also provided herein is a pharmaceutical composition comprising any of the recombinant VACV strains provided herein.

Also provided herein is a pharmaceutical composition comprising any of the recombinant oncolytic viruses provided herein, optionally wherein the recombinant oncolytic virus is a recombinant oncolytic vaccinia virus.

In some of any embodiments, the pharmaceutical composition further comprises a pharmaceutically acceptable carrier.

In some of any embodiments, the pharmaceutical composition is formulated for intravenous administration, intratumoral administration, intraperitoneal administration or intrapleural administration. In some of any embodiments, the pharmaceutical composition is formulated for intravenous administration. In some of any embodiments, the pharmaceutical composition is a liquid composition. In some of any embodiments, the pharmaceutical composition is lyophilized.

Also provided herein is a method of treating a proliferative disorder in a subject comprising administering to the subject any of the recombinant oncolytic viruses provided herein, any of the isolated oncolytic viruses provided herein, or any of the pharmaceutical compositions provided herein.

In some embodiments, the proliferative disorder is a tumor or a metastasis. In some of any embodiments, the proliferative disorder is cancer. In some of any embodiments, the cancer is a pancreatic cancer, ovarian cancer, lung cancer, colon cancer, prostate cancer, cervical cancer, breast cancer, rectal cancer, renal (kidney) cancer, gastric cancer, esophageal cancer, hepatic (liver) cancer, endometrial cancer, bladder cancer, brain cancer, head and neck cancer, oral cancer (e.g., oral cavity cancer), cervical cancer, uterine cancer, thyroid cancer, testicular cancer, prostate cancer, skin cancers, such as melanoma, e.g., malignant melanoma, cholangiocarcinoma (bile duct cancer), thymic epithelial cancer, e.g., thymoma, leukemia, lymphoma, or multiple myeloma. In some of any embodiments, the cancer is Microsatellite Stable (MSS) colorectal cancer.

In some of any embodiments, the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

In some of any embodiments, the recombinant oncolytic virus or the isolated oncolytic virus is administered in an amount from 1×10⁵ pfu to 1×10¹⁴ pfu.

In some of any embodiments, the method further comprises administering a second therapeutic agent for the treatment of the proliferative disorder.

In some of any embodiments, the method further comprises another treatment selected from among surgery, radiation therapy, immunosuppressive therapy and administration of an anticancer agent. In some embodiments, the another treatment is administration of an anticancer agent selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent and a combination of any of the preceding thereof.

In some of any embodiments, the recombinant oncolytic virus or the isolated oncolytic virus is administered intravenously.

In some of any embodiments, the method further comprises administering AP1903 (Rimiducid) to the subject.

In some of any embodiments, the recombinant oncolytic virus administered to the subject comprises a heterologous nucleic acid encoding an apoptosis inducible protein.

In some of any embodiments, the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

In some of any embodiments, the subject exhibits severe immune deficiency and is sensitive to virus infection.

In some of any embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

Also provided herein is a method of inhibiting virus replication, the method comprising contacting cells infected with a recombinant oncolytic virus with AP1903 (Rimiducid), wherein the recombinant oncolytic virus comprises a heterologous nucleic acid encoding an apoptosis inducible protein.

Also provided herein is a method of inhibiting virus replication, the method comprising contacting cells with AP1903 (Rimiducid), wherein the cells are infected with any of the recombinant oncolytic viruses provided herein, any of the isolated oncolytic viruses provided herein, or any of the recombinant oncolytic viruses, e.g., clonal VACV strains, provided herein.

In some of any embodiments, the contacting occurs in vivo in a subject. In some of any embodiments, the AP1903 (Rimiducid) has been administered to a subject previously administered with a recombinant oncolytic virus comprising the heterologous nucleic acid encoding an apoptosis inducible protein. In some of any embodiments, the AP1903 (Rimiducid) has been administered to a subject previously administered with any of the recombinant oncolytic viruses provided herein, or any of the isolated oncolytic viruses provided herein.

Also provided herein is a method of inhibiting virus replication in a subject, the method comprising administering to a subject AP1903 (Rimiducid), wherein the subject has been previously administered a recombinant oncolytic virus comprising a heterologous nucleic acid encoding an apoptosis inducible protein.

Also provided herein is a method of inhibiting virus replication in a subject, the method comprising administering to a subject AP1903 (Rimiducid), wherein the subject has been previously administered any of the recombinant oncolytic viruses provided herein, or any of the isolated oncolytic viruses provided herein.

In some of any embodiments, the method inhibits virus replication preferentially in non-cancer cells. In some of any embodiments, the apoptosis-inducible protein is an inducible DED (iDED). In some of any embodiments, the iDED comprises the amino acid sequence set forth in SEQ ID NO: 27, or an amino acid sequence that has at least 85%, 90% or 95% sequence identity to SEQ ID NO: 27.

In some of any embodiments, the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

In some of any embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

### BRIEF DESCRIPTION OF THE DRAWINGS

FIG. 1 depicts cell survival percentage of BT-549, A549, LOX-IMVI, HCC-2998 and COLO-205 cells after infection with VIP01-06 VACV clonal isolates. For each of VIP01, VIP02, VIP03, VIP04, VIP05, and VIP06, the bars correspond to, from left to right, BT-549, A549, LOX-IMVI, HCC-2998, and COLO-205 cells.

FIG. 2 shows VCP02 and VIP02 percentage of extracellular enveloped virus (EVV) production in 4T1 and B16-F10 infected cells.

FIG. 3 depicts tumor volume in a 4T1 mouse breast cancer model after infection with a single intravenous delivery of VCP02 (squares), VIP01 (triangles), VIP02 (diamonds), and vehicle (circle).

FIGS. 4A and 4B depict cell survival percentage in 2-D (FIG. 4A) and 3-D (FIG. 4B) cell cultures of different cancer cell types infected with VIP02 at MOI=0.01 (lighter bars) and MOI=0.1 (darker bars).

FIG. 5 depicts a series of schematics representing the genomic structures of stealth recombinant clones VIR27, VIR37 and VIR46 derived from the parental VIP02.

FIG. 6 depicts percentage of host complement inhibition in human and mice serum after incubation with stealth oncolytic viral clones VIR27, VIR37 and VIP02.

FIG. 7 depicts tumor volume in a 4T1 mouse breast cancer model after infection with a single intravenous delivery of VIP02, VIR27, and vehicle.

FIG. 8 depicts tumor volume in a 4T1 mouse breast cancer model after infection with a single intravenous delivery of VIR46, VIR52, and vehicle.

FIG. 9 depicts a series of schematics representing the genomic structures of immune-stimulating oncolytic viruses VIR49 and VIR52.

FIG. 10 depicts tumor volume in a 4T1 mouse breast cancer model after infection with a single intravenous delivery of VIR49, and VIR52.

FIG. 11 depicts a series of schematics representing the genomic structure of anti-angiogenesis oncolytic virus VIR71.

FIGS. 12A and 12B depict tumor volume in a 4T1 mouse breast cancer model after infection with a single intravenous delivery of VIR71, VIR52, and vehicle (FIG. 12A), and tumor volume in.

FIG. 13 depicts a series of schematics representing the genomic structures of apoptosis inducing oncolytic viruses VIR40, VIR41 and VIR42, and control virus VIR13.

FIG. 14 depicts a series of graphs quantifying viral replication of the apoptosis-inducing viral clones VIR13, VIR40, VIR41 and VIR42 in primary healthy HBE, HME and MME cells initially infected at MOIs of 0.01 and/or 10 in the presence of Rimiducid or DMSO, as a control.

FIGS. 15A-G depict a series of graphs quantifying viral replication of the apoptosis-inducing viral clones VIR13, VIR40, VIR41 and VIR42 in BT-549 breast cancer cells (FIG. 15A), Hs578T breast cancer cells (FIG. 15B), MCF-7 and 4T1breast cancer cells (FIG. 15C), A549 and M14 lung and melanoma cancer cells (FIG. 15D), HCT-15 MSI colon cancer cells (FIG. 15E), HCT-116 MSI colon cancer cells (FIG. 15F), and KM12 MSI colon cancer cells (FIG. 15G) initially infected at MOIs of 0.01 and/or 10 in the presence of Rimiducid or DMSO as a control.

FIGS. 16A-C depict a series of graphs quantifying viral replication in COLO205 cancer cells (FIG. 16A), HCC-2998 cancer cells (FIG. 16B), and HT-29 cancer cells (FIG. 16C) initially infected at MOIs of 0.01 and/or 10 in the presence of Rimiducid or DMSO as a control.

FIGS. 17A-C depict a series of graphs quantifying the cytotoxicity of the apoptosis-inducing viral clones VIR13, VIR40, VIR41 and VIR42 in human primary bronchial/tracheal epithelial cells (HBE, FIG. 17A), human primary mammary epithelial cells (HME, FIG. 17B), murine primary mammary epithelial cells (MME) and human primary colonic epithelial cells (HCE, FIG. 17C) initially infected at MOIs of 0.01 and/or 0.1 in the presence of Rimiducid or DMSO as a control.

FIGS. 18A-K depict a series of graphs quantifying the cytotoxicity of the apoptosis-inducing viral clones VIR13, VIR40, VIR41 and VIR42 BT-549 breast cancer cells (FIG. 18A), Hs578T breast cancer cells (FIG. 18B), 4T1 breast cancer cells (FIG. 18C), DU-145 prostate cancer cells (FIG. 18D), PC-3 prostate cancer cells (FIG. 18E), A549 lung and melanoma cancer cells (FIG. 18F), M14 lung and melanoma cancer cells (FIG. 18G), COLO 320 DM and HCT-15 MSI colon cancer cells (FIG. 18H), HCT-116 and KM12 MSI colon cancer cells (FIG. 18I), KM12 MSI colon cancer cells (FIG. 18J) and SW48 MSI colon cancer cells (FIG. 18K) initially infected at MOIs of 0.01 and/or 0.1 in the presence of Rimiducid or DMSO as a control.

FIGS. 19A-G depict a series of graphs quantifying the cytotoxicity of the apoptosis-inducing viral clones VIR13, VIR40, VIR41 and VIR42 in COLO205 MSS colon cancer cells (FIG. 19A), HCC-2998 colon cancer cells (FIG. 19B), HT-29 cells (FIG. 19C), LS123 cells (FIG. 19D), LS174T cells (FIG. 19E), SW620 cells (FIG. 19F) and WiDR cells (FIG. 19G) initially infected at MOIs of 0.01 and/or 0.1 in the presence of Rimiducid or DMSO as a control.

FIG. 20 shows complete inhibition of tumor growth in the SL-4 mouse model of colon adenocarcinoma after infection with a single intravenous injection of VIR13.

FIGS. 21A-E depict a series of graphs showing tumor size over time (days post treatment) in mice following administration of VIR13, VIR41, or control (FIG. 21A), VIR13, VIR86, or control (FIG. 21B), VIR13, VIR93, or control (FIG. 21C), VIR13, VIR94, or control (FIG. 21D), VIR 13, VIR96, or control (FIG. 21E). FIG. 21F depicts a graph showing body weight (g) over time (days post treatment) for mice that were administered VIR13, VIR41, VIR86, VIR93, VIR94, VIR96, or control.

FIGS. 22A-H depict a series of graphs showing tumor volume over time (days post-treatment) in mice following administration of VIR94, VIR100, or control (FIG. 22A), VIR94, VIR 103, or control (FIG. 22B), VIR94, VIR105, or control (FIG. 22C), VIR94, VIR106, or control (FIG. 22D), VIR94, VIR 109, or control (FIG. 22E), VIR94, VIR113, or control (FIG. 22F), VIR94, VIR114, or control (FIG. 22G), or VIR94, VIR115, or control (FIG. 22H). *=p≤0.05; **=p≤0.01; ***=p≤0.001.

FIG. 23A depicts a graph showing tumor size over time (days post treatment) in mice following administration of VIR103, VIR111, or VIR113. FIG. 23B shows a schematic for how both MDNA11 and MDNA11T are created from wild-type human IL-2 (wt hIL-2). *=p≤0.05; **=p≤0.01.

FIG. 24A depicts a graph showing tumor volume over time (days post treatment) in mice following administration of VIR106 or control. FIG. 24B depicts a graph showing weight (g) over time (days post treatment) in mice following administration of VIR106 or control. ***=p≤0.001. FIG. 24C shows images of the tumor location on mice that were taken on day 8 following administration of VIR106 or control, which shows the presence of detectable tumors in control mice but a lack of detectable tumors in mice that were treated with VIR106. FIGS. 24D-E depict a graph showing tumor volume over time (days post treatment) (FIG. 24D) and weight (g) (FIG. 24E) in mice administered VIR113 or control. FIGS. 24F-G depict a graph showing tumor volume over time (days post treatment) (FIG. 24F) and weight (g) (FIG. 24G) in mice administered VIR115 or control.

FIGS. 25A-F depict a series of graphs showing tumor volume and weight (g) over time (days post treatment) in mice following administration of VIR106 or control (FIGS. 25A and B), VIR113 or control (FIGS. 25C and D), or VIR115 or control (FIGS. 25E and F). *=p≤0.05; **=p≤0.01; ***=p≤0.001.

FIGS. 26A-B depict Western blot analyses showing expression of human phosphor-IRF3, mouse phosphor-IRF3, human IRF3, mouse IRF3, and beta actin, in B16-F10 cells (FIG. 26A) and in Hela S3 cells (FIG. 26B) infected with mock, iVIR13, VIR13, VIR93, VIR94, VIR100, VIR106, VIR113, VIR115, VIR123, or VIR127.

### DETAILED DESCRIPTION

Provided herein are isolated clonal strains that exhibit superior anti-tumorigenic activity and enhanced potential to evade host immune systems compared to other vaccinia viruses. In particular, the provided clonal strains are clonal isolates from a parent IHD-J obtained from ATCC® Catalog No. VR-156™. Also provided are preparations resulting from propagation of such isolated clonal strain. Also provided are recombinant vaccinia viruses derived from the isolated clonal strains that are attenuated by modification to delete or reduce expression of a viral gene or inactivate a viral protein. Also, provided are recombinant viruses that are further improved to evade host anti-viral defenses or to have further enhanced anti-tumor activities. For example, such recombinant viruses comprise a heterologous nucleic acid encoding proteins to escape inhibition by the complement system, to evade Natural Killer (NK) or T cells, to incorporate immune checkpoint molecules to enhance the immunostimulatory activity, or to provide anti-angiogenic activity. Provided recombinant viruses herein also include those armed with a viral inducible system to inhibit viral replication as a safety strategy, such as by mediating apoptosis in certain undesired infected cells, for example healthy cells. Specifically, provided herein is a recombinant oncolytic vaccinia virus, comprising: an inactivating mutation of B2R; a heterologous nucleic acid encoding interferon regulatory factor 3 (IRF3); and at least one heterologous nucleic acid encoding one or more cytokine and/or chemokine. In some embodiments, the at least one heterologous nucleic acid encoding one or more cytokine and/or chemokine comprises a heterologous nucleic acid encoding chemokine ligand 9 (CXCL9) and/or IL-12. Also specifically provided herein is a recombinant oncolytic virus, comprising: an inactivating mutation of at least one viral gene; and at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing.

Oncolytic viruses (OVs) are viruses that replicate selectively or more efficiently in cancer cells than in non-cancer cells. In some cases, the ability to selectively infect, replicate within, and destroy cancer cells, often times while sparing healthy cells, is due to the ability to exploit biochemical differences between healthy and transformed cells during infection. Cancer cells are characterized by disrupted apoptosis pathways, the acquisition of new abilities to evade the immune system, and the ability to proliferate indefinitely, all characteristics that favor viral replication. Because one of the main challenges of cancer therapy is killing malignant cells while minimizing toxic effects, OVs are an appealing option since they rarely cause off-target toxicities.

Oncolytic viruses can be divided into three main groups: (1) viruses with a natural propensity to preferentially replicate in cancer cells while being non-pathogenic in humans such as parvovirus, myxoma virus, Newcastle disease virus, and reovirus; (2) viruses that are genetically engineered to ensure selective replication in cancer cells such as adenovirus, HSV, and vesicular stomatitis virus; and (3) viruses that have been attenuated by propagation in vitro to be used safely in humans. The latter group includes oncolytic viruses derived from vaccinia, also preferred because of their efficient replication, cell lysis, spread, host range and natural tropism for tumor tissues (Shen et al. (2004) Mol. Ther., 11:180). For example, vaccinia virus is more potent in replication and spread than adenovirus vectors.

Vaccinia virus (VV), the prototypical member of the Orthopoxvirus genus, replicates in the cytoplasm of a host cell. VV is a large, complex, enveloped virus that has a linear, double-stranded DNA genome of approximately 190,000 base pairs in length that is made up of a single continuous polynucleotide chain encoding for approximately 250 genes that can potentially express more than 200 proteins. See, e.g., McCraith al., (1982) PNAS, 97(9): 4879-4884. In general, the nonsegmented, noninfectious genome is arranged such that centrally located genes are essential for virus replication (and are thus conserved), while genes near the two termini effect more peripheral functions such as host range and virulence. Vaccinia viruses practice differential gene expression by utilizing open reading frames (ORFs) arranged in sets that, as a general principle, do not overlap. See, e.g., Traktman, P., Chapter 27, Poxvirus DNA Replication, pp. 775-798, in DNA Replication in Eukaryotic Cells, Cold Spring Harbor Laboratory Press (1996). VV's ability for rapid replication results in efficient lysis of infected cells as well as spread to other tumor cells upon successive rounds of replication, leading to profound localized destruction of the tumor. The VV genome encodes ˜250 genes and can accept as much as 20 kb of foreign DNA, making it ideal as a gene delivery vehicle. The recombinant VV vectors are being developed to deliver eukaryotic genes, such as tumor-associated antigens, to the tumors and thus facilitate an induction of the host immune system directed to kill the cancer cells. However, a limiting factor in the use of VVs as cancer treatment delivery vectors is the strong neutralizing antibody response induced by the injection of VV into the bloodstream that limits the ability of the virus to persist and spread and prevents vector re-dosing. In some cases, neutralizing antibodies recognize and bind viral glycoproteins with high affinity and prevent virus interaction with host cell receptors, leading to virus neutralization.

Vaccinia virus replicates in the cytoplasm of infected cells where assembly of progeny starts in specialized areas called viral factories. During replication, three morphologically and antigenically distinct forms of the virus are produced: the intracellular mature virions (IMV), the intracellular enveloped virions (IEV), and extracellular virions. A subset of IMV, the first infectious progeny produced, are trafficked to the trans-Golgi network (TGN), where they are enveloped with two additional membranes to produce IEV. IEV are transported through the cytoplasm to the cell periphery, where the outermost membrane fuses with the plasma membrane to release a double membraned form, termed EV. EV that remain on the cell surface are called cell-associated enveloped virion (CEV), while EV that are no longer attached to the cell surface are called extracellular enveloped virion (EEV). IMV is the most abundant infectious form and is thought to be responsible for spread between hosts; the CEV is believed to play a role in cell-to-cell spread; and the EEV is thought to be important for long range dissemination within the host organism. In particular, EEV has been implicated in long-range virus spread dissemination in vivo. See, e.g., Blasco et al., (1993) Journal of Virology, 67(6):3319-3325. The outer proteins of EEV may induce protective immunity to the virus (Blaso and Moss (1992) J. Virol., 66:4170-4179). There is, however, a high degree of variation in the amount of EEV generated by vaccinia virus strains.

Attenuated vaccinia virus strains have been developed for therapeutic and diagnostic applications. For example, attenuated viruses include recombinant viruses that are modified in one or more viral genes that results in loss or reduced expression of a viral gene or inactivation of a viral protein. Nevertheless, although vaccinia is a well-studied attenuated virus with anti-tumorigenic properties, many strains of vaccinia, including recombinant strains, exhibit variations in virulence and safety that make many unsuitable for clinical application. Therefore, there is a need for improved vaccinia strains with enhanced anti-tumorigenic properties and low cytotoxicity, as these are highly desirable as an effective oncolytic therapy. The oncolytic viruses and methods described herein address this need.

Various approaches have been studied to improve OV antitumoral activity, mainly focused on virus replication and spread, as replication of the virus is generally correlated with cancer cell killing efficacy. However, other aspects of viral infection such as augmentation of host antitumor immune response, induction of apoptosis, and control of tumor angiogenesis are also important aspects of cancer viral therapy (Davola, M. E. and K. L. Mossman (2019) Oncoimmunology 8(6): e1581528).

Provided herein are isolated clonal viruses derived from the vaccinia virus strain known as IHD-J (ATCC® Catalog No. VR-156™). IHD-J is a vaccinia virus strain that is closely related to Western Reserve (WR) strain but that exhibits 10 to 40 times more EEV and spread to distant cells much more efficiently than did WR (Blaso and Moss, 1992). However, strains that exhibit longer range spread may not exhibit sufficient anti-tumorigeneic activity for oncolytic virus therapy.

The provided embodiments are based on the identification of a particular clonal isolate of IHD-J, designated VIP02, that not only exhibits a high percentage of EEV but also exhibits the highest anti-tumorigenic activity among other clonal isolates from the same strain. Moreover, results demonstrated that a single intravenous delivery of the clonal isolate at a low dose significantly inhibited tumor growth in a mouse syngeneic tumor model, and exhibited potent tumor cell killing in vitro against multiple tumor cells in both 2-D and 3-D cultures. Also provided herein are vaccinia virus strains with sequence features of the VIP02 clonal isolate.

Provided embodiments also relate to recombinant viruses in which heterologous nucleic acid can be introduced in the isolated clonal virus with enhanced anti-tumorigenic properties to further enhance the anti-tumorigenic properties of the isolated clonal virus while minimizing their cytotoxic effects on healthy cells.

In some embodiments, the selected clonal strains and their recombinant derived strains are oncolytic virus candidates for tumor diagnosis and therapy. In some embodiments, the isolated clonal strains of vaccinia and their recombinant derived strains can be used as therapeutic viruses for use in the treatment of proliferative disorders, including cancer, hyperplasia, metastasis and tumors, and for use in other therapeutic and/or diagnostic methods as described herein. In some other embodiments, the clonal strains can be used in methods of vaccination. In other embodiments, the isolated clonal strains and their recombinant derived strains can be used as parental vaccinia viruses to generate recombinant oncolytic viruses.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Isolated Clonal Virus Strains and Attenuated Strains Thereof

Provided herein are isolated clonal vaccinia virus (VACV) strains of vaccinia virus strain IHD-J (ATCC® Catalog No. VR-156™) or that exhibit features of a clonal virus strain isolated therefrom. The parental IHD-J strain is heterogenous in sequence. It is found herein that certain vaccinia virus clones with enhanced anti-tumorigenic properties can be isolated from the IHD-J parental vaccinia virus preparation or mixture.

In some embodiments, the clonal strains provided herein are present in a virus preparation propagated from IHD-J. For example, the clonal strains or preparations thereof can be obtained by isolating IHD-J derived clone isolates from cell cultures in which parental IHD-J, or a variant thereof, has been propagated. The clonal isolates provided herein were obtained by passage of the IHD-J virus in confluent CV-1, from African green monkey kidney fibroblast cell cultures, growing in 6-well plates infected with a series of dilutions of the vaccinia virus strains.

In some embodiments, the clonal strains do not contain non-viral heterologous nucleic acid that contains an open reading frame encoding a non-viral heterologous protein. In other embodiments, the clonal strains can be used as a parental sequence for generating a recombinant virus that is modified with a heterologous nucleic acid encoding a non-viral heterologous protein.

In some embodiments, the IHD-J clonal strain provided herein is designated VIP02 and has the nucleotide sequence set forth in SEQ ID NO: 1.

In some embodiments, provided herein is a recombinant oncolytic vaccinia virus, comprising: an inactivating mutation of B2R; a heterologous nucleic acid encoding interferon regulatory factor 3 (IRF3); and at least one heterologous nucleic acid encoding one or more cytokine and/or chemokine. In some embodiments, the at least one heterologous nucleic acid encoding one or more cytokine and/or chemokine comprises a heterologous nucleic acid encoding chemokine ligand 9 (CXCL9) and/or IL-12.

In some embodiments, also provided herein is a recombinant oncolytic virus, comprising: an inactivating mutation of at least one viral gene; and at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing.

In some embodiments, provided vaccinia virus clonal strains have a nucleic acid genome that has at least 95% sequence identity to SEQ ID NO: 1. In some embodiments, provided vaccinia virus clonal strains have a nucleic acid genome that has at least 96% sequence identity to SEQ ID NO: 1. In some embodiments, provided vaccinia virus clonal strains have a nucleic acid genome that has at least 97% sequence identity to SEQ ID NO: 1. In some embodiments, provided vaccinia virus clonal strains have a nucleic acid genome that has at least 98% sequence identity to SEQ ID NO: 1. In some embodiments, provided vaccinia virus clonal strains have a nucleic acid genome that has at least 99% sequence identity to SEQ ID NO: 1. In some embodiments, provided vaccinia virus clonal strains have a nucleic acid genome that has at least 99.1% sequence identity to SEQ ID NO: 1. In some embodiments, provided vaccinia virus clonal strains have a nucleic acid genome that has at least 99.2% sequence identity to SEQ ID NO: 1. In some embodiments, provided vaccinia virus clonal strains have a nucleic acid genome that has at least 99.3% sequence identity to SEQ ID NO: 1. In some embodiments, provided vaccinia virus clonal strains have a nucleic acid genome that has at least 99.4% sequence identity to SEQ ID NO: 1. In some embodiments, provided vaccinia virus clonal strains have a nucleic acid genome that has at least 99.5% sequence identity to SEQ ID NO: 1. In some embodiments, provided vaccinia virus clonal strains have a nucleic acid genome that has at least 99.6% sequence identity to SEQ ID NO: 1. In some embodiments, provided vaccinia virus clonal strains have a nucleic acid genome that has at least 99.7% sequence identity to SEQ ID NO: 1. In some embodiments, provided vaccinia virus clonal strains have a nucleic acid genome that has at least 99.8% sequence identity to SEQ ID NO: 1. In some embodiments, provided vaccinia virus clonal strains have a nucleic acid genome that has at least 99.9% sequence identity to SEQ ID NO: 1.

In some of any such embodiments, the provided vaccinia virus clonal strain does not have a nucleic acid genome with the sequence of amino acids set forth in SEQ ID NO: 2 (IHD-W1). In some embodiments, the provided clonal strains have a sequence of nucleotides that has less than 100% sequence identity to SEQ ID NO: 2 and at least 85%, 86%, 87%, 88%, 89%, 90%, 91%, 92%, 93%, 94%, 05%, 96%, 97%, 98%, 99%, 99.5% or 99.9% sequence identity to SEQ ID NO: 2. In some embodiments, the provided clonal strains has a sequence of nucleotides that differs from SEQ ID NO: 2 by 1, 2, 3, 4, 5, 6, 7, 8, 9, 10, 11, 12, 13, 14, 15, 16, 17, 18, 19, 20 or more nucleotides. Such IHD-J clonal viruses provided herein include viruses that differ in one or more open reading frames (ORF) compared to the IHD-W1 strain that has a sequence of nucleotides set forth in SEQ ID NO: 2. For example, IHD-J clonal viruses provided herein include viruses that differ in one or more ORF compared to the IHD-W1 strain that has a sequence of amino acids set forth in SEQ ID NO: 2. The IHD-J clonal virus strains provided herein can contain a nucleotide deletion or mutation in any one or more nucleotides in any ORF compared to SEQ ID NO: 2, or can contain an addition or insertion of viral DNA compared to SEQ ID NO: 2.

In some embodiments, the provided vaccinia virus clonal strains have a nucleic acid genome that has at least 95% sequence identity to SEQ ID NO: 1 and exhibits sequence features of SEQ ID NO: 1. For instance, as described herein in Table E1 exemplary VIP02 clonal isolates are characterized by deletion or mutation in one or more nucleotides as compared to SEQ ID NO: 2, including one or more mutations in ORFs of SEQ ID NO: 2. With reference to ORFs, ORFs are numbered in consecutive order starting from 001. In other embodiments, vaccinia virus open reading frames may also be designated by a capital letter indicating a HindIII restriction endonuclease fragment, a number indicating the position in the HindIII fragment, and a letter (L or R) indicating the direction of transcription, e.g., K5L. The corresponding protein is designated by a capital letter and number, e.g., K5. In some embodiments, the nucleotide change is in a non-ORF region of the sequence.

In some embodiments, a provided vaccinia virus clonal strain includes or is characterized by a variant 017 open reading frame (ORF) encoding an amino acid sequence that has at least 95% sequence identity to SEQ ID NO: 57 and that contains an amino acid at position 66 other than alanine. In some embodiments, the amino acid at position 66 is a polar uncharged amino acid. In some embodiments, the amino acid at position 66 is a serine(S), Threonine (T), Asparagine (N) or Glutamine (E). In some embodiments, the amino acid at position 66 is a T. In some embodiments, the provided clonal strain includes a variant 017 ORF with a A66T mutation compared to the 017 ORF set forth in SEQ ID NO: 2. In some embodiments, the variant 017 ORF encodes an amino acid sequence that contains any of the above amino acid changes at position 66 and has at least 96% sequence identity to SEQ ID NO: 57. In some embodiments, the variant 017 ORF encodes an amino acid sequence that contains any of the above amino acid changes at position 66 and has at least 97% sequence identity to SEQ ID NO: 57. In some embodiments, the variant 017 ORF encodes an amino acid sequence that contains any of the above amino acid changes at position 66 and has at least 98% sequence identity to SEQ ID NO: 57. In some embodiments, the variant 017 ORF encodes an amino acid sequence that contains any of the above amino acid changes at position 66 and has at least 99% sequence identity to SEQ ID NO: 57. In some embodiments, the variant 017 ORF has the sequence set forth in SEQ ID NO: 57. In some embodiments, such a vaccinia virus clonal strain has a nucleic acid genome that has at least 95%, 96%, 97%, 98%, 99%, 99.5%, 99.99% sequence identity to SEQ ID NO: 1.

In some embodiments, a provided clonal strain includes or is characterized by a variant 038 (K5L) ORF that has a nucleotide insertion to effect a frameshift mutation, wherein the 038 (K5L) gene product is altered. In some embodiments, the nucleotide insertion is insertion of a guanine (G) corresponding to insertion after nucleotide position 32135 of SEQ ID NO: 1. In some embodiments, the full-length sequence of the 038 (K5L) gene product is set forth in SEQ ID NO: 59. In some embodiments, the variant 038 (K5L) ORF is set forth in SEQ ID NO: 58. In some embodiments, such a vaccinia virus clonal strain has a nucleic acid genome that has at least 95%, 96%, 97%, 98%, 99%, 99.5%, 99.99% sequence identity to SEQ ID NO: 1. In some embodiments, the variant 038 (K5L) ORF is characterized by being altered compared to the nucleic acid set forth in SEQ ID NO: 73, or the amino acid sequence set forth in SEQ ID NO: 74.

In some embodiments, a provided clonal strain includes or is characterized by a variant variant 059 (E2L) encoding an amino acid sequence that has at least 95% sequence identity to SEQ ID NO: 60 and that contains an amino acid at position 419 other than leucine. In some embodiments, the amino acid at position 419 is a hydrophobic amino acid other than leucine. In some embodiments, the amino acid at position 419 is an alanine (A), valine (V), isoleucine (I), methionine (M), phenylalanine (F), tyrosine (Y) or tryptophan (W). In some embodiments, the amino acid at position 419 is F. In some embodiments, the provided clonal strain includes a variant 059 (E2L) ORF with a L419F mutation compared to the 059 (E2L) ORF set forth in SEQ ID NO: 2. In some embodiments, the variant 059 (E2L) ORF encodes an amino acid sequence that contains any of the above amino acid changes at position 419 and has at least 96% sequence identity to SEQ ID NO: 60. In some embodiments, the variant 059 (E2L) ORF encodes an amino acid sequence that contains any of the above amino acid changes at position 66 and has at least 97% sequence identity to SEQ ID NO: 60. In some embodiments, the variant 059 (E2L) ORF encodes an amino acid sequence that contains any of the above amino acid changes at position 419 and has at least 98% sequence identity to SEQ ID NO: 60. In some embodiments, the variant 059 (E2L) ORF encodes an amino acid sequence that contains any of the above amino acid changes at position 66 and has at least 99% sequence identity to SEQ ID NO: 60. In some embodiments, the variant 059 (E2L) ORF has the sequence set forth in SEQ ID NO: 60. In some embodiments, such a vaccinia virus clonal strain has a nucleic acid genome that has at least 95%, 96%, 97%, 98%, 99%, 99.5%, 99.99% sequence identity to SEQ ID NO: 1.

In some embodiments, a provided clonal strain includes or is characterized by a variant 104 (H4L) ORF encoding an amino acid sequence that has at least 95% sequence identity to SEQ ID NO: 61 and that contains an amino acid at position 591 other than asparagine (N). In some embodiments, the amino acid at position 591 is a negatively charged amino acid. In some embodiments, the amino acid at position 591 is aspartic acid (D) or glutamic acid (E). In some embodiments, the amino acid at position 591 is D. In some embodiments, the provided clonal strain includes a variant 104 (H4L) ORF with a N591D mutation compared to the 104 (H4L) ORF set forth in SEQ ID NO: 2. In some embodiments, the variant 104 (H4L) ORF encodes an amino acid sequence that contains any of the above amino acid changes at position 591 and has at least 96% sequence identity to SEQ ID NO: 61. In some embodiments, the variant 104 (H4L) ORF encodes an amino acid sequence that contains any of the above amino acid changes at position 591 and has at least 97% sequence identity to SEQ ID NO: 61. In some embodiments, the variant 104 (H4L) ORF encodes an amino acid sequence that contains any of the above amino acid changes at position 591 and has at least 98% sequence identity to SEQ ID NO: 61. In some embodiments, the variant 104 (H4L) ORF encodes an amino acid sequence that contains any of the above amino acid changes at position 591 and has at least 99% sequence identity to SEQ ID NO: 61. In some embodiments, the variant 104 (H4L) ORF has the sequence set forth in SEQ ID NO: 61. In some embodiments, such a vaccinia virus clonal strain has a nucleic acid genome that has at least 95%, 96%, 97%, 98%, 99%, 99.5%, 99.99% sequence identity to SEQ ID NO: 1.

In some embodiments, a provided clonal strain includes or is characterized by a variant 182 (A56R) ORF that has a nucleotide deletion to effect a frameshift mutation, wherein the 182 (A56R) gene product is altered. In some embodiments, the nucleotide deletion is deletion of two contiguous nucleotides corresponding to nucleotides after nucleotide position 165972 of SEQ ID NO: 2. In some embodiments, the 182 (A56R) gene product is set forth in SEQ ID NO: 63. In some embodiments, the variant 182 (A56R) ORF is set forth in SEQ ID NO: 62. In some embodiments, such a vaccinia virus clonal strain has a nucleic acid genome that has at least 95%, 96%, 97%, 98%, 99%, 99.5%, 99.99% sequence identity to SEQ ID NO: 1. In some embodiments, the variant 182 (A56R) ORF is characterized by being altered compared to the nucleic acid set forth in SEQ ID NO: 75, or the amino acid sequence set forth in SEQ ID NO: 76

In some embodiments, a provided clonal strain is characterized by a nucleic acid genome that includes at least one of any of the above mutations in the 017 ORF, 038 (K5L) ORF, 059 (E2L) ORF, 104 (H4L) ORF and 182 (A56R) ORF. In some embodiments, a provided clonal strain is characterized by a nucleic acid genome that includes at least two of any of the above mutations in the 017 ORF, 038 (K5L) ORF, 059 (E2L) ORF, 104 (H4L) ORF and 182 (A56R) ORF. In some embodiments, a provided clonal strain is characterized by a nucleic acid genome that includes at least three of any of the above mutations in the 017 ORF, 038 (K5L) ORF, 059 (E2L) ORF, 104 (H4L) ORF and 182 (A56R) ORF. In some embodiments, a provided clonal strain is characterized by a nucleic acid genome that includes at least four of any of the above mutations in the 017 ORF, 038 (K5L) ORF, 059 (E2L) ORF, 104 (H4L) ORF and 182 (A56R) ORF. In some embodiments, at least one of the mutation is in the 017 ORF. In some embodiments, at least one of the mutation is in the 038 (K5L) ORF. In some embodiments, at least one of the mutation is in the 059 (E2L) ORF. In some embodiments, at least one of the mutation is in the 104 (H4L) ORF. In some embodiments, at least one of the mutation is in the 182 (A56R) ORF. In some embodiments, such a vaccinia virus clonal strain has a nucleic acid genome that has at least 95%, 96%, 97%, 98%, 99%, 99.5%, 99.99% sequence identity to SEQ ID NO: 1.

In some embodiments, a provided clonal strain is characterized by a nucleic acid genome that includes each of the above mutations in the 017 ORF, 038 (K5L) ORF, 059 (E2L) ORF, 104 (H4L) ORF and 182 (A56R) ORF. In some embodiments, a provided clonal strain is characterized by a nucleic acid genome containing a variant 017 ORF encoding the amino acid sequence set forth in SEQ ID NO: 57, a variant 038 (K5L) ORF set forth in SEQ ID NO: 58, a variant of 038 (K5L) encoding the amino acid sequence set forth in SEQ ID NO: 59, a variant 059 (E2L) ORF encoding the amino acid sequence set forth in SEQ ID NO: 60, a variant 104 (H4L) ORF encoding the amino acid sequence set forth in SEQ ID NO: 61, a variant 182 (A56R) ORF set forth in SEQ ID NO: 62, and a variant of 182 (A56R) encoding the amino acid sequence set forth in SEQ ID NO: 63. In some embodiments, such a vaccinia virus clonal strain has a nucleic acid genome that has at least 95%, 96%, 97%, 98%, 99%, 99.5%, 99.99% sequence identity to SEQ ID NO: 1.

In some embodiments, provided vaccinia virus clonal strains have a nucleic acid genome that has at least 95% sequence identity to SEQ ID NO: 1 and is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine (C) at the position corresponding to position 92969 of SEQ ID NO: 1; (vi) the contiguous sequence of nucleotides CACTTATATAT (set forth in SEQ ID NO: 77) at the positions corresponding to positions 106870 to 106880 of SEQ ID NO: 1; (vii) the nucleic acid sequence GTTTTCATTA (set forth in SEQ ID NO: 78) at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC (set forth in SEQ ID NO: 79) at the positions corresponding to positions 165844 to 185853 of SEQ ID NO: 1; and (x) a C at the position corresponding to position 187805 of SEQ ID NO: 1.

In some embodiments, the vaccinia virus clonal strain provided herein include those that have a nucleotide sequence that is characterized by one point mutation, insertion, and/or deletion selected from any one of (i)-(x) above.

In some embodiments, the vaccinia virus clonal strain provided herein include those that have a nucleotide sequence that is characterized by one point mutation, insertion, and/or deletion selected from any two of (i)-(x) above.

In some embodiments, the vaccinia virus clonal strain provided herein include those that have a nucleotide sequence that is characterized by one point mutation, insertion, and/or deletion selected from any three of (i)-(x) above.

In some embodiments, the vaccinia virus clonal strain provided herein include those that have a nucleotide sequence that is characterized by one point mutation, insertion, and/or deletion selected from any four of (i)-(x) above.

In some embodiments, the vaccinia virus clonal strain provided herein include those that have a nucleotide sequence that is characterized by one point mutation, insertion, and/or deletion selected from any five of (i)-(x) above.

In some embodiments, the vaccinia virus clonal strain provided herein include those that have a nucleotide sequence that is characterized by one point mutation, insertion, and/or deletion selected from any six of (i)-(x) above.

In some embodiments, the vaccinia virus clonal strain provided herein include those that have a nucleotide sequence that is characterized by one point mutation, insertion, and/or deletion selected from any seven of (i)-(x) above.

In some embodiments, the vaccinia virus clonal strain provided herein include those that have a nucleotide sequence that is characterized by one point mutation, insertion, and/or deletion selected from any eight of (i)-(x) above.

In some embodiments, the vaccinia virus clonal strain provided herein include those that have a nucleotide sequence that is characterized by one point mutation, insertion, and/or deletion selected from any nine of (i)-(x) above.

In some embodiments, the vaccinia virus clonal strain provided herein include those that have a nucleotide sequence that is characterized by one point mutation, insertion, and/or deletion selected from each of (i)-(x) above.

### A. Exemplary Features

In some embodiments, the IHD-J derived clones exhibited better anti-tumorigenicity and less pathogenicity/toxicity in in vitro and/or in vivo assays compared to the starting virus preparation or mixture or other reference strain or isolate, including recombinant strains. In some embodiments, the IHD-J derived clones exhibited better anti-tumorigenicity properties compared to the starting virus preparation or mixture or other reference strain or isolate, including recombinant strains. In some embodiments, the IHD-J derived clones exhibited less toxicity compared to the starting virus preparation or mixture or other reference strain or isolate, including recombinant strains. In some embodiments, the IHD-J derived clones exhibited similar anti-tumorigenicity properties and/or similar toxicity compared to the starting virus preparation or mixture or other reference strain or isolate, including recombinant strains.

Provided herein are IHD-J clonal isolate strains that exhibited improved properties compared to the starting virus preparation or mixture or other reference strain or isolate, including recombinant strains in the absence of inserted heterologous DNA. In some embodiments, the IHD-J clonal isolate strains exhibited better anti-tumorigenicity and less toxicity compared to the starting virus preparation or mixture or other reference strain or isolate, including recombinant strains in the absence of inserted heterologous DNA. In some embodiments, the IHD-J clonal isolate strains exhibited improved or better anti-tumorigenic activity compared to the starting virus preparation or mixture or other reference strain or isolate, including recombinant strains in the absence of inserted heterologous DNA. In some embodiments, IHD-J clonal isolate strains exhibited less toxicity compared to the starting virus preparation or mixture or other reference strain or isolate, including recombinant strains in the absence of inserted heterologous DNA. In some embodiments, IHD-J clonal isolate strains exhibited similar toxicity and/or anti-tumorigenic activity compared to the starting virus preparation or mixture or other reference strain or isolate, including recombinant strains in the absence of inserted heterologous DNA.

In some embodiments, clonal isolate strains that exhibited improved or better anti-tumorigenic activity compared to the starting virus preparation or mixture or other reference strain or isolate, including recombinant strains, exhibited at or between 120% to 1000%, for example, at least 120%, 130%, 140%, 150%, 160%, 170%, 180%, 190%, 200%, 250%, 300%, 400%, 500%, 1000% or more of the anti-tumorigenic activity of the reference virus preparation (starting virus preparation or mixture or other reference strain or isolate, including recombinant strains) in an assay or method to assess a parameter indicative of anti-tumorigenicity. The anti-tumorigenicity can be determined using any of the in vitro or in vivo tests for parameters indicative of anti-tumorigenicity as described herein.

In some embodiments, the clonal isolates provided herein exhibited increased production of extracellular enveloped virus (EEV) compared to the starting virus preparation or mixture or other reference strain or isolate, including recombinant strains. Vaccinia virus replicates in cells and produces both intracellular virus (IMV, intracellular mature virus; IEV, intracellular enveloped virus) and extracellular virus (EEV, extracellular enveloped virus; CEV, cell-associated extracellular virus) (Smith et al. (1998) Adv Exp Med Biol. 440:395-414). IMV represents approximately 99% of virus yield following replication by wild-type vaccinia virus strains. The IMV virus form is relatively stable in the outside environment, and is primarily responsible for spread between individuals; however, IMV virus does not spread efficiently within the infected host due to inefficient release from cells and sensitivity to complement and/or antibody neutralization. By contrast, the EEV form is released into the extracellular milieu and typically represents only approximately 1% of the viral yield (Smith et al. (1998) Adv Exp Med Biol. 440:395-414). EEV is responsible for viral spread within the infected host and is relatively easily degraded outside of the host. In addition, the EEV form has developed several mechanisms to inhibit its neutralization within the bloodstream. EEV is relatively resistant to complement (Vanderplasschen et al. (1998) Proc Natl Acad Sci USA. 95(13): 7544-9) due to the incorporation of host cell inhibitors of complement into its outer membrane coat and secretion of vaccinia virus complement control protein (VCP) into local extracellular environment. In addition, EEV is relatively resistant to neutralizing antibody effects compared to IMV (Smith et al. (1997) Immunol Rev. 159:137-54; Vanderplasschen et al. (1997) J Gen Virol. 78 (Pt 8): 2041-8). EEV is released at earlier time points following infection (e.g., 4-6 hours) than is IMV (which is only released during/after cell death), and therefore, spread of the EEV form is faster (Blasco et al. (1993) J Virol. 67(6):3319-25).

Since EEV is relatively resistant to complement effects and to antibody-mediated neutralization, when it is grown in a cell type from the same species, this virus form will have enhanced stability and retain activity longer in the blood following intravenous administration (Smith et al. (1998) Adv Exp Med Biol. 440:395-414; Vanderplasschen et al., (1998) Proc Natl Acad Sci USA. (13): 7544-9). This is particularly important for repeat administration once neutralizing antibody levels have increased and anti-cancer therapies require repeat administration. Therefore, increasing the EEV form of vaccinia, and other poxviruses, may result in enhanced systemic efficacy.

In some embodiments, the clonal isolates provided herein exhibited increased production of extracellular enveloped virus (EEV) compared to other clonal isolates derived from the IDH-J or the Copenhagen strains. In some embodiments, the clonal isolates provided herein exhibited increased production of extracellular enveloped virus (EEV) such as at or between 120% to 1000%, for example, at least 120%, 130%, 140%, 150%, 160%, 170%, 180%, 190%, 200%, 250%, 300%, 400%, 500%, 1000% or more of the production of extracellular enveloped virus (EEV) compared to the starting virus preparation or mixture or other reference strain or isolate, including recombinant strains.

In some embodiments, greater than at or about 5%, 6%, 7%, 8%, 9%, 10%, 11%, 12%, 13%, 14%, 15%, 16%, 17%, 18%, 19%, 20% of the infectious particles after cell infection are EEV. In some embodiments, greater than 5% of the infectious particles after cell infection are EEV. In some embodiments, greater than 10% of the infectious particles after cell infection are EEV. In some embodiments, greater than 15% of the infectious particles after cell infection are EEV. In some embodiments, greater than 20% of the infectious particles after cell infection are EEV.

In other embodiments, the clonal isolates provided herein exhibited decreased tumor and/or metastasis growth or increased tumor and/or metastasis shrinkage in in vitro or in vivo assays or models. Tumors can be harvested from the subjects, weighed, and the weight compared to tumors harvested from tumor-bearing subjects that were infected with virus from the starting virus preparation or mixture or other reference strain or isolate, including recombinant strains. The weight of the tumors can also be compared to tumors harvested from control treated subjects at the same time post-infection. The weight can be presented as tumor volume/weight and/or a ratio of the tumor volume/weight (tumor weight control treated animals/tumor weights of clonal isolate-treated subjects). It is understood that a ratio of tumor weight that is 1.2 or 5, for example, means that the virus effects a decreased tumor/metastasis weight/growth or and increased tumor/metastasis shrinkage, and 120% or 500% of anti-tumorigenicity activity compared to the reference or control.

In some embodiments, the clonal isolates provided herein exhibited decreased tumor and/or metastasis growth or increased tumor and/or metastasis shrinkage. In some embodiments, the tumor/metastasis volume/weight ratio is greater than 1.0, for example, that is greater than 1.1, 1.2, 1.3, 1.4, 1.5, 1.6, 1.7, 1.8, 1.9, 2, 3, 4, 5, 6, 7, 8, 9, 10, 20, 30, 40, 50 or more. In some embodiments, the increased tumor/metastasis shrinkage is at least 120% to 500%, for example, 150%, 200%, 250%, 300%, 350%, 400%, 450%, 500%, or more.

In some embodiments, the clonal isolates provided herein exhibit similar anti-tumorigenic activity compared to the starting virus preparation or mixture or other reference strain or isolate, including recombinant strains such as between 70% to 120%, for example, at least or about or 70%, 80%, 90%, 95%, 100%, 110%, 115% or 120% of the anti-tumorigenic activity of the parental virus preparation or mixture or other reference virus strain in an assay or method to assess a parameter indicative of toxicity.

In some embodiments, the clonal isolates provided herein exhibit decreased tumor and/or metastasis volume, size or weight in in vitro or in vivo assays or models. In some embodiments, the clonal isolates provided herein exhibit decreased tumor and/or metastasis volume such as at or between 0% to 99%, for example, less than 99%, 98%, 97%, 96%, 95%, 90%, 80%, 70%, 60%, 50%, 40%, 30%, 20%, 10%, 5% or less of the toxicity or more of tumor and/or metastasis volume, size or weight compared to the starting virus preparation or mixture or other reference strain or isolate, including recombinant strains.

Parameters indicative of toxicity or virulence include, but are not limited to, reduced percentage of cell survival in 2-D (2 dimensional) and 3-D (3 dimensional) cell cultures, decrease in body weight in a subject, presence of fever, rash or other allergy, fatigue or abdominal pain, tissue distribution of the virus, reduced or decreased survival rate of the subject, induction of an immune response in the subject, amount of tumor antigens that are released and decreased rate of pock formation. The toxicity or virulence can be determined using any in vitro or in vivo tests that are well known to those of skill in the art.

In some embodiments, the clonal isolates provided herein exhibited less toxicity compared to the starting virus preparation or mixture or other reference strain or isolate, including recombinant strains such as at or between 0% to 99%, for example, less than 99%, 98%, 97%, 96%, 95%, 90%, 80%, 70%, 60%, 50%, 40%, 30%, 20%, 10%, 5% or less of the toxicity of the starting virus preparation or mixture or other reference strain or isolate, including recombinant strains in an assay or method to assess a parameter indicative of toxicity. In some embodiments, the IHD-J clonal isolates provided herein exhibit at or between 0% to 99%, for example, less than 99%, 98%, 97%, 96%, 95%, 90%, 80%, 70%, 60%, 50%, 40%, 30%, 20%, 10%, 5% or less of the toxicity compared to other clonal isolates derived from the IHD-J or the Copenhagen strains. In some embodiments, the method to assess a parameter indicative of toxicity includes quantifying the percentage of cell survival in cell cultures. In some embodiments, the method to assess a parameter indicative of toxicity includes quantifying the percentage of cell survival in 2-D (two-dimensional) and 3-D (three-dimensional) cell cultures. (Should I mention all the cell types used in the examples?).

In some embodiments, the clonal isolates provided herein exhibited similar toxicity and/or cytotoxicity compared to the to the starting virus preparation or mixture or other reference strain or isolate, including recombinant strains such as between 70% to 120%, for example, at least or about or 70%, 80%, 90%, 95%, 100%, 110%, 115% or 120% of the anti-tumorigenic activity of the starting virus preparation or mixture or other reference strain or isolate, including recombinant strains in an assay or method to assess a parameter indicative of toxicity.

In particular embodiments, clonal isolates provided herein exhibited improved anti-tumorigenicity and were less toxic (i.e. less virulent) compared to the starting virus preparation or mixture or other reference strain or isolate, including recombinant strains. For example, when administered to a subject in an amount effective to induce anti-tumorigenic activity the clonal strains were less toxic (i.e. less virulent). For treatment of a human subject or other similarly sized subject, exemplary therapeutic amounts of a clonal strain are in the range of about or between 1×10⁶ to 1×10¹⁴ pfu, 1×10⁷ to 1×10¹⁰ pfu, such as 1×10⁹ to 1×10¹⁰ pfu, for example at least or about 1×10⁶, 1×10⁷, 1×10⁸, 1×10⁹, 2×10⁹, 3×10⁹, 4×10⁹, or 5×10⁹ pfu. For treatment of a mouse or other similarly sized subject, exemplary therapeutic amounts of a clonal strain are in the range of about or between 1×10³ to 1×10⁹ pfu, such as 1×10⁵ to 1×10⁷ pfu, for example at least or about or 1×10³, 1×10⁴, 1×10⁵, 1×10⁶, 2×10⁶, 3×10⁶, 4×10⁶ or 5×10⁶ pfu. Such effective amounts can be empirically determined by a person skilled in the art and depend on a variety of factors including the subject, the condition or disease being treated, the stage or progression of the disease, the type of cancer, tumor, metastasis, or hyperplasia, and other factors. Dosage regimes can vary. In some embodiments, the clonal isolates provided herein, over the course of a treatment regime, exhibit 100% survival of subjects and are not associated with effecting decreased or reduced weight of a subject over the course of treatment. In one embodiment, the clonal strains provided herein, when administered to a subject, exhibit a survival rate that is increased compared to the survival rate of subjects administered with the same or similar therapeutic amount of other clonal isolates. In some embodiments, the clonal isolates provided herein, over the course of a treatment regime, exhibited 100% tumor growth inhibition.

Isolated clonal viruses provided herein can be derived from plaque isolation of the IHD-J strain that is propagated through repeated passage in cell lines. In some embodiments, the clonal isolates provided herein can be obtained by passage of virus in embryonated chicken eggs culture, in chicken embryo fibroblasts (CEF), Hela S3 cells, confluent CV-1 cells, or BHK-21 cells. In some embodiments, the clonal isolates provided herein can be obtained by passage of virus in confluent CV-1, African green monkey kidney fibroblast cell cultures, growing in 6-well plates infected with a series of dilutions of the vaccinia virus strains. The clonal isolates provided herein are homogenous in sequence. Exemplary clonal viruses provided herein are clonal isolates that exhibit enhanced anti-tumorigenic properties and reduced toxicity.

### II. Attenuated Vaccinia Virus Strains

Also provided here are recombinant vaccinia virus that exhibit one or more modifications to attenuate virus toxicity compared to the wild-type or parental strain of the virus, such as compared to any of the isolated clonal virus strains described in Section I. In some embodiments, provided herein is a recombinant vaccinia virus that is attenuated, such as has reduced toxicity, compared to the vaccinia virus strain VIP02. In some embodiments, provided herein is a recombinant vaccinia virus that is attenuated, such as has reduced toxicity, compared to the vaccinia virus strain set forth in SEQ ID NO: 1. In some embodiments, an attenuated virus is a virus that has low toxicity to normal cells, such as low or reduced viral replication, cytolytic activity or cytotoxicity to normal cells, such as non-tumor cells.

In some embodiments, the attenuated virus is a recombinant oncolytic vaccinia virus, comprising: an inactivating mutation of B2R; a heterologous nucleic acid encoding interferon regulatory factor 3 (IRF3); and at least one heterologous nucleic acid encoding one or more cytokine and/or chemokine. In some embodiments, the at least one heterologous nucleic acid encoding one or more cytokine and/or chemokine comprises a heterologous nucleic acid encoding chemokine ligand 9 (CXCL9) and/or IL-12.

In some embodiments, the attenuated virus is a recombinant oncolytic virus, comprising: an inactivating mutation of at least one viral gene; and at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing.

In some embodiments, any of the provided vaccinia viruses can be made to be attenuated by modifying the vaccinia virus to be deficient in the function of vaccinia growth factor (VGF) (McCart et al. (2001) Cancer Research 61:8751); thymidine kinase (TK) gene (WO 2005/047458); a hemagglutinin (HA) gene (WO 2005/047458, and Zhang et al. (2007) Cancer Research 67:10038); an F3 gene (also called F14.5L; WO 2005/047458, and Zhang et al. (2007) Cancer Research 67:10038); ribonucleotide reductase (Gammon et al. (2010) PLOS Pathogens 6: e1000984); serine protease inhibitor (e.g., SPI-1, SPI-2) (Guo et al. (2005) Cancer Research 65:9991, and Yang et al. (2007) Gene Therapy 14:638); ribonucleotide reductase genes F4L or I4L (Child et al. (1990) Virology 174:625; Potts et al. (2017) EMBO Mol. Med. 9:638); B2R (Eaglesham et al. (2019) Nature 566:259-263); B18R (Symons et al. (1995) Cell 81:551; Kirn et al. (2007) PLOS Medicine 4: e353); A48R (Hughes et al. (1991) J. Biol. Chem. 266:20103); B8R (Verardi et al. (2001) J. Virol. 75:11); B15R (Spriggs et al. (1992) Cell 71:145); A41R (Ng et al. (2001) Journal of General Virology 82:2095); A52R (Bowie et al. (2000) Proc. Natl. Acad. Sci. USA 97:10162); F1L (Gerlic et al. (2013) Proc. Natl. Acad. Sci. USA 110:7808); E3L (Chang et al. (1992) Proc. Natl. Acad. Sci. USA 89:4825); A44R-A46R (Bowie et al. (2000) Proc. Natl. Acad. Sci. USA 97:10162); K1L (Bravo Cruz et al. (2017) Journal of Virology 91: e00524); A48R, B18R, C11R, and TK (Mejias-Perez et al. (2017) Molecular Therapy: Oncolytics 8:27). In some embodiments, it is known that several nonessential genes, such as J2R (thymidine kinase TK) (Buller et al. 1985), C11R (secreted epidermal growth factor-like) (Buller et al.1988), A56R (hemagglutinin HA) (Shida etal.1988), B8R (solubleinterferon-gamma receptor-like) (Verardi et al.2001) and F14.5L (WO 2005/047458, and Zhang et al. (2007) Cancer Research 67:10038) result in reduced virulence when deleted or disrupted.

In some embodiments, provided herein is a recombinant vaccinia virus strain that has a genome in which any of the above genes has an inactivating mutation that inactivates the gene and thereby attenuates the virus. In some embodiments, the viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L and I4L. In some embodiments, the inactivating mutation is deletion of all of a portion of the viral gene. In some embodiments, the inactivating mutation is a deletion of the entire ORF of the viral gene. In some embodiments, the inactivating mutation is deletion of a portion of the ORF of the viral gene that is renders the encoded gene product non-function. In some embodiments, the portion of the ORF that is deleted is a contiguous sequence of at least 10, 20, 30, 40, 50, 60, 70, 80, 90, 100 or more nucleotides up to the entire sequence of the ORF of the viral gene.

In some embodiments, a gene region or encoded gene product can be made deficient in function by any of a variety of methods known to a skilled artisan. In some embodiments, a gene region, or a gene product, may be made deficient as a result of one or more mutation (e.g. substitution), truncation or deletion of the gene region. In some embodiments, a gene region, or a gene product, may be made deficient as a result of a mutation, truncation or deletion of a promoter region controlling expression of the gene region. In some embodiments, a gene region, or a gene product, may be made deficient by mutation, truncation or deletion of a polyadenylation sequence such that translation of a polypeptide encoded by the gene region is reduced or eliminated.

In some embodiments, an attenuated recombinant vaccinia virus of the present disclosure that is deficient in a given vaccinia virus gene exhibits reduced production and/or activity of a gene product (e.g., mRNA gene product; polypeptide gene product) of the gene. In some embodiments, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by wild-type vaccinia virus, or by a control vaccinia virus that does not comprise the genetic alteration. For example, in some embodiments, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by VIP02 or a vaccinia virus having the nucleic acid genome set forth in SEQ ID NO: 1. In some embodiments, the amount and/or activity of the gene product is less than 75%, less than 60%, less than 50%, less than 40%, less than 30%, less than 25%, less than 20%, less than 15%, less than 10%, less than 5%, or less than 1% of the amount and/or activity of the same gene product produced by IHD-W1 strain or a vaccinia virus having the nucleic acid genome set forth in SEQ ID NO: 2.

In some embodiments, an attenuated recombinant vaccinia virus of the present disclosure that is deficient in a viral gene may have deletion in a region consisting of the specified gene region or the deletion in a neighboring gene region comprising the specified gene region. As an example, a mutation and/or truncation and/or deletion of a promoter region that reduces transcription of a gene region can result in deficiency. A gene region can also be rendered deficient through incorporation of a transcriptional termination element such that translation of a polypeptide encoded by the gene region is reduced or eliminated. A gene region can also be rendered deficient through use of a gene-editing enzyme or a gene-editing complex to reduce or eliminate transcription of the gene region. A gene region can also be rendered deficient through use of competitive reverse promoter/polymerase occupancy to reduce or eliminate transcription of the gene region. A gene region can also be rendered deficient by insertion of a nucleic acid into the gene region, thereby knocking out the gene region. In some cases, a heterologous nucleic acid may be inserted into the viral gene, such as described for exemplary recombinant vaccinia virus strains in Section III.

In some embodiments, an OVV provided by the present disclosure is vaccinia virus thymidine kinase (TK) deficient. In some cases, an OVV of the present disclosure comprises a deletion of all or a portion of the vaccinia virus TK coding region, such that the replication-competent, recombinant oncolytic vaccinia virus is TK deficient. For example, in some cases, an OVV of the present disclosure comprises a deletion in the J2R gene (i.e., gene that encodes viral thymidine kinase). See, e.g., Mejia-Perez et al. (2018) Mol. Ther. Oncolytics 8:27. In some cases, an OVV of the present disclosure comprises an insertion into the J2R region, thereby resulting in reduced vaccinia virus TK expression or activity.

In some embodiments, any of the clonal vaccinia virus strains described in Section I, such as VIP02 or a vaccinia virus strain set forth in SEQ ID NO: 1, may be further modified in their genome to attenuate the virus. In some embodiments, the vaccinia virus strains are modified in one or more of the TK (J2R), hemagglutinin (HA), A35R or B2R genes. In some embodiments, the modification renders the gene product encoded by the locus as non-functional or deficient. In some embodiments, all or a portion of the TK, HA, A35R or B2R ORFs are deleted.

In some embodiments, an attenuated recombinant vaccinia virus provided herein has an inactivation mutation, such as an insertion, mutation or deletion, of the J2R gene encoding thymidine kinase (TK; SEQ ID NO: 66). In some embodiments, the TK locus has been reported to not be essential for virus replication such that its modification can decrease viral virulence, result in the inability of virus to replicate in brain or ovary and retain the ability to replicate preferentially in tumor tissue (e.g. Buller et al. (1985) Nature, 317:813-815). In some embodiments, the nucleic acid genome of the recombinant vaccinia virus strain comprises a sequence of nucleotide that has at least 95%, at least 96%, at least 97%, at least 98% or at least 99% sequence identity to the sequence set forth in SEQ ID NO: 4. In some embodiments, the nucleic acid genome of the recombinant vaccina virus strain has the sequence of nucleotides set forth in SEQ ID NO: 4. In some embodiments, the recombinant vaccinia virus is the vaccinia virus designated VIR13.

In some embodiments, an attenuated recombinant vaccinia virus provided herein has an inactivation mutation, such as an insertion, mutation or deletion, of the B2R locus encoding a cytosolic cGAMP nuclease (poxin) (SEQ ID NO: 54). In some embodiments, the B2R locus has been reported to result in vaccinia virus attenuation in a skin scarification model (Eaglesham et al. 2019, Nature 566:259-263). In some embodiments, the attenuated recombinant vaccina virus provide herein has an inactivation mutation, such as an insertion, mutation or deletion, of the B2R gene and an inactivation mutation, such as an insertion, mutation or deletion, of the J2R gene. In some embodiments, the nucleic acid genome of the recombinant vaccinia virus strain comprises a sequence of nucleotide that has at least 95%, at least 96%, at least 97%, at least 98% or at least 99% sequence identity to the sequence set forth in SEQ ID NO: 48. In some embodiments, the nucleic acid genome of the recombinant vaccina virus strain has the sequence of nucleotides set forth in SEQ ID NO: 48. In some embodiments, the recombinant vaccinia virus is the vaccinia virus designated VIR94.

In some embodiments, an attenuated recombinant vaccinia virus provided herein has an inactivation mutation, such as an insertion, mutation or deletion, of the A35R locus. A35R is a virulence gene that modulates the adaptive immune response, and its inactivation such as by deletion can lead to a decrease in viral replication capacity and reduce viral virulence (Brennan et al. 2015, J. Virol., 89:9986-9997). In some embodiments, the nucleic acid genome of the recombinant vaccinia virus strain comprises a sequence of nucleotide that has at least 95%, at least 96%, at least 97%, at least 98% or at least 99% sequence identity to the sequence set forth in SEQ ID NO: 3. In some embodiments, the nucleic acid genome of the recombinant vaccina virus strain has the sequence of nucleotides set forth in SEQ ID NO: 3. In some embodiments, the recombinant vaccinia virus is the vaccinia virus designated VIR11. In some embodiments, the attenuated recombinant vaccina virus provide herein has an inactivation mutation, such as an insertion, mutation or deletion, of the A35R gene and an inactivation mutation, such as an insertion, mutation or deletion, of the J2R gene. In some embodiments, the nucleic acid genome of the recombinant vaccinia virus strain comprises a sequence of nucleotide that has at least 95%, at least 96%, at least 97%, at least 98% or at least 99% sequence identity to the sequence set forth in SEQ ID NO: 12. In some embodiments, the nucleic acid genome of the recombinant vaccina virus strain has the sequence of nucleotides set forth in SEQ ID NO: 12. In some embodiments, the recombinant vaccinia virus is the vaccinia virus designated VIR52.

In some embodiments, an attenuated recombinant vaccinia virus provided herein has an inactivation mutation, such as an insertion, mutation or deletion, of the A56R locus encoding hemagglutinin (HA; SEQ ID NO: 67). In some embodiments, the HA locus has been reported to not be essential for virus replication such that its modification can decrease viral virulence, result in the inability of virus to replicate in brain or ovary and retain the ability to replicate preferentially in tumor tissue (e.g. Shida et al. (1988) J. Virol., 62:4474-4480).

In some embodiments, an attenuated recombinant vaccinia virus provided herein has an inactivation mutation, such as an insertion, mutation or deletion, of the F14.5L gene (SEQ ID NO: 65). In some embodiments, an attenuated recombinant vaccinia viruses provided herein has an insertion, mutation or deletion of the F3 gene product encoded by the F14.5L gene (SEQ ID NO: 64). In some embodiments, the F14.5L gene (also called F3) has been reported to not be essential for virus replication such that its modification can decrease viral virulence, result in the inability of virus to replicate in brain or ovary and retain the ability to replicate preferentially in tumor tissue (e.g. U.S. patent publication No. US2005/0031643).

A variety of method can be used to assess or determine the level of attenuation of a virus. Such methods for measuring the level of attenuation can be performed in vitro or in vivo and can include assessment of changes in any or all of the following properties of the virus: a) viral mRNA synthesis, b) viral protein expression, c) viral DNA replication, d) viral plaque size, e) viral titer or f) in vivo toxicity. Methods for assessing the level of attenuation of a virus by in vitro and in vivo methods are known in the art and include, but are not limited to, methods such as plaque assays and mouse models of viral pathogenicity. Exemplary methods for studying vaccinia early, intermediate, and late transcription can be found in Broyles et al. Methods Mol Biol. (2004) 269:135-142 and Wright et al. Methods Mol. Biol. (2004) 269:143-150. Method for assaying for viral RNA transcripts and proteins include, but are not limited to, well-known techniques as RNA hybridization and blotting techniques and immunohistochemistry.

III. Recombinant Virus Strains with Heterologous Nucleic Acid

Provided herein are recombinant virus strains that are modified in their genomic sequence. In some embodiments, provided herein is a recombinant oncolytic virus comprising at least one heterologous nucleic acid encoding one or more heterologous gene product. The heterologous gene product is not particularly limited and can be, in some embodiments, a complement inhibitor, a T cell or NK cell evader, an immune stimulating protein, an anti-angiogenic protein, an interferon regulatory factor, an apoptosis inducible protein, or a combination of any of the foregoing. Accordingly, in some embodiments, provided herein is a recombinant oncolytic virus comprising at least one heterologous nucleic acid encoding one or more heterologous gene product, wherein the one or more heterologous gene product is a complement inhibitor, a T cell or NK cell evader, an immune stimulating protein, an anti-angiogenic protein, an interferon regulatory factor, an apoptosis inducible protein, or a combination of any of the foregoing.

Provided herein is a recombinant oncolytic vaccinia virus, comprising: an inactivating mutation of B2R; a heterologous nucleic acid encoding interferon regulatory factor 3 (IRF3); and at least one heterologous nucleic acid encoding one or more cytokine and/or chemokine. In some embodiments, the at least one heterologous nucleic acid encoding one or more cytokine and/or chemokine comprises a heterologous nucleic acid encoding chemokine ligand 9 (CXCL9) and/or IL-12.

Also provided herein is a recombinant oncolytic virus, comprising: an inactivating mutation of at least one viral gene; and at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing. In some embodiments, the at least one heterologous nucleic acid encoding one or more heterologous gene product comprises one or more heterologous nucleic acid each encoding one or more immune modulating proteins, such as one or more immune modulating proteins selected from the group consisting of LIGHT, IRF3, IL-2, IL-12, and CXCL9; and/or the at least one heterologous nucleic acid encoding one or more heterologous gene product comprises one or more heterologous nucleic acid each encoding an apoptosis-inducible protein, such as an iDED, an iFas, or an iCas9; and/or the at least one heterologous nucleic acid encoding one or more heterologous gene product comprises one or more heterologous nucleic acid each encoding one or more T cell or NK cell evader proteins, such as a set of proteins encoded by Cowpox virus ORFs 012, 203 and 018 (CPXV012-203-018); and/or the at least one heterologous nucleic acid encoding one or more heterologous gene product comprises one or more heterologous nucleic acid each encoding one or more complement inhibitor, such as CRASP-2 or miniFH; and/or the one or more heterologous nucleic acid encoding the one or more complement inhibitor is introduced into a viral membrane gene, optionally F14.5L, to produce a fusion gene encoding a fusion protein; and/or the at least one heterologous nucleic acid encoding one or more heterologous gene product comprises one or more heterologous nucleic acid each encoding one or more anti-angiogenic protein, such as a VEGF inhibitor, an angiopoietin inhibitor, or versikine; and/or the at least one heterologous nucleic acid encoding one or more heterologous gene product comprises one or more heterologous nucleic acid each encoding one or more therapeutic agent or diagnostic agent.

An inactivating mutation includes any of several ways of altering expression and/or functionality of a gene product expressed by the viral gene being inactivated, such as by gene disruption. Gene disruption can be achieved by, e.g., a gene deletion, nucleic acid insertion, nucleic acid mutations or substitutions, knockouts, premature stop codons, transcriptional promoter modifications, RNAi, or gene editing, e.g., CRISPR. In some embodiments, an inactivating mutation is by a gene deletion and/or an insertion (also referred to as an introduction) of a heterologous nucleic acid encoding one or more gene product. In particular embodiments, an inactivating mutation combines a gene deletion and insertion of a heterologous nucleic acid into such gene loci. For instance, in some methods of effecting inactivating mutation, such as by homologous recombination and other methods, a heterologous nucleic acid may be inserted within a region of a gene that has been deleted. Thus, it is understood that in some embodiments, reference to a gene loci into which a heterologous nucleic acid is inserted is a deleted loci of a gene that has been inactivated by gene deletion of all or a portion of the gene. In some embodiments, a gene deletion removes the entire sequence of the gene. In other embodiments, a gene deletion is a partial deletion, that is, one that removes portion of the sequence of the gene. In one embodiment, a gene deletion is a partial deletion that removes at least 30%, at least 40%, at least 50%, at least 60%, at least 70%, at least 80%, at least 90% or at least 95% of the sequence of the gene. In one embodiment, a gene deletion is a partial deletion that removes at least 30%, at least 40%, at least 50%, at least 60%, at least 70%, at least 80%, or at least 90% of the protein coding sequence of the gene. In other embodiments, a gene deletion removes 100% of the sequence of the gene. In yet other embodiments, a gene deletion removes 100% of the protein coding sequence of the gene. In one embodiment, a gene deletion removes at least 50, at least 100, at least 200, at least 300, at least 400, at least 500, at least 600, at least 700, at least 800, at least 900, or at least 1000 nucleotides of the sequence of the gene. In another embodiment, a gene deletion is a partial deletion that removes at least 50, at least 100, at least 200, at least 300, at least 400, at least 500, at least 600, at least 700, at least 800, at least 900, or at least 1000 nucleotides of the sequence of the gene. In a specific embodiment, a partial deletion in a gene results in a partial gene.

Also provided herein is a recombinant oncolytic virus, comprising at least one heterologous nucleic acid encoding one or more heterologous gene product, wherein the one or more heterologous gene product is or comprises a complement inhibitor, a T cell or NK cell evader, an immune modulating protein, an anti-angiogenic protein, an interferon regulatory factor, an apoptosis inducible protein, or a combination of any of the foregoing.

Also provided herein is a recombinant oncolytic virus, comprising: a nucleic acid genome that has at least 99% sequence identity with the nucleic acid sequence set forth in SEQ ID NO: 1; and at least one heterologous nucleic acid encoding one or more heterologous gene product inserted in the genome.

Exemplary heterologous proteins are described in the following subsections. In addition to a recombinant virus strain, any of the described heterologous proteins can also be incorporated into a gene therapy vectors (e.g. AAV, lentivirus and retrovirus) or a cell-based therapy (e.g. chimeric antigen receptor-expressing T cell (CAR-T), natural killer (NK) NK cell or tumor infiltrating lymphocyte (TIL) therapy).

Among provided virus strains are recombinant virus strains comprising at least one heterologous nucleic acid encoding one or more heterologous gene product. In some embodiments, the recombinant virus includes, but not limited to, a vaccinia virus, a vesicular stomatitis virus (VSV), a Maraba virus (MARAV), a measles virus (MV), a myxoma virus, an orf virus, a parvovirus, a raccoonpox virus, a coxsackievirus, a reovirus, a Newcastle disease virus, a Seneca valley virus, a Semliki Forest virus, an influenza virus, an echovirus, a poliovirus (PV), adenoviruses (e.g., mastadenovirus and avian adenovirus), herpes viruses (e.g., herpes simplex virus 1, herpes simplex virus 2, herpes simplex virus 5, herpes simplex virus 6, Epstein-Barr virus, HHV6-HHV8 and cytomegalovirus), leviviruses (e.g. levivirus, enterobacterial phase MS2, allolevirus), poxviruses (e.g. the subfamily chordopoxvirus, parapoxvirus, avipoxvirus, capripoxvirus, leporipoxvirus, swipoxvirus, molluscipoxvirus entomopoxivirus), papovaviruses (e.g., poliomavirus and papillomavirus), paramyxoviruses (e.g., paramyxovirus, parainfluenza virus 1 (e.g., measles rubella virus), rubulavirus (e.g., mumps virus), pneumovirus, (pneumovirus, (pneumovirus) human), human respiratory syncytial virus and metapneumovirus (e.g. avian pneumovirus and human metapneumovirus)), picornaviruses (e.g. enterovirus, rhinovirus, hepatovirus (e.g., human hepatitis A virus), cardiovirus and aptovirus), reoviruses (e.g., orthoreovirus, orbivirus, rotavirus, cypovirus, fijivirus, phytoreovirus and oryzavirus), retroviruses (e.g., mammalian type B retroviruses, type C mammalian retroviruses, groups of type D retroviruses, BLV-HTLV retroviruses, lentiviruses (e.g., human immunodeficiency virus type 1 and human immunodeficiency virus type 2 (e.g. HIV gp 160), spumavirus), flaviviruses (e.g. hepatitis C virus, Dengue fever virus, virus and West Nile fever), hepatadaviruses (e.g. hepatitis B virus), togaviruses (e.g. alphavirus (e.g. Sindbis virus) and rubiviruses (e.g. rubella virus)), rhabdoviruses (e.g. vesiculovirus, lissavirus, ephemerovirus, and cytoradovirus), arenaviruses (e.g., arenavirus, lymphocytic choriomeningitis virus, Ippy virus, and lassavirus) and coronaviruses (e.g., coronavirus and torovirus).

In some embodiments, the recombinant virus includes oncolytic viruses. In some embodiments, the recombinant virus is a recombinant oncolytic virus. In some embodiments, the recombinant virus, e.g., recombinant oncolytic virus, is a vaccinia virus, a herpes simplex virus, vesicular stomatitis virus (VSV), a Maraba virus (MARAV), a measles virus (MV), adenovirus, myxoma virus, orf virus, parvovirus, raccoonpox virus, coxsackievirus, reovirus, Newcastle disease virus, Seneca valley virus, Semliki Forest virus, mumps virus, influenza virus, echovirus, or a poliovirus (PV). In some embodiments, the recombinant virus, e.g., recombinant oncolytic virus, is a vaccinia virus.

In some embodiments, the recombinant virus is a virus that is not an oncolytic virus. In some embodiments, the recombinant virus is a virus that is not a vaccinia virus. In some embodiments, the recombinant virus includes a vaccinia virus. In some embodiments, the recombinant virus is derived from the Copenhagen strain.

In particular embodiments, the recombinant virus is an IHD-J derived virus. In some embodiments, the recombinant virus is a VIP02-derived virus. In some embodiments, provided herein are recombinant viruses, e.g., recombinant oncolytic viruses, that comprise one or more mutations, insertions, deletions, or substitutions (replacement) of nucleic acid, or other modification of the genomic sequence of the virus. In some embodiments, provided herein are modified VIP02 strains that are modified in a genomic sequence compared to the genomic sequence set forth in SEQ ID NO: 1. In some embodiments, the recombinant virus is a virus that is derived from a virus that has a nucleic acid genome set forth in SEQ ID NO: 1 in which the genome is modified by insertion of a nucleic acid encoding a heterologous gene product.

Methods for the generation of recombinant viruses using recombinant DNA techniques are well known in the art (e.g., see U.S. Pat. Nos. 4,769,330, 4,603,112, 4,722,848, 4,215,051, 5,110,587, 5,174,993, 5,922,576, 6,319,703, 5,719,054, 6,429,001, 6,589,531, 6,573,090, 6,800,288, 7,045,313, He et al. (1998) PNAS USA. 95 (5): 2509-2514. Racaniello et al., (1981) Science 214:916-919). Methods for the generation of recombinant vaccinia viruses for the methods can also be found in the Examples described herein.

In some embodiments, the recombinant virus has a large carrying capacity for foreign genes where exogenous DNA fragments can be inserted. For instance, the vaccinia virus genome has a large carrying capacity for foreign genes, where up to 25 kb of exogenous DNA fragments can be inserted. The genomes of several of the vaccinia strains have been completely sequenced, and many essential and nonessential genes identified. Due to high sequence homology among different strains, genomic information from one vaccinia strain can be used for designing and generating modified viruses in other strains. Finally, the techniques for production of modified vaccinia strains by genetic engineering are well established (Moss, Curr. Opin. Genet. Dev. 3:86-90 (1993); Broder and Earl, Mol. Biotechnol. 13:223-245 (1999); Timiryasova et al., Biotechniques 31:534-540 (2001).

Sites for the insertion of heterologous nucleic acid molecules are known in the art and have been described for various viral vectors (see e.g., 5,166,057, 5,266,489, 6,338,846, 6,248,320, 6,221,646, 6,841,158, 7,101,685, 7,001,760 and references therein). Heterologous nucleic acid molecules are typically inserted into a non-coding region or in a coding region for a gene that is nonessential for viral replication. For example, in vaccinia virus, sites for insertions of heterologous DNA molecules can be in intergenic regions, non-coding regions, and or nonessential genes or gene regions including, but not limited to, thymidine kinase (TK) gene, hemagglutinin (HA) gene, F14.5L (see, e.g., U.S. Patent Pub. No. 2005-0031-643), VGF gene (see, e.g., U.S. Pat. Pub. No. 2003-0031681), Hind III F, F13L, or Hind III M (see, e.g., U.S. Pat. No. 6,548,068); a hemorrhagic region or an A type inclusion body region (ATI) (see, e.g., U.S. Pat. Nos. 6,265,189 and 6,596,279); A33R, A34R, A36R or B5R genes (see, e.g., Katz et al., (2003) J. Virology 77:12266-12275); SalF7L (see, e.g., Moore et al., (1992) EMBO J. 11:1973-1980); NIL (see, e.g., Kotwal et al. (1989) Virology 171:579-587); M1 lambda (see, e.g., Child et al. (1990) Virology. 174:625-629); HR, HindIIII-MK, HindIII-MKF, HindIII-CNM, RR, or BamF (see, e.g., Lee et al. (1992) J Virol. 66:2617-2630); C21L (see, e.g., Isaacs et al. (1992) Proc Natl Acad Sci USA. 89:628-632), host range region genes K1L and C7L, A35R (see e.g., U.S. Pat. Nos. 6,265,189, 7,045,313; U.S. Patent Pub. Nos. 2005-0244428, 2006-0159706; Coupar et al. J. Gen. Virol. (2000) 81:431-439; Smith et al. (1993) Vaccine 11(1): 43-53). If more than one gene expression cassette is inserted, the insertions can be at the same insertion site or different insertion sites. Alternatively, the heterologous nucleic acid molecules can be inserted into an essential gene, and a cell line for packaging of the virus could be use for the production of the virus.

In some embodiments, the at least one heterologous nucleic acid encoding the one or more heterologous gene product is inserted into or in place of a non-essential gene or region in the genome of the virus. In some embodiments, the at least one heterologous nucleic acid encoding the one or more heterologous gene product is inserted into or in place of the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene locus in the genome of the virus, or any combination thereof. In some embodiments, the at least one heterologous nucleic acid encoding the one or more heterologous gene product is inserted into or in place of the F14.5L gene locus. The F14.5 gene locus encodes a viral membrane protein. In some embodiments, the at least one heterologous nucleic acid encoding the one or more heterologous gene product is inserted into or in place of the A35R gene locus. In some embodiments, the at least one heterologous nucleic acid encoding the one or more heterologous gene product is inserted into or in place of the J2R gene locus. In some embodiments, insertion into a gene loci is an insertion in which the gene loci contains a partial deletion and the heterologous nucleic acid replaces the deleted portion. In some embodiments, insertion into a gene loci is an insertion into the gene loci but in which no portion of the endogenous gene loci is deleted. In some embodiments, insertion in place of the gene loci is an insertion such that all of the gene loci is deleted and replaced by the heterologous nucleic acid.

Mutation of nonessential vaccinia genes can also contribute to increased attenuation of the virus. Thus, insertion of heterologous expression cassettes into a nonessential gene, such as the TK gene, can attenuate the virus in two aspects: by gene mutation and by added transcriptional and/or translational load. For the methods described herein, mutation of nonessential genes is not required; however, one or more nonessential gene can be modified to enhance the attenuating effects of the gene expression cassette. The attenuation of the virus can be subsequently lessened (i.e., the virus exhibits increased replication) by removing the expression cassette and replacing it with noncoding sequence so that the gene remains inactive. Thus, removal or replacement of a gene expression cassette decreases the transcriptional and/or translational load on the virus, resulting in a decrease in attenuation of the virus.

In some embodiments, the at least one heterologous nucleic acid encoding the one or more heterologous gene product is fused with a gene encoding a viral membrane protein in the genome of the virus. In some embodiments, the at least one heterologous nucleic acid encoding the one or more heterologous gene product is fused with a gene encoding a viral membrane protein to produce a fusion protein. In some embodiments, the at least one heterologous nucleic acid encoding the one or more heterologous gene product is fused with a viral membrane protein to produce a fusion protein. In some embodiments, the gene encoding the viral membrane protein that is fused with the at least one heterologous nucleic acid encoding the one or more heterologous gene product is F14.5L. In some embodiments, the viral membrane protein is F14.5L. In some embodiments, the viral membrane protein is F14.5L and the fusion is at the C-terminus of F14.5L. In some embodiments, the fusion protein is incorporated into the outer membrane of the intracellular mature virus (IMV), e.g., of a vaccinia virus. These fusion proteins that comprise the viral membrane protein F14.5L are expected to be incorporated into the outer membrane of IMV viral particles, which can provide them with resistance to inactivation by complement in the blood.

Modifications can include mutations, insertions, deletions, or substitutions (replacement) of nucleic acid or other modification of the genomic sequence of the virus. For example, viruses provided herein can be modified to contain one or more heterologous nucleic acid molecule inserted or replaced into the genome of the virus. The viral gene can be replaced with a homologous gene from another virus or with a different gene. In one embodiment, modifications include insertion or replacement of one or more nucleotides, such as at least 1, 2, 3, 4, 5, 6, 7, 8, 9, 10, 20, 30, 40, 50, 60, 70, 80, 90, 100, 150, 200, 250, 300, 350, 400, 450, 500, 1000, 2000, 3000 or more nucleotides. In some embodiments, modifications include deletion of one or more nucleotides, such as at least 1, 2, 3, 4, 5, 6, 7, 8, 9, 10, 20, 30, 40, 50, 60, 70, 80, 90, 100, 150, 200, 250, 300, 350, 400, 450, 500, 1000, 2000, 3000 or more nucleotides. In some embodiments, modifications include substitution of one or more nucleotides, such as at least 1, 2, 3, 4, 5, 6, 7, 8, 9, 10, 20, 30, 40, 50, 60, 70, 80, 90, 100, 150, 200, 250, 300, 350, 400, 450, 500, 1000, 2000, 3000 or more nucleotides.

Modifications include insertions and/or substitutions (replacement) of nucleic acid or other modification of the genomic sequence of the virus with a heterologous nucleic acid. Generally, the heterologous gene is a gene that encodes a non-viral protein. For example, a heterologous nucleic acid molecule can be inserted that encodes a heterologous gene. In some embodiments, the heterologous nucleic acid replaces all or a portion of a viral gene. In other embodiments, the viruses provided herein can be modified by insertion of one or more heterologous nucleic acid molecules. For example, 1, 2, 3, 4, 5, 6, 7, 8, 9, 10 or more heterologous nucleic acid molecules can be inserted. A heterologous nucleic acid molecule can contain an open reading frame or can be a non-coding sequence. Generally, the heterologous nucleic acid that is inserted is a contiguous sequence of nucleotides that contains an open reading frame and corresponds to a coding region of a gene. Inserted or replaced genes can be transcribed and/or translated from the viral genome following infection of a host cell, such as a tumor cell. As described below, the heterologous nucleic acid can contain a regulatory sequence to control expression of the gene. For example, the heterologous nucleic acid can be operably linked to a promoter for expression of an open reading frame. In some embodiments, the promoter has a sequence identity 70, 80, 90, 100% identical to the sequences set forth in SEQ ID NOs: 68, 69, 70, 71, or 72. In some embodiments, the promoter has a sequence identity identical to the sequences set forth in SEQ ID NOs: 68, 69, 70, 71, or 72.

Modifications to the viral genome provided herein can result in changes of virus characteristics or properties. Example changes include changes in parameters indicative of anti-tumorigenicity and/or toxicity. For example, insertions, mutations or deletions can decrease pathogenicity of the clonal strain, for example, reducing the infectivity, toxicity, ability to replicate or number of non-tumor organs or tissues in which the vaccinia virus can accumulate. Exemplary insertions, deletions, mutations, and/or substitutions of nucleic acids are those that result in a vaccinia virus with better anti-tumorigenic properties and less toxicity relative to the clonal strain not containing the modification and/or the starting virus preparation or mixture or other reference strain or isolate, including recombinant strains. In some embodiments, the insertions, deletions, mutations, and/or substitutions of nucleic acids are those that result in a vaccinia virus with similar anti-tumorigenic properties and toxicity relative to the clonal strain not containing the modification and/or the starting virus preparation or mixture or other reference strain or isolate, including recombinant strains. In some embodiments, the modifications to the viral genome reduce the toxicity relative to the clonal strain not containing the modification and/or the starting virus preparation or mixture or other reference strain or isolate, including recombinant strains. In some embodiments, the insertions, mutations or deletions include, but are not limited to, those that increase anti-tumorigenicity and reduce toxicity of the virus relative to the clonal strain not containing the modification and/or the starting virus preparation or mixture or other reference strain or isolate, including recombinant strains.

In some embodiments, insertions, mutations or deletions include, but are not limited to, those that increase ability of the clonal viral strain to evade host's immune system relative to the clonal strain not containing the modification and/or the starting virus preparation or mixture or other reference strain or isolate, including recombinant strains. In some embodiments, insertions, mutations or deletions include, but are not limited to, those that increase the ability of the clonal viral strain to stimulate host's immune system relative to the clonal strain not containing the modification and/or the starting virus preparation or mixture or other reference strain or isolate, including recombinant strains. In some embodiments, insertions, mutations or deletions include, but are not limited to, those that increase the host's anti-angiogenic activity relative to the clonal strain not containing the modification and/or the starting virus preparation or mixture or other reference strain or isolate, including recombinant strains. In some embodiments, insertions, mutations or deletions include, but are not limited to, those that increase the host's apoptotic activity relative to the clonal strain not containing the modification and/or the starting virus preparation or mixture or other reference strain or isolate, including recombinant strains.

In some embodiments, the one or more heterologous nucleic acid molecules can encode, for example, an anti-apoptotic gene product of fragment thereof, such as gene product that can modify host's apoptotic response; an angiogenesis gene product or fragment thereof, such as a gene product that can modify host's angiogenesis response; an immune system gene product or fragment thereof, such as a gene product that can modify the host's immune response. In some embodiments, the gene product or fragment thereof that can modify the host's immune response, can increase host's immune system's ability to escape inhibition by complement relative to the clonal strain not containing the modification and/or the starting virus preparation or mixture or other reference strain or isolate, including recombinant strains. In some embodiments, the gene product that can modify the host's immune response increases host's immune system's activity relative to the clonal strain not containing the modification and/or the starting virus preparation or mixture or other reference strain or isolate, including recombinant strains.

In some embodiments, the recombinant virus is a vaccinia virus that is modified in a genomic sequence compared to the genomic sequence set forth in SEQ ID NO: 1 or a sequence that has at least 99% sequence identity to SEQ ID NO: 1. In some embodiments, the recombinant virus is a vaccinia virus that is modified in a genomic sequence compared to the genomic sequence set forth in SEQ ID NO: 1. The large genome size of vaccinia viruses provided herein allows insertion of large and/or multiple nucleotide sequences of heterologous DNA into the virus genome (Smith and Moss (1983) Gene 25(1): 21-28). The viruses provided herein can be modified by insertion or substitution of one or more nucleotides. In one embodiment, modifications include insertion or substitution of one or more nucleotides, such as at least of 1, 2, 3, 4, 5, 6, 7, 8, 9, 10, 20, 30, 40, 50, 60, 70, 80, 90, 100, 150, 200, 250, 300, 350, 400, 450, 500, 1000, 2000, 3000, 4000, 5000 or more nucleotides. In some embodiments, the one or more heterologous DNA molecules are inserted into a gene loci of the virus genome, such as any as described herein. In some embodiments, the one or more heterologous DNA molecules are inserted into a non-essential region of the virus genome; for example, the DNA molecules are inserted into a locus that is not essential for viral replication in proliferating cells, such as tumor cells. Exemplary insertion sites are known in the art and provided herein. In some embodiments, the recombinant vaccinia virus provided herein can contain an inactivating mutation in a viral gene such as any as described, such as a gene deletion of all or a portion of a viral gene. In such embodiments, the one or more heterologous nucleic acids may be inserted into or in place of such gene loci. In some embodiments, the recombinant virus is a modified virus compared to the genomic sequence set forth in SEQ ID NO: 1 in which one or more heterologous nucleic acids are inserted and one or more viral gene loci are inactivated, such as by gene deletion. The modified recombinant virus can be any of the virus provided herein having a genome set forth in SEQ ID NO: 1, or a genome that is at least 99% identical to SEQ ID NO: 1, or any other virus, generated by introduction of the heterologous DNA described herein. In some embodiments, the recombinant virus is modified in a genomic sequence compared to the genomic sequence set forth in SEQ ID NO: 1 and has a sequence of amino acids that exhibits at least 85%, 90% or 95% sequence identity to the sequence set forth in SEQ ID NO: 1. In some embodiments, the nucleic acid genome of the recombinant oncolytic vaccinia virus has at least 95%, 96%, 97%, 98%, or 99% sequence identity to the nucleic acid sequence set forth in SEQ ID NO: 1.

In some embodiments, the recombinant virus can be modified to express an exogenous or heterologous gene. Exemplary exogenous gene products include proteins involved in apoptosis, angiogenesis, and/or modulation of the immune system. In some embodiments, gene products include proteins that affect a host's apoptosis pathways such as Caspase-9, DED (death effector domain) of FADD (Fas-associated death domain protein), and Fas. In some embodiments, gene products include proteins that affect host's angiogenesis pathways such as vascular endothelial growth factor (VEGF) and versikine (VK). In some embodiments, gene products include proteins that affect host's immune system such as minimized complement regulator factor H (miniFH), Borrelia burgdorferi complement regulatory-acquiring surface protein-2 (CRASP-2), Cowpox virus ORFs 012, 203 and 018 (CPXV012-203-018), and human LIGHT mutant (hmLIGHT). The characteristics of such gene products are described herein and elsewhere.

In particular, the viruses provided herein can be modified to express genes in vivo and in vitro. In some embodiments, the viruses can be modified to express two or more gene products, such as 2, 3, 4, 5, 6, 7, 8, 9, 10 or more gene products, where any combination of the two or more gene products can be one or more detectable gene products. In one embodiment, a virus can be modified to express an apoptosis related gene product. In another example, a virus can be modified to express two or more gene products for generation of fusion proteins. In some examples, one or more proteins involved in angiogenesis can be expressed together. When two or more heterologous genes are introduced, the genes can be regulated under the same or different regulatory sequences, and the genes can be inserted in the same or different regions of the viral genome, in a single or a plurality of genetic manipulation steps. In some embodiments, one gene can be under the control of a constitutive promoter while a second gene can be under the control of an inducible promoter. Methods for inserting two or more genes into a virus are known in the art and can be readily performed for a wide variety of viruses using a wide variety of exogenous genes, regulatory sequences, and/or other nucleic acid sequences.

The viruses provided herein can be modified by insertion, deletion, substitution or mutation as described herein. Standard methodologies for modifying viruses by inserting, deleting, substituting and mutating nucleic acids are well known in the art. Such methodologies include in vitro recombination techniques, synthetic methods, direct cloning, and in vivo recombination methods. See, for example, Sambrook et al., Molecular Cloning: A Laboratory Manual, 2nd edition, Cold Spring Harbor Laboratory Press, cold Spring Harbor N.Y. (1989), and the Examples disclosed herein. Techniques for the generation of recombinant viruses include nucleic acid transfer protocols, various nucleic acid manipulation techniques, nucleic acid amplification protocols and, typically, involves the generation of gene cassettes or transfer vectors using standard techniques in molecular biology. See, e.g., U.S. Pat. Nos. 5,494,807 and 5,185,146, which describe exemplary methods of generating recombinant vaccinia viruses and other molecular biology techniques known in the art. Methods for the generation of recombinant viruses using recombinant DNA techniques are well known in the art (e.g., see U.S. Pat. Nos. 4,769,330; 4,603,112; 4,722,848; 4,215,051; 5,110,587; 5,174,993; 5,922,576; 6,319,703; 5,719,054; 6,429,001; 6,589,531; 6,573,090; 6,800,288; 7,045,313; He et al. (1998) PNAS 95 (5): 2509-2514; Racaniello et al., (1981) Science 214:916-919; and Hruby et al., (1990) Clin Micro Rev. 3:153-170). Methods for the generation of recombinant vaccinia viruses are well known in the art (e.g., see Hruby et al., (1990) Clin Micro Rev. 3:153-170, U.S. Pat. Pub. No. 2005-0031643, now U.S. Pat. Nos. 7,588,767, 7,588,771, 7,662,398 and 7,045,313).

In some embodiments, homologous recombination can be used to introduce an insertion or deletion of a nucleic acid molecule into a target sequence of interest. Use of nucleic acid tools such as vectors, plasmids, promoters and other regulating sequences, are well known in the art for a large variety of viruses and cellular organisms. Nucleic acid amplification protocols include, but are not limited to, the polymerase chain reaction (PCR), or amplification via viruses or organisms, such as, but not limited to, yeast, bacteria, insect or mammalian cells. Nucleic acid transfer protocols include electroporation, calcium chloride transformation/transfection, liposome mediated nucleic acid transfer, and others. A large variety of tools to modify nucleic acids is available from many different sources, including various commercial sources. For example, point mutations or small insertions or deletions can be introduced into a gene of interest through the use of oligonucleotide mediated site-directed mutagenesis. In another example, homologous recombination can be used to introduce a mutation in the nucleic acid sequence, or to insert or delete a nucleic acid molecule into a target sequence of interest. In some examples, mutations, insertions or deletions of nucleic acids in a particular gene can be selected for using a positive or negative selection pressure. See, e.g., Current Techniques in Molecular Biology, (Ed. Ausubel, et al.). One skilled in the art will be readily able to select the appropriate tools and methods for genetic modifications of any particular virus according to the knowledge in the art and design choice. In some embodiments, a plasmid is used for homologous recombination for constructing a recombinant virus. In some embodiments, the plasmid is constructed using gene splicing for joining two fragments. In some embodiments, primer used to amplify the two fragments comprise 70%, 80%, 90%, or 100% of SEQ ID NOs: 14, 15, 16, 17, 31, 32, 33, 34, 35, 36, 37, 38, 39, 40, 41, 42, 43, 44, 45, 46, 94, 95, 96, or 97.

The insertion, deletion, substitution or mutation can be specifically directed to a particular sequence in the viral genome. Such sequences in the viral genome include, but are not limited to, an intergenic sequence, a regulatory sequence, a sequence without a known role, a gene-encoding sequence, or a non-essential region of the viral genome. Regions of viral genomes that are available for modification are well known in the art for many viruses.

In some embodiments, the recombinant virus, e.g., recombinant oncolytic virus, comprises an inactivation mutation of at least one viral gene. The inactivating mutation is not particularly limited and can, in some embodiments, be any mutation that results in the viral gene's gene product having less function or no function as compared to without the inactivating mutation. In some embodiments, the inactivation mutation is deletion of all or a portion of the at least one viral gene. In some embodiments, the deletion of the at least one viral gene is deletion of the entire gene ORF of a viral gene. In some embodiments, the deletion of the at least one viral gene is a deletion of a portion of the ORF of a viral gene. In some embodiments, the deletion of the at least one viral gene is a deletion of a portion of the ORF of a viral gene that is sufficient to render the encoded gene product non-functional. In some embodiments, the at least one viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, and I4L. In some embodiments, the at least one viral gene comprises two or more viral genes selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, and I4L. In some embodiments, the at least one viral gene is A35R. In some embodiments, the at least one viral gene is J2R. In some embodiments, the at least one viral gene is B2R. In some embodiments, the at least one viral gene is B2R. In some embodiments, the at least one viral gene is B2R. In some embodiments, the at least one viral gene comprises A35R and J2R. In some embodiments, the at least one viral gene is B2R. In some embodiments, the at least one viral gene comprises B2R and J2R.

Heterologous nucleic acid molecules are typically inserted into the viral genome in an intergenic region or in a locus that encodes a nonessential viral gene product. Insertion of heterologous nucleic acid at such sites generally does not significantly affect viral infection or replication in the target tissue. Examples of insertion sites include, but are not limited to, J2R (thymidine kinase (TK)), A56R (hemagglutinin (HA)), F14.5L, vaccinia growth factor (VGF), A35R, NIL, E2L/E3L, K1L/K2L, superoxide dismutase locus, 7.5K, C7-K1L (host range gene region), B13R+B14R (hemorrhagic region), A26L (A type inclusion body region (ATI)) or I4L (large subunit, ribonucleotide reductase) gene loci. Insertion sites for the viruses provided herein also include sites that correspond to intragenic regions described in other poxviruses such as Modified Vaccinia Ankara (MVA) virus (exemplary sites set forth in U.S. Pat. No. 7,550,147), NYVAC (exemplary sites set forth in U.S. Pat. No. 5,762,938). In some embodiments, insertion, deletion, substitution and/or mutations sites include J2R, F14.5L and/or A35R.

For example, generating a recombinant vaccinia virus that expresses a heterologous gene product typically includes the use of a recombination plasmid, which contains the heterologous nucleic acid, optionally operably linked to a promoter, with vaccinia virus DNA sequences flanking the heterologous nucleic acid to facilitate homologous recombination and insertion of the gene into the viral genome. Generally, the viral DNA flanking the heterologous gene is complementary to a non-essential segment of vaccinia virus DNA, such that the gene is inserted into a nonessential location or any other location. The recombination plasmid can be grown in and purified from Escherichia coli and introduced into suitable host cells, such as, for example, but not limited to, CV-1, BSC-40, BSC-1 and TK-143 cells. The transfected cells are then superinfected with vaccinia virus which initiates a replication cycle. The heterologous DNA can be incorporated into the vaccinia viral genome through homologous recombination, and packaged into infection progeny. The recombinant viruses can be identified by methods known in the art, such as by detection of the expression of the heterologous gene product, or by using positive or negative selection methods (U.S. Pat. No. 7,045,313). In some embodiments, a recombinant virus is generated by homologous incorporation of a plasmid into the viral genomic region corresponding to the J2R gene. In some embodiments, a recombinant virus is generated by homologous incorporation of a plasmid into the viral genomic region corresponding to the A35R gene. In some embodiments, a recombinant virus is generated by homologous incorporation of a plasmid into the viral genomic region corresponding to the F14.5L gene. In some embodiments, a recombinant virus is generated by homologous incorporation of one plasmid into the viral genomic region corresponding to the J2R gene and another plasmid into the viral genomic region corresponding to the F14.5L gene. In some embodiments, a recombinant virus is generated by homologous incorporation of one plasmid into the viral genomic region corresponding to the J2R gene and another plasmid into the viral genomic region corresponding to the F14.5L gene. In some embodiments, a recombinant virus is generated by homologous incorporation of one plasmid into the viral genomic region corresponding to the J2R gene and another plasmid into the viral genomic region corresponding to the A35R gene. In some embodiments, a recombinant virus is generated by homologous incorporation of one plasmid into the viral genomic region corresponding to the F14.5L gene and another plasmid into the viral genomic region corresponding to the A35R gene. In some embodiments, a recombinant virus is generated by homologous incorporation of one plasmid into the viral genomic region corresponding to the J2R gene and another plasmid into the viral genomic region corresponding to the A35R gene, and another plasmid into the viral genomic region corresponding to the F14.5L gene.

In another example, the recombinant vaccinia virus that expresses a heterologous gene product can be generated by direct cloning (see, e.g. U.S. Pat. No. 6,265,183 and Scheiflinger et al. (1992) Proc. Natl. Acad. Sci. USA 89:9977-9981). In such methods, the heterologous nucleic acid, optionally operably linked to a promoter, is flanked by restriction endonuclease cleavage sites for insertion into a unique restriction endonuclease site in the target virus. The virus DNA is purified using standard techniques and is cleaved with the sequence-specific restriction endonuclease, where the sequence is a unique site in the virus genome. Any unique site in the virus genome can be employed provided that modification at the site does not interfere with viral replication. Generally, insertion is in a site that is located in a non-essential region of the virus genome. For example, exemplary modifications herein include insertion of a foreign DNA sequence into the NotI digested virus DNA.

In some examples, the heterologous nucleic acid also can contain one or more regulatory sequences to regulate expression of an open reading frame encoding the heterologous RNA and/or protein. Suitable regulatory sequences, which, for example, are functional in a mammalian host cell, are well known in the art. Expression can also be influenced by one or more proteins or RNA molecules expressed by the virus. Gene regulatory elements, such as promoters and enhancers, possess cell-type specific activities and can be activated by certain induction factors (e.g., hormones, growth factors, cytokines, cytostatic agents, irradiation, heat shock) via responsive elements. A controlled and restricted expression of these genes can be achieved using such regulatory elements as internal promoters to drive the expression of genes in viral vector constructs.

In some embodiments, the heterologous nucleic acid encoding the one or more heterologous gene product is operably linked to a promoter. In some embodiments, the one or more heterologous nucleic acid encoding the one or more heterologous gene product is operably linked to a promoter for expression of the heterologous RNA and/or protein. For example, a heterologous nucleic acid that is operably linked to a promoter is also called an expression cassette. Hence, viruses provided herein can have the ability to express one or more heterologous genes. Gene expression can include expression of a protein encoded by a gene and/or expression of an RNA molecule encoded by a gene. In some embodiments, the viruses provided herein can express exogenous genes at levels high enough that permit harvesting products of the exogenous genes from the tumor. Expression of heterologous genes can be controlled by a constitutive promoter, or by an inducible promoter. In other examples, organ or tissue-specific expression can be controlled by regulatory sequences. In order to achieve expression only in the target organ, for example, a tumor to be treated, the foreign nucleotide sequence can be linked to a tissue specific promoter and used for gene therapy. Such promoters are well known to those skilled in the art (see, e.g., Zimmermann et al., Neuron 12:11-24 (1994); Vidal et al., EMBO J. 9:833-840 (1990); Mayford et al., Cell 81:891-904 (1995); and Pinkert et al., Genes & Dev. 1:268-76 (1987)).

Exemplary promoters for the expression of heterologous genes are known in the art. The heterologous nucleic acid can be operatively linked to a native promoter or a heterologous promoter that is not native to the virus. Any suitable promoters, including synthetic, naturally occurring, and modified promoters, can be used. Exemplary promoters include synthetic promoters, including synthetic viral and animal promoters. Native promoter or heterologous promoters include, but are not limited to, viral promoters, such as vaccinia virus and adenovirus promoters.

In some embodiments, the promoter is a poxvirus promoter, such as, for example, a vaccinia virus promoter. As such, in some embodiments, the promoter is a poxviral promoter or is a variant or derivative thereof, such as a vaccinia virus promoter. In some embodiments, the promoter is a vaccinia virus promoter. Vaccinia viral promoters for the expression of one or more heterologous genes can be synthetic or natural promoters, and include vaccinia early, intermediate, early/late and late promoters. Exemplary vaccinia viral promoters for controlling heterologous gene expression include, but are not limited to, 7.5E, 7.5E/L, SSE, 11KL, SSL, SSEL, mH5, LEO, P7.5k, P11k, PSE, PSEL, PSL, H5R, TK, P28, C11R, G8R, F17R, I3L, I8R, A1L, A2L, A3L, H1L, H3L, H5L, H6R, H8R, D1R, D4R, D5R, D9R, D11L, D12L, D13L, M1L, N2L, P4b or K1 promoters. Accordingly, in some embodiments, the nucleic acid encoding the heterologous gene product is operably linked to a promoter selected from the group consisting of 7.5E, 7.5E/L, SSE, 11KL, SSL, SSEL, mH5, LEO, P7.5k, P11k, PSE, PSEL, PSL, H5R, TK, P28, C11R, G8R, F17R, I3L, I8R, A1 L, A2L, A3L, H1L, H3L, H5L, H6R, H8R, D1R, D4R, D5R, D9R, D11L, D12L, D13L, M1L, N2L, P4b, and K1 promoters. Other viral promoters include, but are not limited to, adenovirus late promoter, Cowpox ATI promoter, or T7 promoter. Strong late promoters can be used to achieve high levels of expression of the heterologous genes. Early and intermediate-stage promoters can also be used. In one example, the promoters contain early and late promoter elements, for example, the modified H5 promoter, PmH5, which contains both native early and late vaccinia promoter regions the vaccinia virus, the synthetic early/late vaccinia PSEL promoter, and the PSE synthetic early promoter (Hammond et al., Journal of Virological Methods 66:1, 135-138 (1997); Stritzker et al., Journal of Virology 88:19, 11556-11567 (2014; Kugler et al., Virol J. 16:100 (2019). In some embodiments, the promoter is synthetic strong early promoter (SSE). In some embodiments, the promoter is a strong early/late promoter (SEL).

In some embodiments, the promoter is selected from the group consisting of 7.5E, 7.5E/L, SSE, 11KL, SSL, SSEL, mH5 and LEO. In some embodiments, the promoter has the amino acids sequence set forth in any one of SEQ ID NOS: 29, 53, 55, 68, 69, 70, 71, or 72. In some embodiments, the promoter has the amino acids sequence set forth in SEQ ID NO: 29. In some embodiments, the promoter is synthetic strong early promoter (SSE) and comprises the amino acids sequence set forth in SEQ ID NO: 29. In some embodiments, the promoter has the amino acids sequence set forth in SEQ ID NO: 55. In some embodiments, the promoter is a strong early/late promoter (SEL) and comprises the amino acids sequence set forth in SEQ ID NO: 55. In some embodiments, the promoter is a poxviral promoter, and the poxviral promoter is mH5. In some embodiments, the poxviral promoter is mH5 and comprises the amino acid sequence set forth in SEQ ID NO: 53.

Combinations of different promoters can be used to express different gene products in the same virus or two different viruses. The viruses provided herein can exhibit differences in characteristics, such as attenuation, as a result of using a stronger promoter versus a weaker promoter. For example, in vaccinia, synthetic early/late and late promoters are relatively strong promoters, whereas vaccinia synthetic early promoters are relatively weaker promoters (see e.g., Chakrabarti et al. (1997) BioTechniques 23 (6) 1094-1097).

As is known in the art, regulatory sequences can permit constitutive expression of the exogenous gene or can permit inducible expression of the exogenous gene. Further, the regulatory sequence can permit control of the level of expression of the exogenous gene. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

Hence, expression of heterologous genes can be controlled by a constitutive promoter or by an inducible promoter. Inducible promoters can be used to provide tissue specific expression of the heterologous gene or can be inducible by the addition of a regulatory molecule to provide temporal specific induction of the promoter. In some examples, inducible expression can be under the control of cellular or other factors present in a tumor cell or present in a virus-infected tumor cell. In further examples, inducible expression can be under the control of an administrable substance, including IPTG, RU486 or other known induction compounds. Additional regulatory sequences can be used to control the expression of the one or more heterologous genes inserted the virus. Any of a variety of regulatory sequences are available to one skilled in the art according to known factors and design preferences.

In some embodiments, the one or more heterologous gene product comprise a therapeutic agent or diagnostic agent. In some embodiments, the one or more heterologous gene product, e.g., therapeutic agent or diagnostic agent, is selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging, and genes for MRI imaging. In some embodiments, the one or more heterologous gene product, e.g., therapeutic agent or diagnostic agent, comprise a therapeutic agent selected from among a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein, and a tissue factor.

In some of any of such embodiments, the recombinant virus, e.g., recombinant oncolytic virus, comprises a nucleic acid sequence comprising at least one heterologous nucleic acids encoding one or more heterologous gene product, such as any of the heterologous gene products described herein, e.g., in Section III parts A, B, C, and D, including, e.g., one or more heterologous gene products selected from the group consisting of a complement inhibitor, a T cell evader or an NK cell evader, an immune stimulating protein, an anti-angiogenic protein, an interferon regulatory factor, an apoptosis-inducible protein, or any combination thereof, and, optionally, an inactivating mutation of at least one viral gene, such as one or more of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus, optionally wherein the one or more viral genes is one or more of B2R, J2R, A35R, and A56R, and any combination thereof.

In some of any of such embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93. In some embodiments, the nucleic acid genome of the recombinant oncolytic virus comprises a nucleic acid sequence set forth in any one of SEQ ID NOs: 85, 86, 88, and 90, or a nucleic acid sequence that has at least 95%, 96%, 97%, 98%, or 99% sequence identity to a nucleic acid sequence set forth in any one of SEQ ID NOs 85, 86, 88, and 90. In some embodiments, the nucleic acid genome of the recombinant oncolytic virus comprises a nucleic acid sequence set forth in SEQ ID NO: 85, or a nucleic acid sequence that has at least 95%, 96%, 97%, 98%, or 99% sequence identity to a nucleic acid sequence set forth in SEQ ID NO 85. In some embodiments, the nucleic acid genome of the recombinant oncolytic virus comprises a nucleic acid sequence set forth in SEQ ID NO: 48, or a nucleic acid sequence that has at least 95%, 96%, 97%, 98%, or 99% sequence identity to a nucleic acid sequence set forth in SEQ ID NO 48. In some embodiments, the nucleic acid genome of the recombinant oncolytic virus comprises a nucleic acid sequence set forth in SEQ ID NO: 80, or a nucleic acid sequence that has at least 95%, 96%, 97%, 98%, or 99% sequence identity to a nucleic acid sequence set forth in SEQ ID NO 80. In some embodiments, the nucleic acid genome of the recombinant oncolytic virus comprises a nucleic acid sequence set forth in SEQ ID NO: 82, or a nucleic acid sequence that has at least 95%, 96%, 97%, 98%, or 99% sequence identity to a nucleic acid sequence set forth in SEQ ID NO 82. In some embodiments, the nucleic acid genome of the recombinant oncolytic virus comprises a nucleic acid sequence set forth in SEQ ID NO: 84, or a nucleic acid sequence that has at least 95%, 96%, 97%, 98%, or 99% sequence identity to a nucleic acid sequence set forth in SEQ ID NO 84. In some embodiments, the nucleic acid genome of the recombinant oncolytic virus comprises a nucleic acid sequence set forth in SEQ ID NO: 86, or a nucleic acid sequence that has at least 95%, 96%, 97%, 98%, or 99% sequence identity to a nucleic acid sequence set forth in SEQ ID NO 86. In some embodiments, the nucleic acid genome of the recombinant oncolytic virus comprises a nucleic acid sequence set forth in SEQ ID NO: 87, or a nucleic acid sequence that has at least 95%, 96%, 97%, 98%, or 99% sequence identity to a nucleic acid sequence set forth in SEQ ID NO 87. In some embodiments, the nucleic acid genome of the recombinant oncolytic virus comprises a nucleic acid sequence set forth in SEQ ID NO: 88, or a nucleic acid sequence that has at least 95%, 96%, 97%, 98%, or 99% sequence identity to a nucleic acid sequence set forth in SEQ ID NO 88. In some embodiments, the nucleic acid genome of the recombinant oncolytic virus comprises a nucleic acid sequence set forth in SEQ ID NO: 89, or a nucleic acid sequence that has at least 95%, 96%, 97%, 98%, or 99% sequence identity to a nucleic acid sequence set forth in SEQ ID NO 89. In some embodiments, the nucleic acid genome of the recombinant oncolytic virus comprises a nucleic acid sequence set forth in SEQ ID NO: 90, or a nucleic acid sequence that has at least 95%, 96%, 97%, 98%, or 99% sequence identity to a nucleic acid sequence set forth in SEQ ID NO 90. In some embodiments, the nucleic acid genome of the recombinant oncolytic virus comprises a nucleic acid sequence set forth in SEQ ID NO: 91, or a nucleic acid sequence that has at least 95%, 96%, 97%, 98%, or 99% sequence identity to a nucleic acid sequence set forth in SEQ ID NO 91. In some embodiments, the nucleic acid genome of the recombinant oncolytic virus comprises a nucleic acid sequence set forth in SEQ ID NO: 92, or a nucleic acid sequence that has at least 95%, 96%, 97%, 98%, or 99% sequence identity to a nucleic acid sequence set forth in SEQ ID NO 92. In some embodiments, the nucleic acid genome of the recombinant oncolytic virus comprises a nucleic acid sequence set forth in SEQ ID NO: 93, or a nucleic acid sequence that has at least 95%, 96%, 97%, 98%, or 99% sequence identity to a nucleic acid sequence set forth in SEQ ID NO 93.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises A35R, optionally wherein the nucleic acid genome of the recombinant oncolytic virus comprises the nucleic acid sequence of SEQ ID NO: 3, or a nucleic acid sequence that has at least 95%, 96%, 97%, 98%, or 99% sequence identity to the nucleic acid sequence set forth in SEQ ID NO: 3.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises A35R and J2R, optionally wherein the nucleic acid genome of the recombinant oncolytic virus comprises the nucleic acid sequence of SEQ ID NO: 12, or a nucleic acid sequence that has at least 95%, 96%, 97%, 98%, or 99% sequence identity to the nucleic acid sequence set forth in SEQ ID NO: 12.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, and the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018), and wherein the at least one heterologous nucleic acid encoding one or more heterologous gene product comprises one or more heterologous nucleic acid each encoding one or more complement inhibitor that is introduced into a viral membrane gene to produce a fusion gene encoding a fusion protein, optionally wherein the viral membrane gene is F14.5L, optionally wherein the fusion is at the C-terminus of the F14.5L protein, and optionally wherein the nucleic acid genome of the recombinant oncolytic virus comprises the nucleic acid sequence of SEQ ID NO: 10, or a nucleic acid sequence that has at least 95%, 96%, 97%, 98%, or 99% sequence identity to the nucleic acid sequence set forth in SEQ ID NO: 10.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, optionally wherein the nucleic acid genome of the recombinant oncolytic virus comprises the nucleic acid sequence of SEQ ID NO: 4, or a nucleic acid sequence that has at least 95%, 96%, 97%, 98%, or 99% sequence identity to the nucleic acid sequence set forth in SEQ ID NO: 4.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R and A35R, and the inactivating mutation of A35R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and optionally wherein the nucleic acid genome of the recombinant oncolytic virus comprises the nucleic acid sequence of SEQ ID NO: 11, or a nucleic acid sequence that has at least 95%, 96%, 97%, 98%, or 99% sequence identity to the nucleic acid sequence set forth in SEQ ID NO: 11.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R and A35R, and the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; and optionally wherein the nucleic acid genome of the recombinant oncolytic virus comprises the nucleic acid sequence of SEQ ID NO: 13, or a nucleic acid sequence that has at least 95%, 96%, 97%, 98%, or 99% sequence identity to the nucleic acid sequence set forth in SEQ ID NO: 13.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R and A35R, and the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; and optionally wherein the nucleic acid genome of the recombinant oncolytic virus comprises the nucleic acid sequence of SEQ ID NO: 47, or a nucleic acid sequence that has at least 95%, 96%, 97%, 98%, or 99% sequence identity to the nucleic acid sequence set forth in SEQ ID NO: 47.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, and the inactivating mutation of J2R is by insertion of one or more heterologous nucleic acid each encoding an apoptosis-inducible protein, optionally wherein the apoptosis-inducible protein is an inducible DED (iDED), an inducible Fas (iFas), or an inducible Cas9 (iCas9), optionally wherein the nucleic acid genome of the recombinant oncolytic virus comprises the nucleic acid sequence of SEQ ID NO: 7, 8, or 9, or a nucleic acid sequence that has at least 95%, 96%, 97%, 98%, or 99% sequence identity to the nucleic acid sequence set forth in SEQ ID NO: 7, 8, or 9.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; optionally wherein the nucleic acid genome of the recombinant oncolytic virus comprises the nucleic acid sequence of SEQ ID NO: 49, 50, or 93, or a nucleic acid sequence that has at least 95%, 96%, 97%, 98%, or 99% sequence identity to the nucleic acid sequence set forth in SEQ ID NO: 49, 50, or 93.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R and B2R, optionally wherein the nucleic acid genome of the recombinant oncolytic virus comprises the nucleic acid sequence of SEQ ID NO: 48, or a nucleic acid sequence that has at least 95%, 96%, 97%, 98%, or 99% sequence identity to the nucleic acid sequence set forth in SEQ ID NO: 48.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R and B2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; optionally wherein the nucleic acid genome of the recombinant oncolytic virus comprises the nucleic acid sequence of SEQ ID NO: 80, or a nucleic acid sequence that has at least 95%, 96%, 97%, 98%, or 99% sequence identity to the nucleic acid sequence set forth in SEQ ID NO: 80.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A35R; wherein: the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; the inactivating mutation of B2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and the inactivating mutation of A35R is by insertion of one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and optionally wherein the nucleic acid genome of the recombinant oncolytic virus comprises the nucleic acid sequence of SEQ ID NO: 82, or a nucleic acid sequence that has at least 95%, 96%, 97%, 98%, or 99% sequence identity to the nucleic acid sequence set forth in SEQ ID NO: 82.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A56R; wherein: the inactivating mutation of J2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins is IL-2, optionally wherein the IL-2 is an IL-2 superkine, optionally MDNA11; and optionally wherein the nucleic acid genome of the recombinant oncolytic virus comprises the nucleic acid sequence of SEQ ID NO: 84, or a nucleic acid sequence that has at least 95%, 96%, 97%, 98%, or 99% sequence identity to the nucleic acid sequence set forth in SEQ ID NO: 84.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A56R; wherein: the inactivating mutation of J2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; and optionally wherein the nucleic acid genome of the recombinant oncolytic virus comprises the nucleic acid sequence of SEQ ID NO: 85, or a nucleic acid sequence that has at least 95%, 96%, 97%, 98%, or 99% sequence identity to the nucleic acid sequence set forth in SEQ ID NO: 85.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A56R; wherein: the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; the inactivating mutation of J2R is by insertion of one or more heterologous nucleic acid each encoding an apoptosis-inducible protein, optionally wherein the apoptosis-inducible protein is an inducible DED (IDED); and optionally wherein the nucleic acid genome of the recombinant oncolytic virus comprises the nucleic acid sequence of SEQ ID NO: 86, or a nucleic acid sequence that has at least 95%, 96%, 97%, 98%, or 99% sequence identity to the nucleic acid sequence set forth in SEQ ID NO: 86.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, A35R, and A56R; wherein: the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; the inactivating mutation of B2R is by insertion of one or more heterologous nucleic acid encoding one or more immune modulating proteins, optionally wherein the one or more immune modulating proteins is IRF3; the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; the inactivating mutation of A56R is by insertion of one or more heterologous nucleic acid encoding one or more immune modulating proteins, wherein the one or more immune modulating proteins is IL-2 superkine MDNA11; and optionally wherein the nucleic acid genome of the recombinant oncolytic virus comprises the nucleic acid sequence of SEQ ID NO: 87, or a nucleic acid sequence that has at least 95%, 96%, 97%, 98%, or 99% sequence identity to the nucleic acid sequence set forth in SEQ ID NO: 87.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, A35R, and A56R; wherein: the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; the inactivating mutation of B2R is by insertion of one or more heterologous nucleic acid encoding one or more immune modulating proteins, optionally wherein the one or more immune modulating proteins is IRF3; the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; the inactivating mutation of A56R is by insertion of one or more heterologous nucleic acid encoding one or more immune modulating proteins, wherein the one or more immune modulating proteins is IL-2 superkine MDNA11T, optionally wherein the MDNA11T comprises the amino acid sequence set forth in SEQ ID NO: 98; and optionally wherein the nucleic acid genome of the recombinant oncolytic virus comprises the nucleic acid sequence of SEQ ID NO: 88, or a nucleic acid sequence that has at least 95%, 96%, 97%, 98%, or 99% sequence identity to the nucleic acid sequence set forth in SEQ ID NO: 88.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A56R; wherein: the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018); the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is an IL-2 superkine, optionally MDNA11 or MDNA11T; the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein, optionally wherein the fusion is at the C-terminus of the F14.5L protein; and optionally wherein the nucleic acid genome of the recombinant oncolytic virus comprises the nucleic acid sequence of SEQ ID NO: 89, or a nucleic acid sequence that has at least 95%, 96%, 97%, 98%, or 99% sequence identity to the nucleic acid sequence set forth in SEQ ID NO: 89.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, B2R, and A56R; wherein: the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018); the inactivating mutation of B2R is by insertion of one or more heterologous nucleic acid encoding one or more immune modulating proteins, optionally wherein the one or more immune modulating proteins is IRF3; the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein, optionally wherein the fusion is at the C-terminus of the F14.5L protein; and optionally wherein the nucleic acid genome of the recombinant oncolytic virus comprises the nucleic acid sequence of SEQ ID NO: 90, or a nucleic acid sequence that has at least 95%, 96%, 97%, 98%, or 99% sequence identity to the nucleic acid sequence set forth in SEQ ID NO: 90.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises B2R and J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and optionally wherein the nucleic acid genome of the recombinant oncolytic virus comprises the nucleic acid sequence of SEQ ID NO: 91, or a nucleic acid sequence that has at least 95%, 96%, 97%, 98%, or 99% sequence identity to the nucleic acid sequence set forth in SEQ ID NO: 91.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises B2R, J2R, and A56R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; and optionally wherein the nucleic acid genome of the recombinant oncolytic virus comprises the nucleic acid sequence of SEQ ID NO: 92, or a nucleic acid sequence that has at least 95%, 96%, 97%, 98%, or 99% sequence identity to the nucleic acid sequence set forth in SEQ ID NO: 92.

In some of any of such embodiments, the recombinant oncolytic virus comprises: an inactivating mutation of at least one viral gene; and at least one heterologous nucleic acid encoding one or more heterologous gene product, optionally wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing; and wherein: the at least one viral gene is or comprises J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and optionally wherein the nucleic acid genome of the recombinant oncolytic virus comprises the nucleic acid sequence of SEQ ID NO: 93, or a nucleic acid sequence that has at least 95%, 96%, 97%, 98%, or 99% sequence identity to the nucleic acid sequence set forth in SEQ ID NO: 93.

In some of any of such embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93; and is characterized by one or more of: (i) a variant 017 open reading frame (ORF) encoding an amino acid sequence that has at least 95% sequence identity to SEQ ID NO: 57 and comprises a polar uncharged amino acid at position 66, optionally a threonine (T) at position 66; (ii) a variant 038 (K5L) ORF comprising a nucleotide insertion to effect a frameshift mutation, wherein the 038 (K5L) gene product is altered; (iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419; (iv) a variant 104 (H4L) ORF encoding an amino acid sequence having at least 95% sequence identity to SEQ ID NO: 61 and comprises a negatively charged amino acid at position 591, optionally aspartic acid (D) at position 591; and (v) a variant 182 (A56R) ORF comprising deletion of two nucleotides to effect a frameshift mutation, wherein the 182 (A56R) ORF gene product is altered.

In some of any of such embodiments, the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93; and is characterized by one or more of: (i) a guanine (G) at the position corresponding to position 7770 of SEQ ID NO: 1; (ii) a thymine (T) at the position corresponding to position 15261 of SEQ ID NO: 1; (iii) a G at the position corresponding to position 32136 of SEQ ID NO: 1; (iv) a G at the position corresponding to position 49455 of SEQ ID NO: 1; (v) a cytosine (C) at the position corresponding to position 92969 of SEQ ID NO: 1; (vi) the nucleic acid sequence CACTTATATAT at the positions corresponding to positions 106870 to 106880 of SEQ ID NO: 1; (vii) the nucleic acid sequence GTTTTCATTA at the positions corresponding to positions 111267 to 111276 of SEQ ID NO: 1; (viii) an adenine (A) at the position corresponding to position 162715 of SEQ ID NO: 1; (ix) the nucleic acid sequence TACAGACACC at the positions corresponding to positions 165844 to 185853 of SEQ ID NO: 1; and (x) a C at the position corresponding to position 187805 of SEQ ID NO: 1.

### A. Stealth Viruses

Provided herein, in various embodiments, are recombinant viruses comprising heterologous nucleic acids encoding for “stealth proteins” that can be stably and efficiently expressed in many types of virus infected cells. Such stealth proteins can increase the ability of the virus to evade the host's immune system attack, such as by T cells, e.g., cytotoxic T lymphocytes (CTLs) or natural killer (NK) cells. In some embodiments, such stealth proteins can increase the ability of the recombinant virus to evade the host's complement cascade/system activation.

Accordingly, in some embodiments, provided herein is a recombinant oncolytic virus comprising at least one heterologous nucleic acid encoding one or more heterologous gene product, wherein the one or more heterologous gene product is or comprises a complement inhibitor or a T cell or NK cell evader (also sometimes referred to as a stealth protein).

In some embodiments, the recombinant virus includes oncolytic viruses. In some embodiments, the recombinant virus does not include oncolytic viruses. In some embodiments, the recombinant virus incudes any viruses described herein or incorporated by reference herein. In some embodiments, the recombinant virus includes vaccinia viruses. In some embodiments, the recombinant virus includes VIP02-derived viruses.

Oncolytic viruses (OV) can create a favorable microenvironment for the action of the immune system against unique cancer cell determinants; however, the anti-viral immunity triggered against viral antigens from the resultant infection is also a key player during OV-based therapies. Indeed, induced anti-viral immunity can be detrimental for cancer virotherapy, since the activation of the immune system against the virus itself is expected to restrict the viral replication and spread, leading to a decrease in therapeutic efficacy. Lemos de Matos et al., Mol Ther Methods Clin Dev. 2020 Jun. 12; 17:349-358. The complement system keeps a constant vigil against viruses. Its ability to recognize viruses and virus-infected cells, and trigger an immune response, results in neutralization of viruses and killing of the infected cells. This selection pressure exerted by complement on viruses has made them evolve a multitude of countermeasures. Agrawal et al., Front Microbiol. 2017; 8:1117.

In some embodiments, the stealth protein includes, but it is not limited to, Borrelia burgdorferi complement regulatory-acquiring surface protein-2 (CRASP-2), minimized complement regulator factor H (miniFH) and Cowpox virus ORFs 012, 203 and 018 (CPXV012-203-018). Further details about stealth proteins and mechanisms involved in evasion of the host's immune system, such as evading the host's complement or NK or T cell cytotoxcity, can be found in Monrat Chulanetra and Wanpen Chaicumpa, Front. Cell. Infect. Microbiol., 2021, Front. Cell. Infect. Microbiol. 11:702125, which is hereby incorporated by reference for all purposes.

The complement system is an important component of the innate immunity that helps eliminate pathogens and, consequently, during the course of evolution, pathogens have developed diverse strategies to avoid destruction by complement activation. One of the strategies is the ability to acquire proteins that allow pathogens to control steps involved in activation of host's immune response upon infection, hereinafter “stealth proteins”. See Kraiczy et al., Infect Immun. 2001 December; 69 (12): 7800-7809.

The complement system employs a complex cascade of proteolytic cleavages of more than 30 plasma and cell membrane proteins and leads to induction of an inflammatory response, phagocyte and neutrophil chemotaxis, pathogen neutralization and subsequent opsonization, and lysis of the infected cells. The activation can be initiated via three independent pathways (i) classical, binding of the first component in the cascade C1q to an antibody-antigen complex; (ii) alternative, a spontaneous hydrolysis of the downstream complement component 3 (C3) convertase and its interaction with pathogen surface; (iii) the mannose-binding lectin (MBL) pathway triggered by MBL binding to mannose residues on the pathogen surface. All three pathways converge at the stage of cleavage of C3 into C3a, antimicrobial peptide and C3b, an opsonin that binds to the pathogen and labels it for degradation. Because the effector compounds generated in the complement cascade can be delivered to any surface including host membranes, intact host cells protect themselves by expressing multiple complement regulatory proteins. Janeway et al., Immunobiology: The Immune System in Health and Disease. 5th edition.

Inadequate control of the complement system is the underlying or aggravating factor in many human diseases. The alternative pathway (AP) of the complement has the unique property of remaining continuously and indiscriminatingly activated, albeit at a low level. In the AP, C3b self-propagates via a positive-feedback amplification loop that requires very tight regulation mediated by two key soluble AP regulators Factor H (FH) and its splice product FH like-1 (FHL-1). A stealth protein, an engineered version of FH, miniFH, contains only the N- and C-terminal portions of FH linked by an optimized peptide and shows ˜10-fold higher ex vivo potency to inhibit complement activation when compared with F H. Markus J. Harder, *J Immunol. Author manuscript; available in PMC 2017 Jan. 15. J Immunol. 2016 Jan. 15; 196 (2): 866-876. & Christoph Q. Schmidt/J Immunol. Author manuscript; available in PMC 2014 Jun. 1. J Immunol. 2013 Jun. 1; 190(11): 10.4049/jimmunol. 1203548. Published online 2013 Apr. 24. doi: 10.4049/jimmunol. 1203548.

A microorganism that has developed during evolution the ability to avoid complement by producing stealth proteins is Borrelia burgdorferi, a spirochete that causes Lyme disease (LD), the most common vector-borne disease in the northern hemisphere, transmitted by ticks. Upon tick feeding, spirochetes are exposed to host blood and thus to the first line of innate immunity, which they must overcome to survive. A key evasion mechanism Borrelia burgdorferi has developed is the production of complement- or CRP-binding proteins, including CRASPs, a stealth protein that can facilitate complement inactivation. See Yi-Pin Lin et al., Front Cell Infect Microbiol. 2020; 10:1. US20120142023A1. CRASP-2 (also named CspZ) binds to FH/FHL-1 to confer serum resistance in a gain-of-function B. burgdorferi by inhibiting complement activation on the spirochete surface. Infect Immun. 2001 December; 69(12): 7800-7809. Peter Kraiczy. US20200323972A1 Composition and method for generating immunity to Borrelia burgdorferi.

Downregulation of MHC class I on the cell surface is an immune evasion mechanism shared by many DNA viruses, including cowpox virus. CPXVs are members of the Orthopoxvirus genus that includes variola virus, camelpoxvirus, and monkeypox virus and encode an elaborate arsenal of immune-evasion proteins. The ability of CPXV to infect a wide range of mammalian hosts is likely due to the fact that, among the orthopoxviruses, CPXV encodes the most complete set of open reading frames expected to encode immunomodulatory proteins. Among the proteins encoded are CPXV012 and CPXV203, that can prevent cytotoxic T cell recognition by interfering with MHC I-mediated antigen presentation. While CPXV012 inhibits antigenic peptide transport from the cytosol to the ER, CPXV203 blocks MHCI trafficking to the cell surface. Dina Alzhanova and Klaus Früh* Microbes Infect. 2010 November; 12 (12-13): 900-909. McCoy et al., Molecular Immunology 55 (2013) 156-158. Furthermore, the Birghton Red strain produces OMCP (also named CPXV018) a 171-residue protein that is abundantly secreted from infected cells and that can block NKG2D mediated target cell killing by Natural Killer Cells in vitro. Cell Host & Microbe Volume 6, Issue 5, 19 Nov. 2009, Pages 422-432/Journal home page for Cell Host & Microbe/Two Mechanistically Distinct Immune Evasion Proteins of Cowpox Virus Combine to Avoid Antiviral CD8 T Cells.

In some embodiments, the one or more heterologous gene product comprise a complement inhibitor. In some embodiments, the complement inhibitor is Borrelia burgdorferi complement regulatory-acquiring surface protein-2 (CRASP-2) or minimized complement regulator factor H (miniFH). In some embodiments, the complement inhibitor is a CRASP-2 gene product (UniProtKB-050665). The CRASP-2 protein can increase the ability of the recombinant virus to evade the host's complement. Specifically, in some embodiments, the recombinant virus comprises an expression cassette containing a CRASP-2 cDNA fused to the F14.5L gene locus under the control of vaccinia F14.5L gene promoter. In some embodiments, the CRASP-2 molecule includes a full-length CRASP-2. In some embodiments, the complement inhibitor is CRASP-2 and has a sequence of amino acids that exhibits at least 85%, 90% or 95% sequence identity to the sequence set forth in SEQ ID NO: 18. In some embodiments, the complement inhibitor has the sequence set forth in SEQ ID NO: 18.

In some embodiments, the recombinant virus comprises a heterologous nucleic acid that encodes a CRASP-2 molecule comprising CRASP-2 cDNA fused to the F14.5L gene locus, wherein the CRASP-2 comprises an amino acid sequence having at least 90% sequence identity to the amino acid sequence of SEQ ID NO: 18.

In some embodiments, the recombinant virus comprises an amino acid sequence that has at least 70%, such as at least 75%, 80%, 85% or 90% sequence identity to the amino acid sequence of SEQ ID NO: 18. For example, in some embodiments, the recombinant virus comprises an amino acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9% sequence identity to the amino acid sequence of SEQ ID NO: 18, but is less than 100% identical to the amino acid sequence of SEQ ID NO: 18.

In some embodiments, the heterologous nucleic acid that encodes the CRASP-2 gene product is operably linked to the F14.5L gene promoter. In some embodiments, the recombinant virus which includes a heterologous nucleic acid that encodes a CRASP-2 gene product (e.g., comprising the amino acid sequence of SEQ ID NO: 18) is derived from the clonal VIP02 (comprising the nucleic acid sequence of SEQ ID NO: 1) strain and includes a sequence of nucleotides that has at least 70%, such as at least 75%, 80%, 85% or 90% sequence identity to SEQ ID NO: 5 (also named VIR27). In some embodiments, the heterologous gene product is CRASP-2 and is operably linked to the F14.5L gene promoter in the genome of the virus. In some embodiments, the recombinant virus comprises a nucleic acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 5, but is less than 100% identical to the nucleic acid sequence of SEQ ID NO: 5. In some embodiments, the nucleic acid genome of the recombinant oncolytic virus comprises a sequence of nucleotides that has at least 95%, at least 96%, at least 97%, at least 98% or at least 99% sequence identity to the sequence set forth in SEQ ID NO: 5. In some embodiments, the heterologous gene product is CRASP-2 and is operably linked to the F14.5L gene promoter in the genome of the virus, and the recombinant virus comprises a nucleic acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 5. In some embodiments, the heterologous gene product is CRASP-2 and is operably linked to the F14.5L gene promoter in the genome of the virus, and the recombinant virus comprises the nucleic acid sequence of SEQ ID NO: 5. In some embodiments, the recombinant virus, e.g., recombinant oncolytic virus, comprises the nucleic acid sequence of SEQ ID NO: 5. A recombinant oncolytic virus comprising the nucleic acid sequence of SEQ ID NO: 5 is also referred to herein as VIR27.

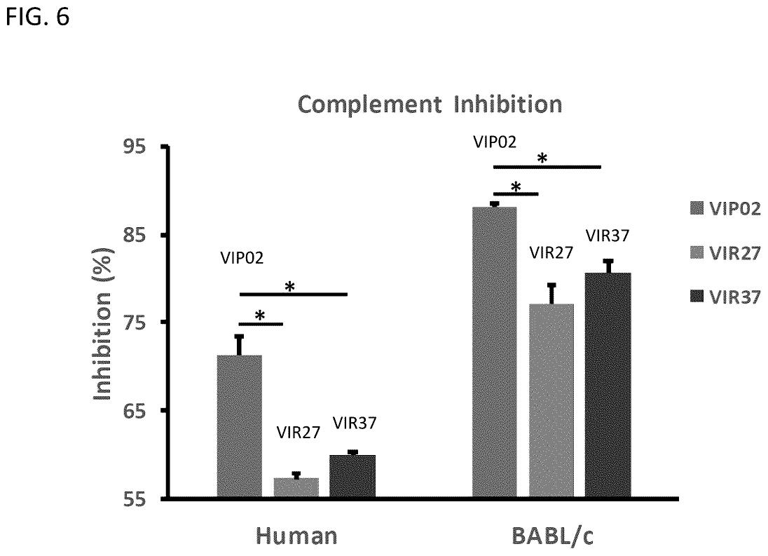

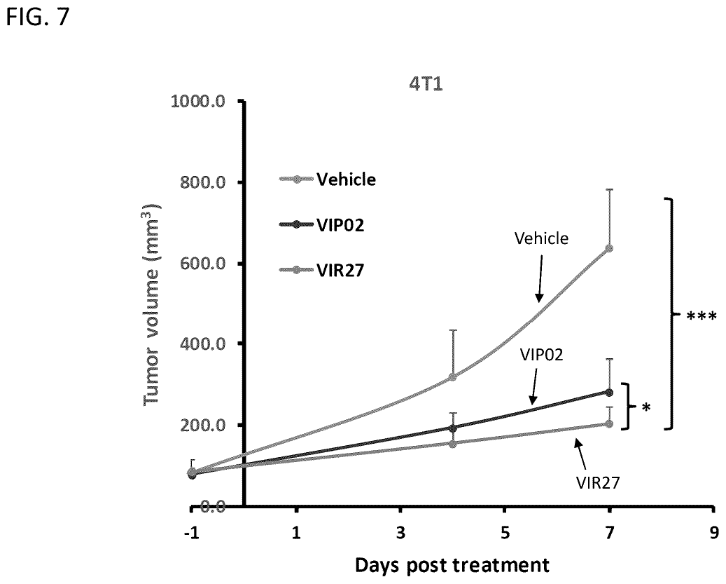

Recombinant viruses provided herein, in various embodiments, exhibit an increased ability to evade the host's complement. In some embodiments, recombinant viruses provided herein can escape complement inhibition in in vivo and in vitro systems. In particular embodiments, VIR27 (comprising the nucleic acid sequence of SEQ ID NO: 5) can escape complement inhibition in an in vitro system of complement inhibition upon incubation of an effective dose of VIR27 (comprising the nucleic acid sequence of SEQ ID NO: 5) with human and/or BABL/c mice serum (FIG. 6). In particular embodiments, administration of an effective dose of VIR27 (comprising the nucleic acid sequence of SEQ ID NO: 5) to a subject inhibits tumor, hyperplasia, or metastasis growth in an in vivo model (FIG. 7).

In some embodiments, the complement inhibitor is a miniFH gene product. Specifically, in some embodiments, the recombinant virus comprises an expression cassette comprising a miniFH cDNA fused to the F14.5L gene locus under the control of vaccinia F14.5L gene promoter. Further details about minFH can be found in Schmidt et al., J Immunol. 2013 Jun. 1; 190(11): 10.4049/jimmunol. 1203548., which is hereby incorporated by reference for all purposes. In some embodiments, the complement inhibitor is a miniFH gene product comprising an amino acid sequence having at least 85%, 90%, or 95% sequence identity to the amino acid sequence set forth in SEQ ID NO: 19. In some embodiments, the complement inhibitor is a miniFH gene product comprising the amino acid sequence set forth in SEQ ID NO: 19.

In some embodiments, the stealth protein includes an FH-based inhibitor, miniFH. In some embodiments, the miniFH gene product can increase the ability of the recombinant virus to evade the host's complement. In some embodiments, a recombinant virus is provided which includes a polynucleotide that encodes a miniFH gene product including miniFH cDNA fused to the F14.5L gene locus, wherein the miniFH polypeptide comprises an amino acid sequence having at least 90% sequence identity to the amino acid sequence of SEQ ID NO: 19. In some embodiments, the recombinant virus comprises a polypeptide comprising an amino acid sequence that has at least 70%, such as at least 75%, 80%, 85%, or 90% sequence identity to the amino acid sequence of SEQ ID NO: 19. For example, in some embodiments, the recombinant virus comprises an amino acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 19, but is less than 100% identical to the amino acid sequence of SEQ ID NO: 19.

In some embodiments, the nucleic acid genome of the recombinant oncolytic virus comprises a sequence of nucleotides that has at least 95%, at least 96%, at least 97%, at least 98% or at least 99% sequence identity to the nucleic acid sequence set forth in SEQ ID NO: 6. In some embodiments, the heterologous gene product is miniFH and is operably linked to the F14.5L gene promoter in the genome of the virus, and the recombinant virus comprises a nucleic acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 6. In some embodiments, the heterologous gene product is miniFH and is operably linked to the F14.5L gene promoter in the genome of the virus, and the recombinant virus comprises the nucleic acid sequence of SEQ ID NO: 6. In some embodiments, the recombinant virus, e.g., recombinant oncolytic virus, comprises the nucleic acid sequence of SEQ ID NO: 6. A recombinant oncolytic virus comprising the nucleic acid sequence of SEQ ID NO: 6 is also referred to herein as VIR37.

In some embodiments, the polynucleotide that encodes the miniFH molecule is operably linked to the F14.5L gene promoter. In some embodiments, the recombinant virus which includes a polynucleotide that encodes an miniFH molecule (e.g., comprising the amino acid sequence of SEQ ID NO: 19) is derived from the clonal VIP02 (comprising the nucleic acid sequence of SEQ ID NO: 1) strain and includes a nucleic acid sequence that has at least 70%, such as at least 75%, 80%, 85% or 90% sequence identity to SEQ ID NO: 6 (also named VIR37). In some embodiments, the recombinant virus comprises a nucleic acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 6, but is less than 100% identical to SEQ ID NO: 6. In some embodiments, the recombinant virus comprises a nucleic acid sequence that comprises the nucleic acid sequence of SEQ ID NO: 6. In some embodiments, the nucleic acid genome of the recombinant oncolytic virus comprises a sequence of nucleotides that has at least 95%, at least 96%, at least 97%, at least 98%, or at least 99% sequence identity to the sequence set forth in SEQ ID NO: 6. In some embodiments, the nucleic acid genome of the recombinant oncolytic virus comprises the nucleic acid sequence of SEQ ID NO: 6.

Recombinant viruses provided herein, in various embodiments, exhibit an increased ability to evade the host's complement. In some embodiments, recombinant viruses provided herein can escape complement inhibition in in vivo and in vitro systems. In particular embodiments, VIR37 (comprising the nucleic acid sequence of SEQ ID NO: 6) can escape complement inhibition in an in vitro system of complement inhibition upon incubation of an effective dose of VIR37 (comprising the nucleic acid sequence of SEQ ID NO: 6) with human and/or BABL/c mice serum (FIG. 6).

In some embodiments, the one or more heterologous gene product is a T cell evader or NK cell evader. A T cell evader or NK cell evader gene product can increase the ability of the virus to evade the host's immune system attack, such as by T cells, e.g., cytotoxic T lymphocytes (CTLs) or natural killer (NK) cells. In particular, such T cell evader or NK cell evader gene products can increase the ability of the recombinant virus to evade the host's complement cascade/system activation.

In some embodiments, the T cell evader or NK cell evader is a set of proteins encoded by Cowpox virus ORFs 012, 203 and 018 (CPXV012-203-018). CPXV012-203-018 is a synthetic DNA fragment. ORFs 012, 203 and 018 are expressed separately under their own promoters within the CPXV012-203-018 synthetic DNA fragment to encode the CPXV012, CPXV203, and CPXV018 proteins. The Cowpox virus evades CTLs by CPXV012 and CPXV203. While CPXV012 inhibits antigenic peptide transport from the cytosol to the endoplasmic reticulum (ER), CPXV203 blocks MHC I trafficking to the cell surface by exploiting the KDEL-receptor recycling pathway. CPXV018 encodes a soluble NKG2D ligand known as the orthopoxvirus major histocompatibility complex (MHC) class I-like protein (OMCP), which can block NKG2D-mediated cytotoxicity.

Recombinant viruses expressing a CRASP-2 gene product (UniProtKB-050665) and the Cowpox virus Open Reading Frames (ORFs) 012, 203 and 018 (CPXV012-203-018) have been generated herein in various embodiments. Specifically, in some embodiments, the recombinant virus, e.g., recombinant oncolytic virus, comprises an expression cassette containing a CRASP-2 cDNA fused to the F14.5L gene locus under the control of vaccinia F14.5L gene promoter and a contiguous polynucleotide sequence including Open Reading Frames (ORFs) 012, 203 and 018 with their own promoters (CPXV012-203-018) inserted into the J2R gene locus.

In some embodiments, the stealth protein includes Borrelia burgdorferi complement regulatory-acquiring surface protein-2 (CRASP-2). In some embodiments, expression of CRASP-2 protein can increase the ability of the recombinant virus to evade the host's complement. In some embodiments, a recombinant virus is provided which includes a polynucleotide that encodes a CRASP-2 molecule including CRASP-2 cDNA fused to the F14.5L gene locus, wherein the CRASP-2 polypeptide comprises an amino acid sequence having at least 90% sequence identity to SEQ ID NO: 18. In some embodiments, the stealth protein comprises Cowpox virus Open Reading Frames (ORFs) 012, 203 and 018 with their own promoters (CPXV012-203-018). In some embodiments, expression of the Cowpox virus Open Reading Frames (ORFs) 012, 203 and 018 (CPXV012-203-018) can increase the ability of the recombinant virus to evade the host's T and NK cells. In some embodiments, expression of stealth proteins including CRASP-2 and Cowpox virus Open Reading Frames (ORFs) 012, 203 and 018 can increase the ability of the recombinant virus to evade the host's complement and T and NK cells.

In some embodiments, the T cell evader or NK cell evader is a set of proteins encoded by Cowpox virus ORFs 012, 203 and 018 (CPXV012-203-018) comprising the amino acid sequences of SEQ ID NOs: 20, 21, and 22, or amino acid sequences having at least 70%, 80%, 85%, 90%, or 95% sequence identity to the amino acid sequences of SEQ ID NOs: 20, 21, and 22. In some embodiments, the recombinant virus comprises a polypeptide that encodes for CPXV012 with a sequence of amino acids that has at least 70%, such as at least 75%, 80%, 85%, or 90% sequence identity to the amino acid sequence of SEQ ID NO: 20. For example, in some embodiments, the recombinant virus comprises a sequence of amino acids that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 20, but is less than 100% identical to the amino acid sequence of SEQ ID NO: 20. In some embodiments, the recombinant virus comprises the amino acid sequence of SEQ ID NO: 20. In some embodiments, the recombinant virus comprises a polypeptide that encodes for CPXV203 with a sequence of amino acids that has at least 70%, such as at least 75%, 80%, 85%, or 90% sequence identity to the amino acid sequence of SEQ ID NO: 21. For example, in some embodiments, the recombinant virus comprises a sequence of amino acids that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 21, but is less than 100% identical to the amino acid sequence of SEQ ID NO: 21. In some embodiments, the recombinant virus comprises the amino acid sequence of SEQ ID NO: 21. In some embodiments, the recombinant virus comprises a polypeptide sequence that encodes for CPXV018 with a sequence of amino acids that has at least 70%, such as at least 75%, 80%, 85%, or 90% sequence identity to the amino acid sequence of SEQ ID NO: 22. For example, in some embodiments, the recombinant virus comprises a sequence of amino acids that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 22, but is less than 100% identical to the amino acid sequence of SEQ ID NO: 22. In some embodiments, the recombinant virus comprises the amino acid sequence of SEQ ID NO: 22.

In some embodiments, the T cell evader or NK cell evader is a set of proteins encoded by Cowpox virus ORFs 012, 203 and 018 (CPXV012-203-018) and the set of proteins encoded by CPXV012-203-018 comprise a sequence of amino acids that exhibits at least 85%, 90% or 95% sequence identity to the sequence set forth in SEQ ID NO: 20 (CPXV012), a sequence of amino acids that exhibits at least 85%, 90% or 95% sequence identity to the sequence set forth in SEQ ID NO: 21 (CPXV0203), and a sequence of amino acids that exhibits at least 85%, 90% or 95% sequence identity to the sequence set forth in SEQ ID NO: 22 (CPXV018). In some embodiments, the set of proteins encoded by CPXV012-203-018 comprise the sequence of amino acids set forth in SEQ ID NO: 20, SEQ ID NO: 21, and SEQ ID NO: 22.

In some embodiments, the recombinant virus comprises a polypeptide that encodes for CRASP-2 with a sequence of amino acids that has at least 70%, such as at least 75%, 80%, 85% or 90% sequence identity to SEQ ID NO: 18. For example, in some embodiments, the recombinant virus comprises a nucleic acid sequence that encodes a polypeptide having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 18, but is less than 100% identical to the amino acid sequence of SEQ ID NO: 18. In some embodiments, the recombinant virus comprises a nucleic acid sequence that encodes the amino acid sequence of SEQ ID NO: 18. In some embodiments, the polynucleotide that encodes the CRASP-2 molecule is operably linked to the F14.5L gene promoter. In some embodiments, the nucleotide sequences encoding CPXV012, CPXV203, and CPXV018 are inserted in the J2R genomic region.

In some embodiments, the recombinant virus comprising nucleic acid sequences that encode for CRASP-2 (e.g., SEQ ID NO: 18), CPXV012 (e.g., SEQ ID NO: 20), CPXV203 (e.g., SEQ ID NO: 21), and CPXV018 (e.g., SEQ ID NO: 22) is derived from the VIR27 (comprising the nucleic acid sequence of SEQ ID NO: 5) strain and comprises a sequence of nucleotides that has at least 70%, such as at least 75%, 80%, 85% or 90% sequence identity to SEQ ID NO: 10 (also named VIR46). For example, in some embodiments, the recombinant virus comprises a nucleic acid sequence that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 10, but is less than 100% identical to the nucleic acid sequence of SEQ ID NO: 10. In some embodiments, the recombinant virus comprises the nucleic acid sequence of SEQ ID NO: 10. A recombinant oncolytic virus comprising the nucleic acid sequence of SEQ ID NO: 10 is also referred to herein as VIR46.

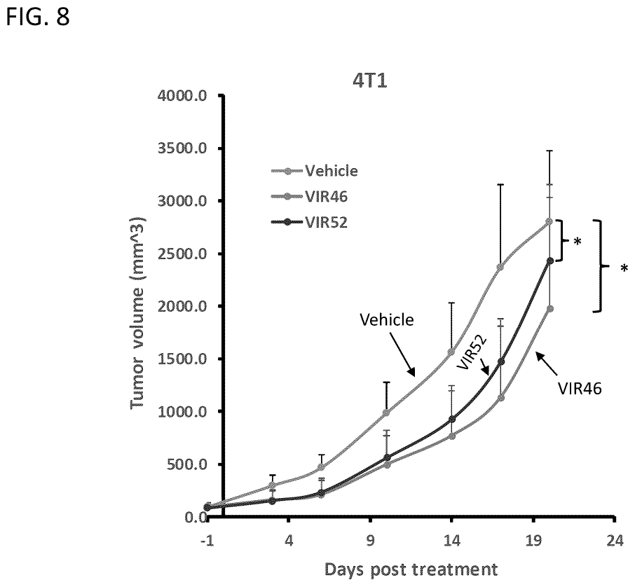

Recombinant viruses provided herein, in various embodiments, exhibit an increased ability to evade the host's complement. In some embodiments, recombinant viruses provided herein can escape complement inhibition in in vivo and in vitro systems. In particular embodiments, administration of an effective dose of VIR46 (comprising the nucleic acid sequence of SEQ ID NO: 10) to a subject inhibits tumor, hyperplasia, or metastasis growth in an in vivo model (FIG. 8).

### B. Immune Modulating Viruses

Provided herein, in various embodiments, are recombinant viruses comprising heterologous nucleic acids encoding for immune modulating proteins that can be stably and efficiently expressed in many types of virus infected cells. In some embodiments, the immune modulating proteins include cytokines, chemokines, immune receptors, antigens to immune receptors, proteins in immune cell activation pathways, signaling proteins within immune cells which stimulate the immune cell activation or secretion of cytokines from the immune cell, and antigens. In some embodiments, the immune modulating proteins comprise one or more cytokines and/or chemokines. In some embodiments, the one or more cytokines and/or chemokines comprises one or more of chemokine ligand 9 (CXCL9), IL-2, and IL-12. In some embodiments, the immune modulating protein is tumor necrosis factor superfamily member 14 (LIGHT). In some embodiments, the immune modulating protein is an interferon regulatory factor that activates the Toll-like receptor 3 (TLR3)-interferon regulatory factor 3 (IRF3) signaling pathway. In some embodiments, the immune modulating protein is interleukin 12 (IL-12). In some embodiments, the immune modulating protein is chemokine ligand 9 (CXCL9). In some embodiments, the immune modulating protein is IL-2 or an IL-2 superkine. In some embodiments, the immune modulating protein is an interleukin 2 (IL-2) superkine. In some embodiments, the immune modulating protein is MDNA11. In some embodiments, the MDNA11 has been mutated to increase the recombinant viruses anti-tumor potency and the immune modulator protein is MDNA11T. In some embodiments, the recombinant virus comprises heterologous nucleic acids encoding for one or more of the following immune modulating proteins; LIGHT, IRF3, IL-12, CXCL9, MDNA11, MDNA11T and other immune modulatory proteins. In some embodiments, one or more immune modulating proteins is an immune stimulating protein, such as LIGHT.

In some embodiments, the at least one heterologous nucleic acid encoding one or more heterologous gene product comprises one or more heterologous nucleic acid each encoding one or more immune modulating proteins. In some embodiments, the one or more immune modulating proteins is or comprises one or more cytokines and/or chemokines. In some embodiments, the one or more immune modulating proteins is or comprises one or more interferon regulatory factors, such as IRF3. In some embodiments, the one or more immune modulating proteins comprises one or more immune modulating proteins selected from the group consisting of LIGHT, IRF3, IL-2, IL-12, and CXCL9, such as any of those discussed in further detail below.

### 1. Tumor Necrosis Factor Superfamily Member 14 (LIGHT)

In some embodiments, the at least one heterologous nucleic acid encoding one or more heterologous gene product comprises one or more heterologous nucleic acid each encoding one or more immune modulating proteins, wherein the one or more immune modulating proteins comprises LIGHT.

LIGHT has been in pre-clinical development for over a decade and has proven to be a promising approach for treating various types of cancer. LIGHT has been successfully used to clear established solid tumors as well as treat metastatic events. When expressed in tumors, LIGHT molecules cause significant changes in the tumor microenvironment that are primarily driven through vascular normalization and generation of tertiary lymphoid structures (TLS). Formation of such lymphoid structures can be induced by activation of lymphotoxin beta-receptor (LTBR/TNFRSF3), a receptor of the TNF superfamily that can be activated by LIGHT. See Schrama et al., Immunity 14:111-121 (2001); Tang et al., Cell. Mol. Immunol. 14:809-18 (2017). Presence of TLS in the tumor microenvironment typically correlates with immune infiltration and it is associated with better prognosis, suggesting that TLS are involved in anti-tumor immune responses. See Dieu-Nosjean et al., J. Clin. Oncol. 26:4410-17 (2008) and Weinstein and Storkus, Adv. Cancer Res. 128:197-233 (2015). For example, an homotrimeric single-chain LIGHT variant with improved stability and with human and mouse cross-reactivity, termed 3×hmLIGHT fused to an EGFR-specific tumor targeting antibody, induced anti-tumor immunity in mouse and human tumor models by increasing lymphocyte infiltration. Therefore, activation of LTBR has the potential to promote TLS formation in the tumor microenvironment, induce anti-tumor immune responses, and improve current cancer immunotherapies. Further details about the anti-tumorigenic properties of LIGHT can be found in U.S. Publication NO. 2021/0188990, which is hereby incorporated by reference for all purposes.

Provided herein, in some embodiments, is a recombinant oncolytic virus comprising at least one heterologous nucleic acid encoding one or more heterologous gene product, wherein the one or more heterologous gene product is or comprises an immune stimulating protein.

In some embodiments, the recombinant virus comprises at least one heterologous nucleic acid encoding for immune stimulating proteins that can be stably and efficiently expressed in many types of virus infected cells. Such immune stimulating proteins can increase the anti-tumorigenic properties of the virus. In particular, such immune stimulating proteins can increase the ability of the recombinant virus to induce tumor lymphocyte infiltration by the host's immune system. In some embodiments, the recombinant virus includes oncolytic virus. In some embodiments, the recombinant virus does not include oncolytic viruses. In some embodiments, the recombinant virus incudes any viruses described herein or incorporated by reference herein. In some embodiments, the virus includes vaccinia viruses. In some embodiments, the virus includes recombinant viruses derived from the VIP02 viral strain.

In some embodiments, the recombinant virus comprises at least one heterologous nucleic acid encoding for immune stimulating proteins that can be stably and efficiently expressed in many types of virus-infected cells and increase the anti-tumorigenic properties of the virus. Accordingly, in some embodiments, the immune stimulating protein increases or enhances the anti-tumorigenic properties of the virus. In some embodiments, the immune modulating proteins include, but are not limited to, tumor necrosis factor superfamily member 14 (LIGHT), e.g., hmLIGHT. In some embodiments, the immune modulating proteins include, but are not limited to, any immune modulating protein hereby included by reference.

In some embodiments, the recombinant virus expresses an hmLIGHT gene product (NP_001363816 XP_016882906). Specifically, the generated recombinant virus of some embodiments comprises an expression cassette containing a full length hmLIGHT cDNA under the control of vaccinia PSE gene promoter and inserted into the A35R genomic region. In some embodiments, the immune modulating protein includes hmLIGHT. In some embodiments, the hmLIGHT protein can increase the anti-tumor properties of the the recombinant virus.

In some embodiments, the immune stimulating protein is LIGHT. In some embodiments, the immune stimulating protein is recombinant LIGHT. In some embodiments, the recombinant LIGHT is a human protein or is a mutant thereof.

In some embodiments, the recombinant LIGHT comprises an amino acid sequence that has at least 95%, at least 96%, at least 97%, at least 98% or at least 99% sequence identity to the sequence set forth in SEQ ID NO: 30. In some embodiments, the recombinant LIGHT comprises the amino acid sequence of SEQ ID NO: 30. In some embodiments, the recombinant LIGHT comprises the amino acid sequence of SEQ ID NO: 30, or an amino acid sequence having at least 90%, 91%, 92%, 93%, 94%, 95%, 96%, 97%, 98%, or 99% sequence identity to the amino acid sequence of SEQ ID NO: 30.

In some embodiments, the recombinant LIGHT is hmLIGHT that is a human LIGHT mutant that binds human and mouse LTBR and HVEM. In some embodiments, the recombinant LIGHT comprises one or more mutation selected from the group consisting of a threonine at position 138, a glycine at position 160, a glycine at position 221 and a lysine at position 222.

In some embodiments, the recombinant virus comprises a nucleic acid sequence that encodes an hmLIGHT, wherein the hmLIGHT comprises an amino acid sequence having at least 90% sequence identity to SEQ ID NO: 25. In some embodiments, the recombinant LIGHT comprises the amino acid sequence of SEQ ID NO: 25, or an amino acid sequence having at least 90%, 91%, 92%, 93%, 94%, 95%, 96%, 97%, 98%, or 99% sequence identity to the amino acid sequence of SEQ ID NO: 25. In some embodiments, the recombinant LIGHT comprises an amino acid sequence that has at least 95%, at least 96%, at least 97%, at least 98% or at least 99% sequence identity to the sequence set forth in SEQ ID NO: 25. In some embodiments, the recombinant LIGHT comprises the sequence set forth in SEQ ID NO: 25.

In some embodiments, the recombinant virus comprises a polypeptide with a sequence of amino acids that has at least 70%, such as at least 75%, 80%, 85% or 90% sequence identity to the amino acid sequence of SEQ ID NO: 25.

In some embodiments, the polynucleotide that encodes the hmLIGHT molecule is operably linked to the PSE gene promoter. In some embodiments, the recombinant virus which includes a polynucleotide that encodes an hmLIGHT gene product (e.g., comprising the amino acid sequence of SEQ ID NO: 25) is derived from the VIR13 (comprising the nucleic acid sequence of SEQ ID NO: 4) strain and includes a sequence of nucleotides that has at least 70%, such as at least 75%, 80%, 85% or 90% sequence identity to SEQ ID NO: 11 (also named VIR49). For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 11, but is less than 100% identical to SEQ ID NO: 11. In some embodiments, the recombinant virus comprises the nucleic acid sequence of SEQ ID NO: 11. A recombinant oncolytic virus comprising the nucleic acid sequence of SEQ ID NO: 11 is also referred to herein as VIR49.

In some embodiments, the recombinant virus provided herein, e.g., VIR49 (comprising the nucleic acid sequence of SEQ ID NO: 11) or a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 11, exhibits increased or enhanced anti-tumorigenic properties. In particular embodiments, administration of an effective dose of VIR49 (comprising the nucleic acid sequence of SEQ ID NO: 11) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 11 to a subject inhibits tumor, hyperplasia, and/or metastasis growth.

In some embodiments, provided herein is a recombinant oncolytic virus comprising at least one heterologous nucleic acid encoding one or more heterologous gene product, wherein the one or more heterologous gene product is or comprises an interferon regulatory factor. As described herein, the clinical usefulness of oncolytic vaccinia viruses (VACVs) is not only related to the virus ability to directly destruct infected cancer cells but also to activate immune responses directed against tumor antigens. One way to elicit a robust antitumor immune response is through activation of the Toll-like receptor 3 (TLR3)-interferon regulatory factor 3 (IRF3) signaling pathway. Thus, virus expressing IRF3 can increase the ability of the virus to activate cellular antitumor immunity. Riederer at al., (2021) Molecular Therapy: Oncolytics, 22: (399-409).

In some embodiments, the recombinant virus comprises one or more heterologous nucleic acids encoding for one or more immune stimulating proteins, e.g., one or more interferon regulatory factor, that can be stably and efficiently expressed in many types of virus-infected cells and increase the anti-tumorigenic properties of the virus. In some embodiments, the immune modulating proteins, e.g., interferon regulatory factors, include, but are not limited to, interferon regulatory factor 3 (IRF3). As such, in some embodiments, the one or more heterologous gene product comprises an interferon regulatory factor. In some embodiments, the interferon regulatory factor is an interferon regulatory factor 3 (IRF3). In some embodiments, the IRF3 is human IRF3. In some embodiments, the IRF3 is a human IRF3 (hIRF3) and the hIRF3 comprises a sequence of amino acids that has at least 95%, at least 96%, at least 97%, at least 98% or at least 99% sequence identity to the amino acid sequence set forth in SEQ ID NO: 51. In some embodiments, the hIRF3 comprises the amino acid sequence of SEQ ID NO: 51.

### 2. Interferon Regulatory Factor 3 (IRF3)

In some embodiments, the at least one heterologous nucleic acid encoding one or more heterologous gene product comprises one or more heterologous nucleic acid each encoding one or more immune modulating proteins, wherein the one or more immune modulating proteins comprises interferon regulatory factor 3 (IRF3).

In some embodiments, the interferon regulatory factor is mouse IRF3. In some embodiments, the recombinant virus comprises an expression cassette containing a full-length mouse IRF3 cDNA under the control of a vaccinia gene promoter and inserted into the J2R genomic region. In some embodiments, the interferon regulatory factor is human IRF3. In some embodiments, the mouse IRF3 protein can increase the anti-tumor properties of the recombinant virus. In some embodiments, a recombinant virus is provided which comprises a polynucleotide that encodes a mouse IRF3 molecule, wherein the IRF3 polypeptide comprises an amino acid sequence having at least 90% sequence identity to SEQ ID NO: 52. In some embodiments, the IRF3 is a mouse IRF3 (mIRF3), and the mIRF3 comprises a sequence of amino acids that has at least 95%, at least 96%, at least 97%, at least 98% or at least 99% sequence identity to the sequence set forth in SEQ ID NO: 52. In some embodiments, the mIRF3 comprises the amino acid sequence of SEQ ID NO: 52.

In some embodiments, the recombinant virus comprises a polypeptide that encodes a sequence of amino acids that has at least 70%, such as at least 75%, 80%, 85% or 90% sequence identity to SEQ ID NO: 52. For example, in some embodiments, the recombinant virus comprises a sequence of nucleotides that encodes a polypeptide having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 52, but is less than 100% identical to SEQ ID NO: 52. In some embodiments, the recombinant virus comprises a sequence of nucleotides that encodes a polypeptide comprising the amino acid sequence of SEQ ID NO: 52.

In some embodiments, the polynucleotide that encodes the IFR3 molecule is operably linked to a gene promoter. In some embodiments, the recombinant virus which includes a polynucleotide that encodes a mIRF3 gene product (e.g., comprising the amino acid sequence of SEQ ID NO: 52) is derived from the VIP02 (comprising the nucleic acid sequence of SEQ ID NO: 1) strain.

In some embodiments, the interferon regulatory factor is mIRF3 and the recombinant virus, e.g., recombinant oncolytic virus, comprises a sequence of nucleotides having at least 95%, 96%, 97%, 98%, or 99% sequence identity to the nucleic acid sequence of SEQ ID NO: 50. In some embodiments, the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 50. In some embodiments, the recombinant virus, e.g., recombinant oncolytic virus, comprises the sequence of nucleotides of SEQ ID NO: 50. A recombinant oncolytic virus comprising the nucleic acid sequence of SEQ ID NO: 50 is also referred to herein as VIR96.

In some embodiments, the generated recombinant viruses include an expression cassette containing a full length human IRF3 cDNA under the control of a vaccinia gene promoter and inserted into the J2R genomic region. In some embodiments, the immune modulating protein includes human IRF3. In some embodiments, the human IRF3 protein can increase the anti-tumor properties of the recombinant virus. In some embodiments, a recombinant virus is provided which includes a polynucleotide that encodes a human IRF3 molecule, wherein the IRF3 polypeptide comprises an amino acid sequence having at least 90%, 91%, 92%, 93%, 94%, 95%, 96%, 97%, 98%, or 99% sequence identity to SEQ ID NO: 51.

In some embodiments, the recombinant virus comprises a polypeptide with a sequence of amino acids that has at least 70%, such as at least 75%, 80%, 85% or 90% sequence identity to the amino acid sequence of SEQ ID NO: 51.

In some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NOs: 49, 80, 81, 82, 84, 85, 86, 88, 87, 89, 90, 91, 92, or 93, but is less than 100% identical to SEQ ID NOs: 49, 80, 81, 82, 84, 85, 86, 87, 88, 89, 90, 91, 92, or 93. In some embodiments, the recombinant virus comprises the nucleic acid sequence of SEQ ID NO: 49, 80, 81, 82, 84, 85, 86, 87, 88, 89, 90, 91, 92, or 93. A recombinant oncolytic virus comprising the nucleic acid sequence of SEQ ID NOs: 49, 80, 81, 82, 84, 85, 86, 87, 88, 89, 90, 91, 92, or 93 is also referred to herein as VIR93, VIR100, VIR102, VIR103, VIR105, VIR106, VIR109, VIR111, VIR113, VIR114, VIR115, VIR123, VIR127, or VIR128 respectively.

Recombinant virus provided herein exhibit increase anti-tumorigenic properties. In particular embodiments, administration of an effective dose of VIR93 (comprising the nucleic acid sequence of SEQ ID NO: 49) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 49 to a subject inhibits tumor, hyperplasia, and/or metastasis growth.

### 3. Interleukin 12 (IL-12)

In some embodiments, the at least one heterologous nucleic acid encoding one or more heterologous gene product comprises one or more heterologous nucleic acid each encoding one or more immune modulating proteins, wherein the one or more immune modulating proteins comprises IL-12.

Interleukin 12 (IL-12) is a cytokine that play a role in shaping the innate and adaptive immune responses and may play a role in controlling cancer and tumor growth. In some embodiments, the IL-12 is human IL-12. In some embodiments, the IL-12 is mouse IL-12. In some embodiments, the recombinant virus comprises an expression cassette containing a full-length mouse IL-12 cDNA under the control of a vaccinia gene promoter and inserted into the A56R genomic region. In some embodiments, the mouse IL-12 protein can increase the anti-tumor properties of the recombinant virus. In some embodiments, a recombinant virus is provided which comprises a polynucleotide that encodes a mouse single chain IL-12 (mscIL-12) molecule, wherein the recombinant virus comprises an nucleic acid sequence having at least 90% sequence identity to SEQ ID NOs: 85, 86, or 90.

In some embodiments, the IL-12 is a single-chain IL-12. In some embodiments, the single-chain IL-12 is a human single-chain IL-12 (hscIL-12). In some embodiments, the human single-chain IL-12 comprises the amino acid sequence set forth in SEQ ID NO: 103, or an amino acid sequence that has at least 95%, 96%, 97%, 98%, or 99% sequence identity to the amino acid sequence set forth in SEQ ID NO: 103. In some embodiments, the IL-12, e.g., human IL-12, comprises the amino acid sequence set forth in SEQ ID NO: 103, or an amino acid sequence that has at least 95%, 96%, 97%, 98%, or 99% sequence identity to the amino acid sequence set forth in SEQ ID NO: 103.

In some embodiments, the single-chain IL-12 is a mouse single-chain IL-12 (mscIL-12). In some embodiments, the mouse single-chain IL-12 comprises the amino acid sequence set forth in SEQ ID NO: 102, or an amino acid sequence that has at least 95%, 96%, 97%, 98%, or 99% sequence identity to the amino acid sequence set forth in SEQ ID NO: 102. In some embodiments, the IL-12, e.g., mouse IL-12, comprises the amino acid sequence set forth in SEQ ID NO: 102, or an amino acid sequence that has at least 95%, 96%, 97%, 98%, or 99% sequence identity to the amino acid sequence set forth in SEQ ID NO: 102.

In some embodiments, the recombinant virus comprises a nucleic acid sequence that that has at least 70%, such as at least 75%, 80%, 85% or 90% sequence identity to SEQ ID NOs: 85, 86, or 90. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NOs: 85, 86, or 90, but is less than 100% identical to SEQ ID NOs: 85, 86, or 90. In some embodiments, the recombinant virus comprises a sequence of nucleotides comprising the sequences of SEQ ID NOs: 85, 86, or 90. In some embodiments, the recombinant viruses comprising mscIL-12 are referred to as VIR106, VIR109, and/or VIR115.

In some embodiments, the polynucleotide that encodes the mscIL-12 molecule is operably linked to a gene promoter. In some embodiments, the recombinant virus which includes a polynucleotide that encodes a mscIL-12gene product is derived from the VIP02 (comprising the nucleic acid sequence of SEQ ID NO: 1) strain. In some embodiments the mscIL-12 is inserted at A56R genomic region. In some embodiments, the mscIL-12 is operably linked to modified H5 (mH5) promoter.

In some embodiments, a recombinant virus is provided which comprises a polynucleotide that encodes a human single chain IL-12 (hscIL-12) molecule, wherein the recombinant virus comprises an nucleic acid sequence having at least 90% sequence identity to SEQ ID NO: 92.

In some embodiments, the recombinant virus comprises a nucleic acid sequence that that has at least 70%, such as at least 75%, 80%, 85% or 90% sequence identity to SEQ ID NO: 92. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 92 but is less than 100% identical to SEQ ID NO: 92. In some embodiments, the recombinant virus comprises a sequence of nucleotides comprising the sequences of SEQ ID NO: 92. In some embodiments, the recombinant virus comprising hscIL-12 is referred to as VIR 115.

In some embodiments, the polynucleotide that encodes the hscIL-12 molecule is operably linked to a gene promoter. In some embodiments, the recombinant virus which includes a polynucleotide that encodes a hscIL-12gene product is derived from the VIP02 (comprising the nucleic acid sequence of SEQ ID NO: 1) strain. In some embodiments the hscIL-12 is inserted at A56R genomic region. In some embodiments, the hscIL-12 is operably linked to modified H5 (mH5) promoter.

In some embodiments, the recombinant viruses comprise hscIL-12 and/or mscIL-12 and other immune modulatory genes. In some embodiments, the other immune modulatory genes may comprise any of the immune modulating genes in this application. In some embodiments, the other immune modulating genes may comprise other cytokines or chemokines or antigens.

In some embodiments recombinant virus provided herein exhibit increase anti-tumorigenic properties. In particular embodiments, administration of an effective dose of VIR106, VIR109, VIR115, and/or VIR127 (comprising the nucleic acid sequence of SEQ ID NOs: 85, 86, 90, and 92 respectively) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequences of SEQ ID NOs: 85, 86, 90, and 92 to a subject inhibits tumor, hyperplasia, and/or metastasis growth.

### 4. Chemokine Ligand 9 (CXCL9)

In some embodiments, the at least one heterologous nucleic acid encoding one or more heterologous gene product comprises one or more heterologous nucleic acid each encoding one or more immune modulating proteins, wherein the one or more immune modulating proteins comprises CXCL9.

Chemokine ligand 9 (CXCL9) is a chemokine capable of orchestrating cell migration and may be important for shaping the tumor microenvironment. In some embodiments, the CXCL9 is human CXCL9. In some embodiments, the CXCL9 is mouse CXCL9. In some embodiments, the recombinant virus comprises an expression cassette containing a full-length mouse CXCL9 cDNA under the control of a vaccinia gene promoter and inserted into the A56R genomic region. In some embodiments, the mouse CXCL9 protein can increase the anti-tumor properties of the recombinant virus. In some embodiments, a recombinant virus is provided which comprises a polynucleotide that encodes a mouse CXCL9 (mCXCL9) molecule, wherein the recombinant virus comprises an nucleic acid sequence having at least 90% sequence identity to SEQ ID NOs: 85, 86, or 90.

In some embodiments, the CXCL9 is human CXCL9 (hCXCL9). In some embodiments, the human CXCL9 comprises the amino acid sequence set forth in SEQ ID NO: 99, or an amino acid sequence that has at least 85%, 96%, 97%, 98%, or 99% sequence identity to the amino acid sequence set forth in SEQ ID NO: 99.

In some embodiments, the CXCL9 is mouse CXCL9 (mCXCL9). In some embodiments, the mouse CXCL9 comprises the amino acid sequence set forth in SEQ ID NO: 106, or an amino acid sequence that has at least 85%, 96%, 97%, 98%, or 99% sequence identity to the amino acid sequence set forth in SEQ ID NO: 106.

In some embodiments, the recombinant virus comprises a nucleic acid sequence that that has at least 70%, such as at least 75%, 80%, 85% or 90% sequence identity to SEQ ID NOs: 85, 86, or 90. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NOs: 85, 86, or 90, but is less than 100% identical to SEQ ID NOs: 85, 86, or 90. In some embodiments, the recombinant virus comprises a sequence of nucleotides comprising the sequences of SEQ ID NOs: 85, 86, or 90. In some embodiments, the recombinant viruses comprising mCXCL9 are referred to as VIR106, VIR109, and/or VIR115.

In some embodiments, the polynucleotide that encodes the mCXCL9 molecule is operably linked to a gene promoter. In some embodiments, the recombinant virus which includes a polynucleotide that encodes a mCXCL9 gene product is derived from the VIP02 (comprising the nucleic acid sequence of SEQ ID NO: 1) strain. In some embodiments the mCXCL9 is inserted at A56R genomic region. In some embodiments, the mCXCL9 is operably linked to modified H5 (mH5) promoter.

In some embodiments, a recombinant virus is provided which comprises a polynucleotide that encodes a human CXCL9 (hCXCL9) molecule, wherein the recombinant virus comprises a nucleic acid sequence having at least 90% sequence identity to SEQ ID NO: 92.

In some embodiments, the recombinant virus comprises a nucleic acid sequence that that has at least 70%, such as at least 75%, 80%, 85% or 90% sequence identity to SEQ ID NO: 92. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 92 but is less than 100% identical to SEQ ID NO: 92. In some embodiments, the recombinant virus comprises a sequence of nucleotides comprising the sequences of SEQ ID NO: 92. In some embodiments, the recombinant virus comprising hCXCL9 is referred to as VIR 115.

In some embodiments, the polynucleotide that encodes the hCXCL9 molecule is operably linked to a gene promoter. In some embodiments, the recombinant virus which includes a polynucleotide that encodes a hCXCL9 gene product is derived from the VIP02 (comprising the nucleic acid sequence of SEQ ID NO: 1) strain. In some embodiments the hCXCL9 is inserted at A56R genomic region. In some embodiments, the hCXCL9 is operably linked to modified H5 (mH5) promoter.

In some embodiments, the recombinant viruses comprise hCXCL9 and/or mCXCL9 and other immune modulatory genes. In some embodiments, the other immune modulatory genes may comprise any of the immune modulating genes in this application. In some embodiments, the other immune modulating genes may comprise other cytokines or chemokines or antigens.

In some embodiments recombinant virus provided herein exhibit increase anti-tumorigenic properties. In particular embodiments, administration of an effective dose of VIR106, VIR109, VIR115, and/or VIR127 (comprising the nucleic acid sequence of SEQ ID NOs: 85, 86, 90, and 92 respectively) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequences of SEQ ID NOs: 85, 86, 90, and 92 to a subject inhibits tumor, hyperplasia, and/or metastasis growth.

### 5. IL-2 and IL-2 Superkines

In some embodiments, the at least one heterologous nucleic acid encoding one or more heterologous gene product comprises one or more heterologous nucleic acid each encoding one or more immune modulating proteins, wherein the one or more immune modulating proteins comprises IL-2.

In some embodiments, the recombinant virus comprises one or more heterologous nucleic acid that encodes an immune modulatory protein that is IL-2. In some embodiments, the IL-2 comprises an amino acid sequence set forth in any one of SEQ ID NOs: 98, 100, 101, 104, and 105, or an amino acid sequence that has at least 95%, 96%, 97%, 98%, or 99% sequence identity to the amino acid sequence of any one of SEQ ID NOs: 98, 100, 101, 104, and 105.

In some embodiments, the IL-2 is an IL-2 superkine, such as H9, H9T, MDNA11, or MDNA11T. Accordingly, in some embodiments, the recombinant virus encodes an immune modulatory protein that is an IL-2 superkine. In some embodiments, the IL-2 superkine comprises the amino acid sequence of any one of SEQ ID NOs: 98, 100, 101, and 104, or an amino acid sequence that has at least 95%, 96%, 97%, 98%, or 99% sequence identity to the amino acid sequence of any one of SEQ ID NOS: 98, 100, 101, and 104.

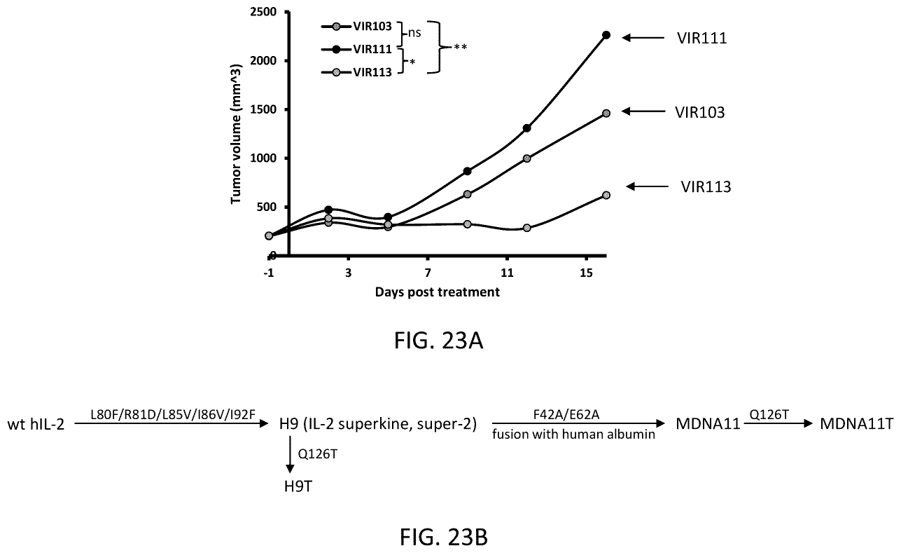

In some embodiments, the IL-2 superkine is the H9 IL-2 superkine. In some embodiments, the H9 IL-2 superkine comprises the amino acid sequence of SEQ ID NO: 100, or comprises an amino acid sequence that has at least 95%, 96%, 97%, 98%, or 99% sequence identity to the amino acid sequence of SEQ ID NO: 100. As depicted in FIG. 23B, the H9 IL-2 superkine is a modified variant of wild-type human IL-2 that was generated by introducing the following amino acid substitutions to wildtype human IL-2: L80F, R81D, L85V, 186V, and 192F. In some embodiments, the wild-type human IL-2 comprises the amino acid sequence set forth in SEQ ID NO: 105.

In some embodiments, the IL-2 superkine is the H9T IL-2 superkine. The H9T IL-2 superkine is generated by introducing a Q126T amino acid substitution into the H9 IL-2 superkine, as depicted in FIG. 23B. In some embodiments, the H9T IL-2 superkine comprises the amino acid sequence of SEQ ID NO: 104, or comprises an amino acid sequence that has at least 95%, 96%, 97%, 98%, or 99% sequence identity to the amino acid sequence of SEQ ID NO: 104.

In some embodiments, the immune modulatory protein, e.g., the IL-2 superkine, is MDNA11. See, e.g., Merchant et al., Journal for Immuno Therapy of Cancer, 2022; 10: e003155. In some embodiments, the immune modulatory protein is an IL-2 superkine that is the MDNA11 IL-2 superkine. In some embodiments, MDNA11 comprises the amino acid sequence of SEQ ID NO: 101, or comprises an amino acid sequence that has at least 95%, 96%, 97%, 98%, or 99% sequence identity to the amino acid sequence of SEQ ID NO: 101. As depicted in FIG. 23B, the MDNA11 IL-2 superkine is generated by introducing the following amino acid substitutions into the H9 IL-2 superkine: F42A and E62A.

In some embodiments, the immune modulatory protein, e.g., the IL-2 superkine, is MDNA11T. In some embodiments, the immune modulatory protein is an IL-2 superkine that is the MDNA11T IL-2 superkine. MDNA11T is generated by introducing a Q126T amino acid substitution into the amino acid sequence of MDNA11, as depicted in FIG. 23B. See, e.g., Mo et al., Nature, 2021, 597 (7877): 544-548. In some embodiments, MDNA11T comprises the amino acid sequence of SEQ ID NO: 98, or comprises an amino acid sequence that has at least 95%, 96%, 97%, 98%, or 99% sequence identity to the amino acid sequence of SEQ ID NO: 98. In some embodiments, the IL-2 superkine is H9, MDNA11, or MDNA11T.

In some embodiments, the recombinant virus comprises an expression cassette containing a full-length MDNA11 cDNA under the control of a vaccinia gene promoter and inserted into the A56R genomic region. In some embodiments, the mouse MDNA11 protein can increase the anti-tumor properties of the recombinant virus. In some embodiments, a recombinant virus is provided which comprises a polynucleotide that encodes a MDNA11 molecule, wherein the recombinant virus comprises an nucleic acid sequence having at least 90% sequence identity to SEQ ID NOs: 84, 87, or 89.

In some embodiments, the recombinant virus comprises a nucleic acid sequence that has at least 70%, such as at least 75%, 80%, 85% or 90% sequence identity to SEQ ID NOs: 84, 87, or 89. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NOs: 84, 87, or 89, but is less than 100% identical to SEQ ID NOs: 84, 87, or 89. In some embodiments, the recombinant virus comprises a sequence of nucleotides comprising the sequences of SEQ ID NOs: 84, 87, or 89. In some embodiments, the recombinant viruses comprising MDNA11 are referred to as VIR105, VIR111, and/or VIR114.

In some embodiments, the polynucleotide that encodes the MDNA11 molecule is operably linked to a gene promoter. In some embodiments, the recombinant virus which includes a polynucleotide that encodes a MDNA11 gene product is derived from the VIP02 (comprising the nucleic acid sequence of SEQ ID NO: 1) strain. In some embodiments the MDNA11 is inserted at A56R genomic region. In some embodiments, the MDNA11 is operably linked to an 11K promoter.

In some embodiments, a recombinant virus is provided which comprises a polynucleotide that encodes mutated MDNA11 molecule. In some embodiments, the mutated MDNA11 molecule is MDNA11T. In some embodiments, the mutation is at amino acid 126 and turns the amino acid into a threonine. In some embodiments, the MDNA11T comprises the amino acid sequence in SEQ ID NO: 98. In some embodiments, the MDNA11T comprises an amino acid sequence that has at least 70%, such as at least 75%, 80%, 85% or 90% sequence identity to SEQ ID NOs: 98. In some embodiments, a recombinant vaccinia virus encoding a mutated MDNA11 increases the anti-tumor potency of the recombinant vaccinia virus compared to a recombinant vaccinia virus encoding an unmutated MDNA11. In some embodiments the recombinant virus comprises a nucleic acid sequence having at least 90% sequence identity to SEQ ID NO: 88.

In some embodiments, the recombinant virus comprises a nucleic acid sequence that has at least 70%, such as at least 75%, 80%, 85% or 90% sequence identity to SEQ ID NO: 88. For example, in some embodiments, the recombinant virus comprises a sequence of least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 88 but is less than 100% identical to SEQ ID NO: 88. In some embodiments, the recombinant virus comprises a sequence of nucleotides comprising the sequences of SEQ ID NO: 88. In some embodiments, the recombinant virus comprising MDNA11T is referred to as VIR 113.

In some embodiments, the polynucleotide that encodes the MDNA11T molecule is operably linked to a gene promoter. In some embodiments, the recombinant virus which includes a polynucleotide that encodes a MDNA11T gene product is derived from the VIP02 (comprising the nucleic acid sequence of SEQ ID NO: 1) strain. In some embodiments the MDNA11T is inserted at A56R genomic region. In some embodiments, the MDNA11T is operably linked to an 11K promoter.

In some embodiments, the recombinant viruses comprise MDNA11T and/or MDNA11 and other immune modulatory genes. In some embodiments, the other immune modulatory genes may comprise any of the immune modulating genes in this application. In some embodiments, the other immune modulating genes may comprise other cytokines or chemokines or antigens.

In some embodiments recombinant virus provided herein exhibit increase anti-tumorigenic properties. In particular embodiments, administration of an effective dose of VIR105, VIR111, VIR114, and/or VIR113 (comprising the nucleic acid sequence of SEQ ID NOs: 84, 87, 89, and 88 respectively) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequences of SEQ ID NOs: 84, 87, 89, and 88 to a subject inhibits tumor, hyperplasia, and/or metastasis growth.

### C. Angiogenesis Modulating Viruses

In some embodiments, the at least one heterologous nucleic acid encoding one or more heterologous gene product comprises one or more heterologous nucleic acid each encoding one or more anti-angiogenic protein, such as a VEGF inhibitor, an angiopoietin inhibitor, and/or versikine.

Accordingly, provided herein, in various embodiments, are recombinant viruses comprising heterologous nucleic acids encoding for an angiogenesis modulating protein that can be stably and efficiently expressed in many types of virus infected cells. Angiogenesis, a multi-step process that leads to the formation and growth of new blood vessels, is tightly regulated by several pro- and anti-angiogenic factors. The balance between these pro and anti-angiogenic factors is usually altered by cancer cells and is an essential step during the progression and metastasis of a malignant tumor, as angiogenesis can provide the oxygen and nutrients required for tumor growth. The mechanism of tumor angiogenesis is complicated and thus, involves different signal pathways including many pro- and anti-angiogenic factors such as Fibroblast Growth Factor (FGF), trombospondins, angiostatin, and endostatin, among others. Further details about anti-angiogenic and pro-angiogenic factors, can be found in Huang and Bao, World J Gastroenterol. 10(4): 463-470 (2004), which is hereby incorporated by reference for all purposes.

One of the many pro-angiogenic factors, Vascular Endothelial Growth Factor (VEGF), is secreted by tumor cells and surrounding stroma, stimulating the proliferation and survival of endothelial cells and leading to the formation of new blood vessels that support tumor and metastasis development and growth. See Rivera-Soto and Damania, Front. Microbiol. 10:1544 (2019). Another pro-angiogenic factor, Angiopoietin-2 (Ang-2), is a key regulator of blood vessel remodeling and maturation, usually up regulated in tumors and an unfavorable prognostic factor. Thus, many strategies to inhibit VEGF and Ang-2 have been attempted as part of cancer treatment protocols, including the generation of antibodies against VEGF and Ang-2. Further details about strategies to inhibit VEGF and Ang-2, can be found in Scheuer et al., MAbs, 8(3): 562-573 (2016), which is hereby incorporated by reference for all purposes.

Progression of inflammatory diseases such as cancer is closely related to the composition of the extracellular matrix surrounding the tumor. One extracellular matrix protein common to many inflammatory diseases is Versican. Papadas et al., Journal of Histochemistry & Cytochemistry 68(12) 871-885 (2020). In addition to the various isoforms of Versican, products from its proteolytic cleavage within the tumor microenvironment termed Versikines are implicated in generating anti-tumor immunity, for example, by promoting tumor cell apoptosis in cancer. In addition, Versican proteolysis can also drive new blood vessel formation (angiogenesis). Hirani et al., Front. Oncol. 11: 712807(2021). Various modifications of Versikine, including point mutations, insertions, deletions and substitutions, and fusions of Versikine to various polypeptides that can affect Versikine anti-tumorigenic abilities have been described and are included herein. Further details about Versikine modifications and fusions, can be found in U.S. Patent Application No. 2017/0258898, which is hereby incorporated by reference for all purposes.

Accordingly, provided herein, in some embodiments, is a recombinant oncolytic virus comprising at least one heterologous nucleic acid encoding one or more heterologous gene product, wherein the one or more heterologous gene product is or comprises one or more anti-angiogenic protein.

In some embodiments, the one or more heterologous nucleic acids encode for anti-angiogenic proteins that can be stably and efficiently expressed in many types of virus infected cells. Such anti-angiogenic proteins can inhibit the host's angiogenic pathways that favor tumor growth and development. In particular, the recombinant viruses including anti-angiogenic proteins provided herein can increase the ability of the recombinant virus to inhibit tumor angiogenesis and reduced tumor and metastasis growth in a host. In some embodiments, the recombinant viruses include oncolytic viruses. In some embodiments, the recombinant viruses do not include oncolytic viruses. In some embodiments, the viruses include vaccinia viruses. In some embodiments, the recombinant viruses include VIP02 derived recombinant viruses. In some embodiments, the recombinant viruses include any viruses described herein or incorporated by reference herein.

In some embodiments, the one or more anti-angiogenic protein is a VEGF inhibitor, an angiopoietin inhibitor, versikine or a fusion protein of any two or more of the foregoing. In some embodiments, the one or more anti-angiogenic protein comprises a VEGF inhibitor and/or an Angiopoietin-2 (Ang2) inhibitor. In some embodiments, the anti-angiogenic proteins include, but are not limited to, an anti-VEGF single chain antibody (anti-VEGF scAb), an anti-Angiopoietin-2 single chain antibody (anti-Ang2 scAb), and/or Versikine. In some embodiments, the VEGF inhibitor is an anti-VEGF antibody, optionally an anti-VEGF-single chain antibody (scAb). In some embodiments, the VEGF inhibitor is an anti-VEGF-single chain antibody (scAb). In some embodiments, the angiopoietin inhibitor is an anti-Angriopoietin-2 (Ang2) antibody, optionally an anti-Ang2 single chain antibody (scAb). In some embodiments, the angiopoietin inhibitor is an anti-Ang2 single chain antibody (scAb). In some embodiments, the anti-angiogenic protein includes any of the proteins and/or factors included herein or incorporated hereby by reference. See Huang and Bao., World J Gastroenterol. 10(4): 463-470 (2004).

In some embodiments, the recombinant viruses including heterologous nucleic acids encoding for anti-angiogenic proteins encode for an anti-VEGF scAb and anti-Ang2 scAb. In some embodiments, the one or more anti-angiogenic protein is a bispecific anti-VEGF/anti-Ang2 antibody. In some embodiments, the bispecific anti-VEGF/anti-Ang2 antibody comprises a sequence of amino acids that has at least 95%, at least 96%, at least 97%, at least 98% or at least 99% sequence identity to the sequence set forth in SEQ ID NO: 23. In some embodiments, the bispecific anti-VEGF/anti-Ang2 antibody comprises the sequence set forth in SEQ ID NO: 23.

In some embodiments, the one or more anti-angiogenic protein further comprises versikine. In some embodiments, the versikine is human versikine. In some embodiments, the versikine has a sequence of amino acids that has at least 95%, at least 96%, at least 97%, at least 98% or at least 99% sequence identity to the sequence set forth in SEQ ID NO: 24. In some embodiments, the versikine has the sequence of amino acids set forth in SEQ ID NO: 24.

In some embodiments, the recombinant viruses comprise heterologous nucleic acids encoding for anti-angiogenic proteins, such as an anti-VEGF scAb, an anti-Ang2 scAb, and/or Versikine. In some embodiments, the recombinant viruses including heterologous nucleic acids encoding for anti-angiogenic proteins encode for an anti-VEGF scAb. In some embodiments, the recombinant viruses including heterologous nucleic acids encoding for anti-angiogenic proteins encode for an anti-Ang2 scAb. In some embodiments, the recombinant viruses including heterologous nucleic acids encoding for anti-angiogenic proteins encode for Versikine. In some embodiments, the recombinant viruses including heterologous nucleic acids encoding for anti-angiogenic proteins encode for an anti-Ang2 scAb and Versikine. In some embodiments, the recombinant viruses including heterologous nucleic acids encoding for anti-angiogenic proteins encode for an anti-VEGF scAb and Versikine.

In some embodiments, the recombinant virus expresses an anti-VEGF single scAb gene product, an anti-Ang2 gene product, and a Versikine gene product (NP_001157569). Specifically, in some embodiments, the recombinant virus comprises an expression cassette containing an anti-VEGF scAb gene product and an anti-Ang2 scAb gene product cDNA under the control of vaccinia modified H5 (mH5) gene promoter, and a cDNA including the Versikine gene product under the control of vaccinia PSEL promoter. The full cassette including the cDNA including an anti-VEGF scAb gene product, an anti-Ang2 gene product, and a Versikine gene product is inserted into vaccinia J2R genomic region. In addition, the recombinant viruses expressing an anti-VEGF scAb gene product, an anti-Ang2 scAb gene product and a Versikine gene product include a deletion of the A35R genomic region.

In some embodiments, the anti-angiogenic protein includes an anti-VEGF-anti-Ang2 scAb fusion single chain antibody polypeptide and a Versikine polypeptide. In some embodiments, the anti-VEGF-anti-Ang2 scAb polypeptide can increase the ability of the recombinant virus to inhibit angiogenesis. In some embodiments, the Versikine polypeptide can increase the ability of the recombinant virus to inhibit angiogenesis. In some embodiments, the Versikine polypeptide can enhance the anti-tumorigenic and/or anti-angiogenic properties of the anti-VEGF-anti-Ang2 scAb polypeptide. In some embodiments, a recombinant virus is provided which includes a polynucleotide that encodes an anti-VEGF-anti-Ang2 scAb molecule, wherein the anti-VEGF-anti-Ang2 scAb molecule comprises an amino acid sequence having at least 90% sequence identity to SEQ ID NO: 23. In some embodiments, a recombinant virus is provided which includes a polynucleotide that encodes a Versikine molecule, wherein the Versikine molecule comprises an amino acid sequence having at least 90%, 91%, 92%, 93%, 94%, 95%, 96%, 97%, 98%, or 99% sequence identity to SEQ ID NO: 24. In some embodiments, a recombinant virus is provided which includes a polynucleotide that encodes an anti-VEGF-anti-Ang2 scAb molecule, wherein the anti-VEGF-anti-Ang2 scAb molecule comprises an amino acid sequence having at least 90%, 91%, 92%, 93%, 94%, 95%, 96%, 97%, 98%, or 99% sequence identity to SEQ ID NO: 23, and a polynucleotide that encodes a Versikine molecule, wherein the Versikine molecule comprises an amino acid sequence having at least 90%, 91%, 92%, 93%, 94%, 95%, 96%, 97%, 98%, or 99% sequence identity to SEQ ID NO: 24.

In some embodiments, the recombinant virus expresses an anti-VEGF single chain antibody (scAb) gene product, an Angiopoietin-2 (Ang-2) gene product, and a Versikine gene product (NP_001157569). Specifically, in some embodiments, the recombinant virus comprises an expression cassette containing an anti-VEGF single chain antibody (scAb) gene product and an Angiopoietin-2 (Ang-2) gene product cDNA under the control of vaccinia modified H5 (mH5) gene promoter, and a cDNA including the Versikine gene product under the control of vaccinia PSEL promoter. The full cassette including the cDNA including an anti-VEGF single chain antibody (scAb) gene product, an Angiopoietin-2 (Ang-2) gene product, and a Versikine gene product is inserted into vaccinia J2R genomic region. In addition, the recombinant viruses expressing an anti-VEGF single chain antibody (scAb) gene product, an Angiopoietin-2 (Ang-2) gene product, and a Varsikine gene product include a deletion of the A35R genomic region.

In some embodiments, the anti-angiogenic protein includes an anti-VEGF-Ang-2 single chain antibody polypeptide and a Versikine polypeptide. In some embodiments, the anti-VEGF-Ang-2 single chain antibody polypeptide can increase the ability of the recombinant virus to inhibit angiogenesis. In some embodiments, the Versikine polypeptide can increase the ability of the recombinant virus to inhibit angiogenesis. In some embodiments, the Versikine polypeptide can enhance the anti-tumorigenic and/or anti-angiogenic properties of the anti-VEGF-Ang-2 single chain antibody polypeptide. In some embodiments, a recombinant virus is provided which includes a polynucleotide that encodes an anti-VEGF-Ang-2 single chain antibody molecule, wherein the anti-VEGF-Ang-2 single chain antibody molecule comprises an amino acid sequence having at least 90%, 91%, 92%, 93%, 94%, 95%, 96%, 97%, 98%, or 99% sequence identity to SEQ ID NO: 23. In some embodiments, a recombinant virus is provided which includes a polynucleotide that encodes a Versikine molecule, wherein the Versikine molecule comprises an amino acid sequence having at least 90%, 91%, 92%, 93%, 94%, 95%, 96%, 97%, 98%, or 99% sequence identity to SEQ ID NO: 24. In some embodiments, a recombinant virus is provided which includes a polynucleotide that encodes an anti-VEGF-Ang-2 single chain antibody molecule, wherein the anti-VEGF-Ang-2 single chain antibody molecule comprises an amino acid sequence having at least 90%, 91%, 92%, 93%, 94%, 95%, 96%, 97%, 98%, or 99% sequence identity to SEQ ID NO: 23, and a polynucleotide that encodes a Versikine molecule, wherein the Versikine molecule comprises an amino acid sequence having at least 90%, 91%, 92%, 93%, 94%, 95%, 96%, 97%, 98%, or 99% sequence identity to SEQ ID NO: 24.

In some embodiments, the recombinant virus comprises a polypeptide with a sequence of amino acids that has at least 70%, such as at least 75%, 80%, 85% or 90% sequence identity to SEQ ID NO: 23. For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 23, but is less than 100% identical to the amino aicd sequence of SEQ ID NO: 23. In some embodiments, the recombinant virus comprises the amino acid sequence of SEQ ID NO: 23. In some embodiments, the recombinant virus comprises a polypeptide with a sequence of amino acids that has at least 70%, such as at least 75%, 80%, 85% or 90% sequence identity to the amino acid sequence of SEQ ID NO: 24. For example, in some embodiments, the recombinant virus comprises a sequence of nucleotides that encodes a polypeptide having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the amino acid sequence of SEQ ID NO: 24, but is less than 100% identical to SEQ ID NO: 24. In some embodiments, the recombinant virus comprises a sequence of nucleotides that encodes the amino acid sequence of SEQ ID NO: 24.

In some embodiments, the recombinant virus which includes a polynucleotide that encodes an anti-VEGF-Ang-2 single chain antibody molecule (e.g., comprising the amino acid sequence of SEQ ID NO: 23) and Versikine molecule (e.g., comprising the amino acid sequence of SEQ ID NO: 24) is derived from the VIR11 (comprising the nucleic acid sequence of SEQ ID NO: 3) strain and includes a sequence of nucleotides that has at least 70%, such as at least 75%, 80%, 85% or 90% sequence identity to SEQ ID NO: 13 (VIR71). For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 13, but is less than 100% identical to SEQ ID NO: 13. In some embodiments, the recombinant virus comprises a sequence of nucleotides comprising the nucleic acid sequence of SEQ ID NO: 13. In some embodiments, the VIR71 virus (comprising the nucleic acid sequence of SEQ ID NO: 13) includes a deletion of the genomic region corresponding to the A35R gene. In some embodiments, the polynucleotide that encodes the anti-VEGF-Ang-2 single chain antibody molecule (comprising the amino acid sequence of SEQ ID NO: 23) is operably linked to the mH5 gene promoter. In some embodiments, the polynucleotide that encodes the Versikine molecule (comprising the amino acid sequence of SEQ ID NO: 24) is operably linked to the PSEL gene promoter.

In some embodiments, the nucleic acid genome of the recombinant virus, e.g., recombinant oncolytic virus, comprises a sequence of nucleotides that has at least 95%, at least 96%, at least 97%, at least 98% or at least 99% sequence identity to the sequence set forth in SEQ ID NO: 13. In some embodiments, the nucleic acid genome of the recombinant virus, e.g., recombinant oncolytic virus, comprises the nucleic acid sequence of SEQ ID NO: 13. A recombinant oncolytic virus comprising the nucleic acid sequence of SEQ ID NO: 13 is also referred to herein as VIR71.

In some embodiments, the recombinant viruses provided herein are derived from the VIR11 (comprising the nucleic acid sequence of SEQ ID NO: 3) strain and comprises a sequence of nucleotides that has at least 70%, such as at least 75%, 80%, 85% or 90% sequence identity to SEQ ID NO: 12 (also named VIR52). For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 12, but is less than 100% identical to the nucleic acid sequence of SEQ ID NO: 12. In some embodiments, the VIR52 virus (comprising the nucleic acid sequence of SEQ ID NO: 12) includes a deletion of the genomic region corresponding to the A35R gene and a deletion of the genomic region corresponding to the J2R gene.

In some embodiments, the recombinant virus provided herein exhibit increased anti-tumor properties, such as by improved anti-angiogenesis inhibiting properties. In some embodiments, recombinant viruses provided herein can inhibit tumor, hyperplasia, or metastasis growth in an in vivo model. In particular embodiments, administration of an effective dose of VIR71 (comprising the nucleic acid sequence of SEQ ID NO: 13) to a subject inhibits tumor, hyperplasia, or metastasis growth in an in vivo model. In some embodiments, administration of an effective dose of a recombinant virus comprising a nucleic acid sequence having at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 13 to a subject inhibits tumor, hyperplasia, and/or metastasis growth.

Also provided herein are recombinant viruses including heterologous nucleic acids encoding for immune stimulating proteins and angiogenesis modulating proteins that can be stably and efficiently expressed in many types of virus-infected cells and increase the anti-tumorigenic properties of the virus. Accordingly, in some embodiments, the recombinant oncolytic virus comprises at least one heterologous nucleic acid encoding one or more heterologous gene product, wherein the one or more heterologous gene product comprises one or more immune stimulating protein and one or more anti-angiogenic protein. In some embodiments, the immune modulating proteins include, but are not limited to, hmLIGHT. In some embodiments, the immune modulating proteins include, but are not limited to, anti-VEGF single chain antibody (scAb) and Angiopoietin-2 (Ang-2). In some embodiments, the immune modulating proteins include, but are not limited to, hmLIGHT and the angiogenesis modulating proteins include, but are not limited to, anti-VEGF single chain antibody (scAb) and Angiopoietin-2 (Ang-2). In some embodiments, the immune modulating proteins include, but are not limited to, any immune modulating protein hereby included by reference. In some embodiments, the angiogenesis modulating proteins include, but are not limited to, any angiogenesis modulating protein hereby included by reference.

Recombinant viruses expressing an hmLIGHT gene product (NP_001363816 XP_016882906), an anti-VEGF single chain antibody (scAb) gene product and an Angiopoietin-2 (Ang-2) gene product have been generated herein in various embodiments. Specifically, the generated recombinant viruses include two expression cassettes; one expression cassette is inserted into the A35R genomic region and contains a full length hmLIGHT cDNA under the control of vaccinia SSE gene promoter; and the second cassette is inserted in the J2R genomic regions and contains an anti-VEGF single chain antibody (scAb) gene product and an Angiopoietin-2 (Ang-2) gene product cDNA under the control of vaccinia modified H5 (mH5) gene promoter and a cDNA including the Versikine gene product under the control of vaccinia SEL promoter.

In some embodiments, the immune modulating protein includes hmLIGHT and the angiogenesis modulating protein includes anti-VEGF single chain antibody (scAb) and Angiopoietin-2 (Ang-2). In some embodiments, the hmLIGHT protein, the anti-VEGF single chain antibody (scAb) protein and/or the Angiopoietin-2 (Ang-2) gene product can increase the anti-tumor properties of the recombinant virus and/or inhibit host's angiogenesis. In some embodiments, a recombinant virus is provided which includes a polynucleotide that encodes an hmLIGHT molecule, anti-VEGF single chain antibody (scAb) molecule, an Angiopoietin-2 (Ang-2) molecule, and a Versikine molecule, wherein the hmLIGHT polypeptide comprises an amino acid sequence having at least 90% sequence identity to SEQ ID NO: 25, the anti-VEGF single chain antibody (scAb) Angiopoietin-2 (Ang-2) polypeptide comprises an amino acid sequence having at least 90% sequence identity to SEQ ID NO: 23, and the Versikine polypeptide comprises an amino acid sequence having at least 90% sequence identity to SEQ ID NO: 24.

In some embodiments, the recombinant virus includes a polypeptide with a sequence of amino acids that has at least 70%, such as at least 75%, 80%, 85% or 90% sequence identity to SEQ ID NO: 25. For example, the recombinant viruses have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 25. In some embodiments, the recombinant virus includes a polypeptide with a sequence of amino acids that has at least 70%, such as at least 75%, 80%, 85% or 90% sequence identity to SEQ ID NO: 23. For example, the recombinant viruses have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 23. In some embodiments, the recombinant virus includes a polypeptide with a sequence of amino acids that has at least 70%, such as at least 75%, 80%, 85% or 90% sequence identity to SEQ ID NO: 24. For example, the recombinant viruses have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 24.

In some embodiments, the polynucleotide that encodes the hmLIGHT molecule is operably linked to the SSE gene promoter and the polynucleotide that encodes the anti-VEGF single chain antibody (scAb)-Angiopoietin-2 (Ang-2) molecule is operably linked to the mH5 promoter, and the Versikine molecule is operably linked to the SEL promoter. In some embodiments, the recombinant virus which includes a polynucleotide that encodes an hmLIGHT molecule (e.g., comprising the sequence of SEQ ID NO: 25), the anti-VEGF single chain antibody (scAb)/Angiopoietin-2 (Ang-2) molecule (e.g., comprising the sequence of SEQ ID NO: 23), and the Versikine molecule (e.g., comprising the sequence of SEQ ID NO: 24) is derived from the VIR49 (comprising the nucleic acid sequence of SEQ ID NO: 11) strain and includes a sequence of nucleotides that has at least 70%, such as at least 75%, 80%, 85% or 90% sequence identity to SEQ ID NO: 47 (also named VIR86). For example, the recombinant virus have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 47. In some embodiments, the nucleic acid genome of the recombinant oncolytic virus comprises a sequence of nucleotides that has at least 95%, at least 96%, at least 97%, at least 98% or at least 99% sequence identity to the sequence set forth in SEQ ID NO: 47. In some embodiments, the nucleic acid genome of the recombinant oncolytic virus comprises the sequence of nucleotides of SEQ ID NO: 47.

Recombinant virus provided herein exhibit increase anti-tumorigenic properties and anti-angiogenesis properties. In particular embodiments, administration of an effective dose of VIR86 (SEQ ID NO: 47) to a subject inhibits tumor, hyperplasia, or metastasis growth in an in vivo model.

### D. Apoptosis Modulating Viruses

In some embodiments, the at least one heterologous nucleic acid encoding one or more heterologous gene product comprises one or more heterologous nucleic acid each encoding an apoptosis-inducible protein, such as iDED, iFas, and/or iCas9.

Accordingly, provided herein are recombinant viruses including heterologous nucleic acids encoding for an inducible “safety switch” mechanism that can be stably and efficiently expressed in many types of virus infected cells upon induction with an inducing agent.

Viral therapies hold great promise for the treatment of human disease. Significant toxicities from the viruses themselves or from the heterologous gene products they carry have hampered clinical investigation. Thus, there is considerable interest in developing “safety switches” by which cells infected with a therapeutic virus may be ablated, or “turned off,” should problems arise with their use. Safety-switch systems have been investigated in a variety of cells. Safety-switch genes have been used, for example, in T lymphocytes to treat viral infections and malignancies and in T-cell infusions to treat recurrent malignancy. Another option is based on the induction of genes that activate apoptosis pathways in human T cells; See Straathof et al., Blood (2005), 105:11, 4247-4254).

One way to eliminate unwanted cells is by inducing cell apoptosis, a type of programed cell death or cellular suicide, a vital component of various cellular processes including normal cell turnover, proper development and functioning of the immune system, hormone-dependent atrophy, embryonic development and chemical-induced cell death. Inappropriate apoptosis (either too little or too much) is a factor in many human conditions including neurodegenerative diseases, ischemic damage, autoimmune disorders and many types of cancer. Therefore, modulation of apoptosis seems a convenient candidate for a “safety switch” system meant to kill a cell upon switching the system on. See S. Elmore, Toxicol Pathol. 2007; 35(4): 495-516.

In humans, apoptosis can be induced through stimulation of the Fas receptor that results in recruitment of the initiator caspase 8, through interaction with the adaptor molecule Fas-associated death domain protein (FADD) by means of its death domains (DDs) and death effector domains (DEDs). In addition, apoptosis can be activated by disruption of the mitochondrial membrane that leads to activation of caspase 9. Both reduced and increased apoptosis can result in pathologies. Furthermore, mutations that affect apoptosis pathways are one of the hallmarks of cancer development. See Hanahan et al., Cell (2011) 144:5, 646-674.

Inducible systems including apoptotic effector proteins have been described; for example, inducible systems have been explored in human T lymphocytes using caspase 9, Fas or the death effector domain (DED) of the Fas-associated death domain-containing protein (FADD). However, no “safety switch” system including apoptosis effectors is believed to have yet been engineered into viral systems.

Provided herein, are recombinant viruses including heterologous nucleic acids encoding for an inducible “safety switch” mechanism including an inducible Caspase 9, and inducible Fas, or an inducible DED gene fused to F36V-FKBP, a mutant version of human FK506 binding protein (FKBP12). This inducible “safety switch” allows for conditional dimerization upon induction with AP1903, an FDA approved drug for use in humans. See Straathof et al., Blood (2005) 105:11, 4247-4254. This inducible system has advantages that can make it a good candidate for viral therapy: it includes human gene products with potential low immunogenicity, and administration of AP1903 has no effects other than the selective elimination of targeted cells. Administration of this small molecule results in cross-linking and activation of the proapoptotic fusion molecules iCasp9 (caspase 9 fused to F36V-FKBP), iDED (DED fused to F36V-FKBP) and iFas (Fas fused to F36V-FKBP).

Accordingly, provided herein, in some embodiments, is a recombinant oncolytic virus comprising at least one heterologous nucleic acid encoding one or more heterologous gene product, wherein the one or more heterologous gene product is or comprises an apoptosis-inducible protein. In some embodiments, the apoptosis-inducible protein comprises a proapoptotic molecule fused with an FKBP variant that is able to bind a chemical inducer of dimerization (CID). In some embodiments, the FKBP variant is FKBP-F36V. In some embodiments, the FKBP variant is FKBP-F36V, and the FKBP-F36V comprises the sequence of amino acids set forth in SEQ ID NO: 56. In some embodiments, the chemical inducer of dimerization is AP1903 (Rimiducid). In some embodiments, the proapoptotic molecule is Fas, the death effector domain (DED) of the Fas-associated death domain-containing protein (FADD), or a caspase, optionally wherein the caspase is caspase 9.

In some embodiments, the apoptosis-inducible protein allows for an inducible system that includes a chimeric polypeptide that includes a multimerization region fused to a proapoptotic polypeptide. Such inducible system can be activated or “switched on” upon binding of a multimeric ligand to the multimerization region. In some embodiments, the apoptosis-inducible protein comprises a multimerization region fused to a proapoptotic polypeptide. In some embodiments, the multimerization region includes any one of FKBP12, F36V-FKBP, cyclophilin receptor, steroid receptor, tetracycline receptor, heavy chain antibody subunit, light chain antibody subunit, single chain antibodies comprised of heavy and light chain variable regions in tandem separated by a flexible linker domain, and mutated sequences thereof. In some embodiments, the multimerization region is a F36V-FKBP region. In some embodiments, the multimerization region is an FKBP12 region. In some embodiments, the FKBP12 region is an FKBP12v36 region. In some embodiments, the multimerization region is Fv′Fvls. In some embodiments, the multimerization region binds a ligand selected from the group consisting of an FK506 dimer and a dimeric FK506 analog ligand. In some embodiments, the ligand is AP1903 (Rimiducid). In some embodiments, the ligand is AP20187.

In some embodiments, the multimerization region includes a ligand-binding domain. Such ligand-binding domain can be any convenient domain that will allow for induction of the inducible system using a natural or unnatural ligand, for example, an unnatural synthetic ligand. Examples of ligand-binding domains include, but are not limited to, cyclophilin receptor ligand-binding domain, steroid receptor ligand-binding domain, cyclophilin receptor ligand-binding domain, and tetracycline receptor ligand-binding domain. In some embodiments, the ligand-binding domain includes a ligand-binding domain of FK506 binding protein (FKBP12). In some embodiments, the ligand-binding domain includes a ligand-binding domain of the FKBP12 variant FK506-BP12. Further details about inducible systems using ligand-binding domains can be found in US Patent Publication No. 2020/0230216, which is hereby incorporated by reference for all purposes. In some embodiments, the FKB12 variant includes, but is not limited to, iCaspase9, iCasp9, iC9, CaspaCIDe. Further details about inducible systems using ligand-binding domains can be found in WIPO Patent Publication WO 2016/100241, which is hereby incorporated by reference for all purposes.

In some embodiments, the inducible system can be activated or “switched on” upon administration of multimeric ligand that results in multimerization of multimerization regions and activation of the inducible system. In some embodiments, the ligand is a small molecule. In some embodiments, the ligand is dimeric. In some embodiments, the ligand is dimeric FK506, or a dimeric FK506-like analog. In certain embodiments, the multimeric ligand is AP1903, a synthetic drug that has proven safe in healthy volunteers. In some embodiments, AP1903 administration results in dimerization and cross-linking of the proapoptotic polypeptide and activation of the inducible system. In some embodiments, the multimeric ligand is AP20187 (ARIAD Pharmaceuticals, Cambridge, MA), a nontoxic synthetic FK506 analog that has been modified to reduce interactions with endogenous FKBP12s, while enhancing binding to this FK506-BP12 variant. Administration of the multimeric ligand results in the aggregation of inducible proapoptotic polypeptides, leading to their activation.

Provided herein, in various embodiments, are recombinant viruses including heterologous nucleic acids encoding for an inducible “safety switch” mechanism including an inducible Caspase 9, and inducible Fas, or an inducible DED gene fused to F36V-FKBP, a mutant version of human FK506 binding protein (FKBP12). This inducible “safety switch” allows for conditional multimerization upon induction with AP1903, an FDA approved drug for use in humans. See Straathof et al., Blood (2005) 105:11, 4247-4254. This inducible system has advantages that can make it a good candidate for viral therapy: it includes human gene products with a potential for low immunogenicity, and administration of AP1903 has no effects other than the selective elimination of targeted cells. Administration of this small molecule results in cross-linking and activation of the proapoptotic fusion molecules iCasp9 (caspase 9 fused to F36V-FKBP), iDED (DED fused to F36V-FKBP) and iFas (Fas fused to F36V-FKBP).

### 1. Casp9 Inducible System

Provided herein, in some embodiments, is a Casp9 inducible system. In some embodiments, the Casp9 inducible “safety switch” mechanism comprises an inducible Caspase 9 gene fused to F36V-FKBP, which is a mutated version of human FK506 binding protein (FKBP12) that allows for conditional dimerization of F36V-FKBP upon induction of the system with AP1903, which is an FDA approved drug for use in humans. See Straathof et al., Blood (2005) 105:11, 4247-4254. Administration of this small molecule results in cross-linking and activation of the proapoptotic fusion molecule iCasp9 (caspase 9 fused to F36V-FKBP). Methods for inducing selective apoptosis using a Caspase-9 system, using ligand-mediated dimerization and activation of a chimeric Caspase-9 polypeptide are discussed in U.S. patent application Ser. No. 13/112,739, filed May 20, 2011, and entitled METHODS FOR INDUCING SELECTIVE APOPTOSIS, naming Malcolm K. Brenner as inventor, which is hereby incorporated by reference herein in its entirety for all purposes. See also Straathof et al., Blood (2005), 105:11, 4247-4254). Further details about inducible systems using ligand-binding domains can be found in U.S. Pat. No. 9,434,935, which is hereby incorporated by reference for all purposes.

In some embodiments, the apoptosis-inducible protein comprises a proapoptotic molecule fused with an FKBP variant that is able to bind a chemical inducer of dimerization (CID). In some embodiments, the FKBP variant is FKBP-F36V. In some embodiments, the FKBP variant is FKBP-F36V, and the FKBP-F36V comprises the sequence of amino acids set forth in SEQ ID NO: 56.

In some embodiments, the chemical inducer of dimerization is AP1903 (Rimiducid).

In some embodiments, the proapoptotic molecule is Fas, the death effector domain (DED) of the Fas-associated death domain-containing protein (FADD), or a caspase, optionally wherein the caspase is caspase 9.

In some embodiments, the proapoptotic molecule is caspase and the caspase is caspase 9. In some embodiments, the apoptosis-inducible protein is an inducible caspase 9 (iCasp9). In some embodiments, the recombinant virus expresses an inducible iCasp9 system. In some embodiments, the iCasp9 comprises a proapoptotic molecule fused with an FKBP variant that is able to bind a chemical inducer of dimerization (CID). Specifically, in some embodiments, the recombinant virus comprises an expression cassette containing an inducible Casp9 cDNA molecule under the control of a vaccinia synthetic early promoter PSE inserted into the J2R gene locus. The iCasp9 molecule includes a caspase 9 (aa 135-416) without its caspase activation and recruitment domain (CARD; GenBank NM001 229) linked to a F36V-FKBP, a mutant version of human FK506 binding protein (FKBP12; GenBank AH002 818) that contain an F36V mutation (F36V-FKBP).

In some embodiments, a recombinant virus is provided which comprises a polynucleotide that encodes an iCasp9 molecule comprising a portion of caspase 9 (amino acid residues 135-416) linked to a F36V-FKBP, wherein the iCasp9 polypeptide comprises an amino acid sequence having at least 90%, 91%, 92%, 93%, 94%, 95%, 95%, 97%, 98%, or 99% sequence identity to the amino acid sequence of SEQ ID NO: 26. In some embodiments, the portion of caspase 9 contains amino acid residues 135 to 416 of full-length caspase 9. Accordingly, in some embodiments, the apoptosis-inducible protein is an inducible caspase 9 (iCas9) comprising the amino acid sequence set forth in SEQ ID NO: 26, or a sequence of amino acids that has at least 85%, 90%, or 95% sequence identity to the amino acid sequence of SEQ ID NO: 26. In some embodiments, the recombinant virus includes a polypeptide with a sequence of amino acids that has at least 70%, such as at least 75%, 80%, 85% or 90% sequence identity to SEQ ID NO: 26. For example, the recombinant viruses have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical SEQ ID NO: 26. In some embodiments, the polynucleotide that encodes the iCasp9 molecule is operably linked to the PSE promoter.

In some embodiments, the recombinant virus which comprises a polynucleotide that encodes an iCasp9 molecule (e.g., comprising the amino acid sequence of SEQ ID NO: 26) is derived from the clonal VIP02 (comprising the nucleic acid sequence of SEQ ID NO: 1) strain, and the recombinant virus comprises a sequence of nucleotides that has at least 70%, such as at least 75%, 80%, 85% or 90% sequence identity to the nucleic acid sequence of SEQ ID NO: 7 (also named VIR40). For example, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 7, but is less than 100% identical to the nucleic acid sequence of SEQ ID NO: 7. In some embodiments, the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 7. In some embodiments, the recombinant virus, e.g., recombinant oncolytic virus, comprises the nucleic acid sequence of SEQ ID NO: 7. A recombinant oncolytic virus comprising the nucleic acid sequence of SEQ ID NO: 7 is also referred to herein as VIR40.

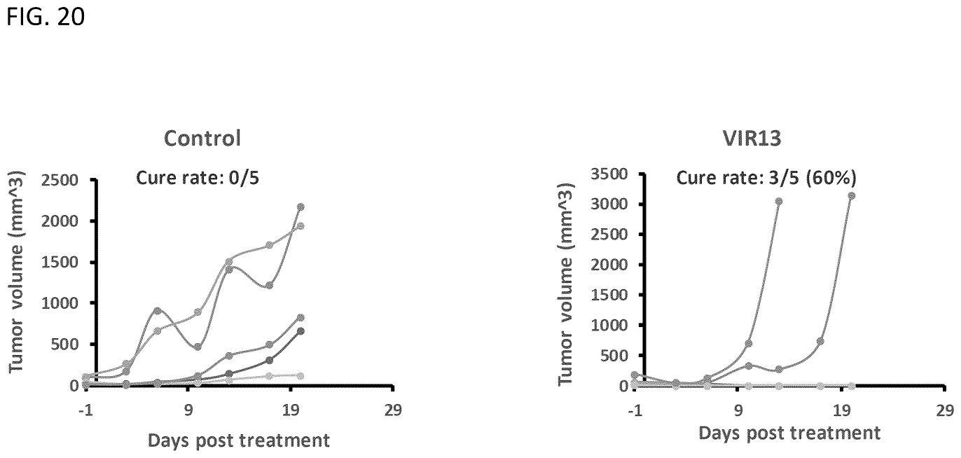

In some embodiments, the recombinant virus is derived from the clonal VIP02 (comprising the nucleic acid sequence of SEQ ID NO: 1) strain and includes a deletion of the J2R gene. In some embodiments, the recombinant virus is derived from the clonal VIP02 (comprising the nucleic acid sequence of SEQ ID NO: 1) strain and virus includes a sequence of nucleotides that has at least 70%, such as at least 75%, 80%, 85% or 90% sequence identity to SEQ ID NO: 4 (also named VIR13). For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 4. In some embodiments, subjects infected with an effective dose of VIR13 exhibit a remarkable reduction of tumor progression as compared to subjects infected with a vehicle solution as shown in FIG. 20.

In some embodiments, the recombinant virus is derived from the VIR13 (SEQ ID NO: 4) strain and includes a deletion of the J2R gene and a deletion of the B2R gene. In some embodiments, the recombinant virus is derived from the VIR13 (SEQ ID NO: 4) strain and virus includes a sequence of nucleotides that has at least 70%, such as at least 75%, 80%, 85% or 90% sequence identity to SEQ ID NO: 48 (also named VIR94). For example, the recombinant virus have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9%, but are less than 100% identical to SEQ ID NO: 48. In some embodiments, subjects infected with an effective dose of VIR94 exhibit a remarkable reduction of tumor progression as compared to subjects infected with a vehicle solution.

In some embodiments, VIR40 exhibits enhanced anti-tumorigenic activity compared to VIR13 after induction with an effective dose of Rimiducid. In some embodiments, VIR40 exhibits less anti-tumorigenic activity compared to VIR13 after induction with an effective dose of Rimiducid. In some embodiments, VIR40 exhibits similar anti-tumorigenic activity compared to VIR13 after induction with an effective dose of Rimiducid.

In some embodiments, the recombinant virus includes a nucleic acid having a nucleotide sequence that encodes a chimeric protein comprising a multimeric ligand binding region and a modified Caspase-9 polypeptide, and comprises at least one amino acid substitution selected from the group consisting of N405Q, D330A, F404Y, F406L, F406T, F404W, T317A, S144A, S144D, S196A, S183A, S195A, F404T, F404W, N405F, F406T, D315A, A316G, T3175, F319W, 5307A, Y153A, and Y153F. In some embodiments, the chimeric protein has a basal activity less than that of the wild type Caspase-9 polypeptide. In some embodiments, the chimeric protein comprising the modified Caspase-9 polypeptide has a basal activity of less than 5%, 10%, 15%, 20%, 25%, 30%, 35%, 40%, 45%, 50%, 55%, 60%, 65%, 70%, 75%, 80%, 85%, or 90% that of a chimeric protein comprising a non-modified Caspase-9. Further details about inducible systems using ligand-binding domains can be found in U.S. Pat. No. 9,932,572, which is hereby incorporated by reference for all purposes.

Caspase polypeptides other than Caspase-9 that may be encoded by the chimeric polypeptides of the current technology include, for example, Caspase-1, Caspase-3, and Caspase-8. Discussions of these Caspase polypeptides may be found in, for example, MacCorkle, R. A., et al., Proc. Natl. Acad. Sci. U.S.A. (1998) 95:3655-3660; and Fan, L., et al. (1999) Human Gene Therapy 10:2273-2285). Further details about inducible systems using ligand-binding domains can be found in U.S. Pat. No. 10,525,110, which is hereby incorporated by reference for all purposes.

2. iDED Inducible System

The Death effector domains (DED) are found in inactive procaspases (cysteine proteases) and proteins that regulate caspase activation in the apoptosis cascade such as FAS-associating death domain-containing protein (FADD). FADD is a signaling adaptor protein, which mediates the activation of caspase 8 during death receptor-induced apoptosis. In addition to their role in apoptosis and cancer, these proteins have been shown to have important roles in regulating other forms of cell death, including necroptosis, and in regulating other important cellular processes, including autophagy and inflammation. Moreover, these proteins also have prominent roles in innate and adaptive immunity and during embryonic development. See J S Riley, A Malik, C Holohan & D B Longley Cell Death & Disease volume 6, pagee 1866 (2015) and Imtiyaz et al., J Immunol. 2006 Jun. 1; 176(11): 6852-6861.

Accordingly, provided herein, in some embodiments, is a recombinant oncolytic virus comprising at least one heterologous nucleic acid encoding one or more heterologous gene product, wherein the one or more heterologous gene product is or comprises an apoptosis-inducible protein. In some embodiments, the apoptosis-inducible protein comprises a proapoptotic molecule fused with an FKBP variant that is able to bind a chemical inducer of dimerization (CID). In some embodiments, the FKBP variant is FKBP-F36V. In some embodiments, the FKBP variant is FKBP-F36V, and the FKBP-F36V comprises the sequence of amino acids set forth in SEQ ID NO: 56. In some embodiments, the chemical inducer of dimerization is AP1903 (Rimiducid). In some embodiments, the proapoptotic molecule is the death effector domain (DED) of the Fas-associated death domain-containing protein (FADD).

In some embodiments, the apoptosis-inducible protein is an inducible DED (iDED). In some embodiments, the iDED comprises a proapoptotic molecule fused with an FKBP variant that is able to bind a chemical inducer of dimerization (CID).

In some embodiments, the recombinant virus comprises heterologous nucleic acids encoding for an inducible “safety switch” mechanism comprising an inducible DED fused to two copies of F36V-FKBP, a mutant version of the human FKBP506-binding protein, and v-src (amino acid residues 1-14), thereby providing an inducible mechanism that can be triggered upon the addition of chemical inducers of dimerization, such as AP1903, an FDA approved drug for use in humans. Further details about iDED inducible systems are discussed in Junker et al., (2003) Gene Therapy, 10, 1189-1197, which is hereby incorporated by reference for all purposes.

Recombinant viruses expressing an inducible iDED system have been generated herein. Specifically, in some embodiments, the generated recombinant viruses comprise an expression cassette containing an inducible DED (death effector domain (DED) of the Fas-associated protein with death Domain, FADD) cDNA molecule under the control of a vaccinia synthetic early promoter PSE inserted into the J2R gene locus. The iDED molecule includes a death effector domain (DED) of the Fas-associated protein with death Domain, FADD, fused to two copies of F36V-FKBP, a mutated version of human FKBP12 (e.g., GenBank AH002 818) that contain an F36V mutation and a v-src (amino acid residues 1-14).

In some embodiments, the recombinant virus comprises a polynucleotide that encodes an iDED molecule comprising a death effector domain (DED) of the Fas-associated protein with death Domain, FADD, linked to 2 F36V-FKBP, and a mutated version of human FKBP12, wherein the iDED polypeptide comprises an amino acid sequence having at least 90% sequence identity to SEQ ID NO: 27. In some embodiments, the apoptosis-inducible protein is an inducible DED (iDED) comprising the sequence set forth in SEQ ID NO: 27 or a sequence of amino acids that has at least 85%, 90%, or 95% sequence identity to SEQ ID NO: 27. In some embodiments, the apoptosis-inducible protein is an inducible DED (iDED) comprising the sequence set forth in SEQ ID NO: 27. In some embodiments, the recombinant virus comprises a polypeptide, e.g., an apoptosis-inducible protein, comprising a sequence of amino acids that has at least 70%, such as at least 75%, 80%, 85%, or 90% sequence identity to the amino acid sequence of SEQ ID NO: 27. In some embodiments, the polynucleotide that encodes the iDED molecule is operably linked to the PSE promoter.

In some embodiments, the recombinant virus, which comprises a polynucleotide that encodes an iDED molecule is derived from the clonal VIP02 (comprising the nucleic acid sequence of SEQ ID NO: 1) strain and includes a sequence of nucleotides that has at least 70%, such as at least 75%, 80%, 85% or 90% sequence identity to SEQ ID NO: 8 (also named VIR41). For example, in some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 8, but is less than 100% identical SEQ ID NO: 8. In some embodiments, the recombinant virus comprises the nucleic acid sequence of SEQ ID NO: 8. In some embodiments, the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8. In some embodiments, the nucleic acid genome of the recombinant oncolytic virus comprises the nucleic acid sequence of SEQ ID NO: 8. In some embodiments, the recombinant virus, e.g., recombinant oncolytic virus, comprises an iDED, and the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8. A recombinant oncolytic virus comprising the nucleic acid sequence of SEQ ID NO: 8 is also referred to herein as VIR41.

Viral therapies hold great promise for the treatment of human diseases including cancer; however, significant toxic levels from the viruses themselves or from the heterologous gene products they carry still remains a challenge for clinical application of such therapies. Thus, a “safety switch” that allows for selective inactivation of virus replication and propagation in healthy cells upon activation of the “safety switch” but that exhibits strong anti-tumorigenic properties in malignant cells upon infection, although unexpected, would constitute an important improvement to existing methods of treatment not only of cancer, but also of other conditions as well. Provided herein, in various embodiments, are recombinant viruses that upon treatment with Rimiducid, healthy cells infected with said viruses exhibit a significant inhibition of virus replication while cancer cells do not show a significant inhibition of virus-mediated cytotoxicity.

In particular, as discussed in Example 6, upon induction of iDED with Rimiducid, healthy cells infected with VIR41 exhibited a significant inhibition of virus replication while cancer cells did not show a significant inhibition of virus-mediated cytotoxicity. Therefore, the viruses described herein in various embodiments, and VIR41 particularly, are an improvement to the current methodologies for the treatment of cancer, hyperplasia, tumors, metastasis and other conditions thereof.

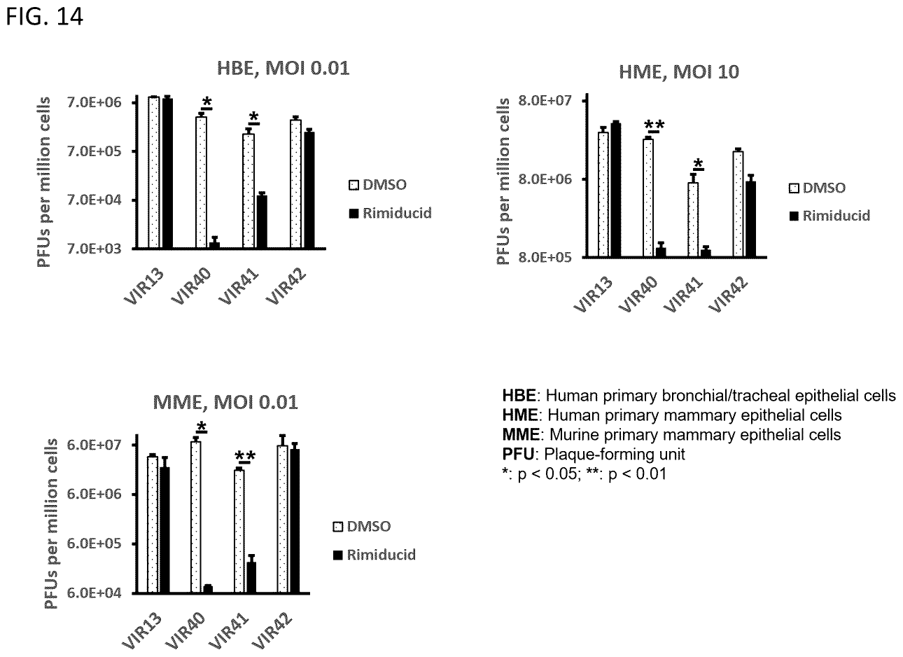

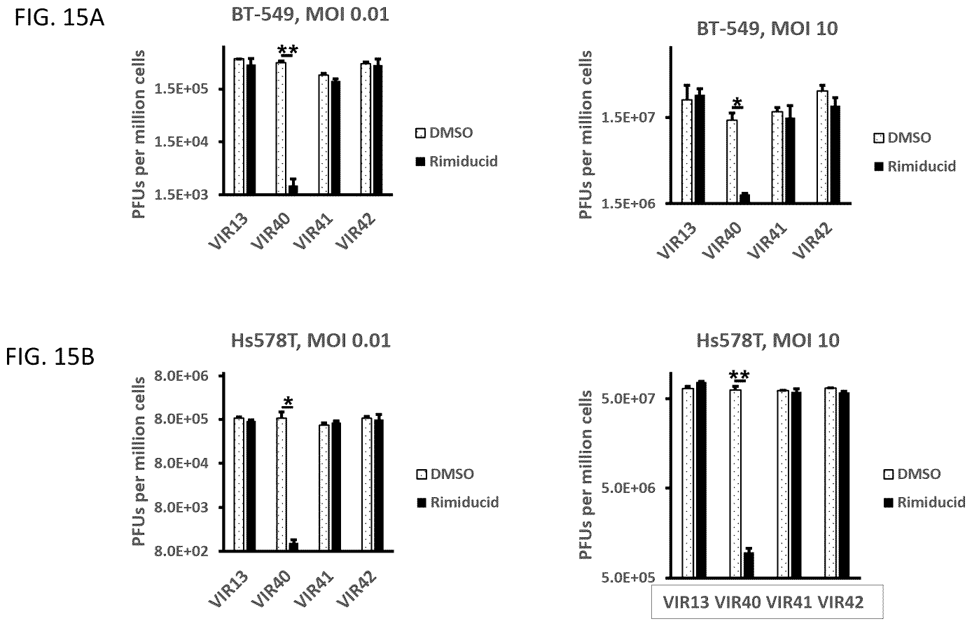

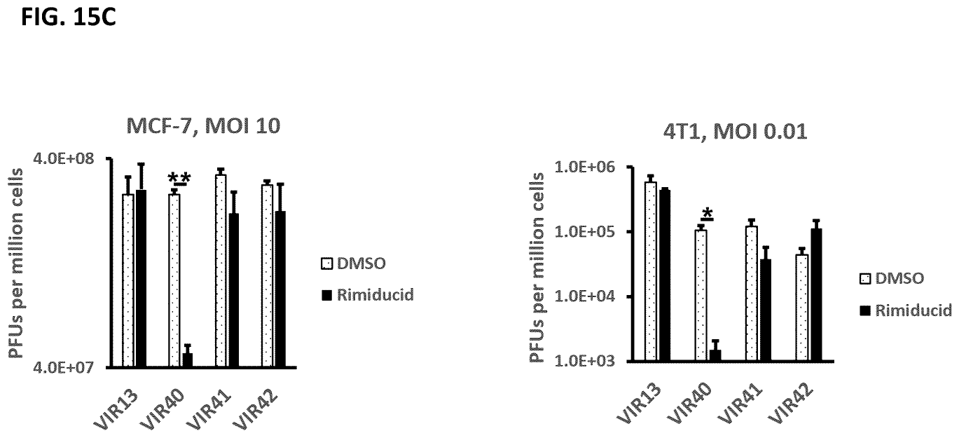

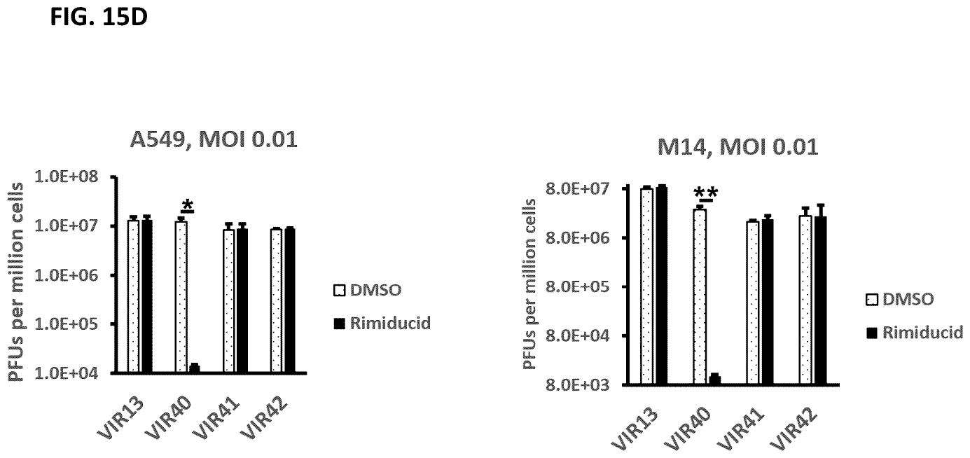

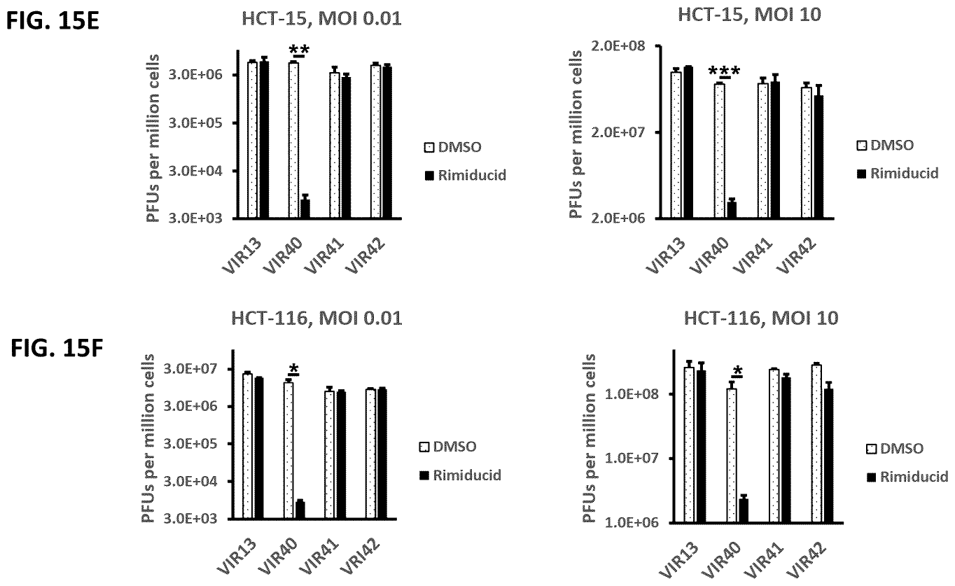

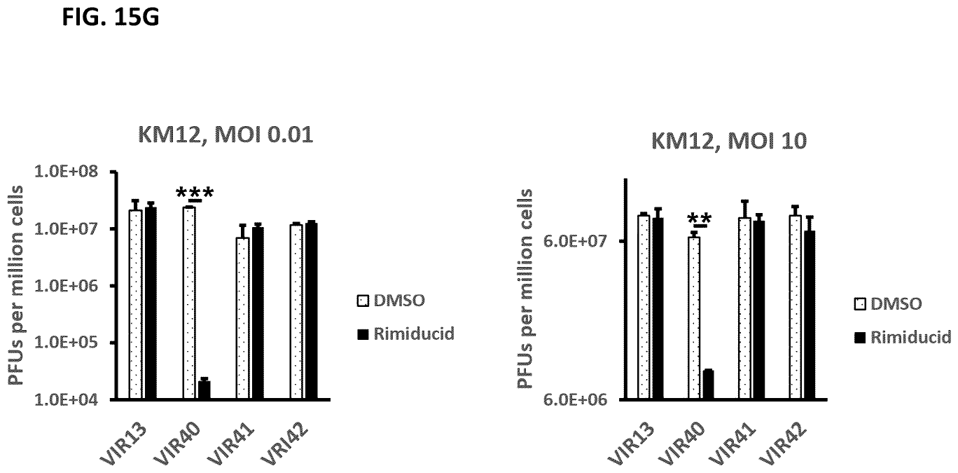

Specifically, in some embodiments, induction of iDED mediated apoptosis in cultures of human primary bronchial/tracheal epithelial cells, human primary mammary epithelial cells, and murine primary mammary epithelial cells, lead to a significant inhibition of virus replication in cells infected with VIR41, indicating that iDED inhibits viral replication in healthy cells (Example 6.A. and FIG. 14). In addition, Rimiducid treatment did not significantly inhibit virus replication in breast, lung, melanoma and microsatellite instable (MSI) colorectal cancer cells infected with VIR41 (Example 6.B) as demonstrated by BT-549 breast cancer cells (FIG. 15A), Hs578T breast cancer cells (FIG. 15B), MCF-7 and 4T1breast cancer cells (FIG. 15C), A549 and M14 lung and melanoma cancer cells (FIG. 15D), HCT-15 MSI colon cancer cells (FIG. 15E), HCT-116 MSI colon cancer cells (FIG. 15F), and KM12 MSI colon cancer cells (FIG. 15G). Therefore, activation of iDED mediated apoptosis in breast, lung, melanoma and MSI colon cancer cells does not inhibit viral replication.

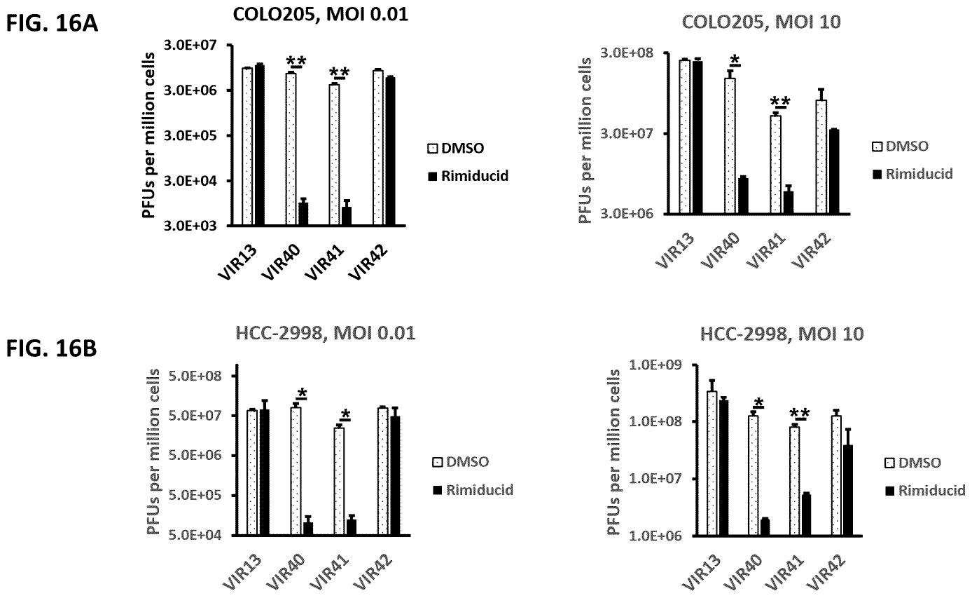

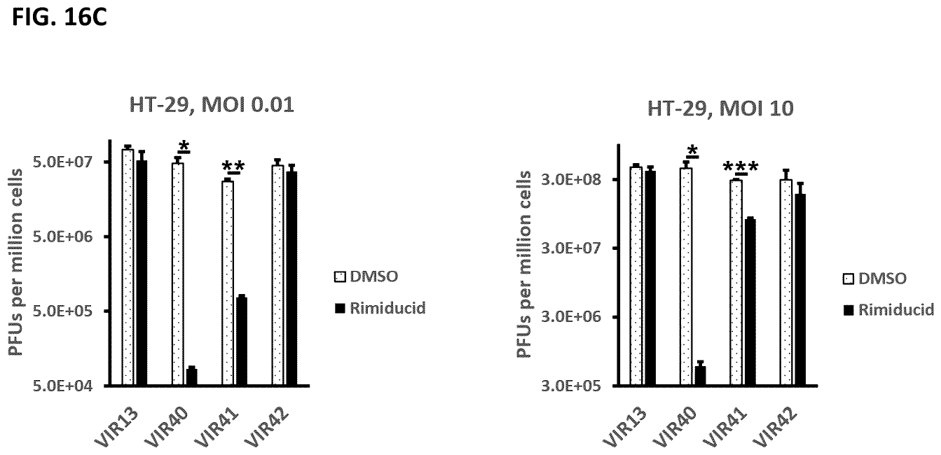

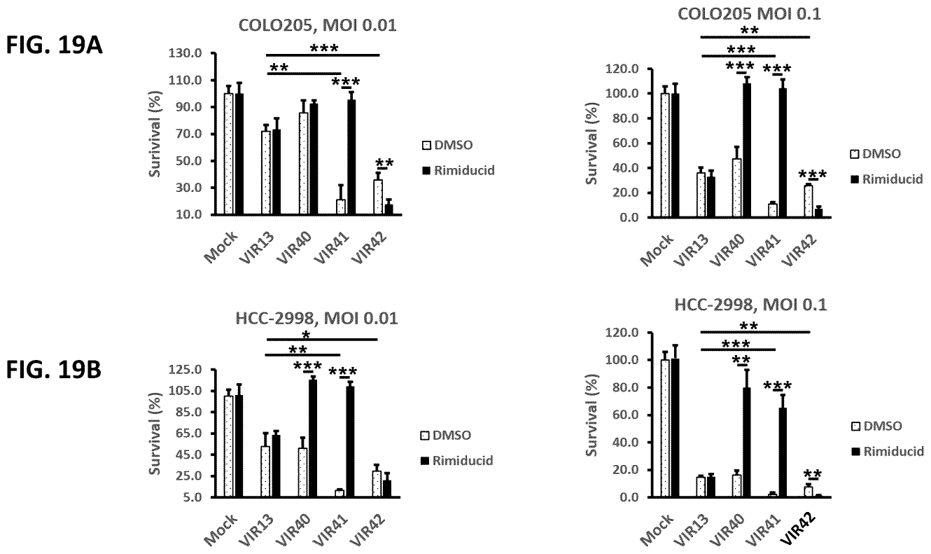

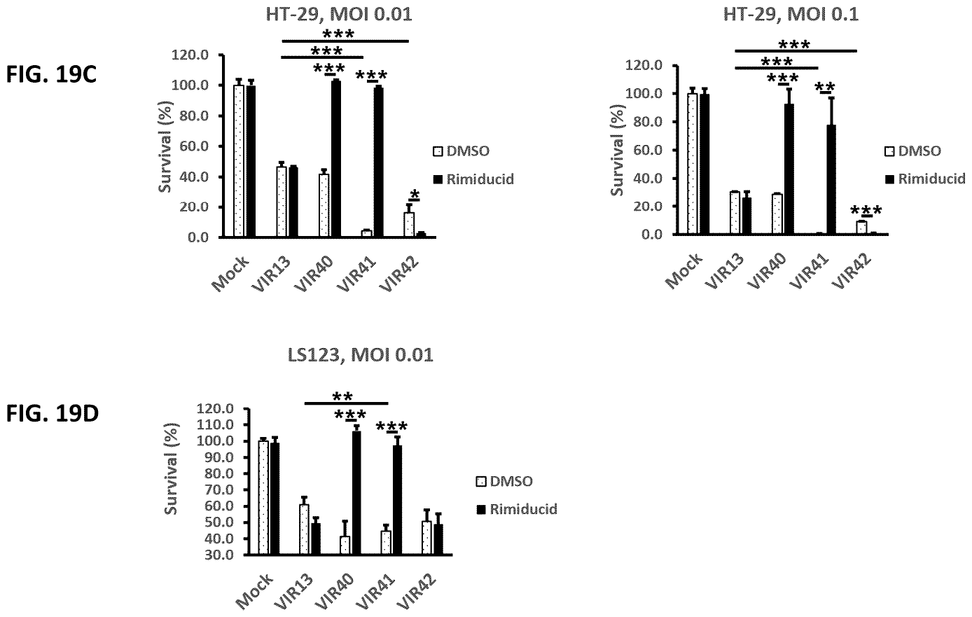

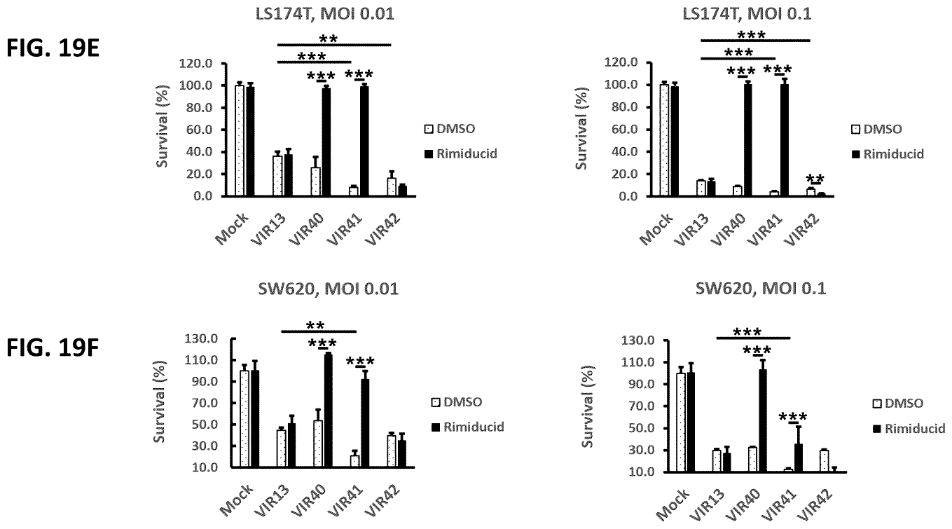

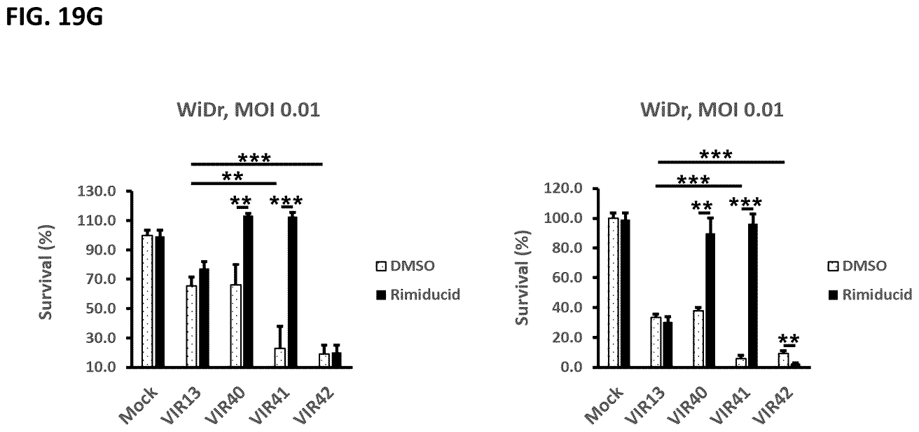

Microsatellite Stable (MSS) colon cancer is a type of difficult to treat cancer that does not respond to checkpoint inhibitors. As discussed in Example 6 and shown in FIG. 16, MSS colon cells infected with VIR41 responded differently than MSI colon cancer cells upon induction of iDED mediated apoptosis with Rimiducid. Rimiducid treatment significantly inhibited virus replication in MSS colorectal cells infected with VIR41, as demonstrated by COLO205 cancer cells (FIG. 16A), HCC-2998 cancer cells (FIG. 16B), and HT-29 cancer cells (FIG. 16C), indicating iDED mediated apoptosis activation inhibits virus replication in MSS colorectal cancer cells after treatment with Rimiducid, an unexpected finding. Although the exact mechanism is unknown it is likely that MSS colorectal cancer cells contain a relatively intact apoptosis pathway since it is known that MSS colorectal cancer cells have a lot less genomic mutations than MSI colorectal cancer cells. Consistent with this, the cytotoxicity experiments described in FIG. 19 show that VIR41 can kill MSS colorectal cancer cells much better than the control virus VIR13. It may be that the low level of activated iDED in the absence of Rimiducid can cause apoptosis in MSS colorectal cancer cells.

Moreover, as shown in Example 6, in the absence of Rimiducid, VIR41 also demonstrated unexpected strong oncolytic activity against MSS colorectal cancer. Since MSS colorectal cancer does not respond to immune checkpoint inhibitors, VIR41 is an ideal agent to treat MSS colorectal cancer, in addition to being suitable for use in treating various other types of cancers. In the presence of Rimiducid, the replication of VIR41 is selectively inhibited in healthy cells, but not in cancer cells, as also shown in Example 6. This supports that VIR41 can be used to safely treat cancer patients with severe immune deficiency who are sensitive to infection from vaccinia virus or other types of viruses. In such a situation, VIR41 and Rimiducid can be simultaneously administered into these patients.

3. iFas Inducible System

The Fas receptor (Fas) is a membrane signaling protein that mediates a death signal following its aggregation by the Fas ligand (FasL), a member of the tumor necrosis factor family that initiates apoptosis by activation of caspases and the release of cytochrome c from the mitochondria, that leads to additional caspase activation followed by cellular degradation and death. See Savurma et al., Cell Death & Differentiation volume 10, pages 36-44 (2003).

Provided herein, in various embodiments, are recombinant viruses including heterologous nucleic acids encoding for an inducible “safety switch” mechanism including an inducible iFas gene fused to F36V-FKBP, a mutated version of human FK506 binding protein (FKBP12) that allows for conditional dimerization of F36V-FKBP upon induction of the system with AP1903, an FDA approved drug for use in humans. See Straathof et al., Blood (2005) 105:11, 4247-4254. Administration of this small molecule results in cross-linking and activation of the proapoptotic fusion molecule iFas (Fas fused to F36V-FKBP). Further details about iFas inducible systems are discussed in Belshaw et al. Chemistry & Biology September 1996, 3:731-738, which is hereby incorporated by reference for all purposes.

Accordingly, provided herein, in some embodiments, is a recombinant oncolytic virus comprising at least one heterologous nucleic acid encoding one or more heterologous gene product, wherein the one or more heterologous gene product is or comprises an apoptosis-inducible protein. In some embodiments, the apoptosis-inducible protein comprises a proapoptotic molecule fused with an FKBP variant that is able to bind a chemical inducer of dimerization (CID). In some embodiments, the FKBP variant is FKBP-F36V. In some embodiments, the FKBP variant is FKBP-F36V, and the FKBP-F36V comprises the sequence of amino acids set forth in SEQ ID NO: 56. In some embodiments, the chemical inducer of dimerization is AP1903 (Rimiducid). In some embodiments, the proapoptotic molecule is Fas.

In some embodiments, the apoptosis-inducible protein is an inducible Fas (iFas). In some embodiments, the iFas comprises a proapoptotic molecule fused with an FKBP variant that is able to bind a chemical inducer of dimerization (CID). In some embodiments, the apoptosis-inducible protein is an inducible Fas (iFas) comprising the sequence set forth in SEQ ID NO: 28, or is a sequence of amino acids that has at least 85%, 90% or 95% sequence identity to the amino acid sequence of SEQ ID NO: 28.

As such, in some embodiments, the recombinant virus expresses an inducible iFas system. Specifically, in some embodiments, the recombinant viruses comprise an expression cassette containing an inducible Fas (Fas receptor) cDNA molecule under the control of a vaccinia synthetic early promoter PSE inserted into the J2R gene locus. The iFas molecule includes a Fas receptor protein linked to 2 F36V-FKBP, a mutated version of human FK506 binding protein (FKBP12; GenBank AH002 818) that contain an F36V mutation.

In some embodiments, the recombinant virus comprises a polynucleotide that encodes an Fas molecule linked to 2 F36V-FKBP, wherein the iFas polypeptide comprises an amino acid sequence having at least 90%, 91%, 92%, 93%, 94%, 95%, 95%, 97%, 98%, or 99% sequence identity to the amino acid sequence of SEQ ID NO: 28. In some embodiments, the recombinant virus includes a polypeptide with a sequence of amino acids that has at least 70%, such as at least 75%, 80%, 85% or 90% sequence identity to SEQ ID NO: 28.

In some embodiments, the recombinant virus comprises a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, or 99.9% sequence identity to the nucleic acid sequence of SEQ ID NO: 9, but is less than 100% identical SEQ ID NO: 9. In some embodiments, the recombinant virus comprises the sequence of nucleotides of SEQ ID NO: 9. In some embodiments, the polynucleotide that encodes the iFas molecule is operably linked to the PSE promoter.

In some embodiments, the recombinant virus, which includes a polynucleotide that encodes an iFas molecule (SEQ ID NO: 28) is derived from the clonal VIP02 (SEQ ID NO: 1) strain and includes a sequence of nucleotides that has at least 70%, such as at least 75%, 80%, 85% or 90% sequence identity to SEQ ID NO: 9 (also named VIR42).

In some embodiments, the recombinant virus, e.g., recombinant oncolytic virus, comprises an iFas, and the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 9. A recombinant oncolytic virus comprising the nucleic acid sequence of SEQ ID NO: 9 is also referred to herein as VIR42.

In some embodiments, VIR42 exhibits enhanced anti-tumorigenic activity compared to VIR13 after induction with an effective dose of Rimiducid. In some embodiments, VIR42 exhibits less anti-tumorigenic activity compared to VIR13 after induction with an effective dose of Rimiducid. In some embodiments, VIR42 exhibits similar anti-tumorigenic activity compared to VIR13 after induction with an effective dose of Rimiducid.

### IV. Propagation and Production of Viruses

The clonal virus strains or recombinant virus strains thereof provided herein can be propagated in an appropriate host cell. Such cells can be a group of a single type of cells or a mixture of different types of cells. Host cells can include cultured cell lines primary cells, and proliferative cells. These host cells can include any of a variety of animal cells, such as mammalian, avian and insect cells and tissues that are susceptible to the virus, such as vaccinia virus, infection, including chicken embryo, rabbit, hamster, and monkey kidney cells. Suitable host cells include, but are not limited to, hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, and cell lines including, for example, CV-1, BSC40, Vero, and BSC-1, and human HeLa cells. Typically, viruses are propagated in cell lines that can be grown at monolayers or in suspension. For example, exemplary cell lines for the propagation of vaccinia viruses include, but are not limited to, CV-1, BSC40, Vero, BGM, BSC-1 and RK-13 cells. Exemplary cell lines for the propagation of adenovirus include, but are not limited to, HeLa, MK, HEK 293 and HDF cells. Exemplary cell lines for the propagation of herpesviruses include, but are not limited to, WI-38 and HeLa cells. Other cell lines suitable for the propagation of a variety of viruses are well known in the art. Purification of the cultured strain from the system can be effected using standard methods.

The concentration of virus in a solution, or virus titer, can be determined by a variety of methods known in the art. In some methods, a determination of the number of infectious virus particles is made (typically termed plaque forming units, or PFU), while in other methods, a determination of the total number of viral particles, either infectious or not, is made. Methods that calculate the number of infectious virions include, but are not limited to, the endpoint dilution method, which determines the titer within a certain range, such as one log, and the plaque assay, in which titrations of the virus are grown on cell monolayers and the number of plaques is counted after several days to several weeks. Methods that determine the total number of viral particles, including infectious and non-infectious, include, but are not limited to, immunohistochemical staining methods that utilize antibodies that recognize a viral antigen and which can be visualized by microscopy or FACS™ analysis; optical absorbance, such as at 260 nm; and measurement of viral nucleic acid, such as by PCR, RT-PCR, or quantitation by labeling with a fluorescent dye.

Once the virus has been purified (or to a desired purity) and the titer has been determined, the virus can be stored in conditions which optimally maintain its infectious integrity. Typically, viruses are stored in the dark, because light serves to inactivate the viruses over time. Viral stability in storage is usually dependent upon temperatures. Although some viruses are thermostable, most viruses are not stable for more than a day at room temperature, exhibiting reduced viability (Newman et al., (2003) J. Inf. Dis. 187:1319-1322). For short-term storage of viruses, for example, 1 day, 2 days, 4 days or 7 days, temperatures of approximately 4° C. are generally recommended. For long-term storage, most viruses can be kept at −20° C., −70° C., or −80° C. When frozen in a simple solution such as PBS or Tris solution (20 mM Tris pH 8.0, 200 NaCl, 2-3% glycerol or sucrose) at these temperatures, the virus can be stable for 6 months to a year, or even longer. Repeated freeze-thaw cycles are generally avoided, however, since it can cause a decrease in viral titer. The virus also can be frozen in media containing other supplements in the storage solution, which can further preserve the integrity of the virus. For example, the addition of serum or bovine serum albumin (BSA) to a viral solution stored at −80° C. can help retain virus viability for longer periods of time and through several freeze-thaw cycles. In other examples, the virus sample is dried for long-term storage at ambient temperatures. Viruses can be dried using various techniques including, but not limited to, freeze-drying, foam-drying, spray-drying and desiccation. Other methods for the storage of viruses at ambient, refrigerated or freezing temperatures are known in the art, and include, but are not limited to, those described in U.S. Pat. Nos. 5,149,653; 6,165,779; 6,255,289; 6,664,099; 6,872,357; and 7,091,030; and in U.S. Pat. Pub. Nos. 2003-0153065 and 2005-0032044.

Viruses can react differently to each storage method. For example, polio virus is readily degraded at room temperature in aqueous suspension, is stable for only two weeks at 0° C., and is destroyed by lyophilization. For this particular virus, methods of storage typically involve freezing at −70° C. or refrigeration at 4° C. In contrast, vaccinia virus is considered very stable, and can be stored in solution at 4° C., frozen at, for example −20° C., −70° C. or −80° C., or lyophilized with little loss of viability (Newman et al., (2003) J. Inf. Dis. 187:1319-1322, Hruby et al., (1990) Clin. Microb. Rev. 3:153-170). Methods and conditions suitable for the storage of particular viruses are known in the art, and can be used to store the viruses used in the methods presented herein.

Water is a reactant in nearly all of the destructive pathways that degrade viruses in storage. Further, water acts as a plasticizer, which allows unfolding and aggregation of proteins. Since water is a participant in almost all degradation pathways, reduction of the aqueous solution of viruses to a dry powder provides an alternative formulation methodology to enhance the stability of such samples. Lyophilization, or freeze-drying, is a drying technique used for storing viruses (see, e.g., Cryole et al., (1998) Pharm. Dev. Technol., 3(3), 973-383). There are three stages to freeze-drying: freezing, primary drying, and secondary drying. During these stages, the material is rapidly frozen and dehydrated under high vacuum. Once lyophilized, the dried virus can be stored for long periods of time at ambient temperatures, and reconstituted with an aqueous solution when needed. Various stabilizers can be included in the solution prior to freeze-drying to enhance the preservation of the virus. For example, it is known that high molecular weight structural additives, such as serum, serum albumin or gelatin, aid in preventing viral aggregation during freezing, and provide structural and nutritional support in the lyophilized or dried state. Amino acids such as arginine and glutamate, sugars, such as trehalose, and alcohols such as mannitol, sorbitol and inositol, can enhance the preservation of viral infectivity during lyophilization and in the lyophilized state. When added to the viral solution prior to lyophilization, urea and ascorbic acid can stabilize the hydration state and maintain osmotic balance during the dehydration period. Typically, a relatively constant pH of about 7.0 is maintained throughout lyophilization.

Immediately prior to use, the virus can be prepared at an appropriate concentration in suitable media, and can be maintained at a cool temperature, such as on ice, until use. If the virus was lyophilized or otherwise dried for storage, then it can be reconstituted in an appropriate aqueous solution. The aqueous solution in which the virus is prepared is typically the medium used in the assay (e.g., DMEM or RPMI) or one that is compatible, such as a buffered saline solution (e.g., PBS, TBS, Hepes solution). For pharmaceutical applications, the virus can be immediately prepared or reconstituted in a pharmaceutical solution. Numerous pharmaceutically acceptable solutions for use are well known in the art (see e.g. Remington's Pharmaceutical Sciences (18th edition) ed. A. Gennaro, 1990, Mack Publishing Co., Easton, Pa.). In one example, the viruses can be diluted in a physiologically acceptable solution, such as sterile saline or sterile buffered saline, with or without an adjuvant or carrier. In other examples, the pharmaceutical solution can contain a component that provides viscosity (e.g. glycerol) and/or component that has bactericidal properties (e.g. phenol). In some examples, the virus is prepared in a relatively concentrated solution so that only a small volume is required in the assay. For example, if 1×106 pfu of virus is being added to tumor cells in a 96 well plate, then the virus can be prepared at a concentration of 1×10⁸ pfu/mL so that only 10 μl is added to each well. The particular concentration can be empirically determined by one of skill in the art depending on the particular application.

In some embodiments, the recombinant oncolytic virus or the clonal VACV strain, such as any of those provided herein, exhibits enhanced production of extracellular enveloped virions (EEV) after cell infection, optionally as determined by percentage of EEV, wherein the percentage of EEV is determined by the formula: viral titer in supernatant/(viral titer in supernatant+viral titer in cell lysate)*100. Enhanced production can be determined, e.g., in comparison to a normal virus counterpart, wildtype virus counterpart, or unmodified counterpart of the recombinant oncolytic virus, or by having a percentage of at least 5%, 10%, or 15% of infectious particles being EEV.

### V. Pharmaceutical Compositions, Combinations and Kits

Provided herein are pharmaceutical compositions, combinations and kits containing a virus, e.g., recombinant virus or recombinant oncolytic virus, provided herein. Pharmaceutical compositions can include a virus provided herein and a pharmaceutical carrier. Combinations can include, for example, two or more viruses, a virus and a detectable compound, a virus and a therapeutic compound, a virus and a viral expression modulating compound, or any combination thereof. Kits can include one or more pharmaceutical compositions or combinations provided herein, and one or more components, such as instructions for use, a device for administering the pharmaceutical composition or combination to a subject, a device for administering a therapeutic or diagnostic compound to a subject or a device for detecting a virus in a subject.

A virus contained in a pharmaceutical composition, combination or kit can include any virus provided herein, including any isolated clonal virus strain described herein, and any virus, e.g., recombinant virus or recombinant oncolytic virus as described in Section III. The pharmaceutical compositions, combinations or kits can include one or more additional viruses that can be selected from a virus provided herein or other therapeutic or diagnostic virus.

### A. Pharmaceutical Compositions

Provided herein are pharmaceutical compositions containing a virus provided herein and a suitable pharmaceutical carrier. A pharmaceutically acceptable carrier includes a solid, semi-solid or liquid material that acts as a vehicle carrier or medium for the virus. Pharmaceutical compositions provided herein can be formulated in various forms, for example in solid, semi-solid, aqueous, liquid, powder or lyophilized form. Exemplary pharmaceutical compositions containing a virus provided herein include, but are not limited to, sterile injectable solutions, sterile packaged powders, eye drops, tablets, pills, powders, lozenges, sachets, cachets, elixirs, suspensions, emulsions, solutions, syrups, aerosols (as a solid or in a liquid medium), ointments, soft and hard gelatin capsules, and suppositories.

Examples of suitable pharmaceutical carriers are known in the art and include, but are not limited to, water, buffers, saline solutions, phosphate buffered saline solutions, various types of wetting agents, sterile solutions, alcohols, gum arabic, vegetable oils, benzyl alcohols, gelatin, glycerin, carbohydrates, such as lactose, sucrose, dextrose, amylose or starch, sorbitol, mannitol, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preserving agents, analgesic agents, binders, disintegrants, coloring, diluents, excipients, extenders, glidants, solubilizers, stabilizers, tonicity agents, vehicles, viscosity agents, flavoring agents, sweetening agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as, but not limited to, crystalline cellulose, microcrystalline cellulose, citric acid, dextrin, liquid glucose, lactic acid, lactose, magnesium chloride, potassium metaphosphate, starch, among others. Such carriers and/or additives can be formulated by conventional methods and can be administered to the subject at a suitable dose. Stabilizing agents such as lipids, nuclease inhibitors, polymers, and chelating agents can preserve the compositions from degradation within the body. Other suitable formulations for use in a pharmaceutical composition can be found, for example, in Remington: The Science and Practice of Pharmacy (2005, Twenty-first edition, Gennaro & Gennaro, eds., Lippencott Williams and Wilkins).

Pharmaceutical formulations that include a virus provided herein for injection or mucosal delivery typically include aqueous solutions of the virus provided in a suitable buffer for injection or mucosal administration or lyophilized forms of the virus for reconstitution in a suitable buffer for injection or mucosal administration. Such formulations optionally can contain one or more pharmaceutically acceptable carriers and/or additives as described herein or known in the art. Liquid compositions for oral administration generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Pharmaceutical compositions provided herein can be formulated to provide quick, sustained or delayed released of a virus as described herein by employing procedures known in the art. For preparing solid compositions such as tablets, a virus provided herein is mixed with a pharmaceutical carrier to form a solid composition. Optionally, tablets or pills are coated or otherwise compounded to provide a dosage form affording the advantage of prolonged action in the subject. For example, a tablet or pill comprises an inner dosage and an outer dosage component, the latter being in the form of an envelope over the former. The two components can be separated by an enteric layer, for example, which serves to resist disintegration in the stomach and permit the inner component to pass intact into the duodenum or to be delayed in release. A variety of materials are used for such enteric layers or coatings, including, for example, a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. These liquid or solid compositions optionally can contain suitable pharmaceutically acceptable excipients and/or additives as described herein or known in the art. Such compositions are administered, for example, by the oral or nasal respiratory route for local or systemic effect. Compositions in pharmaceutically acceptable solvents are nebulized by use of inert gases. Nebulized solutions are inhaled, for example, directly from the nebulizing device, from an attached facemask tent, or from an intermittent positive pressure-breathing machine. Solution, suspension, or powder compositions are administered, orally or nasally, for example, from devices, which deliver the formulation in an appropriate manner such as, for example, use of an inhaler.

Pharmaceutical compositions provided herein can be formulated for transdermal delivery via transdermal delivery devices (“patches”). Such transdermal patches are used to provide continuous or discontinuous infusion of a virus provided herein. The construction and use of transdermal patches for the delivery of pharmaceutical agents are performed according to methods known in the art. See, for example, U.S. Pat. No. 5,023,252. Such patches are constructed for continuous, pulsatile, or on-demand delivery of a virus provided herein.

Colloidal dispersion systems that can be used for delivery of viruses include macromolecule complexes, nanocapsules, microspheres, beads and lipid-based systems including oil-in-water emulsions (mixed), micelles, liposomes and lipoplexes. An exemplary colloidal system is a liposome. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example, by coupling the liposome to a specific ligand, for example, an antibody, a receptor, sugar, glycolipid and protein by methods know to those of skill in the art). Monoclonal antibodies can be used to target liposomes to specific tissues, for example, tumor tissue, via specific cell-surface ligands.

### B. Host Cells

Host cells that contain a virus provided herein are provided. Such cells can be employed in vitro use or in vivo use, for example, as described in the diagnostic or therapeutic methods provided herein. The host cells can be a group of a single type of cells or a mixture of different types of cells. Host cells can include cultured cell lines, primary cells and proliferative cells. The host cells can include any of a variety of animal cells, such as mammalian, avian and insect cells and tissues that are susceptible to infection by the virus, including, but not limited to, human, primate, rodent (e.g. mouse, rate, hamster, or rabbit) and chicken embryo cells. Suitable host cells include, but are not limited to, hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, tumor cells and cell lines including, for example, CV-1, BSC40, Vero, BSC40 and BSC-1, and human HeLa cells. Methods for infecting and/or transforming host cells, phenotypically selecting infected cells or transformants, and other such methods are known in the art.

### C. Combinations

Provided are combinations of a virus provided herein and a second agent, such as a second virus or other therapeutic or diagnostic agent.

Accordingly, in some embodiments, the methods, e.g., therapeutic methods, provided herein further comprise administering a second therapeutic agent for the treatment of the proliferative disorder.

In some embodiments, the methods provided herein further comprise another treatment. In some embodiments, the another treatment is selected from among surgery, radiation therapy, immunosuppressive therapy and administration of an anticancer agent. In some embodiments, the another (further) treatment is administration of an anticancer agent selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, or an immunotherapeutic agent, and a combination of any of the preceding thereof.

A combination can include a virus provided herein with one or more additional viruses, including, for example, one or more additional diagnostic or therapeutic viruses. A combination can contain pharmaceutical compositions containing a virus provided herein or host cells containing a virus as described herein. A combination can also include any virus or reagent for effecting the anti-tumorigenic and toxicity reducing activities described herein in accordance with the methods provided herein. Combinations can also contain a compound used for the modulation of gene expression from endogenous or heterologous genes encoded by the virus. Combinations can also contain a compound used for the modulation of protein activation such as compounds that can induce protein multimerization including AP1903 and AP20187.

Combinations provided herein can contain a virus and a therapeutic compound. Therapeutic compounds for the compositions provided herein can be, for example, an anti-cancer or chemotherapeutic agent or compound. Exemplary therapeutic agents or compounds include, for example, cytokines, growth factors, photosensitizing agents, radionuclides, toxins, siRNA molecules, enzyme/prodrug pairs, anti-metabolites, signaling modulators, anti-cancer antibiotics, anti-cancer antibodies, checkpoint inhibitors (e.g. as anti-programmed death 1 (PD-1) receptor and anti-programmed death ligand 1 (PD-L1)/L2 inhibitors), angiogenesis inhibitors, chemotherapeutic compounds, antimetastatic compounds or a combination of any thereof. Viruses provided herein can be combined with an anti-cancer compound, such as a platinum coordination complex. Exemplary platinum coordination complexes include, for example, cisplatin, carboplatin, oxaliplatin, DWA2114R, NK121, IS 3 295, and 254-S. Exemplary chemotherapeutic agents also include, but are not limited to, methotrexate, vincristine, adriamycin, non-sugar containing chloroethylnitrosoureas, 5-fluorouracil, mitomycin C, bleomycin, doxorubicin, dacarbazine, Taxol®, fragyline, Meglumine GLA, valrubicin, carmustine, polifeprosan, MM1270, BAY 12-9566, RAS farnesyl transferase inhibitor, farnesyl transferase inhibitor, MMP, MTA/LY231514, lometrexol/LY264618, Glamolec, CI-994, TNP-470, Hycamtin®/topotecan, PKC412, Valspodar/PSC833, Novantrone®/mitoxantrone, Metaret®/suramin, BB-94/batimastat, E7070, BCH-4556, CS-682, 9-AC, AG3340, AG3433, Incel™/VX-710, VX-853, ZD0101, IS1641, ODN 698, TA 2516/marimastat, BB2516/marimastat, CDP 845, D2163, PD183805, DX8951f, Lemonal DP 2202, FK 317, picibanil/OK-432, valrubicin/AD 32, strontium-89/Metastron®, Temodal®/temozolomide, Yewtaxan/paclitaxel, Taxol®/paclitaxel, Paxex/paclitaxel, Cyclopax/oral paclitaxel, Xeloda®/capecitabine, Furtulon™/doxifluridine, oral taxoids, SPU-077/cisplatin, HMR 1275/flavopiridol, CP-358 (774)/EGFR, CP-609 (754)/RAS oncogene inhibitor, BMS-182751/oral platinum, UFT (Tegafur/Uracil), Ergamisol®/levamisole, Campto®/levamisole, Eniluracil/776C85/5FU enhancer, Camptosar®/irinotecan, Tomudex®/raltitrexed, Leustatin®/cladribine, Caelyx®/liposomal doxorubicin, Myocetliposomal doxorubicin, Doxilliposomal doxorubicin, Evacet™/liposomal doxorubicin, Fludara®/fludarabine, Pharmorubicinepirubicin, DepoCyt®, ZD1839, LU 79553/Bis-Naphthalimide, LU 103793/Dolastain, Gemzar®/gemcitabine, ZD 0473/Anormed, YM 116, Iodine seeds, CDK4 and CDK2 inhibitors, PARP inhibitors, D4809/dexifosfamide, Ifex®/Mesnex®/ifosfamide, Vumon®/teniposide, Paraplatin®/carboplatin, Platinol®/cisplatin, VePesid®/Eposin®/Etopophos®/etoposide, ZD 9331, Taxotere®/docetaxel, prodrugs of guanine arabinoside, taxane analogs, nitrosoureas, alkylating agents such as melphalan and cyclophosphamide, aminoglutethimide, asparaginase, busulfan, carboplatin, chlorambucil, cytarabine HCl, dactinomycin, daunorubicin HCl, estramustine phosphate sodium, etoposide (VP16-213), floxuridine, fluorouracil (5-FU), flutamide, hydroxyurea (hydroxycarbamide), ifosfamide, interferon alfa-2a, interferon alfa-2b, leuprolide acetate (LHRH-releasing factor analogue), lomustine (CCNU), mechlorethamine HCl (nitrogen mustard), mercaptopurine, mesna, mitotane (o,p′-DDD), mitoxantrone HCl, octreotide, plicamycin, procarbazine HCl, streptozocin, tamoxifen citrate, thioguanine, thiotepa, vinblastine sulfate, amsacrine (m-AMSA), azacitidine, erythropoietin, hexamethylmelamine (HMM), interleukin 2, mitoguazone (methyl-GAG; methyl glyoxal bis-guanylhydrazone; MGBG), pentostatin (2′deoxycoformycin), semustine (methyl-CCNU), teniposide (VM-26) and vindesine sulfate. Additional exemplary therapeutic compounds for the use in pharmaceutical compositions and combinations provided herein can be found elsewhere herein (see e.g., Section I for exemplary cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, and chemotherapeutic compounds).

In some examples, the combination can include additional therapeutic agents or compounds such as, for example, agents or compounds that are substrates for enzymes encoded and expressed by the virus, or other therapeutic compounds provided herein or known in the art to act in concert with a virus. For example, the virus can express an enzyme that converts a prodrug into an active chemotherapy drug for killing the cancer cell. Hence, combinations provided herein can contain a therapeutic agent or compound, such as a prodrug. An exemplary virus/therapeutic agent or compound combination can include a virus encoding Herpes simplex virus thymidine kinase with the prodrug ganciclovir. Additional exemplary enzyme/pro-drug pairs, for the use in combinations provided include, but are not limited to, varicella zoster thymidine kinase/ganciclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid, cytochrome P450/acetominophen, horseradish peroxidase/indole-3-acetic acid, nitroreductase/C B1954, rabbit carboxylesterase/7-ethyl-10-[4-(1-piperidino)-1-piperidino]carbonyloxycamptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin-glucuronide, thymidine phosphorylase/5′-deoxy-5-fluorouridine, deoxycytidine kinase/cytosine arabinoside, beta-lactamase and linamerase/linamarin. Additional exemplary prodrugs, for the use in combinations can also be found elsewhere herein (see, e.g., Section I). Any of a variety of known combinations provided herein or otherwise known in the art can be included in the combinations provided herein.

In some examples, the combination can include agents or compounds that can kill or inhibit viral growth or toxicity. Such agents or compounds can be used to alleviate one or more adverse side effects that can result from viral infection (see, e.g. U.S. Patent Pub. No. US 2009-016228-A1). Combinations provided herein can contain antibiotic, antifungal, anti-parasitic or antiviral compounds for treatment of infections. In some examples, the antiviral compound is a chemotherapeutic agent that inhibits viral growth or toxicity. Exemplary antibiotics which can be included in a combination with a virus provided herein include, but are not limited to, ceftazidime, cefepime, imipenem, aminoglycoside, vancomycin and antipseudomonal β-lactam. Exemplary antifungal agents which can be included in a combination with a virus provided herein include, but are not limited to, amphotericin B, dapsone, fluconazole, flucytosine, griseofulvin, itraconazole, ketoconazole, miconazole, clotrimazole, nystatin, and combinations thereof. Exemplary antiviral agents can be included in a combination with a virus provided herein include, but are not limited to, cidofovir, alkoxyalkyl esters of cidofovir (CDV), cyclic CDV, and (S)-9-(3-hydroxy-2 phosphonylmethoxypropyl)adenine, 5-(dimethoxymethyl)-2′-deoxyuridine, isatin-beta-thiosemicarbazone, N-methanocarbathymidine, brivudine, 7-deazaneplanocin A, ST-246, Gleevec®, 2′-beta-fluoro-2′,3′-dideoxyadenosine, indinavir, nelfinavir, ritonavir, nevirapine, AZT, ddI, ddC, and combinations thereof. Typically, combinations with an antiviral agent contain an antiviral agent known to be effective against the virus of the combination. For example, combinations can contain a vaccinia virus with an antiviral compound, such as cidofovir, alkoxyalkyl esters of cidofovir, ganciclovir, acyclovir, ST-246, Gleevec®, and derivatives thereof.

In some examples, the combination can include a detectable compound. A detectable compound can include, for example, a ligand, substrate or other compound that can interact with and/or bind specifically to a protein or RNA encoded and expressed by the virus, and can provide a detectable signal, such as a signal detectable by tomographic, spectroscopic, magnetic resonance, or other known techniques. In some examples, the protein or RNA is an exogenous protein or RNA. In some examples, the protein or RNA expressed by the virus modifies the detectable compound where the modified compound emits a detectable signal. Exemplary detectable compounds can be, or can contain, an imaging agent such as a magnetic resonance, ultrasound or tomographic imaging agent, including a radionuclide. The detectable compound can include any of a variety of compounds as provided elsewhere herein or are otherwise known in the art. Exemplary proteins that can be expressed by the virus and a detectable compound combinations employed for detection include, but are not limited to luciferase and luciferin, β-galactosidase and (4,7,10-tri(acetic acid)-1-(2-β-galactopyranosylethoxy)-1,4,7,10-tetraazacyclododecane) gadolinium (Egad), and other combinations known in the art.

In some examples, the combination can include a gene expression modulating compound that regulates expression of one or more genes encoded by the virus. Compounds that modulate gene expression are known in the art, and include, but are not limited to, transcriptional activators, inducers, transcriptional suppressors, RNA polymerase inhibitors and RNA binding compounds such as siRNA or ribozymes. Any of a variety of gene expression modulating compounds known in the art can be included in the combinations provided herein. Typically, the gene expression modulating compound included with a virus in the combinations provided herein will be a compound that can bind, inhibit or react with one or more compounds, active in gene expression such as a transcription factor or RNA of the virus of the combination. An exemplary virus/expression modulator combinations can be a virus encoding a chimeric transcription factor complex having a mutant human progesterone receptor fused to a yeast GAL4 DNA-binding domain an activation domain of the herpes simplex virus protein VP16 and also containing a synthetic promoter containing a series of GAL4 recognition sequences upstream of the adenovirus major late E1B TATA box, where the compound can be RU486 (see, e.g., Yu et al., (2002) Mol Genet Genomics 268:169-178). A variety of other virus/expression modulator combinations known in the art also can be included in the combinations provided herein.

In some examples, the combination can contain nanoparticles. Nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen.

In some examples, the combination can contain one or more additional therapeutic and/or diagnostic viruses or other therapeutic and/or diagnostic microorganism (e.g. therapeutic and/or diagnostic bacteria) for diagnosis or treatment. Exemplary therapeutic and/or diagnostic viruses are known in the art and include, but are not limited to, therapeutic and/or diagnostic poxviruses, herpesviruses, adenoviruses, adeno-associated viruses, and reoviruses.

### D. Kits

The viruses, cells, pharmaceutical compositions or combinations provided herein can be packaged as kits. Kits can optionally include one or more components such as instructions for use, devices and additional reagents, and components, such as tubes, containers and syringes for practice of the methods. Exemplary kits can include any virus provided herein, and can optionally include instructions for use, a device for detecting a virus in a subject, a device for administering the virus to a subject, or a device for administering an additional agent or compound to a subject.

In one example, a kit can contain instructions. Instructions typically include a tangible expression describing the virus and, optionally, other components included in the kit, and methods for administration, including methods for determining the proper state of the subject, the proper dosage amount, and the proper administration method, for administering the virus. Instructions can also include guidance for monitoring the subject over the duration of the treatment time.

In another example, a kit can contain a device for detecting a virus in a subject. Devices for detecting a virus in a subject can include a low light imaging device for detecting light, for example, emitted from luciferase, or fluoresced from fluorescent protein, such as a green or red fluorescent protein, a magnetic resonance measuring device such as an MRI or NMR device, a tomographic scanner, such as a PET, CT, CAT, SPECT or other related scanner, an ultrasound device, or other device that can be used to detect a protein expressed by the virus within the subject. Typically, the device of the kit will be able to detect one or more proteins expressed by the virus of the kit. Any of a variety of kits containing viruses and detection devices can be included in the kits provided herein, for example, a virus expressing luciferase and a low light imager or a virus expressing fluorescent protein, such as a green or red fluorescent protein, and a low light imager.

Kits provided herein also can include a device for administering a virus to a subject. Any of a variety of devices known in the art for administering medications, pharmaceutical compositions and vaccines can be included in the kits provided herein. Exemplary devices include, but are not limited to, a hypodermic needle, an intravenous needle, a catheter, a needle-less injection device, an inhaler and a liquid dispenser, such as an eyedropper. For example, a virus to be delivered systemically, for example, by intravenous injection, can be included in a kit with a hypodermic needle and syringe. Typically, the device for administering a virus of the kit will be compatible with the virus of the kit; for example, a needle-less injection device such as a high-pressure injection device can be included in kits with viruses not damaged by high-pressure injection, but is typically not included in kits with viruses damaged by high-pressure injection.

Kits provided herein can also include a device for administering an additional agent or compound to a subject. Any of a variety of devices known in the art for administering medications to a subject can be included in the kits provided herein. Exemplary devices include, but are not limited to, a hypodermic needle, an intravenous needle, a catheter, a needle-less injection device, an inhaler and a liquid dispenser, such as an eyedropper. Typically, the device for administering the compound of the kit will be compatible with the desired method of administration of the compound. For example, a compound to be delivered systemically or subcutaneously can be included in a kit with a hypodermic needle and syringe.

### VI. Therapeutic, Diagnostic and Monitoring Methods

The viruses provided herein, including the clonal virus strains and the recombinant viral strains provided herein, can be used in diagnostic, monitoring, and therapeutic methods. For example, in therapeutic methods the viruses provided herein, including the clonal virus strains and recombinant viral strains, can be used for the treatment of proliferative disorders or conditions, including the treatment of cancerous cells, hyperplasia, neoplasms, tumors, metastases and other immunoprivileged cells or tissues, such as wounded or inflamed tissues. The viruses provided herein, including the clonal virus strains and the recombinant viral strains provided herein, can be used in diagnostic methods for detecting and imaging of cancerous cells, hyperplasia, tumors and metastases monitoring treatment. In other examples, the viruses provided herein, including the clonal virus strains and recombinant strains, can be used in diagnostic or monitoring methods to detect virus activity in the host. The diagnostic and therapeutic methods provided herein include, but are not limited to, administering a virus provided herein to a subject containing a tumor, an hyperplasia, a cancer, and/or metastases. In other examples, the viruses provided herein, including the clonal virus strains and the recombinant viral strains provided herein, can be used as vaccines in vaccination methods.

The administered viruses possess one or more characteristics including attenuated pathogenicity, low toxicity, enhanced anti-tumorigenicity, preferential accumulation in tumors, ability to activate an immune response against tumor cells, replication competence, ability to express additional exogenous diagnostic and/or therapeutic genes, ability to inhibit tumor, hyperplasia, metastasis and/or cancer in a subject, ability to induce apoptosis in an infected cell and/or subject, ability to avoid the host's complement, ability to avoid the immunogenic response from a subject, and ability to enhance a subject's immune response against an hyperplasia, tumor, cancer and/or metastasis. The viruses can be administered for diagnosis, monitoring, such as monitoring therapy, and/or therapy of subjects, such as, but not limited to humans and other mammals, including, but not limited to, rodents, dogs, cats, primates and livestock. The viruses provided herein can be used or modified for use in any known methods (or uses) in which viruses have been employed or can be employed. Any virus, including the VIP02, VIR11, VIR13, VIR 25, VIR27, VIR37, VIR40, VIR41, VIR42, VIR46, VIR49, VIR52, VIR57, VIR71, VIR86, VIR93, VIR94, VIR96, VIR100, VIR103, VIR105, VIR106, VIR109, VIR111, VIR113, VIR114, VIR115, VIR123, VIR127, and/or VIR128 viruses and derivatives thereof, including a virus comprising a nucleic acid sequence having at least 95%, 96%, 97%, 98%, 99%, 99.1%, 99.2%, 99.3%, 99.4%, 99.5%, 99.6%, 99.7%, 99.8%, or 99.9% sequence identity to the genomic sequence of virus selected from the group consisting of VIP02, VIR11, VIR13, VIR 25, VIR27, VIR37, VIR40, VIR41, VIR42, VIR46, VIR49, VIR52, VIR57, VIR71, VIR86, VIR93, VIR94, VIR96, VIR100, VIR103, VIR105, VIR106, VIR109, VIR111, VIR113, VIR114, VIR115, VIR123, VIR127, and/or VIR128 can be used and/or modified for use in the therapeutic and diagnostic methods described below and discussed throughout the disclosure herein.

### A. Therapeutic Methods

The viruses provided herein, including the clonal virus strains and the recombinant viral strains provided herein, including any of the viruses, e.g., recombinant viruses or recombinant oncolytic viruses as described in Section III, for example, can be used for the treatment of proliferative disorders or conditions, including the treatment (such as inhibition) of cancerous cells, hyperplasia, neoplasms, tumors, metastases, cancer stem cells, and other immunoprivileged cells or tissues, such as wounded or inflamed tissues.

Accordingly, provided herein, in some embodiments, is a method of treating a proliferative disorder in a subject, comprising administering to the subject a virus, e.g., a recombinant virus or a recombinant oncolytic virus or an oncolytic virus, as described herein.

Also provided herein, in some embodiments, is a method of treating a proliferative disorder in a subject, comprising administering to the subject any composition, e.g., any of the pharmaceutical compositions, as described herein.

Also provided herein is a method of inhibiting virus replication, the method comprising contacting cells infected with a virus, e.g., a recombinant oncolytic virus, with AP1903 (Rimiducid), wherein the recombinant oncolytic virus comprises a heterologous nucleic acid encoding an apoptosis inducible protein. Also provided herein is a method of inhibiting virus replication in a subject, the method comprising administering to a subject AP1903 (Rimiducid), wherein the subject has been previously administered a recombinant oncolytic virus comprising a heterologous nucleic acid encoding an apoptosis inducible protein. The apoptosis-inducible protein can be any apoptosis-inducible protein as described herein, e.g., as described in Section III(D). In some embodiments, the contacting occurs in vivo in a subject, wherein the AP1903 (Rimiducid) has been administered to a subject previously administered with a recombinant oncolytic virus comprising the heterologous nucleic acid encoding an apoptosis inducible protein.

In some embodiments, the viruses provided herein preferentially accumulate in tumors or metastases. In some embodiments, the method inhibits virus replication preferentially in non-cancer cells. In some embodiments, the administration of a virus provided herein results in a slowing of tumor growth, without significantly affecting healthy, non-cancerous, and/or normal cells. In other embodiments, the administration of a virus provided herein results in a decrease in tumor volume without significantly affecting healthy, non-cancerous, and/or normal cells, including elimination or eradication of the tumor. The therapeutic methods and uses provided herein, however, do not require the administered virus to kill tumor cells or decrease the tumor size. Instead, the methods provided herein include, in some embodiments, administering to a subject a virus provided herein that can cause or enhance an anti-tumor immune response in the subject without significantly affecting healthy, non-cancerous, and/or normal cells. In some embodiments, the viruses provided herein can be administered to a subject without causing viral-induced disease in the subject. In some embodiments, the viruses provided herein can be administered to a subject without causing viral-induced disease in the subject, for example, without significantly affecting healthy, non-cancerous, and/or normal cells. In some embodiments, the viruses can elicit an anti-tumor immune response in the subject, without significantly affecting healthy, non-cancerous, and/or normal cells, where typically the viral-mediated anti-tumor immune response can develop, for example, over several days, a week or more, 10 days or more, two weeks or more, or a month or more. In some exemplary methods, the virus can be present in the tumor, and can cause an anti-tumor immune response without the virus itself causing enough tumor cell death to prevent tumor growth. In some embodiments, the tumor is a monotherapeutic tumor or monotherapeutic cancer, where the tumor or cancer does not decrease in volume when treated with the virus or a therapeutic agent alone.

In some examples, the therapeutic methods provided herein inhibit tumor growth in a subject, without significantly affecting healthy, non-cancerous, and/or normal cells, where the methods include administering to a subject a virus that can accumulate in a tumor and/or metastasis, and can cause or enhance an anti-tumor immune response. The anti-tumor immune response induced as a result of tumor or metastases-accumulated viruses can result in inhibition of tumor growth.

In some embodiments, the therapeutic methods provided herein inhibit growth or formation of a metastases in a subject, without significantly affecting healthy, non-cancerous, and/or normal cells, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in inhibition of metastasis growth or formation.

In other embodiments, the therapeutic methods provided herein decrease the size of a tumor and/or metastasis in a subject, without significantly affecting healthy, non-cancerous, and/or normal cells, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in a decrease in the size of the tumor and/or metastasis.

In some embodiments, the therapeutic methods provided herein eliminate a tumor and/or metastasis from a subject, without significantly affecting healthy, non-cancerous, and/or normal cells, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in elimination of the tumor and/or metastasis from the subject. In some examples, the therapeutic methods provided herein induce apoptosis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance apoptosis. The increased apoptosis induced as a result of tumor or metastases-accumulated viruses can result in inhibition of tumor growth. In some embodiments, the therapeutic methods provided herein induce apoptosis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance apoptosis. The increased apoptosis induced as a result of tumor or metastases-accumulated viruses can result in inhibition of metastasis growth or formation. In some examples, the therapeutic methods provided herein induce apoptosis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance apoptosis. The increased apoptosis induced as a result of tumor or metastases-accumulated viruses can result in decrease in the size of the tumor and/or metastasis.

In some embodiments, the therapeutic methods provided herein inhibits angiogenesis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or inhibit angiogenesis. The reduced angiogenesis induced as a result of tumor or metastases-accumulated viruses can result in inhibition of tumor growth. In some examples, the therapeutic methods provided herein inhibit angiogenesis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can inhibit angiogenesis. The reduced angiogenesis induced as a result of tumor or metastases-accumulated viruses can result in inhibition of metastasis growth or formation. In some embodiments, the therapeutic methods provided herein inhibit angiogenesis in a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can inhibit angiogenesis. The reduced angiogenesis induced as a result of tumor or metastases-accumulated viruses can result in decrease in the size of the tumor and/or metastasis.

In some embodiments, the therapeutic methods provided herein enhance the immune response against a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance an immune response. The enhanced immune response induced as a result of tumor or metastases-accumulated viruses can result in inhibition of tumor growth. In some examples, the therapeutic methods provided herein enhance the immune response against a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance an immune response. The enhanced immune response induced as a result of tumor or metastases-accumulated viruses can result in inhibition of metastasis growth or formation. In some examples, the therapeutic methods provided herein enhance the immune response against a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance an immune response. The enhanced immune response induced as a result of tumor or metastases-accumulated viruses can result in decrease in the size of the tumor and/or metastasis. In some embodiments, the therapeutic methods provided herein enhance the immune response against a tumor, cancer, hyperplasia, and or metastasis in a subject, without significantly affecting healthy, non-cancerous, and/or normal cells, where the methods include administering to a subject a virus that can accumulate in a tumor, cancer, hyperplasia, and or metastasis, and can cause or enhance an immune response. The enhanced immune response induced as a result of tumor or metastases-accumulated viruses can result in elimination of the tumor and/or metastasis from the subject.

In some embodiments, the therapeutic methods provided herein reduce virus toxicity to healthy cells while effecting a toxic and/or anti-tumorigenic effect in a tumor and/or metastasis from a subject, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in elimination of the tumor and/or metastasis from the subject without significantly affecting non-cancerous, healthy, and/or normal cells.

Methods of reducing or inhibiting tumor growth, inhibiting metastasis growth and/or formation, decreasing the size of a tumor or metastasis, eliminating a tumor or metastasis and/or cancer stem cell, without significantly affecting healthy, non-cancerous, and/or normal cells, or other tumor therapeutic methods provided herein include causing or enhancing an anti-tumor immune response, inducing apoptosis, and/or inhibiting angiogenesis in the host without significantly affecting healthy, non-cancerous, and/or normal cells in the host. The immune response of the host, being anti-tumor in nature, can be mounted against tumors and/or metastases in which viruses have accumulated, and can also be mounted against tumors and/or metastases in which viruses have not accumulated, including tumors and/or metastases that form after administration of the virus to the subject. Accordingly, a tumor and/or metastasis whose growth or formation is inhibited, or whose size is decreased, or that is eliminated, can be a tumor and/or metastasis in which the viruses have accumulated, or can also be a tumor and/or metastasis in which the viruses have not accumulated. Accordingly, provided herein are methods of reducing or inhibiting tumor growth, inhibiting metastasis growth and/or formation, decreasing the size of a tumor or metastasis, eliminating a tumor or metastasis, without significantly affecting healthy, non-cancerous, and/or normal cells, or other tumor therapeutic methods provided herein include causing or enhancing an anti-tumor immune response, inducing apoptosis, and/or inhibiting angiogenesis in the host without significantly affecting healthy, non-cancerous, and/or normal cells in the host, where the method includes administering to a subject a virus provided herein, where the virus accumulates in at least one tumor or metastasis and causes or enhances an anti-tumor immune response in the subject, and the immune response is also mounted against a tumor and/or metastasis in which the virus cell did not accumulate. In another embodiments, methods are provided for inhibiting or preventing recurrence of a neoplastic disease or inhibiting or preventing new tumor growth, where the methods include administering to a subject a virus provided herein that can accumulate in a tumor and/or metastasis, and can cause or enhance an anti-tumor immune response, and the anti-tumor immune response can inhibit or prevent recurrence of a neoplastic disease or inhibit or prevent new tumor growth.

The tumor or neoplastic disease therapeutic methods provided herein, such as methods of reducing or inhibiting tumor growth, inhibiting metastasis growth and/or formation, decreasing the size of a tumor or metastasis, eliminating a tumor or metastasis, without significantly affecting healthy, non-cancerous, and/or normal cells, or other tumor therapeutic methods provided herein include causing or enhancing an anti-tumor immune response, inducing apoptosis, avoiding host's complement, and/or inhibiting angiogenesis in the host without significantly affecting healthy, non-cancerous, and/or normal cells in the host, can also include administering to a subject a virus provided herein that can cause tumor cell lysis or tumor cell death, including cell suicide by apoptosis. Such a virus can be the same virus as the virus that can cause or enhance an anti-tumor immune response in the subject. Viruses, such as the viruses provided herein, can cause cell lysis or tumor cell death as a result of expression of an endogenous gene or as a result of an exogenous gene. Endogenous or exogenous genes can cause tumor cell lysis or inhibit cell growth as a result of direct or indirect actions, as is known in the art, including lytic channel formation or activation of an apoptotic pathway. Gene products, such as exogenous gene products can function to activate a prodrug to an active, cytotoxic form, resulting in cell death where such genes are expressed.

Such methods of tumor and/or metastasis treatment can include administration of a virus provided herein for therapy, such as for gene therapy, for cancer gene therapy, or for vaccine therapy. Such a virus can be used to stimulate humoral and/or cellular immune response, induce strong cytotoxic T lymphocytes responses in subjects who can benefit from such responses. For example, the virus can provide prophylactic and therapeutic effects against a tumor infected by the virus or other infectious diseases, by rejection of cells from tumors or lesions using viruses that express immunoreactive antigens (Earl et al., Science 234:728-831 (1986); Lathe et al., Nature (London) 32:878-880 (1987)), cellular tumor-associated antigens (Bernards et al., Proc. Natl. Acad. Sci. USA 84:6854-6858 (1987); Estin et al., Proc. Natl. Acad. Sci. USA 85:1052-1056 (1988); Kantor et al., J. Natl. Cancer Inst. 84:1084-1091 (1992); Roth et al., Proc. Natl. Acad. Sci. USA 93:4781-4786 (1996)) and/or cytokines (e.g., IL-2, IL-12), costimulatory molecules (B7-1, B7-2) (Rao et al., J. Immunol. 156:3357-3365 (1996); Chamberlain et al., Cancer Res. 56:2832-2836 (1996); Oertli et al., J. Gen. Virol. 77:3121-3125 (1996); Qin and Chatterjee, Human Gene Ther. 7:1853-1860 (1996); McAneny et al., Ann. Surg. Oncol.3:495-500 (1996)), or other therapeutic proteins.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005-0031643, now U.S. Pat. Nos. 7,588,767, 7,588,771, 7,662,398), which provide and exemplify the GLV-1h68 virus and derivatives thereof. Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production. Thus, the viruses provided herein and the viruses generated using the methods provided herein can be administered in a complete process that can be applied to all tumor systems with immunoprivileged tumor sites as site of privileged viral growth, which can lead to tumor elimination by the host's own immune system.

In some embodiments, the proliferative disorder is a tumor or a metastasis. In some embodiments, the proliferative disorder is a cancer. The cancer is not limited in this respect, and other metastatic diseases can be treated by the combinations provided herein. In some embodiments, the tumor or metastasis treated can be a solid tumor, such as of the lung and bronchus, breast, colon and rectum, kidney, stomach, esophagus, liver and intrahepatic bile duct, urinary bladder, brain and other nervous system, head and neck, oral cavity and pharynx, cervix, uterine corpus, thyroid, ovary, testes, prostate, malignant melanoma, cholangiocarcinoma, thymoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS. Exemplary tumors include, for example, pancreatic tumors, ovarian tumors, lung tumors, colon tumors, prostate tumors, cervical tumors and breast tumors, and metastases thereof.

In some embodiments, the tumor or the metastasis is a tumor or metastasis of a cancer selected from the group consisting of pancreatic cancer, ovarian cancer, lung cancer, colon cancer, prostate cancer, cervical cancer, breast cancer, rectal cancer, renal (kidney) cancer, gastric cancer, esophageal cancer, hepatic (liver) cancer, endometrial cancer, bladder cancer, brain cancer, head and neck cancer, oral cancer (e.g., oral cavity cancer), cervical cancer, uterine cancer, thyroid cancer, testicular cancer, prostate cancer, skin cancers, such as melanoma, e.g., malignant melanoma, cholangiocarcinoma (bile duct cancer), thymic epithelial cancer, e.g., thymoma, leukemia, lymphoma, multiple myeloma, Hodgkin's lymphoma, and non-Hodgkin's lymphoma.

In some embodiments, the cancer is a breast cancer, prostate cancer, ovarian cancer, lung cancer, colon cancer, or pancreatic cancer. In some embodiments, the cancer is MSS colorectal cancer. In some embodiments, the tumor or metastasis treated is a cancer such as pancreatic cancer, non-small cell lung cancer, multiple myeloma or leukemia. In some embodiments, the tumor is a carcinoma such as, for example, an ovarian tumor or a pancreatic tumor.

In other examples, methods are provided for treating a subject, where the methods include administering to the subject a virus that expresses one or more of the constructs described herein against which antigens the subject will develop an immune response. The immunizing antigens can be endogenous to the virus, such as vaccinia antigens on a vaccinia virus used to immunize against smallpox, measles, mumps, or the immunizing antigens can be exogenous antigens expressed by the virus, such as influenza or HIV antigens expressed on a viral capsid surface. In the case of smallpox, for example, a tumor specific protein antigen can be carried by an attenuated vaccinia virus (encoded by the viral genome) for a smallpox vaccine. Thus, the viruses provided herein, including the modified vaccinia viruses can be used as vaccines.

In some examples, provided herein are methods for eliciting or enhancing antibody production against a selected antigen or a selected antigen type in a subject, where the methods include administering to a subject a virus that can accumulate in a tumor and/or metastasis, and can cause release of a selected antigen or selected antigen type from the tumor, resulting in antibody production against the selected antigen or selected antigen type. Any of a variety of antigens can be targeted in the methods provided herein, including a selected antigen such as an exogenous gene product expressed by the virus, or a selected antigen type such as one or more tumor antigens release from the tumor as a result of viral infection of the tumor (e.g., by lysis, apoptosis, secretion or other mechanism of causing antigen release from the tumor).

In some examples, it can be desirable to maintain release of the selected antigen or selected antigen type over a series of days, for example, at least a week, at least ten days, at least two weeks or at least a month. Provided herein are methods for providing a sustained antigen release within a subject, where the methods include administering to a subject a virus that can accumulate in a tumor and/or metastasis, and can cause sustained release of an antigen, resulting in antibody production against the antigen. The sustained release of antigen can result in an immune response by the viral-infected host, in which the host can develop antibodies against the antigen, and/or the host can mount an immune response against cells expressing the antigen, including an immune response against tumor cells. Thus, the sustained release of antigen can result in immunization against tumor cells. In some examples, the viral-mediated sustained antigen release-induced immune response against tumor cells can result in complete removal or killing of all tumor cells.

In some embodiments, the subject has a cancer selected from the group consisting of pancreatic cancer, ovarian cancer, lung cancer, colon cancer, prostate cancer, cervical cancer, breast cancer, rectal cancer, renal (kidney) cancer, gastric cancer, esophageal cancer, hepatic (liver) cancer, endometrial cancer, bladder cancer, brain cancer, head and neck cancer, oral cancer (e.g., oral cavity cancer), cervical cancer, uterine cancer, thyroid cancer, testicular cancer, prostate cancer, skin cancers, such as melanoma, e.g., malignant melanoma, cholangiocarcinoma (bile duct cancer), thymic epithelial cancer, e.g., thymoma, leukemia, lymphoma, multiple myeloma, Hodgkin's lymphoma, and non-Hodgkin's lymphoma.

In some embodiments, the subject exhibits severe immune deficiency and is sensitive to virus infection.

In some embodiments, the proliferative disorder is MSS colorectal cancer and the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8. In some embodiments, the proliferative disorder is MSS colorectal cancer and the nucleic acid genome of the recombinant oncolytic virus comprises the sequence of nucleotides set forth in SEQ ID NO: 8. In some embodiments, the subject exhibits severe immune deficiency and is sensitive to virus infection, the proliferative disorder is MSS colorectal cancer, and the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8.

In some embodiments, the methods provided herein, e.g., methods of treatment, further comprise administering AP1903 (Rimiducid) to the subject.

### B. Administration

A virus provided herein can be administered to a subject, including a subject having a tumor, cancer, hyperplasia, metastasis, or having neoplastic cells, or a subject to be immunized. An administered virus can be a virus provided herein or any other virus generated using the methods provided herein. In some examples, the virus administered is a virus containing a characteristic such as attenuated pathogenicity, low toxicity, preferential accumulation in tumor, ability to activate an immune response against tumor cells, ability to induce apoptosis, high immunogenicity, replication competence and ability to express exogenous proteins, and combinations thereof.

### 1. Steps Prior to Administering the Virus

In some examples, one or more steps can be performed prior to administration of the virus to the subject. Any of a variety of preceding steps can be performed, including, but not limited to diagnosing the subject with a condition appropriate for virus administration, determining the immunocompetence of the subject, immunizing the subject, treating the subject with a chemotherapeutic agent, treating the subject with radiation, or surgically treating the subject.

For examples that include administering a virus to a tumor-bearing subject for therapeutic purposes, the subject has typically been previously diagnosed with a neoplastic condition. Diagnostic methods also can include determining the type of neoplastic condition, determining the stage of the neoplastic conditions, determining the size of one or more tumors in the subject, determining the presence or absence of metastatic or neoplastic cells in the lymph nodes of the subject, or determining the presence of metastases of the subject. Some examples of therapeutic methods for administering a virus to a subject can include a step of determination of the size of the primary tumor or the stage of the neoplastic disease, and if the size of the primary tumor is equal to or above a threshold volume, or if the stage of the neoplastic disease is at or above a threshold stage, a virus is administered to the subject. In a similar example, if the size of the primary tumor is below a threshold volume, or if the stage of the neoplastic disease is at or below a threshold stage, the virus is not yet administered to the subject; such methods can include monitoring the subject until the tumor size or neoplastic disease stage reaches a threshold amount, and then administering the virus to the subject. Threshold sizes can vary according to several factors, including rate of growth of the tumor, ability of the virus to infect a tumor, and immunocompetence of the subject. Generally the threshold size will be a size sufficient for a virus to accumulate and replicate in or near the tumor without being completely removed by the host's immune system, and will typically also be a size sufficient to sustain a virus infection for a time long enough for the host to mount an immune response against the tumor cells, typically about one week or more, about ten days or more, or about two weeks or more. Exemplary threshold tumor sizes for viruses, such as vaccinia viruses, are at least about 100 mm³, at least about 200 mm³, at least about 300 mm³, at least about 400 mm³, at least about 500 mm³, at least about 750 mm³, at least about 1000 mm³, or at least about 1500 mm³. Threshold neoplastic disease stages also can vary according to several factors, including specific requirement for staging a particular neoplastic disease, aggressiveness of growth of the neoplastic disease, ability of the virus to infect a tumor or metastasis, and immunocompetence of the subject. Generally the threshold stage will be a stage sufficient for a virus to accumulate and replicate in a tumor or metastasis without being completely removed by the host's immune system, and will typically also be a size sufficient to sustain a virus infection for a time long enough for the host to mount an immune response against the neoplastic cells, typically about one week or more, about ten days or more, or about two weeks or more. Exemplary threshold stages are any stage beyond the lowest stage (e.g., Stage I or equivalent), or any stage where the primary tumor is larger than a threshold size, or any stage where metastatic cells are detected.

In other embodiments, prior to administering to the subject a virus, the immunocompetence of the subject can be determined. The methods of administering a virus to a subject provided herein can include causing or enhancing an immune response in a subject. Accordingly, prior to administering a virus to a subject, the ability of a subject to mount an immune response can be determined. Any of a variety of tests of immunocompetence known in the art can be performed in the methods provided herein. Exemplary immunocompetence tests can examine ABO hemagglutination titers (IgM), leukocyte adhesion deficiency (LAD), granulocyte function (NBT), T and B cell quantitation, tetanus antibody titers, salivary IgA, skin test, tonsil test, complement C3 levels, factor B levels, and lymphocyte count. One skilled in the art can determine the desirability to administer a virus to a subject according to the level of immunocompetence of the subject, according to the immunogenicity of the virus, and, optionally, according to the immunogenicity of the neoplastic disease to be treated. Typically, a subject can be considered immunocompetent if the skilled artisan can determine that the subject is sufficiently competent to mount an immune response against the virus.

In some embodiments, the subject can be immunized prior to administering to the subject a virus according to the methods provided herein. Immunization can serve to increase the ability of a subject to mount an immune response against the virus, or increase the speed at which the subject can mount an immune response against a virus. Immunization also can serve to decrease the risk to the subject of pathogenicity of the virus. In some embodiments, the immunization can be performed with an immunization virus that is similar to the therapeutic virus to be administered. For example, the immunization virus can be a replication-incompetent variant of the therapeutic virus. In other embodiments, the immunization material can be digests of the therapeutic virus to be administered. Any of a variety of methods for immunizing a subject against a known virus are known in the art and can be used herein. In one embodiment, vaccinia viruses treated with, for example, 1 microgram of psoralen and ultraviolet light at 365 nm for 4 minutes, can be rendered replication incompetent. In another embodiment, the virus can be selected as the same or similar to a virus against which the subject has been previously immunized, e.g., in a childhood vaccination.

In another embodiment, the subject can have administered thereto a virus without any previous steps of cancer treatment such as chemotherapy, radiation therapy or surgical removal of a tumor and/or metastases. The methods provided herein take advantage of the ability of the viruses to enter or localize near a tumor, where the tumor cells can be protected from the subject's immune system; the viruses can then proliferate in such an immune-protected region and can also cause the release, typically a sustained release, of tumor antigens from the tumor to a location in which the subject's immune system can recognize the tumor antigens and mount an immune response. In such methods, existence of a tumor of sufficient size or sufficiently developed immunoprotected state can be advantageous for successful administration of the virus to the tumor, and for sufficient tumor antigen production. If a tumor is surgically removed, the viruses may not be able to localize to other neoplastic cells (e.g., small metastases) because such cells have not yet have matured sufficiently to create an immune-protective environment in which the viruses can survive and proliferate, or even if the viruses can localize to neoplastic cells, the number of cells or size of the mass can be too small for the viruses to cause a sustained release of tumor antigens in order for the host to mount an anti-tumor immune response. Thus, for example, provided herein are methods of treating a tumor, cancer, metastasis, or neoplastic disease in which viruses are administered to a subject with a tumor or neoplastic disease without removing the primary tumor, or to a subject with a tumor or neoplastic disease in which at least some tumors or neoplastic cells are intentionally permitted to remain in the subject. In other typical cancer treatment methods such as chemotherapy or radiation therapy, such methods typically have a side effect of weakening the subject's immune system. This treatment of a subject by chemotherapy or radiation therapy can reduce the subject's ability to mount an anti-tumor immune response. Thus, for example, provided herein are methods of treating a hyperplasia, tumor, cancer, metastasis, or neoplastic disease in which viruses are administered to a subject with a tumor or neoplastic disease without treating the subject with an immune system-weakening therapy, such as chemotherapy or radiation therapy.

In an alternative embodiment, prior to administration of a virus to the subject, the subject can be treated in one or more cancer treatment steps that do not remove the primary tumor or that do not weaken the immune system of the subject. A variety of more sophisticated cancer treatment methods are being developed in which the tumor can be treated without surgical removal or immune-system weakening therapy. Exemplary methods include administering a compound that decreases the rate of proliferation of the tumor, cancer, metastasis, or neoplastic cells without weakening the immune system (e.g., by administering tumor suppressor compounds, such as apoptosis inducing compounds, or by administering tumor cell-specific compounds, such as anti-programmed death 1 (PD-1) receptor and anti-programmed death ligand 1 (PD-L1)/L2 inhibitors) or administering an angiogenesis-inhibiting compound. Thus, combined methods that include administering a virus to a subject can further improve cancer therapy. Thus, provided herein are methods of administering a virus to a subject, along with prior to or subsequent to, for example, administering a compound that slows tumor growth without weakening the subject's immune system or a compound that inhibits vascularization of the tumor.

### 2. Mode of Administration

Any mode of administration of a virus to a subject can be used, provided the mode of administration permits the virus to enter a tumor, hyperplasia, cancer, or metastasis. Modes of administration can include, but are not limited to, systemic, parenteral, intravenous, intraperitoneal, subcutaneous, intramuscular, transdermal, intradermal, intra-arterial (e.g., hepatic artery infusion), intravesical perfusion, intrapleural, intraarticular, topical, intratumoral, intralesional, endoscopic, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, or ocular administration. In some embodiments, a diagnostic or therapeutic agent as described elsewhere herein also can be similarly administered. In particular embodiments, the virus is administered intravenously.

One skilled in the art can select any mode of administration compatible with the subject and the virus, and that also is likely to result in the virus reaching tumors and/or metastases. The route of administration can be selected by one skilled in the art according to any of a variety of factors, including the nature of the disease, the kind of tumor, and the particular virus contained in the pharmaceutical composition. Administration to the target site can be performed, for example, by ballistic delivery, as a colloidal dispersion system, or systemic administration can be performed by injection into an artery.

### 3. Dosages and Dosage Regime

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art.

In the present methods, appropriate minimum dosage levels and dosage regimes of viruses can be levels sufficient for the virus to survive, grow and replicate in a tumor or metastasis. Generally, the virus is administered in an amount that is at least or about or 1×10⁵ pfu at least one time over a cycle of administration. Exemplary minimum levels for administering a virus to a 65 kg human can include at least about 1×10⁵ plaque forming units (pfu), at least about 5×10⁵ pfu, at least about 1×10⁶ pfu, at least about 5×10⁶ pfu, at least about 1×10⁷ pfu, at least about 1×10⁸ pfu, at least about 1×10⁹ pfu, or at least about 1×10¹⁰ pfu. For example, in some embodiments, the virus is administered in an amount that is at least or about or is 1×10⁵ pfu, 1×10⁶ pfu, 1×10⁷ pfu, 1×10⁸ pfu, 1×10⁹ pfu, 1×10¹⁰ pfu, 1×10¹¹ pfu, 1×10¹² pfu, 1×10¹³ pfu, or 1×10¹⁴ pfu at least one time over a cycle of administration. In some embodiments, the virus is administered in an amount from 1×10⁵ pfu to 1×10¹⁴ pfu.

In the dosage regime, the amount of virus can be administered as a single administration or multiple times over the cycle of administration. Hence, the methods provided herein can include a single administration of a virus to a subject or multiple administrations of a virus to a subject. In some examples, a single administration is sufficient to establish a virus in a tumor, where the virus can proliferate and can cause or enhance an anti-tumor response in the subject; such methods do not require additional administrations of a virus in order to cause or enhance an anti-tumor response in a subject, which can result, for example in inhibition of tumor growth, inhibition of metastasis growth or formation, reduction in tumor or size, elimination of a tumor or metastasis, inhibition or prevention of recurrence of a neoplastic disease or new tumor formation, or other cancer therapeutic effects.

In other examples, a virus can be administered on different occasions, separated in time typically by at least one day. For example, a virus can be administered two times, three times, four times, five times, or six times or more, with one day or more, two days or more, one week or more, or one month or more time between administrations. Separate administrations can increase the likelihood of delivering a virus to a tumor or metastasis, where a previous administration has been ineffective in delivering a virus to a tumor or metastasis. Separate administrations can increase the locations on a tumor or metastasis where virus proliferation can occur or can otherwise increase the titer of virus accumulated in the tumor, which can increase the scale of release of antigens or other compounds from the tumor in eliciting or enhancing a host's anti-tumor immune response, and also can, optionally, increase the level of virus-based tumor lysis or tumor cell death. Separate administrations of a virus can further extend a subject's immune response against viral antigens, which can extend the host's immune response to tumors or metastases in which viruses have accumulated, and can increase the likelihood of a host mounting an anti-tumor immune response.

When separate administrations are performed, each administration can be a dosage amount that is the same or different relative to other administration dosage amounts. In one example, all administration dosage amounts are the same. In other examples, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same, smaller amount relative to the first administration.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a virus, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject's anti-virus antibody titer, the subject's anti-tumor antibody titer, the overall health of the subject, the weight of the subject, the presence of virus solely in tumor and/or metastases, the presence of virus in normal tissues or organs.

The time period between administrations can be any of a variety of time periods. The time period between administrations can be a function of any of a variety of factors, including monitoring steps, as described in relation to the number of administrations, the time period for a subject to mount an immune response, the time period for a subject to clear the virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

For example, an amount of virus is administered two times, three times, four times, five times, six times or seven times over a cycle of administration. The amount of virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. Generally, the cycle of administration is 7 days, 14 days, 21 days or 28 days. Depending on the responsiveness or prognosis of the patient, the cycle of administration is repeated over the course of several months or years.

Generally, appropriate maximum dosage levels or dosage regimes of viruses are levels that are not toxic to the host, levels that do not cause splenomegaly of 3 times or more, and/or levels that do not result in colonies or plaques in normal tissues or organs after about 1 day or after about 3 days or after about 7 days.

### 4. Co-Administrations

Also provided are methods in which an additional therapeutic substance, such as a different therapeutic virus or a therapeutic compound is administered. These can be administered simultaneously, sequentially or intermittently with the first virus. The additional therapeutic substance can interact with the virus or a gene product thereof, or the additional therapeutic substance can act independently of the virus.

Combination therapy treatment has advantages in that: 1) it avoids single agent resistance; 2) in a heterogeneous tumor population, it can kill cells by different mechanisms; and 3) by selecting drugs with non-overlapping toxicities, each agent can be used at full dose to elicit maximal efficacy and synergistic effect. Combination therapy can be done by combining a diagnostic/therapeutic virus with one or more of the following anti-cancer agents: chemotherapeutic agents, therapeutic antibodies, siRNAs, toxins, enzyme-prodrug pairs or radiation.

Accordingly, also provided herein, in some embodiments, are methods of treatment and methods of inhibiting virus replication that further comprise administering an additional therapeutic substance, such as any therapeutic agent, additional virus, or therapeutic compound as described herein.

a. i. Administering a Plurality of Viruses

Methods are provided for administering to a subject two or more viruses. Administration can be effected simultaneously, sequentially or intermittently. The plurality of viruses can be administered as a single composition or as two or more compositions. The two or more viruses can include at least two viruses. In a particular embodiment, where there are two viruses, both viruses are vaccinia viruses. In another embodiment, one virus is a vaccinia virus and the second virus is any one of an adenovirus, an adeno-associated virus, a retrovirus, a herpes simplex virus, a reovirus, a mumps virus, a foamy virus, an influenza virus, a myxoma virus, a vesicular stomatitis virus, or any other virus described herein or known in the art. Viruses can be chosen based on the pathway on which they act. For example, a virus that targets an apoptotic pathway can be combined with a virus that targets angiogenesis pathways. In another example, a virus that targets host's complement can be combined with a virus that stimulates the host's immune system.

The plurality of viruses can be provided as combinations of compositions containing and/or as kits that include the viruses packaged for administration and optionally including instructions therefore. The compositions can contain the viruses formulated for single dosage administration (i.e., for direct administration) and can require dilution or other additions.

In one embodiment, at least one of the viruses is a modified virus such as those provided herein, having a characteristic such as low pathogenicity, low toxicity, preferential accumulation in tumor, ability to activate an immune response against tumor cells, immunogenic, replication competent, ability to express exogenous proteins, and combinations thereof. The viruses can be administered at approximately the same time, or can be administered at different times. The viruses can be administered in the same composition or in the same administration method, or can be administered in separate composition or by different administration methods.

The time period between administrations can be any time period that achieves the desired effects, as can be determined by one skilled in the art. Selection of a time period between administrations of different viruses can be determined according to parameters similar to those for selecting the time period between administrations of the same virus, including results from monitoring steps, the time period for a subject to mount an immune response, the time period for a subject to clear virus from normal tissue, or the time period for virus proliferation in the tumor or metastasis. In one embodiment, the time period can be a function of the time period for a subject to mount an immune response; for example, the time period can be more than the time period for a subject to mount an immune response, such as more than about one week, more than about ten days, more than about two weeks, or more than about a month; in another embodiment, the time period can be less than the time period for a subject to mount an immune response, such as less than about one week, less than about ten days, less than about two weeks, or less than about a month. In another embodiment, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another embodiment, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

b. ii. Therapeutic Compounds

Any therapeutic or anti-cancer agent can be used as the second, therapeutic or anti-cancer agent in the combined cancer treatment methods provided herein. The methods can include administering one or more therapeutic compounds to the subject in addition to administering a virus or plurality thereof to a subject. Therapeutic compounds can act independently, or in conjunction with the virus, for tumor therapeutic effects.

Therapeutic compounds that can act independently include any of a variety of known chemotherapeutic compounds that can inhibit tumor growth, inhibit metastasis growth and/or formation, decrease the size of a tumor or metastasis, eliminate a tumor or metastasis, without reducing the ability of a virus to accumulate in a tumor, replicate in the tumor, and cause or enhance an anti-tumor immune response in the subject.

Therapeutic compounds that act in conjunction with the viruses include, for example, compounds that alter the expression of the viruses or compounds that can interact with a virally-expressed gene, or compounds that can inhibit virus proliferation, including compounds toxic to the virus. Therapeutic compounds that can act in conjunction with the virus include, for example, therapeutic compounds that increase the proliferation, toxicity, tumor cell killing or immune response eliciting properties of a virus, and also can include, for example, therapeutic compounds that decrease the proliferation, toxicity or cell killing properties of a virus. Optionally, the therapeutic agent can exhibit or manifest additional properties, such as, properties that permit its use as an imaging agent, as described elsewhere herein.

Therapeutic compounds also include, but are not limited to, chemotherapeutic agents, nanoparticles, radiation therapy, siRNA molecules, enzyme/pro-drug pairs, photosensitizing agents, toxins, microwaves, a radionuclide, an angiogenesis inhibitor, a mitosis inhibitor protein (e.g., cdc6), an antitumor oligopeptide (e.g., antimitotic oligopeptides, high affinity tumor-selective binding peptides), a signaling modulator, anti-cancer antibiotics, or a combination thereof.

Exemplary photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl) chlorin or tin(IV) chlorin e6, purpurins such as tin ethyl etiopurpurin, purpurinimides, bacteriochlorins, pheophorbides, pyropheophorbides or cationic dyes. In one example, a vaccinia virus, such as a vaccinia virus provided herein, is administered to a subject having a tumor, cancer or metastasis in combination with a photosensitizing agent.

Radionuclides, which depending up the radionuclide, amount and application can be used for diagnosis and/or for treatment. They include, but are not limited to, for example, a compound or molecule containing 32Phosphorus, 60Cobalt, 90Yttrium, 99Technitium, 103Palladium, 106Ruthenium, 111Indium, 117Lutetium, 125Iodine, 131Iodine, 137Cesium, 153Samarium, 186Rhenium, 188Rhenium, 192Iridium, 198Gold, 211Astatine, 212Bismuth or 213Bismuth. In one example, a vaccinia virus, such as a vaccinia virus provided herein, is administered to a subject having a tumor, cancer or metastasis in combination with a radionuclide.

Toxins include, but are not limited to, chemotherapeutic compounds such as, but not limited to, 5-fluorouridine, calicheamicin and maytansine. Signaling modulators include, but are not limited to, for example, inhibitors of macrophage inhibitory factor, toll-like receptor agonists and stat3 inhibitors. In one embodiment, a vaccinia virus, such as a vaccinia virus provided herein, is administered to a subject having a tumor, cancer or metastasis in combination with a toxin or a signaling modulator.

Combination therapy between chemotherapeutic agents and therapeutic viruses can be effective/curative in situations when single agent treatment is not effective. Chemotherapeutic compounds include, but are not limited to, alkylating agents such as thiotepa and cyclophosphamide; alkyl sulfonates such as busulfan, improsulfan and piposulfan; aziridines such as benzodepa, carboquone, meturedepa and uredepa; ethylenimine and methylmelamines, including altretamine, triethylenemelamine, triethylenephosphoramide, triethylenethiophosphoramide and trimethylmelamine nitrogen mustards such as chlorambucil, chlornaphazine, chlorophosphamide, estramustine, ifosfamide, mechlorethamine, mechlorethamine oxide hydrochloride, melphalan, novobiocin, phenesterine, prednimustine, trofosfamide, uracil mustard; nitrosoureas such as carmustine, chlorozotocin, fotemustine, lomustine, nimustine, ranimustine; antibiotics such as aclacinomycins, actinomycin, anthramycin, azaserine, bleomycins, cactinomycin, calicheamicin, carubicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defosfamide; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, e.g., paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; Navelbine®; Novantrone®; teniposide; daunomycin; aminopterin; Xeloda®; ibandronate; CPT11; topoisomerase inhibitor RFS 2000; difluoromethylornithine (DMFO); retinoic acid; esperamycins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone and toremifene (Fareston®); and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. Chemotherapeutic agents also include new classes of targeted chemotherapeutic agents such as, for example, imatinib (sold by Novartis under the trade name Gleevec® in the United States), gefitinib (developed by AstraZeneca under the trade name Iressa®) and erlotinib (developed by Genentech under the trade name Tarceva®). Particular chemotherapeutic agents include, but are not limited to, cisplatin, carboplatin, oxaliplatin, DWA2114R, NK121, IS 3 295, and 254-S vincristine, prednisone, doxorubicin and L-asparaginase; mechlorethamine, vincristine, procarbazine and prednisone (MOPP), cyclophosphamide, vincristine, procarbazine and prednisone (C-MOPP), bleomycin, vinblastine, gemcitabine and 5-flurouracil. Exemplary chemotherapeutic agents are, for example, cisplatin, carboplatin, oxaliplatin, DWA2114R, NK121, IS 3 295, and 254-S. In a non-limiting example, a vaccinia virus, such as a vaccinia virus provided herein, is administered to a subject having a tumor, cancer or metastasis in combination with a platinum coordination complex, such as cisplatin, carboplatin, oxaliplatin, DWA2114R, NK121, IS 3 295, and 254-S. Tumors, cancers and metastasis can be any of those provided herein, and in particular, can be a pancreatic tumor, an ovarian tumor, a lung tumor, a colon tumor, a prostate tumor, a cervical tumor or a breast tumor; exemplary tumors are pancreatic and ovarian tumors. Tumors, cancers and metastasis can be a monotherapy-resistant tumor such as, for example, one that does not respond to therapy with virus alone or anti-cancer agent alone, but that does respond to therapy with a combination of virus and anti-cancer agent. Typically, a therapeutically effective amount of virus is systemically administered to the subject and the virus localizes and accumulates in the tumor. Subsequent to administering the virus, the subject is administered a therapeutically effective amount of an anti-cancer agent, such as cisplatin. In one embodiment, cisplatin is administered once-daily for five consecutive days. One of skill in the art could determine when to administer the anti-cancer agent subsequent to the virus using, for example, in vivo animal models. Using the methods provided herein, administration of a virus and anti-cancer agent, such as cisplatin can cause a reduction in tumor volume, can cause tumor growth to stop or be delayed or can cause the tumor to be eliminated from the subject. The status of tumors, cancers and metastasis following treatment can be monitored using any of the methods provided herein and known in the art.

Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and pirarubicin hydrochloride, phleomycins such as phleomycin and peplomycin sulfate, mitomycins such as mitomycin C, actinomycins such as actinomycin D, zinostatinstimalamer and polypeptides such as neocarzinostatin. In one embodiment, a vaccinia virus, such as a vaccinia virus provided herein, is administered to a subject having a tumor, cancer or metastasis in combination with an anti-cancer antibiotic.

In one embodiment, nanoparticles can be designed such that they carry one or more therapeutic agents provided herein. Additionally, nanoparticles can be designed to carry a molecule that targets the nanoparticle to the tumor cells. In one non-limiting embodiment, nanoparticles can be coated with a radionuclide and, optionally, an antibody immunoreactive with a tumor-associated antigen. In one embodiment, a vaccinia virus, such as a vaccinia virus provided herein, is administered to a subject having a tumor, cancer or metastasis in combination with a nanoparticle carrying any of the therapeutic agents provided herein.

Radiation therapy has become a foremost choice of treatment for a majority of cancer patients. The wide use of radiation treatment stems from the ability of gamma-irradiation to induce irreversible damage in targeted cells with the preservation of normal tissue function. Ionizing radiation triggers apoptosis, the intrinsic cellular death machinery in cancer cells, and the activation of apoptosis seems to be the principal mode by which cancer cells die following exposure to ionizing radiation. In one embodiment, a vaccinia virus, such as a vaccinia virus provided herein, is administered to a subject having a tumor, cancer or metastasis in combination with radiation therapy.

Thus, provided herein are methods of administering to a subject one or more therapeutic compounds that can act in conjunction with the virus to increase the proliferation, toxicity, tumor cell killing, host's complement evasion, or immune response eliciting properties of a virus. Also provided herein are methods of administering to a subject one or more therapeutic compounds that can act in conjunction with the virus to decrease the proliferation, toxicity, or cell killing properties of a virus. Therapeutic compounds to be administered can be any of those provided herein or in the art.

Therapeutic compounds that can act in conjunction with the virus to increase the proliferation, toxicity, tumor cell killing, host's complement evasion, or immune response eliciting properties of a virus are compounds that can alter gene expression, where the altered gene expression can result in an increased killing of tumor cells or an increased anti-tumor immune response in the subject. A gene expression-altering compound can, for example, cause an increase or decrease in expression of one or more viral genes, including endogenous viral genes and/or exogenous viral genes. For example, a gene expression-altering compound can induce or increase transcription of a gene in a virus such as an exogenous gene that can cause cell lysis or cell death that can provoke an immune response that can catalyze conversion of a prodrug-like compound, or that can inhibit expression of a tumor cell gene. Any of a wide variety of compounds that can alter gene expression are known in the art, including IPTG and RU486. Exemplary genes whose expression can be up-regulated include proteins and RNA molecules, including toxins, enzymes that can convert a prodrug to an anti-tumor drug, cytokines, transcription regulating proteins, siRNA and ribozymes. In another example, a gene expression-altering compound can inhibit or decrease transcription of a gene in a virus such as a heterologous gene that can reduce viral toxicity or reduces viral proliferation. Any of a variety of compounds that can reduce or inhibit gene expression can be used in the methods provided herein, including siRNA compounds, transcriptional inhibitors or inhibitors of transcriptional activators. Exemplary genes whose expression can be down-regulated include proteins and RNA molecules, including viral proteins or RNA that suppress lysis, nucleotide synthesis or proliferation, and cellular proteins or RNA molecules that suppress cell death, immunoreactivity, lysis, or viral replication.

In another example, therapeutic compounds that can act in conjunction with the virus to increase the proliferation, toxicity, tumor cell killing, host's complement evasion, or immune response eliciting properties of a virus are compounds that can interact with a virally expressed gene product, and such interaction can result in an increased killing of tumor cells or an increased anti-tumor immune response in the subject. A therapeutic compound that can interact with a virally-expressed gene product can include, for example a prodrug or other compound that has little or no toxicity or other biological activity in its subject-administered form, but after interaction with a virally expressed gene product, the compound can develop a property that results in tumor cell death, including but not limited to, cytotoxicity, ability to induce apoptosis, or ability to trigger an immune response. In one non-limiting example, the virus carries an enzyme into the cancer cells. Once the enzyme is introduced into the cancer cells, an inactive form of a chemotherapy drug (i.e., a prodrug) is administered. When the inactive prodrug reaches the cancer cells, the enzyme converts the prodrug into the active chemotherapy drug, so that it can kill the cancer cell. Thus, the treatment is targeted only to cancer cells and does not affect normal cells. The prodrug can be administered concurrently with, or sequentially to, the virus. A variety of prodrug-like substances are known in the art and an exemplary set of such compounds are disclosed elsewhere herein, where such compounds can include gancyclovir, 5-fluorouracil, 6-methylpurine deoxyriboside, cephalosporin-doxorubicin, 4-[(2-chloroethyl)(2-mesyloxyethyl)amino]benzoyl-L-glutamic acid, acetaminophen, indole-3-acetic acid, CB1954, 7-ethyl-10-[4-(1-piperidino)-1-piperidino]carbonyloxycamptothecin, bis-(2-chloroethyl)amino-4-hydroxyphenyl-aminomethanone 28, 1-chloromethyl-5-hydroxy-1,2-dihydro-3,4-benz[e]indole, epirubicin-glucuronide, 5′-deoxy-5-fluorouridine, cytosine arabinoside, linamarin, and a nucleoside analogue (e.g., fluorouridine, fluorodeoxyuridine, fluorouridine arabinoside, cytosine arabinoside, adenine arabinoside, guanine arabinoside, hypoxanthine arabinoside, 6-mercaptopurineriboside, theoguanosine riboside, nebularine, 5-iodouridine, 5-iododeoxyuridine, 5-bromodeoxyuridine, 5-vinyldeoxyuridine, 9-[(2-hydroxy)ethoxy]methylguanine (acyclovir), 9-[(2-hydroxy-1-hydroxymethyl)-ethoxy]methylguanine (DHPG), azauridien, azacytidine, azidothymidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, 3′-deoxythymidine).

In another example, therapeutic compounds that can act in conjunction with the virus to decrease the proliferation, toxicity or cell killing properties of a virus are compounds that can inhibit viral replication, inhibit viral toxins or cause viral death. A therapeutic compound that can inhibit viral replication, inhibit viral toxins, or cause viral death can generally include a compound that can block one or more steps in the viral life cycle, including, but not limited to, compounds that can inhibit viral DNA replication, viral RNA transcription, viral coat protein assembly, outer membrane or polysaccharide assembly. Any of a variety of compounds that can block one or more steps in a viral life cycle are known in the art, including any known antiviral compound (e.g., cidofovir), viral DNA polymerase inhibitors, viral RNA polymerase inhibitors, inhibitors of proteins that regulate viral DNA replication or RNA transcription. In another example, a virus can contain a gene encoding a viral life cycle protein, such as DNA polymerase or RNA polymerase that can be inhibited by a compound that is, optionally, non-toxic to the host organism.

In addition to combination therapy between chemotherapeutic agents and a virus provided herein, other more complex combination therapy strategies could be applied as well. For example, a combination therapy can include chemotherapeutic agents, therapeutic antibodies including checkpoint inhibitors (e.g. anti-PD-1 or anti-PD-L1 antibody), and a virus provided herein. Alternatively, another combination therapy can be the combination of radiation, therapeutic antibodies such as a checkpoint inhibitor (e.g. anti-PD-1 or anti-PD-L1 antibody), and a virus provided herein. Therefore, the concept of combination therapy also can be based on the application of a virus provided herein virus along with one or more of the following therapeutic modalities, namely, chemotherapeutic agents, radiation therapy, therapeutic antibodies including checkpoint inhibitors (e.g. anti-PD-1 or anti-PD-L1 antibody), hyper- or hypothermia therapy, siRNA, diagnostic/therapeutic bacteria, diagnostic/therapeutic mammalian cells, immunotherapy, and/or targeted toxins (delivered by antibodies, liposomes and nanoparticles).

Effective delivery of each component of the combination therapy is an important aspect of the methods provided herein. In accordance with one aspect, the modes of administration discussed below exploit one of more of the key features: (i) delivery of a virus provided herein to the tumors by a mode of administration effect to achieve highest titer of virus and highest therapeutic effect; (ii) delivery of any other mentioned therapeutic modalities to the tumor by a mode of administration to achieve the optimal therapeutic effect. The dose scheme of the combination therapy administered is such that the combination of the two or more therapeutic modalities is therapeutically effective. Dosages will vary in accordance with such factors as the age, health, sex, size and weight of the patient, the route of administration, the toxicity of the drugs, frequency of treatment and the relative susceptibilities of the cancer to each of the therapeutic modalities.

For combination therapies with chemotherapeutic compounds, dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, for example, tumor size, and other viruses, treatments, or compounds, such as other chemotherapeutic drugs, being administered concurrently). In addition to the above factors, such levels can be affected by the infectivity of the virus, and the nature of the virus, as can be determined by one skilled in the art. For example, cisplatin (also called cis-platinum, platinol; cis-diamminedichloroplatinum; and cDDP) is representative of a broad class of water-soluble, platinum coordination compounds frequently employed in the therapy of testicular cancer, ovarian tumors and a variety of other cancers. (See, e.g., Blumenreich et al. Cancer 55 (5): 1118-1122 (1985); Forastiere et al. J. Clin. Oncol. 19 (4): 1088-1095 (2001)). Methods of employing cisplatin clinically are well known in the art. For example, cisplatin has been administered in a single day over a six hour period, once per month, by slow intravenous infusion. For localized lesions, cisplatin can be administered by local injection. Intraperitoneal infusion can also be employed. Cisplatin can be administered in doses as low as 10 mg/m2 per treatment if part of a multi-drug regimen, or if the patient has an adverse reaction to higher dosing. In general, a clinical dose is from about 30 to about 120 or 150 mg/m2 per treatment.

Typically, platinum-containing chemotherapeutic agents are administered parenterally, for example by slow intravenous infusion, or by local injection, as discussed above. The effects of intralesional (intra-tumoral) and IP administration of cisplatin is described in (Nagase et al. Cancer Treat. Rep. 71 (9): 825-829 (1987); and Theon et al. J. Am. Vet. Med. Assoc. 202 (2): 261-7. (1993)).

In one exemplary embodiment, the virus is administered once, 2-6 times or more with 0-60 days apart each administration, followed by 1-30 days where no anti-cancer treatment, then cisplatin is administered daily for 1-5 days, followed by 1-30 days where no anti-cancer treatment is administered. Each component of the therapy, virus or cisplatin treatment, or the virus and cisplatin combination therapy can be repeated. In another exemplary example, cisplatin is administered daily for 1 to 5 days, followed by 1-10 days where no anti-cancer treatment is administered, then the virus is administered once or 2-6 times with 0-60 days apart. Such treatment scheme can be repeated. In another exemplary example, cisplatin is administered daily for 1 to 5 days, followed by 1-10 days where no anti-cancer treatment is administered, then the virus is administered once or 2-6 times with 0-60 days apart. This is followed by 5-60 days where no anti-cancer treatment is administered, then cisplatin is administered again for 1-5 days. Such treatment scheme can be repeated.

Gemcitabine (GEMZAR®) is another compound employed in the therapy of breast cancer, non-small cell lung cancer, and pancreatic cancer. Gemcitabine is a nucleoside analogue that exhibits antitumor activity. Methods of employing gemcitabine clinically are well known in the art. For example, gemcitabine has been administered by intravenous infusion at a dose of 1000 mg/m2 over 30 minutes once weekly for up to 7 weeks (or until toxicity necessitates reducing or holding a dose), followed by a week of rest from treatment of pancreatic cancer. Subsequent cycles can include infusions once weekly for 3 consecutive weeks out of every 4 weeks. Gemcitabine has also been employed in combination with cisplatin in cancer therapy.

In one exemplary example, the virus is administered once or 2-6 times with 0-60 days apart, followed by 1-30 days where no anti-cancer treatment is administered, then gemcitabine is administered 1-7 times with 0-30 days apart, followed by 1-30 days where no anti-cancer treatment is administered. Such treatment scheme can be repeated. In another exemplary example, gemcitabine is administered 1-7 times with 0-30 days apart, followed by 1-10 days where no anti-cancer treatment is administered, then the virus is administered once or 2-6 times with 0-60 days apart. This is followed by 5-60 days where no anti-cancer treatment is administered. Such treatment scheme can be repeated. In another exemplary embodiment, gemcitabine is administered 1-7 times with 0-30 days apart, followed by 1-10 days where no anti-cancer treatment is administered, then the virus is administered once or 2-6 times with 0-60 days apart. This is followed by 5-60 days where no anti-cancer treatment is administered, then gemcitabine is administered again for 1-7 times with 0-30 days apart. Such treatment scheme can be repeated.

As will be understood by one of skill in the art, the optimal treatment regimen will vary and it is within the scope of the treatment methods to evaluate the status of the disease under treatment and the general health of the patient prior to, and following one or more cycles of combination therapy in order to determine the optimal therapeutic combination.

c. iii. Immunotherapies and Biological Therapies

Therapeutic compounds also include, but are not limited to, compounds that exert an immunotherapeutic effect, stimulate or suppress the immune system, carry a therapeutic compound, or a combination thereof. Optionally, the therapeutic agent can exhibit or manifest additional properties, such as, properties that permit its use as an imaging agent, as described elsewhere herein. Such therapeutic compounds include, but are not limited to, anti-cancer antibodies, radiation therapy, siRNA molecules and compounds that suppress the immune system (i.e. immunosuppressors, immunosuppressive agents). In some cases, it is desirable to administer an immunosuppressive agent to a subject to suppress the immune system prior to the administration of the virus in order to minimize any adverse reactions to the virus. Exemplary immunosuppressive agents include, but are not limited to, glucocorticoids, alkylating agents, antimetabolites, interferons and immunosuppressive antibodies (e.g., anti-CD3 and anti-IL2 receptor antibodies).

Immunotherapy also includes for example, immune-stimulating molecules (protein-based or non-protein-based), cells and antibodies. Immunotherapy treatments can include stimulating immune cells to act more effectively or to make the tumor cells or tumor associated antigens recognizable to the immune system (i.e., break tolerance).

Cytokines and growth factors include, but are not limited to, interleukins, such as, for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factors (GM-CSF), angiogenins, and tissue factors.

Anti-cancer antibodies include, but are not limited to, Rituximab, ADEPT, Trastuzumab (Herceptin®), Tositumomab (Bexxar®), Cetuximab (Erbitux®), Ibritumomab (Zevalin®), Alemtuzumab (Campath®-1H), Epratuzumab (LymphoCide®), Gemtuzumab ozogamicin (Mylotarg®), Bevacimab (Avastin®), Tarceva® (Erlotinib), SUTENT® (sunitinib malate), Panorex® (Edrecolomab), RITUXAN® (Rituximab), Zevalin® (90Y-ibritumomab tiuexetan), Mylotarg® (Gemtuzumab Ozogamicin) and Campath® (Alemtuzumab).

Thus, provided herein are methods of administering to a subject one or more therapeutic compounds that can act in conjunction with the virus to stimulate or enhance the immune system, thereby enhancing the effect of the virus. Such immunotherapy can be either delivered as a separate therapeutic modality or could be encoded (if the immunotherapy is protein-based) by the administered virus.

Biological therapies are treatments that use natural body substances or drugs made from natural body substances. They can help treat a cancer and control side effects caused by other cancer treatments such as chemotherapy. Biological therapies are also sometimes called Biological Response Modifiers (BRM's), biologic agents or simply “biologics” because they stimulate the body to respond biologically (or naturally) to cancer. Immunotherapy is treatment using natural substances that the body uses to fight infection and disease. Because it uses natural substances, immunotherapy is also a biological therapy. There are several types of drugs that come under the term biological therapy: these include, for example, monoclonal antibodies (mAbs), cancer vaccines, growth factors for blood cells, cancer growth inhibitors, anti-angiogenic factors, interferon alpha, interleukin-2 (IL-2), gene therapy and BCG vaccine for bladder cancer

Monoclonal antibodies (mAbs) are of particular interest for treating cancer because of the specificity of binding to a unique antigen and the ability to produce large quantities in the laboratory for mass distribution. Monoclonal antibodies can be engineered to act in the same way as immune system proteins: that is, to seek out and kill foreign matter in your body, such as viruses. Monoclonal antibodies can be designed to recognize epitopes on the surface of cancer cells. The antibodies target specifically bind to the epitopes and either kill the cancer cells or deliver a therapeutic agent to the cancer cell. Methods of conjugating therapeutic agents to antibodies are well-known in the art. Different antibodies have to be made for different types of cancer; for example, Rituximab recognizes CD20 protein on the outside of non-Hodgkin's lymphoma cells; ADEPT is a treatment using antibodies that recognize bowel (colon) cancer; and Trastuzumab (Herceptin®) recognizes breast cancer cells that produce too much of the protein HER 2 (“HER 2 positive”). Other antibodies include, for example, Tositumomab (Bexxar®), Cetuximab (Erbitux®), Ibritumomab (Zevalin®), Alemtuzumab (Campath®-1H), Epratuzumab (LymphoCide®), Gemtuzumab ozogamicin (Mylotarg®) and Bevacimab (Avastin®). Thus, the viruses provided herein can be administered concurrently with, or sequentially to, one or more monoclonal antibodies in the treatment of cancer. In one example, additional therapy is administered in the form of one or more of any of the other treatment modalities provided herein.

Rather than attempting to prevent infection, such as is the case with the influenza virus, cancer vaccines help treat the cancer once it has developed. The aim of cancer vaccines is to stimulate the immune response. Cancer vaccines include, for example, antigen vaccines, whole cell vaccines, dendritic cell vaccines, DNA vaccines and anti-idiotype vaccines. Antigen vaccines are vaccines made from tumor-associated antigens in, or produced by, cancer cells. Antigen vaccines stimulate a subject's immune system to attack the cancer. Whole cell vaccines are vaccines that use the whole cancer cell, not just a specific antigen from it, to make the vaccine. The vaccine is made from a subject's own cancer cells, another subject's cancer cells or cancer cells grown in a laboratory. The cells are treated in the laboratory, usually with radiation, so that they can't grow, and are administered to the subject via injection or through an intravenous drip into the bloodstream so they can stimulate the immune system to attack the cancer. One type of whole cell vaccine is a dendritic cell vaccine, which helps the immune system to recognize and attack abnormal cells, such as cancer cells. Dendritic cell vaccines are made by growing dendritic cells alongside the cancer cells in the lab. The vaccine is administered to stimulate the immune system to attack the cancer. Anti-idiotype vaccines are vaccines that stimulate the body to make antibodies against cancer cells. Cancer cells make some tumor-associated antigens that the immune system recognizes as foreign. However, because cancer cells are similar to non-cancer cells, the immune system can respond weakly. DNA vaccines boost the immune response. DNA vaccines are made from DNA from cancer cells that carry the genes for the tumor-associated antigens. When a DNA vaccine is injected, it enables the cells of the immune system to recognize the tumor-associated antigens, and activates the cells in the immune system (i.e., breaking tolerance). The most promising results from using DNA vaccines are in treating melanoma. Thus, the viruses provided herein can be administered concurrently with, or sequentially to, a whole cell vaccine in the treatment of cancer. In one embodiment, additional therapy is administered in the form of one or more of any of the other treatment modalities provided herein.

Growth factors are natural substances that stimulate the bone marrow to make blood cells. Recombinant technology can be used to generate growth factors, which can be administered to a subject to increase the number of white blood cells, red blood cells and stem cells in the blood. Growth factors used in cancer treatment to boost white blood cells include Granulocyte Colony Stimulating Factor (G-CSF) also called filgrastim (Neupogen®) or lenograstim (Granocyte®) and Granulocyte and Macrophage Colony Stimulating Factor (GM-CSF), also called molgramostim. A growth factor to help treat anemia is erythropoietin (EPO). EPO encourages the body to make more red blood cells, which in turn, increases hemoglobin levels and the levels of oxygen in body tissues. Other growth factors are being developed which can boost platelets. Thus, the viruses provided herein can be administered concurrently with, or sequentially to, a growth factor such as GM-CSF, in the treatment of cancer. In one example, additional therapy is administered in the form of one or more of any of the other treatment modalities provided herein.

Cancer growth inhibitors use cell-signaling molecules which control the growth and multiplication of cells, such as cancer cells. Drugs that block these signaling molecules can stop cancers from growing and dividing. Cancer growth factors include, but are not limited to, tyrosine kinases. Thus, drugs that block tyrosine kinases are tyrosine kinase inhibitors (TKIs). Examples of TKIs include, but are not limited to, Erlotinib (Tarceva®, OSI-774), Gefitinib (Iressa®, ZD 1839) and Imatinib (Glivec®, STI 571). Another type of growth inhibitor is Bortezomib (Velcade®) for multiple myeloma and for some other cancers. Velcade® is a proteasome inhibitor. Proteasomes are found in all cells and help break down proteins in cells. Interfering with the action of proteosomes causes a buildup of proteins in the cell to toxic levels; thereby killing the cancer cells. Cancer cells are more sensitive to Velcade® than normal cells. Thus, the viruses provided herein can be administered concurrently with, or sequentially to, a cancer growth inhibitor, such as Velcade®, in the treatment of cancer. In one embodiment, additional therapy is administered in the form of one or more of any of the other treatment modalities provided herein.

Cancers need a blood supply to expand and grow their own blood vessels as they get bigger. Without its own blood supply, a cancer cannot grow due to lack of nutrients and oxygen. Anti-angiogenic drugs stop tumors from developing their own blood vessels. Examples of these types of drugs include, but are not limited to, Thalidomide, mainly for treating myeloma but also in trials for other types of cancer, and Bevacizumab (Avastin®), a type of monoclonal antibody that has been investigated for bowel cancer. Thus, the viruses provided herein can be administered concurrently with, or sequentially to, an anti-angiogenic drug in the treatment of cancer. In one example, additional therapy is administered in the form of one or more of any of the other treatment modalities provided herein.

Cancer growth inhibitors use cell-signaling molecules which control the growth and multiplication of cells, such as cancer cells. Drugs that block these signaling molecules can stop cancers from growing and dividing. Cancer growth factors include, but are not limited to, tyrosine kinases. Thus, drugs that block tyrosine kinases are tyrosine kinase inhibitors (TKIs). Examples of TKIs include, but are not limited to, Erlotinib (Tarceva, OSI-774), Iressa (Gefitinib, ZD 1839) and Imatinib (Glivec, STI 571). Another type of growth inhibitor is Bortezomib (Velcade) for multiple myeloma and for some other cancers. Velcade is a proteasome inhibitor. Proteasomes are found in all cells and help break down proteins in cells. Interfering with the action of proteosomes causes a buildup of proteins in the cell to toxic levels; thereby killing the cancer cells. Cancer cells are more sensitive to Velcade than normal cells. Thus, the viruses provided herein can be administered concurrently with, or sequentially to, a cancer growth inhibitor, such as Velcade, in the treatment of cancer. In one embodiment, additional therapy is administered in the form of one or more of any of the other treatment modalities provided herein.

Cancers need a blood supply to expand and grow their own blood vessels as they get bigger. Without its own blood supply, a cancer cannot grow due to lack of nutrients and oxygen. Anti-angiogenic drugs stop tumors from developing their own blood vessels. Examples of these types of drugs include, but are not limited to, Thalidomide, mainly for treating myeloma but also in trials for other types of cancer, and Bevacizumab (Avastin), a type of monoclonal antibody that has been investigated for bowel cancer. Thus, the viruses provided herein can be administered concurrently with, or sequentially to, an anti-angiogenic drug in the treatment of cancer. In one example, additional therapy is administered in the form of one or more of any of the other treatment modalities provided herein.

Interferon-alpha (IFN-α) is a natural substance produced in the body, in very small amounts, as part of the immune response. IFN-α is administered as a treatment to boost the immune system and help fight cancers such as renal cell (kidney) cancer, malignant melanoma, multiple myeloma and some types of leukemias. IFN-α works in several ways: it can help to stop cancer cells growing, it can also boost the immune system to help it attack the cancer, and it can affect the blood supply to the cancer cells. Thus, the viruses provided herein can be administered concurrently with, or sequentially to, IFN-α in the treatment of cancer. In one embodiment, additional therapy is administered in the form of one or more of any of the other treatment modalities provided herein.

Administration of IL-2 is a biological therapy drug because it is naturally produced by the immune system. Thus, it is also an immunotherapy. Interleukin 2 is used in treating renal cell (kidney) cancer, and is being tested in clinical trials for several other types of cancers. IL-2 works directly on cancer cells by interfering with cell grow and proliferation; it stimulates the immune system by promoting the growth of killer T cells and other cells that attack cancer cells; and it also stimulates cancer cells to secrete chemoattractants that attract immune system cells. IL-2 is generally administered as a subcutaneous injection just under the skin once daily for 5 days, followed by 2 days rest. The cycle of injections is repeated for 4 weeks followed by a week without treatment. The treatment regimen and the number of cycles administered depend on the type of cancer and how it responds to the treatment. IL-2 can be self-administered or administered by a health professional. Alternatively, IL-2 can be administered intravenously via injection or drip. Thus, the viruses provided herein can be administered concurrently with, or sequentially to, IL-2 in the treatment of cancer. In one embodiment, additional therapy is administered in the form of one or more of any of the other treatment modalities provided herein.

Gene therapy involves treating cancer by blocking abnormal genes in cancer cells, repairing or replacing abnormal genes in cancer cells, encouraging even more genes to become abnormal in cancer cells so that they die or become sensitive to treatment, using viruses to carry treatment-activating enzymes into the cancer cells, or a combination thereof. As a result, cancer cells die due to damage in the cell. Cancer cells develop as a result of several types of mutations in several of their genes. Targeted genes include, but are not limited to, those that encourage the cell to multiply (i.e., oncogenes), genes that stop the cell from multiplying (i.e., tumor suppressor genes) and genes that repair other damaged genes. Gene therapy can involve repair of damaged oncogenes or blocking the proteins that the oncogenes produce. The tumor suppressor gene, p53, is damaged in many human cancers. Viruses have been used to deliver an undamaged p53 gene into cancer cells, and early clinical trials are now in progress looking at treating cancers with modified p53-producing viruses. Gene therapy could be used to replace the damaged DNA repairing genes. In an alternative example, methods of increasing DNA damage within a tumor cell can promote death of the tumor cell or cause increased susceptibility of the tumor cell to other cancer treatments, such as radiotherapy or chemotherapy. Thus, the viruses provided herein can be administered concurrently with, or sequentially to, any of the gene therapy methods provided herein or known in the art in the treatment of cancer. In one embodiment, additional therapy is administered in the form of one or more of any of the other treatment modalities provided herein.

Treatment of early stage bladder cancer is called intravesical treatment, which is mainly used to treat stage T1 bladder cancers that are high grade (grade 3 or G3) or carcinoma in situ of the bladder (also known as T is or CIS). BCG is a vaccine for tuberculosis (TB), which also has been found to be effective in treating CIS and preventing bladder cancers from recurring. In some cases, BCG vaccines have been used for treating grade 2 early bladder cancer. Because bladder cancer can occur anywhere in the bladder lining, it cannot be removed in the same way as the papillary early bladder cancers. Rather a BCG vaccine is administered using intravesical therapy; that is, first, a catheter (tube) put is inserted into the bladder, followed by intra-catheter administration of a BCG vaccine and/or a chemotherapy. BCG treatment occurs weekly for 6 weeks or more depending on the effect on the bladder cancer. BCG treatment of bladder cancer can be combined with other types of treatments, such as administration of chemotherapy (intravesical), IL-2, treatment with drugs that make cells sensitive to light, vitamins, and photodynamic therapy. Thus, the viruses provided herein can be administered concurrently with, or sequentially to, BCG vaccines in the treatment of cancer. In one embodiment, additional therapy is administered in the form of one or more of any of the other treatment modalities provided herein.

### C. Monitoring

The methods provided herein can further include one or more steps of monitoring the subject, monitoring the tumor, and/or monitoring the virus administered to the subject. Any of a variety of monitoring steps can be included in the methods provided herein, including, but not limited to, monitoring tumor size, monitoring anti-(tumor antigen) antibody titer, monitoring the presence and/or size of metastases, monitoring the subject's lymph nodes, monitoring the subject's weight or other health indicators including blood or urine markers, monitoring anti-(viral antigen) antibody titer, monitoring viral expression of a detectable gene product, and directly monitoring viral titer in a tumor, tissue or organ of a subject.

The purpose of the monitoring can be simply for assessing the health state of the subject or the progress of therapeutic treatment of the subject, or can be for determining whether or not further administration of the same or a different virus is warranted, or for determining when or whether or not to administer a compound to the subject where the compound can act to increase the efficacy of the therapeutic method, or the compound can act to decrease the pathogenicity of the virus administered to the subject.

### 1. Monitoring Viral Gene Expression

In some embodiments, the methods provided herein can include monitoring one or more virally expressed genes. Viruses can express one or more detectable gene products, including but not limited to, detectable proteins (e.g. luminescent or fluorescent proteins) or proteins that induce a detectable signal (e.g. proteins that bind or transport detectable compounds or modify substrates to produce a signal). The infected cells/tissue can thus be imaged by one more optical or non-optical imaging methods.

As provided herein, measurement of a detectable gene product expressed by a virus can provide an accurate determination of the level of virus present in the subject. As further provided herein, measurement of the location of the detectable gene product, for example, by imaging methods including, but not limited to, magnetic resonance, fluorescence, and tomographic methods, can determine the localization of the virus in the subject. Accordingly, the methods provided herein that include monitoring a detectable viral gene product can be used to determine the presence or absence of the virus in one or more organs or tissues of a subject, and/or the presence or absence of the virus in a tumor or metastases of a subject. Further, the methods provided herein that include monitoring a detectable viral gene product can be used to determine the titer of virus present in one or more organs, tissues, tumors or metastases. Methods that include monitoring the localization and/or titer of viruses in a subject can be used for determining the pathogenicity of a virus; since viral infection, and particularly the level of infection, of normal tissues and organs can indicate the pathogenicity of the probe, methods of monitoring the localization and/or amount of viruses in a subject can be used to determine the pathogenicity of a virus. Since methods provided herein can be used to monitor the amount of viruses at any particular location in a subject, the methods that include monitoring the localization and/or titer of viruses in a subject can be performed at multiple time points, and, accordingly can determine the rate of viral replication in a subject, including the rate of viral replication in one or more organs or tissues of a subject; accordingly, the methods of monitoring a viral gene product can be used for determining the replication competence of a virus. The methods provided herein also can be used to quantitate the amount of virus present in a variety of organs or tissues, and tumors or metastases, and can thereby indicate the degree of preferential accumulation of the virus in a subject; accordingly, the viral gene product monitoring methods provided herein can be used in methods of determining the ability of a virus to accumulate in tumor or metastases in preference to normal tissues or organs. Since the viruses used in the methods provided herein can accumulate in an entire tumor or can accumulate at multiple sites in a tumor, and can also accumulate in metastases, the methods provided herein for monitoring a viral gene product can be used to determine the size of a tumor or the number of metastases that are present in a subject. Monitoring such presence of viral gene product in tumor or metastasis over a range of time can be used to assess changes in the tumor or metastasis, including growth or shrinking of a tumor, or development of new metastases or disappearance of metastases, and also can be used to determine the rate of growth or shrinking of a tumor, or development of new metastases or disappearance of metastases, or the change in the rate of growth or shrinking of a tumor, or development of new metastases or disappearance of metastases. Accordingly, the methods of monitoring a viral gene product can be used for monitoring a neoplastic disease in a subject, or for determining the efficacy of treatment of a neoplastic disease, by determining rate of growth or shrinking of a tumor, or development of new metastases or disappearance of metastases, or the change in the rate of growth or shrinking of a tumor, or development of new metastases or disappearance of metastases.

Any of a variety of detectable proteins can be detected in the monitoring methods provided herein; an exemplary, non-limiting list of such detectable proteins includes any of a variety of fluorescent proteins (e.g., green or red fluorescent proteins), any of a variety of luciferases, transferrin or other iron binding proteins; or receptors, binding proteins, and antibodies, where a compound that specifically binds the receptor, binding protein or antibody can be a detectable agent or can be labeled with a detectable substance (e.g., a radionuclide or imaging agent); or transporter proteins (e.g. hNET or hNIS) that can bind to and transport detectable molecules into the cell. Viruses expressing a detectable protein can be detected by a combination of the method provided herein and know in the art. Viruses expressing more than one detectable protein or two or more viruses expressing various detectable protein can be detected and distinguished by dual imaging methods. For example, a virus expressing a fluorescent protein and an iron binding protein can be detected in vitro or in vivo by low light fluorescence imaging and magnetic resonance, respectively. In another example, a virus expressing two or more fluorescent proteins can be detected by fluorescence imaging at different wavelength. In vivo dual imaging can be performed on a subject that has been administered a virus expressing two or more detectable gene products or two or more viruses each expressing one or more detectable gene products.

### 2. Monitoring Tumor Size

Also provided herein are methods of monitoring tumor and/or metastasis size and location. Tumor and or metastasis size can be monitored by any of a variety of methods known in the art, including external assessment methods or tomographic or magnetic imaging methods. In addition to the methods known in the art, methods provided herein, for example, monitoring viral gene expression, can be used for monitoring tumor and/or metastasis size.

Monitoring size over several time points can provide information regarding the increase or decrease in size of a tumor or metastasis, and can also provide information regarding the presence of additional tumors and/or metastases in the subject. Monitoring tumor size over several time points can provide information regarding the development of a neoplastic disease in a subject, including the efficacy of treatment of a neoplastic disease in a subject.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, IHD-J virus strain refers to a virus strain mixture provided by ATCC® Catalog No. VR-156™ derived from the New York City Department of Health IHD strain. See Gallwitz et al., (2003) J Clin Microbiol. 41 (9): 4068-4070.

As used herein, a VIP02 clonal strain or VIP02 clonal isolate refers to a virus that is derived from the IHD-J virus strain by plaque isolation, or other method in which a single clone is propagated, and that has a genome that is homogenous in sequence. Hence, a VIP02 clonal strain includes a virus whose genome can be present in a virus preparation propagated from an IHD-J strain mixture provided by ATCC® Catalog No. VR-156™ derived from the New York City Department of Health IHD strain. A VIP02 clonal strain does not include a recombinant VIP02 virus that is genetically engineered by recombinant means using recombinant DNA methods to introduce heterologous nucleic acid. In particular, a VIP02 clonal strain has a genome that does not contain heterologous nucleic acid that contains an open reading frame encoding a heterologous protein, e.g., a heterologous gene product. For example, a VIP02 clonal strain has a genome that does not contain non-viral heterologous nucleic acid that contains an open reading frame encoding a non-viral heterologous protein, e.g., a heterologous gene product. As described herein, however, it is understood that any of the VIP02 clonal strains provided herein can be modified in its genome by recombinant means to generate a recombinant virus. For example, a VIP02 clonal strain can be modified to generate a recombinant VIP02 virus that contains insertion of nucleotides that contain an open reading frame encoding a heterologous protein, e.g., a heterologous gene product.

As used herein, VIP02 is an IHD-J derived clonal strain that has a genome having a sequence of nucleotides set forth in SEQ ID NO: 1, or a genome having a sequence of nucleotides that has at least 99% sequence identity to the sequence of nucleotides set forth in SEQ ID NO: 1.

As used herein, a modified VIP02 virus strain refers to a VIP02 virus that has a genome that comprises one or more modifications, such as one or more modifications selected from among a substitution, an insertion, and a deletion of one or more nucleotides, including combinations thereof, as compared to an unmodified parental VIP02 virus. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein, e.g., a heterologous gene product. For example, modified viruses herein can contain one or more heterologous nucleic acid sequences in the form of a gene expression cassette for the expression of a heterologous gene.

As used herein, VIR11 is a modified VIP02 clonal strain that has a genome having a sequence of nucleotides set forth in SEQ ID NO: 3, or a genome having a sequence of nucleotides that has at least 99% sequence identity to the sequence of nucleotides set forth in SEQ ID NO: 3.

As used herein, VIR13 is a modified VIP02 clonal strain that has a genome having a sequence of nucleotides set forth in SEQ ID NO: 4, or a genome having a sequence of nucleotides that has at least 99% sequence identity to the sequence of nucleotides set forth in SEQ ID NO: 4.

As used herein, VIR37 is a modified VIP02 clonal strain that has a genome having a sequence of nucleotides set forth in SEQ ID NO: 6, or a genome having a sequence of nucleotides that has at least 99% sequence identity to the sequence of nucleotides set forth in SEQ ID NO: 6.

As used herein, VIR40 is a modified VIP02 clonal strain that has a genome having a sequence of nucleotides set forth in SEQ ID NO: 7, or a genome having a sequence of nucleotides that has at least 99% sequence identity to the sequence of nucleotides set forth in SEQ ID NO: 7.

As used herein, VIR41 is a modified VIP02 clonal strain that has a genome having a sequence of nucleotides set forth in SEQ ID NO: 8, or a genome having a sequence of nucleotides that has at least 99% sequence identity to the sequence of nucleotides set forth in SEQ ID NO: 8.

As used herein, VIR42 is a modified VIP02 clonal strain that has a genome having a sequence of nucleotides set forth in SEQ ID NO: 9, or a genome having a sequence of nucleotides that has at least 99% sequence identity to the sequence of nucleotides set forth in SEQ ID NO: 9.

As used herein, VIR46 is a modified VIP02 clonal strain that has a genome having a sequence of nucleotides set forth in SEQ ID NO: 10, or a genome having a sequence of nucleotides that has at least 99% sequence identity to the sequence of nucleotides set forth in SEQ ID NO: 10.

As used herein, VIR49 is a modified VIP02 clonal strain that has a genome having a sequence of nucleotides set forth in SEQ ID NO: 11, or a genome having a sequence of nucleotides that has at least 99% sequence identity to the sequence of nucleotides set forth in SEQ ID NO: 11.

As used herein, VIR52 is a modified VIP02 clonal strain that has a genome having a sequence of nucleotides set forth in SEQ ID NO: 12, or a genome having a sequence of nucleotides that has at least 99% sequence identity to the sequence of nucleotides set forth in SEQ ID NO: 12.

As used herein, VIR71 is a modified VIP02 clonal strain that has a genome having a sequence of nucleotides set forth in SEQ ID NO: 13, or a genome having a sequence of nucleotides that has at least 99% sequence identity to the sequence of nucleotides set forth in SEQ ID NO: 13.

As used herein, VIR86 is a modified VIP02 clonal strain that has a genome having a sequence of nucleotides set forth in SEQ ID NO: 47, or a genome having a sequence of nucleotides that has at least 99% sequence identity to the sequence of nucleotides set forth in SEQ ID NO: 47.

As used herein, VIR93 is a modified VIP02 clonal strain that has a genome having a sequence of nucleotides set forth in SEQ ID NO: 49, or a genome having a sequence of nucleotides that has at least 99% sequence identity to the sequence of nucleotides set forth in SEQ ID NO: 49.

As used herein, VIR94 is a modified VIP02 clonal strain that has a genome having a sequence of nucleotides set forth in SEQ ID NO: 48, or a genome having a sequence of nucleotides that has at least 99% sequence identity to the sequence of nucleotides set forth in SEQ ID NO: 48.

As used herein, VIR96 is a modified VIP02 clonal strain that has a genome having a sequence of nucleotides set forth in SEQ ID NO: 50, or a genome having a sequence of nucleotides that has at least 99% sequence identity to the sequence of nucleotides set forth in SEQ ID NO: 50.

As used herein, VIR100 is a modified VIP02 clonal strain that has a genome having a sequence of nucleotides set forth in SEQ ID NO: 80, or a genome having a sequence of nucleotides that has at least 99% sequence identity to the sequence of nucleotides set forth in SEQ ID NO: 80.

As used herein, VIR102 is a modified VIP02 clonal strain that has a genome having a sequence of nucleotides set forth in SEQ ID NO: 81, or a genome having a sequence of nucleotides that has at least 99% sequence identity to the sequence of nucleotides set forth in SEQ ID NO: 81.

As used herein, VIR103 is a modified VIP02 clonal strain that has a genome having a sequence of nucleotides set forth in SEQ ID NO: 82, or a genome having a sequence of nucleotides that has at least 99% sequence identity to the sequence of nucleotides set forth in SEQ ID NO: 82.

As used herein, VIR104 is a modified VIP02 clonal strain that has a genome having a sequence of nucleotides set forth in SEQ ID NO: 83, or a genome having a sequence of nucleotides that has at least 99% sequence identity to the sequence of nucleotides set forth in SEQ ID NO: 83.

As used herein, VIR105 is a modified VIP02 clonal strain that has a genome having a sequence of nucleotides set forth in SEQ ID NO: 84, or a genome having a sequence of nucleotides that has at least 99% sequence identity to the sequence of nucleotides set forth in SEQ ID NO: 84.

As used herein, VIR106 is a modified VIP02 clonal strain that has a genome having a sequence of nucleotides set forth in SEQ ID NO: 85, or a genome having a sequence of nucleotides that has at least 99% sequence identity to the sequence of nucleotides set forth in SEQ ID NO: 85.

As used herein, VIR109 is a modified VIP02 clonal strain that has a genome having a sequence of nucleotides set forth in SEQ ID NO: 86, or a genome having a sequence of nucleotides that has at least 99% sequence identity to the sequence of nucleotides set forth in SEQ ID NO: 86.

As used herein, VIR111 is a modified VIP02 clonal strain that has a genome having a sequence of nucleotides set forth in SEQ ID NO: 87, or a genome having a sequence of nucleotides that has at least 99% sequence identity to the sequence of nucleotides set forth in SEQ ID NO: 87.

As used herein, VIR113 is a modified VIP02 clonal strain that has a genome having a sequence of nucleotides set forth in SEQ ID NO: 88, or a genome having a sequence of nucleotides that has at least 99% sequence identity to the sequence of nucleotides set forth in SEQ ID NO: 88.

As used herein, VIR114 is a modified VIP02 clonal strain that has a genome having a sequence of nucleotides set forth in SEQ ID NO: 89, or a genome having a sequence of nucleotides that has at least 99% sequence identity to the sequence of nucleotides set forth in SEQ ID NO: 89.

As used herein, VIR115 is a modified VIP02 clonal strain that has a genome having a sequence of nucleotides set forth in SEQ ID NO: 90, or a genome having a sequence of nucleotides that has at least 99% sequence identity to the sequence of nucleotides set forth in SEQ ID NO: 90.

As used herein, VIR123 is a modified VIP02 clonal strain that has a genome having a sequence of nucleotides set forth in SEQ ID NO: 91, or a genome having a sequence of nucleotides that has at least 99% sequence identity to the sequence of nucleotides set forth in SEQ ID NO: 91.

As used herein, VIR127 is a modified VIP02 clonal strain that has a genome having a sequence of nucleotides set forth in SEQ ID NO: 92, or a genome having a sequence of nucleotides that has at least 99% sequence identity to the sequence of nucleotides set forth in SEQ ID NO: 92.

As used herein, VIR128 is a modified VIP02 clonal strain that has a genome having a sequence of nucleotides set forth in SEQ ID NO: 93, or a genome having a sequence of nucleotides that has at least 99% sequence identity to the sequence of nucleotides set forth in SEQ ID NO: 93.

As used herein, “production by recombinant methods” or “methods using recombinant DNA methods” or variations thereof refers to the use of the well-known methods of molecular biology for expressing proteins encoded by cloned DNA.

As used herein, a “gene expression cassette” or “expression cassette” is a nucleic acid construct, containing nucleic acid elements that are capable of effecting expression of a gene in hosts that are compatible with such sequences. Expression cassettes include at least promoters and, optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a virus preparation, for example an IHD-J virus preparation, a VIP02 virus preparation or a preparation of a recombinant or modified virus, refers to a virus composition obtained by propagation of a virus strain, for example an IHD-J virus strain, an VIP02 clonal strain or a modified or recombinant virus strain, in vivo or in vitro in a culture system. For example, an IHD-J/VIP02 virus preparation refers to a viral composition obtained by propagation of a virus strain in host cells, typically upon purification from the culture system using standard methods known in the art. A virus preparation generally is made up of a number of virus particles or virions. If desired, the number of virus particles in the sample or preparation can be determined using a plaque assay to calculate the number of plaque forming units per sample unit volume (pfu/mL), assuming that each plaque formed is representative of one infective virus particle. Each virus particle or virion in a preparation can have the same genomic sequence compared to other virus particles (i.e. the preparation is homogenous in sequence) or can have different genomic sequences (i.e. the preparation is heterogenous in sequence). It is understood to those of skill in the art that, in the absence of clonal isolation, heterogeneity or diversity in the genome of a virus can occur as the virus reproduces, such as by homologous recombination events that occur in the natural selection processes of virus strains (Plotkin & Orenstein (eds) “Recombinant Vaccinia Virus Vaccines” in Vaccines, 3rd edition (1999)).

As used herein, a virus mixture is a virus preparation that contains a number of virus particles that differ in their genomic sequences. The virus mixture can be obtained by infecting a culture system, for example host cells, with two or more different virus strains, or one virus strain and genomic DNA or cloned DNA, followed by propagation and purification of the resulting virus. For purposes herein, a VIP02 virus preparation can include a virus mixture obtained by propagation of cells infected with a VIP02 strain and another virus, genomic DNA or cloned DNA, followed by isolation of a virus preparation from the culture, where the preparation contains progeny viruses produced by the infection. For example, the other virus strain can be poxvirus, such as avipox virus, myxoma virus or other vaccinia virus; a herpesvirus such as herpes simplex virus (HSV), cytomegalovirus (CMV), Epstein-Barr virus (EBV), hepadnaviruses (e.g., hepatitis B virus), polyoma viruses, papillomaviruses, adenoviruses and adeno-associated viruses; and single-stranded DNA viruses, such as parvoviruses. The other virus can be an attenuated virus, oncolytic virus or other virus with known anti-tumor activity and/or moderate to mild toxicity.

As used herein, “virus” refers to any of a large group of infectious entities that cannot grow or replicate without a host cell, including recombinant viruses and recombinant oncolytic viruses as described herein. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, sindbis virus, papillomavirus, parvovirus, reovirus, coxsackievirus, influenza virus, mumps virus, poliovirus, and Semliki Forest virus.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein, “toxicity” (also referred to as virulence or pathogenicity herein) with reference to a virus refers to the deleterious or toxic effects to a host upon administration of the virus. For an oncolytic virus, such as IDH-J/VIP02, the toxicity of a virus is associated with its accumulation in non-tumorous organs or tissues, which can affect the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on weight.

As used herein, “reduced toxicity” means that the toxic or deleterious effects upon administration of the virus to a host are attenuated or lessened compared to a host not treated with the virus or compared to a host that is administered with another reference or control virus. For purposes herein, exemplary of a reference or control virus is the VIP02 virus strain. Whether toxicity is reduced or lessened can be determined by assessing the effect of a virus and, if necessary, a control or reference virus, on a parameter indicative of toxicity. It is understood that when comparing the activity of two or more different viruses, the amount of virus (e.g. pfu) used in an in vitro assay or administered in vivo is the same or similar and the conditions (e.g. in vivo dosage regime) of the in vitro assay or in vivo assessment are the same or similar. For example, when comparing effects upon in vivo administration of a virus and a control or reference virus the subjects are the same species, size, gender and the virus is administered in the same or similar amount under the same or similar dosage regime. In particular, a virus with reduced toxicity can mean that upon administration of the virus to a host, such as for the treatment of a disease, the virus does not accumulate in non-tumorous organs and tissues in the host to an extent that results in damage or harm to the host, or that impacts survival of the host to a greater extent than the disease being treated does or to a greater extent than a control or reference virus does. For example, a virus with reduced toxicity includes a virus that does not result in death of the subject over the course of treatment.

As used herein, accumulation of a virus in a particular tissue refers to the distribution of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s), the immunocompetence of the host and dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, “preferential accumulation” refers to accumulation of a virus at a first location at a higher level than accumulation at a second location (i.e., the concentration of viral particles, or titer, at the first location is higher than the concentration of viral particles at the second location). Thus, a virus that preferentially accumulates in immunoprivileged tissue (tissue that is sheltered from the immune system), such as inflamed tissue, and tumor tissue, relative to normal tissues or organs, refers to a virus that accumulates in immunoprivileged tissue, such as tumor, at a higher level (i.e., concentration or viral titer) than the virus accumulates in normal tissues or organs.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, “greater” or “improved” activity with reference to anti-tumor activity or anti-tumorigenicity means that a virus strain is capable of preventing or inhibiting the formation or growth of tumors in vitro or in vivo in a subject to a greater extent than a reference or control virus or to a greater extent than absence of treatment with the virus. For purposes herein, exemplary of a reference or control virus is the VIP02 virus. Whether anti-tumor activity is “greater” or “improved” can be determined by assessing the effect of a virus and, if necessary, a control or reference virus, on a parameter indicative of anti-tumor activity. It is understood that when comparing the activity of two or more different viruses, the amount of virus (e.g. pfu) used in an in vitro assay or administered in vivo is the same or similar, and the conditions (e.g. in vivo dosage regime) of the in vitro assay or in vivo assessment are the same or similar.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acid include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, the expressions “nucleic acid sequence” or “nucleotide sequence” or “sequence of nucleotides” can be used interchangeably.

As used herein, a viral clonal strain or virus strain preparation that contains heterologous nucleic acid refers to such strains that contain nucleic acid not present in the parental clonal strain. For example, the virus whose sequence is set forth in SEQ ID NO: 1 (VIP02) is a clonal strain, but the virus of SEQ ID NO: 7, designated VIR40, contains heterologous nucleic acid, such as the insert designated iCasp9.

As used herein, a heterologous protein or heterologous gene product or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266:19867-19870 (1991) and Shine and Delgamo, Nature 254 (5495): 34-38 (1975)). The desirability of (or need for) such modification can be empirically determined.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter can also be a hybrid promoter composed of different elements derived from different native promoters.

As used herein, dosing regimen refers to the amount of agent, for example, a virus or other agent, administered, and the frequency of administration over the course of a cycle of administration. The dosing regimen is a function of the disease or condition to be treated, and thus can vary.

As used herein, frequency of administration refers to the number of times an agent is administered during the cycle of administration. For example, frequency can be days, weeks or months. For example, frequency can be administration once during a cycle of administration, two times, three times, four times, five times, six times or seven times. The frequency can refer to consecutive days during the cycle of administration. The particular frequency is a function of the particular disease or condition treated.

As used herein, a “cycle of administration” refers to the repeated schedule of the dosing regimen of administration of a virus that is repeated over successive administrations. For example, an exemplary cycle of administration is a 28 day cycle.

As used herein, treatment of a subject that has a condition, disorder or disease means any manner of treatment in which the symptoms of the condition, disorder or disease are ameliorated or otherwise beneficially altered. Treatment encompasses any pharmaceutical use of the viruses described and provided herein.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, neoplastic disease refers to any disorder involving cancer, including hyperplasia and tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, leukemia, lymphoma, pancreatic cancer, lung cancer, ovarian cancer, breast cancer, cervical cancer, bladder cancer, prostate cancer, glioma tumors, adenocarcinomas, liver cancer and skin cancer. Exemplary cancers in humans include a bladder tumor, breast tumor, prostate tumor, basal cell carcinoma, biliary tract cancer, bladder cancer, bone cancer, brain and CNS cancer (e.g., glioma tumor), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g., small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, an effective amount, or therapeutically effective amount, of a virus or compound for treating a particular disease is an amount to ameliorate, or in some manner reduce the symptoms associated with the disease. The amount will vary from one individual to another and will depend upon a number of factors, including, but not limited to, age, weight, the overall physical condition of the patient and the severity of the disease. A therapeutically effective amount can be administered as a single dosage or can be administered in multiple dosages according to a regimen, whereby it is effective. The amount can cure the disease but, typically, is administered in order to ameliorate the symptoms of the disease. Repeated administration can be required to achieve the desired amelioration of symptoms.

As used herein, an effective amount, or therapeutically effective amount, of a virus or compound for treating a neoplastic disease, including a tumor, a hyperplasia, or a metastasis is an amount to ameliorate, or in some manner reduce the symptoms associated with the neoplastic disease, including, but not limited to slowing of tumor growth, lysis of tumor cells, reduction in the size of the tumor and/or the size of metastasis, prevention of new tumor growth, or prevention of metastasis of a primary tumor.

As used herein, a tumor, also known as a neoplasm, is an abnormal mass of tissue that results when cells proliferate at an abnormally high rate and/or cells have lost the ability to die due to apoptotic signals. Tumors may show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, and pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and blastic tumors.

As used herein, proliferative disorders include any disorders involving abnormal proliferation of cells (i.e. cells proliferate more rapidly compared to normal tissue growth), such as, but not limited to, neoplastic diseases.

As used herein, a “tumor cell” is any cell that is part of a tumor. Typically, the viruses provided herein preferentially infect tumor cells in a subject compared to normal cells.

As used herein, a “metastatic cell” is a cell that has the potential for can form metastasis. Metastatic cells have the ability to metastasize from a first tumor in a subject and can colonize tissue at a different site in the subject to form a second tumor at the site.

As used herein, “tumorigenic cell” is a cell that, when introduced into a suitable site in a subject, can form a tumor. The cell can be non-metastatic or metastatic.

As used herein, a “normal cell” is a cell that is not derived from a tumor.

As used herein, the term “cell” refers to the basic unit of structure and function of a living organism as is commonly understood in the biological sciences. A cell can be a unicellular organism that is self-sufficient and that can exist as a functional whole independently of other cells. A cell can also be one that, when not isolated from the environment in which it occurs in nature, is part of a multicellular organism made up of more than one type of cell. Such a cell, which can be thought of as a “non-organism” or “non-organismal” cell, generally is specialized in that it performs only a subset of the functions performed by the multicellular organism as whole. Thus, this type of cell is not a unicellular organism. Such a cell can be a prokaryotic or eukaryotic cell, including animal cells, such as mammalian cells, human cells and non-human animal cells or non-human mammalian cells. Animal cells include any cell of animal origin that can be found in an animal. Thus, animal cells include, for example, cells that make up the various organs, tissues and systems of an animal.

As used herein an “isolated cell” is a cell that exists in vitro and is separate from the organism from which it was originally derived.

As used herein, a “cell line” is a population of cells derived from a primary cell that is capable of stable growth in vitro for many generations. Cell lines are commonly referred to as “immortalized” cell lines to describe their ability to continuously propagate in vitro.

As used herein a “tumor cell line” is a population of cells that is initially derived from a tumor. Such cells typically have undergone some change in vivo such that they theoretically have indefinite growth in culture; unlike primary cells, which can be cultured only for a finite period of time. Moreover, such cells preferably can form tumors after they are injected into susceptible animals.

As used herein, a “primary cell” is a cell that has been isolated from a subject.

As used herein, a “host cell” or “target cell” are used interchangeably to mean a cell that can be infected by a virus.

As used herein, the term “tissue” refers to a group, collection or aggregate of similar cells generally acting to perform a specific function within an organism.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues.

As used herein, therapeutic agents are agents that ameliorate the symptoms of a disease or disorder or ameliorate the disease or disorder. Therapeutic agent, therapeutic compound, or therapeutic regimens include conventional drugs and drug therapies, including vaccines for treatment or prevention (i.e., reducing the risk of getting a particular disease or disorder), which are known to those skilled in the art and described elsewhere herein. Therapeutic agents for the treatment of neoplastic disease include, but are not limited to, moieties that inhibit cell growth or promote cell death, that can be activated to inhibit cell growth or promote cell death, or that activate another agent to inhibit cell growth or promote cell death. Therapeutic agents for use in the methods provided herein can be, for example, an anticancer agent. Exemplary therapeutic agents include, for example, therapeutic microorganisms, such as therapeutic viruses and bacteria, cytokines, growth factors, photosensitizing agents, radionuclides, toxins, antimetabolites, signaling modulators, anticancer antibiotics, anticancer antibodies, angiogenesis inhibitors, radiation therapy, chemotherapeutic compounds or a combination thereof.

As used herein, an anticancer agent or compound (used interchangeably with “antitumor or antineoplastic agent”) refers to any agents, or compounds, used in anticancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein. Anticancer agents include antimetastatic agents. Exemplary anticancer agents include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. As used herein, reference to an anticancer or chemotherapeutic agent includes combinations or a plurality of anticancer or chemotherapeutic agents unless otherwise indicated.

As used herein, a “chemosensitizing agent” is an agent which modulates, attenuates, reverses, or affects a cell's or organism's resistance to a given chemotherapeutic drug or compound. The terms “modulator”, “modulating agent”, “attenuator”, “attenuating agent”, or “chemosensitizer” can be used interchangeably to mean “chemosensitizing agent.” In some examples, a chemosensitizing agent can also be a chemotherapeutic agent. Examples of chemosensitizing agents include, but are not limited to, radiation, calcium channel blockers (e.g., verapamil), calmodulin inhibitors (e.g., trifluoperazine), indole alkaloids (e.g., reserpine), quinolines (e.g., quinine), lysosomotropic agents (e.g., chloroquine), steroids (e.g., progesterone), triparanol analogs (e.g., tamoxifen), detergents (e.g., Cremophor® EL), texaphyrins, and cyclic antibiotics (e.g., cyclosporine).

As used herein, a compound produced in a tumor or other immunoprivileged site refers to any compound that is produced in the tumor or tumor environment by virtue of the presence of an introduced virus, generally a recombinant virus, expressing one or more gene products. For example, a compound produced in a tumor can be, for example, an encoded polypeptide or RNA, a metabolite, or compound that is generated by a recombinant polypeptide and the cellular machinery of the tumor or immunoprivileged tissue or cells.

As used herein, a subject includes any organism, including an animal for whom diagnosis, screening, monitoring or treatment is contemplated. Animals include mammals such as primates and domesticated animals. An exemplary primate is human. A patient refers to a subject, such as a mammal, primate, human, or livestock subject afflicted with a disease condition or for which a disease condition is to be determined or risk of a disease condition is to be determined.

As used herein, a delivery vehicle for administration refers to a lipid-based or other polymer-based composition, such as liposome, micelle or reverse micelle, that associates with an agent, such as a virus provided herein, for delivery into a host subject.

As used herein, vector (or plasmid) refers to a nucleic acid construct that contains discrete elements that are used to introduce heterologous nucleic acid into cells for either expression of the nucleic acid or replication thereof. The vectors typically remain episomal, but can be designed to effect stable integration of a gene or portion thereof into a chromosome of the genome. Selection and use of such vectors are well known to those of skill in the art. Expression vectors include vectors capable of expressing DNA that is operatively linked with regulatory sequences, such as promoter regions, that are capable of effecting expression of the DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, nucleic acids include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a sequence complementary to at least a portion of an RNA, with reference to antisense oligonucleotides, means a sequence of nucleotides having sufficient complementarity to be able to hybridize with the RNA, generally under moderate or high stringency conditions, forming a stable duplex; in the case of double-stranded antisense nucleic acids, a single strand of the duplex DNA (i.e., dsRNA) can thus be assayed, or triplex formation can be assayed. The ability to hybridize depends on the degree of complementarity and the length of the antisense nucleic acid. Generally, the longer the hybridizing nucleic acid, the more base mismatches with an encoding RNA it can contain and still form a stable duplex (or triplex, as the case can be). One skilled in the art can ascertain a tolerable degree of mismatch by use of standard procedures to determine the melting point of the hybridized complex.

As used herein, an in vivo method refers to a method performed within the living body of a subject.

As used herein, genetic therapy or gene therapy involves the transfer of heterologous nucleic acid, such as DNA or RNA, into certain cells, target cells, of a mammal, particularly a human, with a disorder or conditions for which such therapy is sought. As used herein, genetic therapy or gene therapy can involve the transfer of heterologous nucleic acid, such as DNA, into a microorganism (e.g., a virus), which microorganism can be transferred to a mammal, particularly a human, with a disorder or conditions for which such therapy is sought. The nucleic acid, such as DNA, is introduced into the selected target cells, such as directly or indirectly, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA (e.g., RNAi, including siRNA) that is in some manner a therapeutic product, or which mediates, directly or indirectly, expression of a therapeutic product. Genetic therapy also can be used to deliver nucleic acid encoding a gene product that replaces a defective gene or supplements a gene product produced by the mammal or the cell in which it is introduced. The introduced nucleic acid can encode a therapeutic compound. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, an agent or compound that modulates the activity of a protein or expression of a gene or nucleic acid either decreases or increases or otherwise alters the activity of the protein or, in some manner, up- or down-regulates or otherwise alters expression of the nucleic acid in a cell.

As used herein, “nucleic acids” include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§ 1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH, or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-isostereomers of amino acids. Exemplary non-natural amino acids are described herein and are known to those of skill in the art.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, recitation that nucleotides or amino acids “correspond to” nucleotides or amino acids in a disclosed sequence, such as set forth in the Sequence listing, refers to nucleotides or amino acids identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073).

As used herein, “sequence identity” refers to the number of identical or similar amino acids or nucleotide bases in a comparison between a test and a reference poly-peptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g. terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48:443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2:482 (1981)). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non identities) and the weighted comparison matrix of Gribskov et al. Nucl. Acids Res. 14:6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences (or any two polypeptides have amino acid sequences) that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Generally, when comparing nucleotide sequences herein, an alignment with no penalty for end gaps (e.g. terminal gaps are not penalized) is used.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein equivalent, when referring to two sequences of nucleic acids, means that the two sequences in question encode the same sequence of amino acids or equivalent proteins. When equivalent is used in referring to two proteins or peptides or other molecules, it means that the two proteins or peptides have substantially the same amino acid sequence with only amino acid substitutions (such as, but not limited to, conservative changes) or structure and that any changes do not substantially alter the activity or function of the protein or peptide. When equivalent refers to a property, the property does not need to be present to the same extent (e.g., two peptides can exhibit different rates of the same type of enzymatic activity), but the activities are usually substantially the same. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially pure means sufficiently homogeneous to appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

As used herein, the term assessing or determining is intended to include quantitative and qualitative determination in the sense of obtaining an absolute value for the activity of a product, and also of obtaining an index, ratio, percentage, visual or other value indicative of the level of the activity. Assessment can be direct or indirect.

As used herein, the terms “inactivating mutation” and “inactivation mutation” can be used interchangeably and are to be interpreted to have the same meaning.

As used herein, activity refers to the in vitro or in vivo activities of a compound or virus provided herein. For example, in vivo activities refer to physiological responses that result following in vivo administration thereof (or of a composition or other mixture). Activity, thus, encompasses resulting therapeutic effects and pharmaceutical activity of such compounds, compositions and mixtures. Activities can be observed in in vitro and/or in vivo systems designed to test or use such activities.

As used herein, a “composition” refers to any mixture of two or more products or compounds. It can be a solution, a suspension, liquid, powder, a paste, aqueous, non-aqueous, or any combination thereof.

As used herein, “a combination” refers to any association between two or among more items or elements. Exemplary combinations include, but are not limited to, two or more pharmaceutical compositions, a composition containing two or more active ingredients, such as two viruses, or a virus and an anticancer agent, such as a chemotherapeutic compound, two or more viruses, a virus and a therapeutic agent, a virus and an imaging agent, a virus and a plurality therapeutic and/or imaging agents, or any association thereof. Such combinations can be packaged as kits.

As used herein, a kit is a packaged combination, optionally, including instructions for use of the combination and/or other reactions and components for such use.

As used herein, a “control” or “standard” refers to a sample that is substantially identical to the test sample, except that it is not treated with a test parameter, or, if it is a plasma sample, it can be from a normal volunteer not affected with the condition of interest. A control also can be an internal control. For example, a control can be a sample, such as a virus, that has a known property or activity.

As used herein, the singular forms “a,” “an” and “the” include plural referents unless the context clearly dictates otherwise. Thus, for example, reference to “an” agent includes one or more agents.

As used herein, the term “or” is used to mean “and/or” unless explicitly indicated to refer to alternatives only or the alternatives are mutually exclusive.

As used herein, ranges and amounts can be expressed as “about” a particular value or range. About also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### VIII. Exemplary Embodiments

Among provided embodiments are:

1. An isolated clonal vaccinia virus (VACV) strain comprising a nucleic acid genome that has at least 95% sequence identity with the nucleic acid sequence set forth in SEQ ID NO: 1 and wherein the nucleic acid genome is characterized by one or more of:
(i) a variant 017 open reading frame (ORF) encoding an amino acid sequence that has at least 95% sequence identity to SEQ ID NO: 57 and comprises a polar uncharged amino acid at position 66, optionally a threonine (T) at position 66;
(ii) a variant 038 (K5L) ORF comprising a nucleotide insertion to effect a frameshift mutation, wherein the 038 (K5L) gene product is altered;
(iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419;
(iv) a variant 104 (H4L) ORF encoding an amino acid sequence having at least 95% sequence identity to SEQ ID NO: 61 and comprises a negatively charged amino acid at position 591, optionally aspartic acid (D) at position 591; and
(v) a variant 182 (A56R) ORF comprising deletion of two nucleotides to effect a frameshift mutation, wherein the 182 (A56R) ORF gene product is altered.

2. The isolated clonal VACV strain of embodiment 1, wherein the nucleic acid genome is characterized by (i) and the variant 017 ORF encodes an amino acid sequence that has at least 96%, at least 97%, at least 98%, at least 99% or 100% sequence identity to SEQ ID NO: 57.

3. The isolated clonal VACV strain of embodiment 1 or embodiment 2, wherein the nucleic acid genome is characterized by (i) and the variant 017 ORF encodes the amino acid sequence set forth in SEQ ID NO: 57.

4. The isolated clonal VACV strain of any of embodiments 1-4, wherein the nucleic acid genome is characterized by (ii) and the nucleotide insertion is guanine (G) corresponding to insertion after nucleotide position 32135 of SEQ ID NO: 1, optionally wherein the variant 038 (K5L) ORF is set forth in SEQ ID NO: 58.

5. The isolated clonal VACV strain of any of embodiments 1-4, wherein the nucleic acid genome is characterized by (ii) and the 038 (K5L) gene product is set forth in SEQ ID NO: 59.

6. The isolated clonal VACV strain of any of embodiments 1-5, wherein the nucleic acid genome is characterized by (iii) and the variant 059 (E2L) ORF encodes an amino acid sequence that has at least 96%, at least 97%, at least 98%, at least 99% or 100% sequence identity to SEQ ID NO: 60.

7. The isolated clonal VACV strain of any of embodiments 1-6, wherein the nucleic acid genome is characterized by (iii) and the variant 059 (E2L) ORF encodes the amino acid sequence set forth in SEQ ID NO: 60.

8 The isolated clonal VACV strain of any of embodiments 1-7, wherein the nucleic acid genome is characterized by (iv) and the 104 (H4L) ORF encodes an amino acid sequence that has at least 96%, at least 97%, at least 98%, at least 99% or 100% sequence identity to SEQ ID NO: 61.

9. The isolated clonal VACV strain of any of embodiments 1-8, wherein the nucleic acid genome is characterized by (iv) and wherein the variant 104 (H4L) ORF encodes the amino acid sequence set forth in SEQ ID NO: 61.

10. The isolated clonal VACV strain of any of embodiments 1-9, wherein the nucleic acid genome is characterized by (v) and the deletion of two nucleotides is deletion of two contiguous nucleotides corresponding to nucleotides after nucleotide position 165972 of SEQ ID NO: 2, optionally wherein the variant 182 (A56R) is set forth in SEQ ID NO: 62.

11. The isolated clonal VACV strain of any of embodiments 1-10, wherein the nucleic acid genome is characterized by (v) and the VACV protein is set forth in SEQ ID NO: 63.

12. The isolated clonal VACV strain of any of embodiments 1-11, wherein the nucleic acid genome is characterized by any two of (i)-(v).

13. The isolated clonal VACV strain of any of embodiments 1-11, wherein the nucleic acid genome is characterized by any three of (i)-(v).

14. The isolated clonal VACV strain of any of embodiments 1-11, wherein the nucleic acid genome is characterized by any four of (i)-(v).

15. The isolated clonal VACV strain of any of embodiments 1-11, wherein the nucleic acid genome is characterized by each of (i)-(v).

16. An isolated clonal vaccinia virus (VACV) strain comprising a nucleic acid genome that has at least 95% sequence identity with the nucleic acid sequence set forth in SEQ ID NO: 1, and wherein the nucleic acid genome is characterized by one or more of:
(i) a guanine (G) at the position corresponding to position 7770 of SEQ ID NO: 1;
(ii) a thymine (T) at the position corresponding to position 15261 of SEQ ID NO: 1;
(iii) a G at the position corresponding to position 32136 of SEQ ID NO: 1;
(iv) a G at the position corresponding to position 49455 of SEQ ID NO: 1;
(v) a cytosine (C) at the position corresponding to position 92969 of SEQ ID NO: 1;
(vi) the nucleic acid sequence CACTTATATAT at the positions corresponding to positions 106870 to 106880 of SEQ ID NO: 1;
(vii) the nucleic acid sequence GTTTTCATTA at the positions corresponding to positions 111267 to 111276 of SEQ ID NO: 1;
(viii) an adenine (A) at the position corresponding to position 162715 of SEQ ID NO: 1;
(ix) the nucleic acid sequence TACAGACACC at the positions corresponding to positions 165844 to 185853 of SEQ ID NO: 1; and
(x) a C at the position corresponding to position 187805 of SEQ ID NO: 1.

17. The isolated clonal VACV strain of embodiment 16, wherein the nucleic acid genome is characterized by any two of (i)-(x).

18. The isolated clonal VACV strain of embodiment 16, wherein the nucleic acid genome is characterized by any three of (i)-(x).

19. The isolated clonal VACV strain of embodiment 16, wherein the nucleic acid genome is characterized by any four of (i)-(x).

20. The isolated clonal VACV strain of embodiment 16, wherein the nucleic acid genome is characterized by any five of (i)-(x).

21. The isolated clonal VACV strain of embodiment 16, wherein the nucleic acid genome is characterized by any six of (i)-(x).

22. The isolated clonal VACV strain of embodiment 16, wherein the nucleic acid genome is characterized by any seven of (i)-(x).

23. The isolated clonal VACV strain of embodiment 16, wherein the nucleic acid genome is characterized by any eight of (i)-(x).

24. The isolated clonal VACV strain of embodiment 16, wherein the nucleic acid genome is characterized by any nine of (i)-(x).

25. The isolated clonal VACV strain of embodiment 16, wherein the nucleic acid genome is characterized by each of (i)-(x).

26. The isolated clonal VACV strain of any of embodiments 1-25, wherein the nucleic acid genome has at least 96% sequence identity with the sequence of nucleotides set forth in SEQ. ID NO: 1.

27. The isolated clonal VACV strain of any of embodiments 1-26, wherein the nucleic acid genome has at least 97% sequence identity with the sequence of nucleotides set forth in SEQ. ID NO: 1.

28. The isolated clonal VACV strain of any of embodiments 1-27, wherein the nucleic acid genome has at least 98% sequence identity with the sequence of nucleotides set forth in SEQ. ID NO: 1.

29. The isolated clonal VACV strain of any of embodiments 1-28, wherein the nucleic acid genome has at a least 99% sequence identity with the sequence of nucleotides set forth in SEQ. ID NO: 1.

30. An isolated clonal vaccinia virus (VACV) strain comprising a nucleic acid genome that has at least 99% sequence identity with the sequence of nucleotides set forth in SEQ. ID NO: 1.

31. The isolated clonal VACV strain of any of embodiments 1-30, wherein the nucleic acid genome has at least 99.5% sequence identity with the sequence of nucleotides set forth in SEQ. ID NO: 1.

32. The isolated clonal VACV strain of any of embodiments 1-31, wherein the nucleic acid genome has at least 99.9% sequence identity with the sequence of nucleotides set forth in SEQ. ID NO: 1.

33. The isolated clonal VACV strain of any of embodiments 1-32, wherein the nucleic acid genome has at least 99.95% sequence identity with the sequence of nucleotides set forth in SEQ. ID NO: 1.

34. The isolated clonal VACV strain of any of embodiments 1-33, wherein the nucleic acid genome does not comprise the sequence of nucleotides set forth in SEQ ID NO: 2.

35. The isolated clonal VACV strain of any of embodiments 1-34, wherein the nucleic acid genome is not modified to contain non-viral heterologous nucleic acid containing an open reading frame encoding a non-viral heterologous protein.

36. The isolated clonal VACV strain of any of embodiments 1-35, wherein the nucleic acid genome is set forth in SEQ ID NO: 1.

37. The isolated clonal VACV strain of any of embodiments 1-36, wherein the clonal VACV strain exhibits enhanced production of extracellular enveloped virions (EEV) after cell infection, optionally as determined by the assay in Example 1.

38. The isolated clonal VACV strain of embodiment 37, wherein greater than 5% of infectious particles after cell infection are EEV.

39. The isolated clonal VACV strain of embodiment 37, wherein greater than 10% of infectious particles after cell infection are EEV.

40. The isolated clonal VACV strain of embodiment 37, wherein greater than 15% of infectious particles after cell infection are EEV.

41. The isolated clonal VACV strain of any of embodiments 1-40 that exhibits oncolytic activity to kill tumor cells.

42. A VACV preparation comprising the isolated clonal VACV strain of any of embodiments 1-41.

43. The VACV preparation of embodiment 42 that is substantially homogenous wherein a plurality of the virus particles in the preparation have the genomic sequence of the clonal VACV strain.

44. The VACV preparation of embodiment 42 or embodiment 43, wherein at least 70% of the virus particles in the preparation have the genomic sequence of the clonal VACV strain.

45. The VACV preparation of embodiment 42 or embodiment 43, wherein at least 80% of the virus particles in the preparation have the genomic sequence of the clonal VACV strain.

46. The VACV preparation of embodiment 42 or embodiment 43, wherein at least 90% of the virus particles in the preparation have the genomic sequence of the clonal VACV strain.

47. The VACV preparation of embodiment 42 or embodiment 43, wherein at least 95% of the virus particles in the preparation have the genomic sequence of the clonal VACV strain.

48. The VACV preparation of embodiment 42 or embodiment 43, wherein at least 98% of the virus particles in the preparation have the genomic sequence of the clonal VACV strain.

49. A pharmaceutical composition comprising the isolated VACV clonal strain of any of embodiments 1-41.

50. A pharmaceutical composition comprising the VACV preparation of any of embodiments 42-48.

51. A recombinant vaccinia virus (VACV) strain comprising a nucleic acid genome of the VACV clonal strain of any of embodiments 1-41 that comprises an inactivating mutation in at least one viral gene.

52. The recombinant VACV strain of embodiment 51, wherein the viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L and I4L.

53. The recombinant VACV of embodiment 51 or embodiment 52, wherein the inactivating mutation is a deletion of all or a portion of the at least one viral gene.

54. The recombinant VACV strain of embodiment 53, wherein the deletion of the at least one viral gene is deletion of the entire gene ORF of a viral gene.

55. The recombinant VACV strain of embodiment 53, wherein the deletion of the at least one viral gene is a deletion of a portion of the ORF of a viral gene, and wherein said deletion is sufficient to render the encoded gene product non-functional.

56. The recombinant VACV strain of any of embodiments 51-55, wherein the at least one viral gene is A35R gene.

57. The recombinant VACV strain of embodiment 56, wherein the nucleic acid genome of the recombinant VACV strain comprises a sequence of nucleotide that has at least 95%, at least 96%, at least 97%, at least 98% or at least 99% sequence identity to the sequence set forth in SEQ ID NO: 3.

58. The recombinant VACV strain of embodiment 56 or embodiment 57, comprising the sequence of nucleotides set forth in SEQ ID NO: 3.

59. The recombinant VACV strain of any of embodiments 51-55, wherein the at least one viral gene is J2R gene.

60. The recombinant VACV strain of embodiment 59, wherein the nucleic acid genome of the recombinant VACV strain comprises a sequence of nucleotide that has at least 95%, at least 96%, at least 97%, at least 98% or at least 99% sequence identity to the sequence set forth in SEQ ID NO: 4.

61. The recombinant VACV strain of embodiment 59 or embodiment 60, comprising the sequence of nucleotides set forth in SEQ ID NO: 4.

62. The recombinant VACV strain of any of embodiments 51-55, wherein the at least one viral gene is B2R.

63. The recombinant VACV strain of any of embodiments 51-56 and 59, wherein the at least one viral gene comprises A35R gene and J2R gene.

64. The recombinant VACV strain of embodiment 63, wherein the nucleic acid genome of the recombinant VACV strain comprises a sequence of nucleotide that has at least 95%, at least 96%, at least 97%, at least 98% or at least 99% sequence identity to the sequence set forth in SEQ ID NO: 12.

65. The recombinant VACV strain of embodiment 63 or embodiment 64 comprising the sequence of nucleotides set forth in SEQ ID NO: 12.

66. The recombinant VACV strain of any of embodiments 51-55 and 62, wherein the at least one viral gene comprises B2R gene and J2R gene.

67. The recombinant VACV strain of embodiment 66, wherein the nucleic acid genome of the recombinant VACV strain comprises a sequence of nucleotide that has at least 95%, at least 96%, at least 97%, at least 98% or at least 99% sequence identity to the sequence set forth in SEQ ID NO: 48.

68. The recombinant VACV strain of embodiment 66 or embodiment 67, comprising the sequence of nucleotides set forth in SEQ ID NO: 48.

69. A recombinant oncolytic virus, wherein the recombinant virus is a recombinant vaccinia virus (VACV) strain, comprising:
a genome of the VACV clonal strain of any of embodiments 1-41; and
at least one heterologous nucleic acid encoding one or more heterologous gene product inserted in the genome.

70. A recombinant oncolytic virus comprising at least one heterologous nucleic acid encoding one or more heterologous gene product, wherein the one or more heterologous gene product is a complement inhibitor, a T cell or NK cell evader, an immune stimulating protein, an anti-angiogenic protein, an interferon regulatory factor, an apoptosis inducible protein or a combination of any of the foregoing.

71. The recombinant oncolytic virus of embodiment 70, wherein the oncolytic virus is a vaccinia virus, a herpes simplex virus, vesicular stomatitis virus (VSV), a Maraba virus (MARAV), a measles virus (MV), adenovirus, myxoma virus, orf virus, parvovirus, raccoonpox virus, coxsackievirus, reovirus, Newcastle disease virus, Seneca valley virus, Semliki Forest virus, mumps virus, influenza virus, echovirus, and a poliovirus (PV).

72. The recombinant oncolytic virus of embodiment 70 or embodiment 71, wherein the oncolytic virus is a vaccinia virus.

73. The recombinant oncolytic virus of any of embodiments 69-72, wherein the at least one heterologous nucleic acid encoding the one or more heterologous gene product is inserted into or in place of a non-essential gene or region in the genome of the virus.

74. The recombinant oncolytic virus of any of embodiments 69-73, wherein the at least one heterologous nucleic acid encoding the one or more heterologous gene product is inserted into or in place of the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus.

75. The recombinant oncolytic virus of any of embodiments 69-74, wherein the insertion is into or in place of F14.5L.

76. The recombinant oncolytic virus of any of embodiments 69-74, wherein the insertion is into or in place of A35R.

77. The recombinant oncolytic virus of any of embodiments 69-74, wherein the insertion is into or in place of J2R.

78. The recombinant oncolytic virus of any of embodiments 69-72, wherein the at least one heterologous nucleic acid encoding the one or more heterologous gene product is fused with a viral membrane protein to produce a fusion protein.

79. The recombinant oncolytic virus of embodiment 78, wherein the viral membrane protein is F14.5L, optionally wherein the fusion is at the C-terminus of F14.5L.

80. The recombinant oncolytic virus of embodiment 78 or embodiment 79, wherein the fusion protein is incorporated into the outer membrane of the intracellular mature virus (IMV).

81. The recombinant oncolytic virus of any of embodiments 69-80, comprising an inactivation mutation of at least one viral gene.

82. The recombinant oncolytic virus of embodiment 81, wherein the inactivation mutation is deletion of all or a portion of the at least one viral gene.

83. The recombinant VACV of embodiment 82, wherein the deletion of the at least one viral gene is deletion of the entire gene ORF of a viral gene.

84. The recombinant oncolytic virus of embodiment 82, wherein the deletion of the at least one viral gene is a deletion of a portion of the ORF of a viral gene, and wherein said deletion is sufficient to render the encoded gene product non-functional.

85. The recombinant oncolytic virus of any of embodiments 81-84, wherein the at least one viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, and I4L.

86. The recombinant oncolytic virus of any of embodiments 81-84, wherein the at least one viral gene is A35R.

87. The recombinant oncolytic virus of any of embodiments 81-84, wherein the at least one viral gene is J2R.

88. The recombinant oncolytic virus of any of embodiments 81-84, wherein the at least one viral gene is B2R.

89. The recombinant oncolytic virus of any of embodiments 81-84, wherein the at least one viral gene comprises A35R and J2R.

90. The recombinant oncolytic virus of any of embodiments 81-84, wherein the at least one viral gene comprises B2R and J2R.

91. The recombinant oncolytic virus of any of embodiments 69-90, wherein the nucleic acid encoding a heterologous gene product is operably linked to a promoter.

92. The recombinant oncolytic virus of embodiment 91, wherein the promoter is a poxviral promoter or is a variant or derivative thereof.

93. The recombinant oncolytic virus of embodiment 91 or embodiment 92, wherein the promoter is a vaccinia virus promoter.

94. The recombinant oncolytic virus of any of embodiments 91-93, wherein the promoter is selected from the group consisting of 7.5E, 7.5E/L, SSE, 11KL, SSL, SSEL, mH5, and LEO.

95. The recombinant oncolytic virus of any of embodiments 91-94, wherein the promoter has the sequence of amino acids set forth in any one of SEQ ID NOS: 29, 53, 55, 68, 69, 70, 71, or 72.

96. The recombinant oncolytic virus of any of embodiments 91-94, wherein the promoter is synthetic strong early promoter (SSE).

97. The recombinant VACV strain of embodiment 96, wherein the promoter comprises the sequence set forth in SEQ ID NO: 29.

98. The recombinant oncolytic virus of any of embodiments 91-94, wherein the promoter is a strong early/late promoter (SEL).

99. The recombinant oncolytic virus of embodiment 98, wherein the promoter comprises the sequence set forth in SEQ ID NO: 55.

100. The recombinant oncolytic virus of any of embodiments 91-94, wherein the promoter is mH5.

101. The recombinant oncolytic virus of embodiment 100, wherein the mH5 promoter comprises the sequence set forth in SEQ ID NO: 53

102. The recombinant oncolytic virus of any of embodiments 69-101, wherein the one or more heterologous gene product comprise a therapeutic agent or diagnostic agent.

103. The recombinant oncolytic virus of any of embodiments 69-102, wherein the one or more heterologous gene product are selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging.

104. The recombinant oncolytic virus of any of embodiments 69-103, wherein the one or more heterologous gene product comprise a therapeutic agent selected from among a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor.

105. The recombinant oncolytic virus of any of embodiments 69-103, wherein the one or more heterologous gene product comprise a complement inhibitor.

106. The recombinant oncolytic virus of embodiment 105, wherein the complement inhibitor is Borrelia burgdorferi complement regulatory-acquiring surface protein-2 (CRASP-2) or minimized complement regulator factor H (miniFH).

107. The recombinant oncolytic virus of embodiment 106, wherein the complement inhibitor is CRASP-2 and has a sequence of amino acids that exhibits at least 85%, 90% or 95% sequence identity to the sequence set forth in SEQ ID NO: 18.

108. The recombinant oncolytic virus of embodiment 106 or embodiment 107, wherein the complement inhibitor has the sequence set forth in SEQ ID NO: 18.

109. The recombinant oncolytic virus of any of embodiments 69-108, wherein the nucleic acid genome of the recombinant oncolytic virus comprises a sequence of nucleotides that has at least 95%, at least 96%, at least 97%, at least 98% or at least 99% sequence identity to the sequence set forth in SEQ ID NO: 5.

110. The recombinant oncolytic virus of any of embodiments 69-108, comprising the sequence of nucleotides set forth in SEQ ID NO: 5.

111. The recombinant oncolytic virus of embodiment 106, wherein the complement inhibitor is miniFH.

112. The recombinant oncolytic virus of embodiment 105, 106, or 111, wherein the complement inhibitor has a sequence of amino acids that exhibits at least 85%, 90% or 95% sequence identity to the sequence set forth in SEQ ID NO: 19.

113. The recombinant oncolytic virus of any of embodiments 105, 106, 111, or 112, wherein the complement inhibitor has the sequence set forth in SEQ ID NO: 19.

114. The recombinant oncolytic virus of any of embodiments 69-106 and 111-113, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 6.

115. The recombinant oncolytic virus of any of embodiments 69-106 and 111-114 comprising the sequence of nucleotides set forth in SEQ ID NO: 6.

116. The recombinant oncolytic virus of any of embodiment 69-115, wherein the one or more heterologous gene product comprises a T cell evader or an NK cell evader.

117. The recombinant oncolytic virus of embodiment 116, wherein the T cell evader or NK cell evader is a set of proteins encoded by Cowpox virus ORFs 012, 203 and 018 (CPXV012-203-018).

118. The recombinant oncolytic virus of embodiment 117, wherein the T cell evader or NK cell evader is a set of proteins that is or comprises the CPXV012, CPXV203, and CPXV018 proteins.

119. The recombinant oncolytic virus of embodiment 118, wherein the set of proteins encoded by CPXV012-203-018 comprises the sequence of amino acids that exhibits at least 85%, 90% or 95% sequence identity to the sequence set forth in SEQ ID NO: 20 (CPXV012), the sequence of amino acids that exhibits at least 85%, 90% or 95% sequence identity to the sequence set forth in SEQ ID NO: 21 (CPXV0203), and the sequence of amino acids that exhibits at least 85%, 90% or 95% sequence identity to the sequence set forth in SEQ ID NO: 22 (CPXV018).

120. The recombinant oncolytic virus of embodiment 117 or embodiment 118, wherein the set of proteins encoded by CPXV012-203-018 comprises the sequence of amino acids set forth in SEQ ID NO: 20, SEQ ID NO: 21, and SEQ ID NO: 22.

121. The recombinant oncolytic virus of any of embodiments 69-108 and 117-120, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 10.

122. The recombinant oncolytic virus of any of embodiments 69-108 and 117-121, wherein the nucleic acid genome of the recombinant oncolytic virus comprises the sequence of nucleotides set forth in SEQ ID NO: 10.

123. The recombinant oncolytic virus of any of embodiments 69-104, wherein the one or more heterologous gene product comprise an immune stimulating protein.

124. The recombinant oncolytic virus of embodiment 123, wherein the immune stimulating protein is recombinant LIGHT.

125. The recombinant oncolytic virus of embodiment 124, wherein the recombinant LIGHT is a human protein or is a mutant thereof.

126. The recombinant oncolytic virus of embodiment 125, wherein the recombinant LIGHT comprises an amino acid sequence that has at least 95%, at least 96%, at least 97%, at least 98% or at least 99% sequence identity to the sequence set forth in SEQ ID NO: 30.

127 The recombinant oncolytic virus of any of embodiments 124-126, wherein the recombinant LIGHT is hmLIGHT that is a human LIGHT mutant that binds human and mouse LTBR and HVEM.

128. The recombinant oncolytic virus of embodiment 126 or embodiment 127, wherein the recombinant LIGHT comprises one or more mutation selected from the group consisting of a threonine at position 138, a glycine at position 160, a glycine at position 221 and a lysine at position 222.

129. The recombinant oncolytic virus of embodiment 128, wherein the recombinant LIGHT comprises an amino acid sequence that has at least 95%, at least 96%, at least 97%, at least 98% or at least 99% sequence identity to the sequence set forth in SEQ ID NO: 25.

130. The recombinant oncolytic virus of embodiment 128, wherein the recombinant LIGHT comprises the sequence set forth in SEQ ID NO: 25.

131. The recombinant oncolytic virus of any of embodiments 69-104 and 123-130, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 11.

132. The recombinant oncolytic virus of any of embodiments 69-104 and 123-132, comprising the sequence of nucleotides set forth in SEQ ID NO: 11.

133. The recombinant oncolytic virus of any of embodiments 69-132, wherein the one or more heterologous gene product comprise one or more anti-angiogenic protein.

134. The recombinant oncolytic virus of embodiment 133, wherein the one or more anti-angiogenic protein is a VEGF inhibitor, an angiopoietin inhibitor, versikine or a fusion protein of any two or more of the foregoing.

135. The recombinant oncolytic virus of embodiment 134, wherein the VEGF inhibitor is an anti-VEGF antibody, optionally an anti-VEGF-single chain antibody (scAb).

136. The recombinant oncolytic virus of embodiment 134, wherein the angiopoietin inhibitor is an anti-Angriopoietin-2 (Ang2) antibody, optionally an anti-Ang2 single chain antibody (scAb).

137. The recombinant oncolytic virus of any of embodiments 133-136, wherein the one or more anti-angiogenic protein is a bispecific anti-VEGF/anti-Ang2 antibody.

138. The recombinant oncolytic virus of embodiment 137, wherein the bispecific anti-VEGF/anti-Ang2 antibody comprises a sequence of amino acids that has at least 95%, at least 96%, at least 97%, at least 98% or at least 99% sequence identity to the sequence set forth in SEQ ID NO: 23.

139. The recombinant oncolytic virus of embodiment 137 or embodiment 138, wherein the bispecific anti-VEGF/anti-Ang2 antibody comprises the sequence set forth in SEQ ID NO: 23.

140. The recombinant oncolytic virus of any of embodiment 137-139, wherein the one or more anti-angiogenic protein further comprise versikine.

141. The recombinant oncolytic virus of embodiment 134 or embodiment 140, wherein the versikine has a sequence of amino acids that has at least 95%, at least 96%, at least 97%, at least 98% or at least 99% sequence identity to the sequence set forth in SEQ ID NO: 24.

142. The recombinant oncolytic virus of embodiment 134, embodiment 140 or embodiment 141, wherein the versikine has the sequence of amino acids set forth in SEQ ID NO: 24.

143. The recombinant oncolytic virus of any of embodiments 69-104 and 133-142, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 13.

144. The recombinant oncolytic virus of any of embodiments 69-104 and 133-143 comprising the sequence of nucleotides set forth in SEQ ID NO: 13.

145. The recombinant oncolytic virus of any of embodiments 69-104, 123-130 and 133-142, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 47.

146. The recombinant oncolytic virus of any of embodiments 69-104, 123-130, 133-142 and 145 comprising the sequence of nucleotides set forth in SEQ ID NO: 47.

147. The recombinant oncolytic virus of any of embodiments 69-104, wherein the one or more heterologous gene product comprise an interferon regulatory factor.

148. The recombinant oncolytic virus of embodiment 147, wherein the interferon regulatory factor is an interferon regulatory factor 3 (IRF3).

149. The recombinant oncolytic virus of embodiment 148, wherein the IRF3 is a human IRF3, optionally wherein the hIRF3 comprises a sequence of amino acids that has at least 95%, at least 96%, at least 97%, at least 98% or at least 99% sequence identity to the sequence set forth in SEQ ID NO: 51.

150. The recombinant oncolytic virus of embodiment 148, wherein the IRF3 is a mouse IRF3, optionally wherein the mIRF3 comprises a sequence of amino acids that has at least 95%, at least 96%, at least 97%, at least 98% or at least 99% sequence identity to the sequence set forth in SEQ ID NO: 52.

151. The recombinant oncolytic virus of any of embodiments 69-104 and 147-150, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 49.

152. The recombinant oncolytic virus of any of embodiments 69-104 and 147-151, comprising the sequence of nucleotides set forth in SEQ ID NO: 49.

153. The recombinant oncolytic virus of any of embodiments 69-104 and 147-150, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 50.

154. The recombinant oncolytic virus of any of embodiments 69-104, 147-150, and 153 comprising the sequence of nucleotides set forth in SEQ ID NO: 50.

155. The recombinant oncolytic virus of any of embodiments 69-104, wherein the one or more heterologous gene product comprise an apoptosis-inducible protein.

156. The recombinant oncolytic virus of embodiment 155, wherein the apoptosis-inducible protein comprises a proapoptotic molecule fused with an FKBP variant that is able to bind a chemical inducer of dimerization (CID).

157. The recombinant oncolytic virus of embodiment 156, wherein the FKBP variant is FKBP-F36V, optionally wherein the FKBP-F36V comprises the sequence of amino acids set forth in SEQ ID NO: 56.

158. The recombinant oncolytic virus of embodiment 156 or embodiment 157, wherein the chemical inducer of dimerization is AP1903 (Rimiducid).

159. The recombinant oncolytic virus of any of embodiments 156-158, wherein the proapoptotic molecule is using Fas, the death effector domain (DED) of the Fas-associated death domain-containing protein (FADD) or a caspase, optionally wherein the caspase is caspase 9.

160. The recombinant oncolytic virus of any of embodiments 155-159, wherein the apoptosis-inducible protein is an inducible DED (iDED) comprising the sequence set forth in SEQ ID NO: 27 or a sequence of amino acids that has at least 85%, 90% or 95% sequence identity to SEQ ID NO: 27.

161. The recombinant oncolytic virus of any of embodiments 69-104 and 155-160, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8.

162. The recombinant oncolytic virus of any of embodiments 69-104 and 155-161 comprising the sequence of nucleotides set forth in SEQ ID NO: 8.

163. The recombinant oncolytic virus of any of embodiments 155-159, wherein the apoptosis-inducible protein is an inducible Fas (iFas) comprising the sequence set forth in SEQ ID NO: 28 or a sequence of amino acids that has at least 85%, 90% or 95% sequence identity to SEQ ID NO: 28.

164. The recombinant oncolytic virus of any of embodiments 69-104, 155-160 and 163, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 9.

165. The recombinant oncolytic virus of any of embodiments 69-104, 155-160, 163 and 164 comprising the sequence of nucleotides set forth in SEQ ID NO: 9.

166. The recombinant oncolytic virus of any of embodiments 155-159, wherein the apoptosis-inducible protein is an inducible caspase 9 (iCas9) comprising the sequence set forth in SEQ ID NO: 26 or a sequence of amino acids that has at least 85%, 90% or 95% sequence identity to SEQ ID NO: 26.

167. The recombinant oncolytic virus of any of embodiments 69-104, 155-160 and 166, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 7.

168. The recombinant oncolytic virus of any of embodiments 69-104, 155-160, 166, and 167 comprising the sequence of nucleotides set forth in SEQ ID NO: 7.

169. A pharmaceutical composition comprising the recombinant VACV strain of any of embodiments 51-168.

170. The pharmaceutical composition of embodiments 49, 50 and 169, further comprising a pharmaceutically acceptable carrier.

171. The pharmaceutical composition of embodiments 49, 50, 169 and 170 that is formulated for intravenous administration, intratumoral administration, intraperitoneal administration or intrapleural administration.

172. The pharmaceutical composition of embodiments 49, 50, 169, or 170 that is formulated for intravenous administration.

173. The pharmaceutical composition of any of embodiments 49, 50, and 169-172, wherein the pharmaceutical composition is a liquid composition.

174. The pharmaceutical composition of embodiments 49, 50 and 169-172, wherein the pharmaceutical composition is lyophilized.

175. A method of treating a proliferative disorder in a subject comprising administering to the subject an oncolytic virus of any of embodiments 1-41, the recombinant oncolytic virus of any of embodiments 51-168, or the pharmaceutical composition of any of embodiments 49, 50 and 169-174.

176. The method of embodiment 175, wherein the proliferative disorder is a tumor or a metastasis.

177. The method of embodiment 175 or embodiment 176, wherein the proliferative disease is cancer.

178. The method of embodiment 177, wherein the cancer is a pancreatic cancer, ovarian cancer, lung cancer, colon cancer, prostate cancer, cervical cancer, breast cancer, rectal cancer, renal (kidney) cancer, gastric cancer, esophageal cancer, hepatic (liver) cancer, endometrial cancer, bladder cancer, brain cancer, head and neck cancer, oral cancer (e.g., oral cavity cancer), cervical cancer, uterine cancer, thyroid cancer, testicular cancer, prostate cancer, skin cancers, such as melanoma, e.g., malignant melanoma, cholangiocarcinoma (bile duct cancer), thymic epithelial cancer, e.g., thymoma, leukemia, lymphoma, or multiple myeloma.

179. The method of embodiment 177 or embodiment 178, wherein the cancer is MSS colorectal cancer.

180. The method of embodiment 179, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8.

181. The method of embodiment 179 or embodiment 180, wherein the nucleic acid genome of the recombinant oncolytic virus comprises the sequence of nucleotides set forth in SEQ ID NO: 8.

182. The method of any of embodiments 175-181, wherein the virus is administered in an amount from 1×10⁵ pfu to 1×10¹⁴ pfu.

183. The method of any of embodiments 175-182, further comprising administering a second therapeutic agent for the treatment of the proliferative disorder.

184. The method of any of embodiments 175-183, further comprising another treatment selected from among surgery, radiation therapy, immunosuppressive therapy and administration of an anticancer agent.

185. The method of embodiment 184, wherein the further treatment is administration of an anticancer agent selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent and a combination of any of the preceding thereof.

186. The method of any of embodiments 175-185, wherein the virus is administered intravenously.

187. The method of any of embodiments 175-186, further comprising administering AP1903 (Rimiducid) to the subject.

188. The method of any of embodiments 175-187, wherein the recombinant oncolytic virus administered to the subject comprises a heterologous nucleic acid encoding an apoptosis inducible protein.

189. The method of embodiment 187 or embodiment 188, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8.

190. The method of any of embodiments 187-189, wherein the nucleic acid genome of the recombinant oncolytic virus comprises the sequence of nucleotides set forth in SEQ ID NO: 8.

191. The method of any of embodiments 187-190, wherein the subject exhibits severe immune deficiency and is sensitive to virus infection.

192. A method of inhibiting virus replication, the method comprising contacting cells infected with a recombinant oncolytic virus with AP1903 (Rimiducid), wherein the recombinant oncolytic virus comprises a heterologous nucleic acid encoding an apoptosis inducible protein.

193. The method of embodiment 192, wherein the contacting occurs in vivo in a subject, wherein the AP1903 (Rimiducid) has been administered to a subject previously administered with a recombinant oncolytic virus comprising the heterologous nucleic acid encoding an apoptosis inducible protein.

194. A method of inhibiting virus replication in a subject, the method comprising administering to a subject AP1903 (Rimiducid), wherein the subject has been previously administered a recombinant oncolytic virus comprising a heterologous nucleic acid encoding an apoptosis inducible protein.

195. The method of embodiment any of embodiments 187-194, wherein the method inhibits virus replication preferentially in non-cancer cells.

196. The method of any of embodiments 187-195, wherein the apoptosis-inducible protein is an inducible DED (iDED).

197. The method of embodiment 196, wherein the iDED comprises the sequence set forth in SEQ ID NO: 27 or a sequence of amino acids that has at least 85%, 90% or 95% sequence identity to SEQ ID NO: 27.

198. The method of embodiment 196 and embodiment 197, wherein the nucleic acid genome of the recombinant oncolytic virus comprises a sequence of nucleotide that has at least 95%, at least 96%, at least 97%, at least 98% or at least 99% sequence identity to the sequence set forth in SEQ ID NO: 8.

199. The method of any of embodiments 196-198, wherein the nucleic acid genome of the recombinant oncolytic virus comprises the sequence of nucleotides set forth in SEQ ID NO: 8.

Also among the provided embodiments are the following:

1. A recombinant oncolytic vaccinia virus, comprising:
an inactivating mutation of B2R;
a heterologous nucleic acid encoding interferon regulatory factor 3 (IRF3); and
at least one heterologous nucleic acid encoding one or more cytokine and/or chemokine.

2. The recombinant oncolytic vaccinia virus of embodiment 1, wherein the at least one heterologous nucleic acid encoding one or more cytokine and/or chemokine comprises a heterologous nucleic acid encoding chemokine ligand 9 (CXCL9) and/or IL-12.

3. The recombinant oncolytic vaccinia virus of embodiment 1 or embodiment 2, wherein the at least one heterologous nucleic acid encoding one or more cytokine and/or chemokine comprises a heterologous nucleic acid encoding CXCL9 and IL-12.

4. The recombinant oncolytic vaccinia virus of embodiment 1, wherein the at least one heterologous nucleic acid encoding one or more cytokine and/or chemokine comprises a heterologous nucleic acid encoding CXCL9 and a heterologous nucleic acid encoding IL-12.

5. The recombinant oncolytic vaccinia virus of any one of embodiments 1-4, wherein:
the CXCL9 is human CXCL9 and comprises the amino acid sequence set forth in SEQ ID NO: 99, or an amino acid sequence that has at least 95%, 96%, 97%, 98%, or 99% sequence identity to the amino acid sequence of SEQ ID NO: 99; or
the CXCL9 is mouse CXCL9 and comprises the amino acid sequence set forth in SEQ ID NO: 106, or an amino acid sequence that has at least 95%, 96%, 97%, 98%, or 99% sequence identity to the amino acid sequence of SEQ ID NO: 106.

6. The recombinant oncolytic vaccinia virus of any one of embodiments 1-5, wherein:
the IL-12 is a human single-chain IL-12 and comprises the amino acid sequence set forth in SEQ ID NO: 103, or an amino acid sequence that has at least 95%, 96%, 97%, 98%, or 99% sequence identity to the amino acid sequence of SEQ ID NO: 103; or
the IL-12 is a mouse single-chain IL-12 and comprises the amino acid sequence set forth in SEQ ID NO: 102, or an amino acid sequence that has at least 95%, 96%, 97%, 98%, or 99% sequence identity to the amino acid sequence of SEQ ID NO: 102.

7. The recombinant oncolytic vaccinia virus of any one of embodiments 1-6, wherein the at least one heterologous nucleic acid encoding one or more cytokine and/or chemokine comprises a heterologous nucleic acid encoding IL-2.

8. The recombinant oncolytic vaccinia virus of embodiment 7, wherein the IL-2 comprises the IL-2 comprises an amino acid sequence set forth in any one of SEQ ID NOs: 98, 100, 101, 104, and 105, or an amino acid sequence that has at least 95%, 96%, 97%, 98%, or 99% sequence identity to the amino acid sequence of any one of SEQ ID NOs: 98, 100, 101, 104, and 105.

9. The recombinant oncolytic vaccinia virus of embodiment 7 or embodiment 8, wherein the IL-2 is an IL-2 superkine.

10. The recombinant oncolytic vaccinia virus of embodiment 9, wherein the IL-2 superkine is H9, H9T, MDNA11, or MDNA11T, and wherein:
the H9 IL-2 superkine comprises the amino acid sequence of SEQ ID NO: 100, or comprises an amino acid sequence that has at least 95%, 96%, 97%, 98%, or 99% sequence identity to the amino acid sequence of SEQ ID NO: 100; or
the H9T IL-2 superkine comprises the amino acid sequence of SEQ ID NO: 104, or comprises an amino acid sequence that has at least 95%, 96%, 97%, 98%, or 99% sequence identity to the amino acid sequence of SEQ ID NO: 104; or
the MDNA11 IL-2 superkine comprises the amino acid sequence of SEQ ID NO: 101, or comprises an amino acid sequence that has at least 95%, 96%, 97%, 98%, or 99% sequence identity to the amino acid sequence of SEQ ID NO: 101; or
the MDNA11T IL-2 superkine comprises the amino acid sequence of SEQ ID NO: 98, or comprises an amino acid sequence that has at least 95%, 96%, 97%, 98%, or 99% sequence identity to the amino acid sequence of SEQ ID NO: 98.

11. The recombinant oncolytic vaccinia virus of embodiment 6 or embodiment 7, wherein the IL-2 superkine is MDNA11T, and the MDNA11T comprises the amino acid sequence set forth in SEQ ID NO: 98, or an amino acid sequence that has at least 95%, 96%, 97%, 98%, or 99% sequence identity to the amino acid sequence set forth in SEQ ID NO: 98.

12. The recombinant oncolytic vaccinia virus of any one of embodiments 1-11, further comprising one or more heterologous gene product selected from the group consisting of a complement inhibitor, a T cell or NK cell evader, an immune stimulating protein, an anti-angiogenic protein, an interferon regulatory factor, an apoptosis inducible protein or a combination of any of the foregoing.

13. The recombinant oncolytic vaccinia virus of any one of embodiments 1-12, wherein the inactivating mutation of B2R is a deletion of all or a portion of the B2R gene loci.

14. The recombinant oncolytic vaccinia virus of embodiment 13, wherein said deletion is sufficient to render the encoded B2R gene product non-functional.

15. The recombinant oncolytic virus of any of embodiments 1-14, wherein the inactivating mutation of B2R is characterized by insertion of the heterologous nucleic acid encoding IRF3 and/or at least one of the at least one heterologous nucleic acid encoding one or more cytokine and/or chemokine into the B2R gene loci.

16. The recombinant oncolytic virus of any of embodiments 1-15, wherein the inactivating mutation of B2R is characterized by insertion of the heterologous nucleic acid encoding chemokine ligand 9 (CXCL9) and/or IL-12 into the B2R gene loci.

17. The recombinant oncolytic vaccinia virus of any one of embodiments 1-16, wherein:
the heterologous nucleic acid encoding IRF3 is inserted into the hemagglutinin (HA), J2R (thymidine kinase), F14.5L, A56R (hemagglutinin), vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus; and/or
the at least one of the at least one heterologous nucleic acid encoding one or more cytokine and/or chemokine is inserted into the HA, J2R, F14.5L, A56R, vaccinia growth factor, A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus.

18. The recombinant oncolytic vaccinia virus of any one of embodiments 1-17, wherein the nucleic acid genome of the recombinant oncolytic vaccinia virus is modified from a parental vaccinia virus that has a nucleic acid genome that has at least 99% sequence identity to the nucleic acid sequence set forth in SEQ ID NO: 1, optionally the nucleic acid genome set forth in SEQ ID NO: 1.

19. The recombinant oncolytic vaccinia virus of any one of embodiments 1-18, wherein the nucleic acid genome of the parental vaccinia virus is characterized by one or more of:
(i) a variant 017 open reading frame (ORF) encoding an amino acid sequence that has at least 95% sequence identity to SEQ ID NO: 57 and comprises a polar uncharged amino acid at position 66, optionally a threonine (T) at position 66;
(ii) a variant 038 (K5L) ORF comprising a nucleotide insertion to effect a frameshift mutation, wherein the 038 (K5L) gene product is altered;
(iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419;
(iv) a variant 104 (H4L) ORF encoding an amino acid sequence having at least 95% sequence identity to SEQ ID NO: 61 and comprises a negatively charged amino acid at position 591, optionally aspartic acid (D) at position 591; and
(v) a variant 182 (A56R) ORF comprising deletion of two nucleotides to effect a frameshift mutation, wherein the 182 (A56R) ORF gene product is altered.

20. The recombinant oncolytic vaccinia virus of any one of embodiments 1-19, wherein the nucleic acid genome of the parental virus is characterized by one or more of:
(i) a guanine (G) at the position corresponding to position 7770 of SEQ ID NO: 1;
(ii) a thymine (T) at the position corresponding to position 15261 of SEQ ID NO: 1;
(iii) a G at the position corresponding to position 32136 of SEQ ID NO: 1;
(iv) a G at the position corresponding to position 49455 of SEQ ID NO: 1;
(v) a cytosine (C) at the position corresponding to position 92969 of SEQ ID NO: 1;
(vi) the nucleic acid sequence CACTTATATAT at the positions corresponding to positions 106870 to 106880 of SEQ ID NO: 1;
(vii) the nucleic acid sequence GTTTTCATTA at the positions corresponding to positions 111267 to 111276 of SEQ ID NO: 1;
(viii) an adenine (A) at the position corresponding to position 162715 of SEQ ID NO: 1;
(ix) the nucleic acid sequence TACAGACACC at the positions corresponding to positions 165844 to 185853 of SEQ ID NO: 1; and
(x) a C at the position corresponding to position 187805 of SEQ ID NO: 1.

21. The recombinant oncolytic vaccinia virus of any one of embodiments 1-20, wherein the nucleic acid genome of the recombinant oncolytic vaccinia virus has at least 95%, 96%, 97%, 98%, or 99% sequence identity to the nucleic acid sequence set forth in SEQ ID NO: 1.

22. The recombinant oncolytic vaccinia virus of any one of embodiments 1-14 and 17-21, wherein:
the heterologous nucleic acid encoding IRF3 is inserted into the J2R (thymidine kinase) gene locus in the genome of the virus; and
the at least one heterologous nucleic acid encoding one or more cytokine and/or chemokine comprises a heterologous nucleic acid encoding CXCL9 and IL-12, wherein the heterologous nucleic acid encoding CXCL9 and IL-12 is inserted into the A56R gene locus in the genome of the virus.

23. The recombinant oncolytic vaccinia virus of any one of embodiments 1-14 and 17-22, wherein the nucleic acid genome of the recombinant oncolytic vaccinia virus comprises the nucleic acid sequence of SEQ ID NO: 85, or a nucleic acid sequence that has at least 95%, 96%, 97%, 98%, or 99% sequence identity to the nucleic acid sequence set forth in SEQ ID NO: 85.

24. The recombinant oncolytic vaccinia virus of any one of embodiments 1-21, wherein the heterologous nucleic acid encoding IRF3 is inserted into the B2R (viral cGAMP-specific nuclease) gene locus in the genome of the virus; and
the at least one heterologous nucleic acid encoding one or more cytokine and/or chemokine comprises a heterologous nucleic acid encoding CXCL9 and IL-12, wherein the heterologous nucleic acid encoding CXCL9 and IL-12 is inserted into the A56R gene locus in the genome of the virus.

25. The recombinant oncolytic vaccinia virus of any one of embodiments 1-24, further comprising a heterologous nucleic acid encoding an apoptosis-inducible protein.

26. The recombinant oncolytic vaccinia virus of embodiment 25, wherein the apoptosis-inducible protein is an inducible death effector domain (iDED).

27. The recombinant oncolytic vaccinia virus of embodiment 26, wherein the iDED comprises the amino acid sequence set forth in SEQ ID NO: 27 or a sequence of amino acids that has at least 85%, 90%, 91%, 92%, 93%, 94%, 95%, 96%, 97%, 98%, or 99% sequence identity to the amino acid sequence set forth in SEQ ID NO: 27.

28. The recombinant oncolytic vaccinia virus of embodiment 26 or embodiment 27, wherein the heterologous nucleic acid encoding an iDED is inserted into or in place of the J2R gene locus in the genome of the virus.

29. The recombinant oncolytic vaccinia virus of any one of embodiments 1-21 and 24-28, wherein the nucleic acid genome of the recombinant oncolytic vaccinia virus comprises the nucleic acid sequence of SEQ ID NO: 86, or a nucleic acid sequence that has at least 95%, 96%, 97%, 98%, or 99% sequence identity to the nucleic acid sequence set forth in SEQ ID NO: 86.

30. The recombinant oncolytic vaccinia virus of any one of embodiments 1-29, further comprising a heterologous nucleic acid encoding one or more T cell or NK cell evader proteins.

31. The recombinant oncolytic vaccinia virus of embodiment 30, wherein the one or more T cell or NK cell evader proteins comprises a set of proteins encoded by Cowpox virus ORFs 012, 203 and 018 (CPXV012-203-018).

32. The recombinant oncolytic vaccinia virus of embodiment 31, wherein the set of proteins encoded by CPXV012-203-018 comprises:
(i) the amino acid sequence set forth in SEQ ID NO: 20 (CPXV012) or an amino acid sequence that has at least 85%, 90%, 91%, 92%, 93%, 94%, 95%, 96%, 97%, 98%, or 99% sequence identity to the amino acid sequence set forth in SEQ ID NO: 20,
(ii) the amino acid sequence set forth in SEQ ID NO: 21 (CPXV0203) or an amino acid sequence that has at least 85%, 90%, 91%, 92%, 93%, 94%, 95%, 96%, 97%, 98%, or 99% sequence identity to the amino acid sequence set forth in SEQ ID NO: 21, and
(iii) the amino acid sequence set forth in SEQ ID NO: 22 (CPXV018) or an amino acid sequence that has at least 85%, 90%, 91%, 92%, 93%, 94%, 95%, 96%, 97%, 98%, or 99% sequence identity to the amino acid sequence set forth in SEQ ID NO: 22.

33. The recombinant oncolytic vaccinia virus of any one of embodiments 1-32, further comprising a heterologous nucleic acid encoding a complement inhibitor.

34. The recombinant oncolytic vaccinia virus of embodiment 33, wherein the complement inhibitor is Borrelia burgdorferi complement regulatory-acquiring surface protein-2 (CRASP-2).

35. The recombinant oncolytic vaccinia virus of embodiment 34, wherein the heterologous nucleic acid encoding CRASP-2 is fused with a viral membrane gene, optionally F14.5L, to produce a fusion gene encoding a fusion protein.

36. The recombinant oncolytic vaccinia virus of embodiment 35, wherein the fusion protein comprises the CRASP-2 fused to a viral membrane protein encoded by the viral membrane gene.

37. The recombinant oncolytic virus of embodiment 36, wherein the viral membrane protein is F14.5L, optionally wherein the fusion is at the C-terminus of F14.5L.

38. The recombinant oncolytic vaccinia virus of any one of embodiments 1-22, 24-28 and 30-37, wherein the nucleic acid genome of the recombinant oncolytic vaccinia virus comprises the nucleic acid sequence of SEQ ID NO: 90, or a nucleic acid sequence that has at least 95%, 96%, 97%, 98%, or 99% sequence identity to the nucleic acid sequence set forth in SEQ ID NO: 90.

39. The recombinant oncolytic vaccinia virus of any one of embodiments 1-21, 25-28, and 30-37, wherein the heterologous nucleic acid encoding IRF3 is inserted into or in place of the B2R (viral cGAMP-specific nuclease) gene locus in the genome of the virus; and
the at least one heterologous nucleic acid encoding one or more cytokine and/or chemokine comprises a heterologous nucleic acid encoding IL-2, wherein the IL-2 is an IL-2 superkine that is MDNA11T.

40. The recombinant oncolytic vaccinia virus of any one of embodiments 1-39, further comprising a heterologous nucleic acid encoding an immune stimulating protein, and/or a heterologous nucleic acid encoding one or more anti-angiogenic protein.

41. The recombinant oncolytic vaccinia virus of embodiment 40, wherein the immune stimulating protein is recombinant LIGHT.

42. The recombinant oncolytic vaccinia virus of embodiment 41, wherein the recombinant LIGHT comprises the amino acid sequence set forth in SEQ ID NO: 30, or an amino acid sequence that has at least 85%, 90%, 91%, 92%, 93%, 94%, 95%, 96%, 97%, 98%, or 99% sequence identity to the amino acid sequence set forth in SEQ ID NO: 30.

43. The recombinant oncolytic vaccinia virus of any one of embodiments 40-42, wherein the one or more anti-angiogenic protein comprises a VEGF inhibitor, an angiopoietin inhibitor, versikine, or a fusion protein of any two or more of the foregoing.

44. The recombinant oncolytic vaccinia virus of embodiment 43, wherein the one or more anti-angiogenic protein comprises an anti-VEGF antibody and/or an anti-Ang2 antibody.

45. The recombinant oncolytic vaccinia virus of embodiment 43 or embodiment 44, wherein the one or more anti-angiogenic protein is a bispecific anti-VEGF/anti-Ang2 antibody.

46. The recombinant oncolytic vaccinia virus of embodiment 45, wherein the bispecific anti-VEGF/anti-Ang2 antibody comprises the amino acid sequence set forth in SEQ ID NO: 23, or an amino acid sequence that has at least 85%, 90%, 91%, 92%, 93%, 94%, 95%, 96%, 97%, 98%, or 99% sequence identity to the sequence set forth in SEQ ID NO: 23.

47. The recombinant oncolytic vaccinia virus of any one of embodiments 1-22, 24-28, 30-37 and 39-46, wherein the nucleic acid genome of the recombinant oncolytic vaccinia virus comprises the nucleic acid sequence of SEQ ID NO: 88, or a nucleic acid sequence that has at least 95%, 96%, 97%, 98%, or 99% sequence identity to the nucleic acid sequence set forth in SEQ ID NO: 88.

48. A recombinant oncolytic virus, comprising:
an inactivating mutation of at least one viral gene; and
at least one heterologous nucleic acid encoding one or more heterologous gene product, wherein the one or more heterologous gene product is or comprises an immune modulating protein, a complement inhibitor, a T cell or NK cell evader, an anti-angiogenic protein, an interferon regulatory factor, or an apoptosis inducible protein, or a combination of any of the foregoing.

49. A recombinant oncolytic virus, comprising at least one heterologous nucleic acid encoding one or more heterologous gene product, wherein the one or more heterologous gene product is or comprises a complement inhibitor, a T cell or NK cell evader, an immune modulating protein, an anti-angiogenic protein, an interferon regulatory factor, an apoptosis inducible protein, or a combination of any of the foregoing.

50. The recombinant oncolytic virus of embodiment 48 or embodiment 50, wherein the oncolytic virus is a vaccinia virus, a herpes simplex virus, vesicular stomatitis virus (VSV), a Maraba virus (MARAV), a measles virus (MV), adenovirus, myxoma virus, orf virus, parvovirus, raccoonpox virus, coxsackievirus, reovirus, Newcastle disease virus, Seneca valley virus, Semliki Forest virus, mumps virus, influenza virus, echovirus, and a poliovirus (PV).

51. The recombinant oncolytic virus of any one of embodiments 48-50, wherein the oncolytic virus is a vaccinia virus.

52. The recombinant oncolytic vaccinia virus of any one of embodiments 48-51, wherein the nucleic acid genome of the recombinant oncolytic vaccinia virus is modified from a parental vaccinia virus that has a nucleic acid genome that has at least 99% sequence identity to the nucleic acid sequence set forth in SEQ ID NO: 1, optionally the nucleic acid genome set forth in SEQ ID NO: 1.

53. A recombinant oncolytic virus, comprising a nucleic acid genome that is modified from a parental vaccinia virus genome that has at least 99% sequence identity with the nucleic acid sequence set forth in SEQ ID NO: 1, and comprises at least one heterologous nucleic acid encoding one or more heterologous gene product inserted in the genome.

54. The recombinant oncolytic vaccinia virus of embodiment 52 or embodiment 53, wherein the nucleic acid genome of the parental vaccinia virus is characterized by one or more of:
(i) a variant 017 open reading frame (ORF) encoding an amino acid sequence that has at least 95% sequence identity to SEQ ID NO: 57 and comprises a polar uncharged amino acid at position 66, optionally a threonine (T) at position 66;
(ii) a variant 038 (K5L) ORF comprising a nucleotide insertion to effect a frameshift mutation, wherein the 038 (K5L) gene product is altered;
(iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419;
(iv) a variant 104 (H4L) ORF encoding an amino acid sequence having at least 95% sequence identity to SEQ ID NO: 61 and comprises a negatively charged amino acid at position 591, optionally aspartic acid (D) at position 591; and
(v) a variant 182 (A56R) ORF comprising deletion of two nucleotides to effect a frameshift mutation, wherein the 182 (A56R) ORF gene product is altered.

55. The recombinant oncolytic virus of any one of embodiments 52-54, wherein the parental vaccinia virus genome is characterized by one or more of:
(i) a guanine (G) at the position corresponding to position 7770 of SEQ ID NO: 1;
(ii) a thymine (T) at the position corresponding to position 15261 of SEQ ID NO: 1;
(iii) a G at the position corresponding to position 32136 of SEQ ID NO: 1;
(iv) a G at the position corresponding to position 49455 of SEQ ID NO: 1;
(v) a cytosine @ at the position corresponding to position 92969 of SEQ ID NO: 1;
(vi) the nucleic acid sequence CACTTATATAT at the positions corresponding to positions 106870 to 106880 of SEQ ID NO: 1;
(vii) the nucleic acid sequence GTTTTCATTA at the positions corresponding to positions 111267 to 111276 of SEQ ID NO: 1;
(viii) an adenine (A) at the position corresponding to position 162715 of SEQ ID NO: 1;
(ix) the nucleic acid sequence TACAGACACC at the positions corresponding to positions 165844 to 185853 of SEQ ID NO: 1; and
(x) a C at the position corresponding to position 187805 of SEQ ID NO: 1.

56. The recombinant oncolytic vaccinia virus of any one of embodiments 52-55, wherein the nucleic acid genome of the recombinant oncolytic vaccinia virus has at least 95%, 96%, 97%, 98%, or 99% sequence identity to the nucleic acid sequence set forth in SEQ ID NO: 1.

57. The recombinant oncolytic virus of any of embodiments 1-56, wherein the recombinant oncolytic virus is a recombinant oncolytic vaccinia virus, and wherein the nucleic acid genome of the recombinant oncolytic vaccinia virus is characterized by one or more of:
(i) a variant 017 open reading frame (ORF) encoding an amino acid sequence that has at least 95% sequence identity to SEQ ID NO: 57 and comprises a polar uncharged amino acid at position 66, optionally a threonine (T) at position 66;
(ii) a variant 038 (K5L) ORF comprising a nucleotide insertion to effect a frameshift mutation, wherein the 038 (K5L) gene product is altered;
(iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419;
(iv) a variant 104 (H4L) ORF encoding an amino acid sequence having at least 95% sequence identity to SEQ ID NO: 61 and comprises a negatively charged amino acid at position 591, optionally aspartic acid (D) at position 591; and
(v) a variant 182 (A56R) ORF comprising deletion of two nucleotides to effect a frameshift mutation, wherein the 182 (A56R) ORF gene product is altered.

58. The recombinant oncolytic virus of any one of embodiments 1-57, wherein the recombinant oncolytic virus is a recombinant oncolytic vaccinia virus, and wherein the nucleic acid genome of the recombinant oncolytic vaccinia virus is characterized by one or more of:
(i) a guanine (G) at the position corresponding to position 7770 of SEQ ID NO: 1;
(ii) a thymine (T) at the position corresponding to position 15261 of SEQ ID NO: 1;
(iii) a G at the position corresponding to position 32136 of SEQ ID NO: 1;
(iv) a G at the position corresponding to position 49455 of SEQ ID NO: 1;
(v) a cytosine (C) at the position corresponding to position 92969 of SEQ ID NO: 1;
(vi) the nucleic acid sequence CACTTATATAT at the positions corresponding to positions 106870 to 106880 of SEQ ID NO: 1;
(vii) the nucleic acid sequence GTTTTCATTA at the positions corresponding to positions 111267 to 111276 of SEQ ID NO: 1;
(viii) an adenine (A) at the position corresponding to position 162715 of SEQ ID NO: 1;
(ix) the nucleic acid sequence TACAGACACC at the positions corresponding to positions 165844 to 185853 of SEQ ID NO: 1; and
(x) a C at the position corresponding to position 187805 of SEQ ID NO: 1.

59. The recombinant oncolytic virus of any of embodiments 48-58, wherein at least one of the at least one heterologous nucleic acid encoding the one or more heterologous gene product is inserted into a non-essential gene or region in the genome of the virus.

60. The recombinant oncolytic virus of any of embodiments 48-59, wherein at least one of the at least one heterologous nucleic acid encoding the one or more heterologous gene product is inserted into the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus.

61. The recombinant oncolytic virus of embodiment 59, wherein each of the at least one heterologous nucleic acid encoding the one or more heterologous gene product that is inserted into a non-essential gene or region in the genome of the virus is each independently inserted into the hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, or I4L gene loci in the genome of the virus.

62. The recombinant oncolytic virus of any one of embodiments 48, 50-52, and 54-59, wherein the at least one viral gene comprises one or more viral genes selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L, and I4L, and any combination thereof.

63. The recombinant oncolytic virus of any one of embodiments 48, 50-52, 54-59 and 62, wherein the at least one viral gene is or comprises:
(i) B2R;
(ii) A35R;
(iii) A35R and J2R;
(iv) J2R;
(v) B2R and J2R;
(vi) A35R, B2R, and J2R;
(vii) B2R, J2R, and A56R; or
(viii) A35R, B2R, J2R, and A56R.

64. The recombinant oncolytic virus of any one of embodiments 48, 50-52, and 54-63, wherein the inactivating mutation of one or more of the at least one viral gene is, independently, by:
insertion of at least one of the at least one heterologous nucleic acid encoding one or more heterologous gene product into a gene loci in the genome of the virus;
a deletion of all or a portion of the at least one viral gene; and/or
one or more nucleic acid substitutions in the at least one viral gene.

65. The recombinant oncolytic virus of any one of embodiments 48, 50-52, and 54-64, wherein the inactivating mutation is a deletion of all or a portion of the at least one viral gene.

66. The recombinant oncolytic virus of embodiment 64 or embodiment 65, wherein: the deletion of the at least one viral gene is deletion of the entire gene ORF of a viral gene.

67. The recombinant oncolytic virus of any one of embodiments 64-66, wherein:
the deletion is sufficient to render the encoded viral gene product non-functional.

68. The recombinant oncolytic virus of embodiment 64, wherein the one or more nucleic acid substitutions is sufficient to render the encoded viral gene product non-functional.

70. The recombinant oncolytic virus of any one of embodiments 48, 50-52, and 54-68, wherein the inactivating mutation of one or more of the at least one viral gene is characterized by insertion of at least one of the at least one heterologous nucleic acid encoding one or more heterologous gene product into the viral gene loci.

71. The recombinant oncolytic virus of embodiment 70, wherein the at least one viral gene comprises B2R.

72. The recombinant oncolytic virus of embodiment 70 or embodiment 71, wherein the at least one viral gene comprises J2R.

73. The recombinant oncolytic virus of any one of embodiments 70-72, wherein the at least one viral gene comprises A35R.

74. The recombinant oncolytic virus of any one of embodiments 70-73, wherein the at least one viral gene comprises A56R.

75. The recombinant oncolytic virus of any one of embodiments 70-74, wherein the at least one viral gene comprises B2R, J2R, and A35R.

76. The recombinant oncolytic virus of any one of embodiments 70-75, wherein the at least one viral gene comprises B2R, J2R, A35R, and A56R.

77. The recombinant oncolytic virus of any one of embodiments 70-76, wherein the at least one viral gene comprises B2R, J2R, and A56R.

78. The recombinant oncolytic virus of any of embodiments 48-77, wherein:
at least one heterologous nucleic acid encoding the one or more heterologous gene product is inserted into or in place of F14.5L.

79. The recombinant oncolytic virus of any of embodiments 48-78, wherein at least one heterologous nucleic acid encoding the one or more heterologous gene product is inserted into or in place of A35R.

80. The recombinant oncolytic virus of any of embodiments 48-78, wherein at least one heterologous nucleic acid encoding the one or more heterologous gene product is inserted into or in place of J2R.

81. The recombinant oncolytic virus of any one of embodiments 48-80, wherein the at least one heterologous nucleic acid encoding one or more heterologous gene product comprises one or more heterologous nucleic acid each encoding one or more immune modulating proteins.

82. The recombinant oncolytic virus of any one of embodiments 48, 50-52, and 54-81, wherein the inactivating mutation of one or more of the at least one viral gene is by insertion of one or more heterologous nucleic acid each encoding one or more immune modulating proteins.

83. The recombinant oncolytic virus of embodiment 81 or embodiment 82, wherein the one or more immune modulating proteins comprises one or more immune stimulating proteins.

84. The recombinant oncolytic virus of any one of embodiments 81-83, wherein the one or more immune modulating proteins comprises one or more cytokines and/or chemokines.

85. The recombinant oncolytic virus of any one of embodiments 81-84, wherein the one or more immune modulating proteins comprises one or more interferon regulatory factors, optionally IRF3.

86. The recombinant oncolytic virus of embodiment 85, wherein the one or more interferon regulatory factors is or comprises interferon regulatory factor 3 (IRF3).

87. The recombinant oncolytic virus of any one of embodiments 81-86, wherein the one or more immune modulating proteins comprises interferon regulatory factor 3 (IRF3) and one or more cytokines and/or chemokines.

88. The recombinant oncolytic virus of any one of embodiments 81-87, wherein the one or more immune modulating proteins comprises one or more immune modulating proteins selected from the group consisting of LIGHT, IRF3, IL-2, IL-12, and CXCL9.

89. The recombinant oncolytic virus of embodiment 88, wherein the CXCL9 is human CXCL9 and comprises the amino acid sequence set forth in SEQ ID NO: 99, or an amino acid sequence that has at least 95%, 96%, 97%, 98%, or 99% sequence identity to the amino acid sequence of SEQ ID NO: 99.

90. The recombinant oncolytic virus of embodiment 88, wherein the CXCL9 is mouse CXCL9 and comprises the amino acid sequence set forth in SEQ ID NO: 106, or an amino acid sequence that has at least 95%, 96%, 97%, 98%, or 99% sequence identity to the amino acid sequence of SEQ ID NO: 106.

91. The recombinant oncolytic virus of embodiment 88, wherein the IL-12 is a human single-chain IL-12 and comprises the amino acid sequence set forth in SEQ ID NO: 103, or an amino acid sequence that has at least 95%, 96%, 97%, 98%, or 99% sequence identity to the amino acid sequence of SEQ ID NO: 103.

92. The recombinant oncolytic virus of embodiment 88, wherein the IL-12 is a mouse single-chain IL-12 and comprises the amino acid sequence set forth in SEQ ID NO: 102, or an amino acid sequence that has at least 95%, 96%, 97%, 98%, or 99% sequence identity to the amino acid sequence of SEQ ID NO: 102.

93. The recombinant oncolytic virus of any one of embodiments 81-92, wherein the one or more immune modulating proteins comprises IRF3.

94. The recombinant oncolytic virus of embodiment 93, wherein the IRF3 is a human IRF3 (hIRF3).

95. The recombinant oncolytic virus of embodiment 94, wherein the hIRF3 comprises the amino acid sequence set forth in SEQ ID NO: 51, or an amino acid sequence that has at least 95%, 96%, 97%, 98%, or 99% sequence identity to the amino acid sequence set forth in SEQ ID NO: 51.

96. The recombinant oncolytic virus of embodiment 93, wherein the IRF3 is a mouse IRF3 (mIRF3).

97. The recombinant oncolytic virus of embodiment 96, wherein the mIRF3 comprises the amino acid sequence set forth in SEQ ID NO: 52, or an amino acid sequence that has at least 95%, 96%, 97%, 98%, or 99% sequence identity to the amino acid sequence set forth in SEQ ID NO: 52.

98. The recombinant oncolytic virus of any one of embodiments 81-97, wherein the nucleic acid genome of the recombinant oncolytic virus comprises the nucleic acid sequence set forth in any one of SEQ ID NOs: 49, 50, 80, 82, and 84-93, or a nucleic acid sequence that has at least 95%, 96%, 97%, 98%, or 99% sequence identity to the nucleic acid sequence set forth in any one of SEQ ID NOs: 49, 50, 80, 82, and 84-93.

99. The recombinant oncolytic virus of any one of embodiments 81-98, wherein the one or more immune modulating proteins comprises IRF3 and one or more immune modulating proteins selected from the group consisting of LIGHT, IL-2, IL-12, and CXCL9.

100. The recombinant oncolytic virus of any one of embodiments 81-99, wherein the one or more immune modulating proteins comprises IL-2.

101. The recombinant oncolytic virus of any one of embodiments 81-100, wherein the one or more immune modulating proteins comprises IL-12.

102. The recombinant oncolytic virus of any one of embodiments 81-101, wherein the one or more immune modulating proteins comprises LIGHT.

103. The recombinant oncolytic virus of any one of embodiments 81-102, wherein the one or more immune modulating proteins comprises CXCL9.

104. The recombinant oncolytic virus of any one of embodiments 81-103, wherein the one or more immune modulating proteins is or comprises:
(i) IRF3;
(ii) LIGHT;
(iii) IRF3 and LIGHT;
(iv) IRF3 and IL-2;
(v) IRF3, CXCL9, and IL-12;
(vi) IRF3, LIGHT, and IL-2;
(vii) IRF3 and CXCL9; or
(viii) IRF3, CXCL9, and IL-2.

105. The recombinant oncolytic virus of any one of embodiments 88-104, wherein the IL-2 is a human IL-2.

106. The recombinant oncolytic virus of any one of embodiments 88-105, wherein the IL-2 is an IL-2 superkine.

107. The recombinant oncolytic virus of embodiment 106, wherein the IL-2 superkine is H9, H9T, MDNA11, or MDNA11T.

108. The recombinant oncolytic virus of embodiment 106, wherein:
the H9 IL-2 superkine comprises the amino acid sequence of SEQ ID NO: 100, or comprises an amino acid sequence that has at least 95%, 96%, 97%, 98%, or 99% sequence identity to the amino acid sequence of SEQ ID NO: 100; or
the H9T IL-2 superkine comprises the amino acid sequence of SEQ ID NO: 104, or comprises an amino acid sequence that has at least 95%, 96%, 97%, 98%, or 99% sequence identity to the amino acid sequence of SEQ ID NO: 104; or
the MDNA11 IL-2 superkine comprises the amino acid sequence of SEQ ID NO: 101, or comprises an amino acid sequence that has at least 95%, 96%, 97%, 98%, or 99% sequence identity to the amino acid sequence of SEQ ID NO: 101; or
the MDNA11T IL-2 superkine comprises the amino acid sequence of SEQ ID NO: 98, or comprises an amino acid sequence that has at least 95%, 96%, 97%, 98%, or 99% sequence identity to the amino acid sequence of SEQ ID NO: 98.

109. The recombinant oncolytic vaccinia virus of any one of embodiments 106-108, wherein the IL-2 superkine is MDNA11 or MDNA11T.

110. The recombinant oncolytic vaccinia virus of any one of embodiments 106-109, wherein the IL-2 superkine is MDNA11T.
the MDNA11T comprises the amino acid sequence set forth in SEQ ID NO: 98, or an amino acid sequence that has at least 95%, 96%, 97%, 98%, or 99% sequence identity to the amino acid sequence set forth in SEQ ID NO: 98.

111. The recombinant oncolytic virus of any one of embodiments 88-110, wherein the LIGHT is recombinant LIGHT.

112. The recombinant oncolytic virus of embodiment 111, wherein the recombinant LIGHT is a human LIGHT protein or is a mutant thereof.

113. The recombinant oncolytic virus of embodiment 111 or embodiment 112, wherein the recombinant LIGHT comprises an amino acid sequence that has at least 95%, 96%, 97%, 98%, or 99% sequence identity to the amino acid sequence set forth in SEQ ID NO: 30.

114. The recombinant oncolytic virus of any of embodiments 111-113, wherein the recombinant LIGHT is human LIGHT mutant (hmLIGHT) that is a human LIGHT mutant that binds human and mouse LTBR and HVEM.

115. The recombinant oncolytic virus of any one of embodiments 111-114, wherein the recombinant LIGHT comprises one or more mutations selected from the group consisting of a threonine at position 138, a glycine at position 160, a glycine at position 221, and a lysine at position 222.

116. The recombinant oncolytic virus of any one of embodiments 111-115, wherein the recombinant LIGHT comprises the amino acid sequence set forth in SEQ ID NO: 25, or an amino acid sequence that has at least 95%, 96%, 97%, 98%, or 99% sequence identity to the amino acid sequence set forth in SEQ ID NO: 25.

117. The recombinant oncolytic virus of any one of embodiments 111-116, wherein the recombinant LIGHT comprises the sequence set forth in SEQ ID NO: 25.

118. The recombinant oncolytic virus of any one of embodiments 111-117, wherein the nucleic acid genome of the recombinant oncolytic virus comprises the nucleic acid sequence set forth in any one of SEQ ID NOs: 11, 82, 87, and 88, or a nucleic acid sequence that has at least 95%, 96%, 97%, 98%, or 99% sequence identity to the nucleic acid sequence set forth in any one of SEQ ID NOs: 11, 82, 87, and 88.

119. The recombinant oncolytic virus of any one of embodiments 88-118, wherein the IL-12 is a human IL-12.

120. The recombinant oncolytic virus of embodiment 119, wherein the human IL-12 is a human single chain IL-12 (hscIL-12).

121. The recombinant oncolytic virus of embodiment 120, wherein the hscIL-12 comprises the amino acid sequence set forth in SEQ ID NO: 103, or an amino acid sequence that has at least 95%, 96%, 97%, 98%, or 99% sequence identity to the amino acid sequence set forth in SEQ ID NO: 103.

122. The recombinant oncolytic virus of any one of embodiments 88-121, wherein the CXCL9 is a human CXCL9.

123. The recombinant oncolytic virus of embodiment 122, wherein the human CXCL9 comprises the amino acid sequence set forth in SEQ ID NO: 99, or an amino acid sequence that has at least 95%, 96%, 97%, 98%, or 99% sequence identity to the amino acid sequence set forth in SEQ ID NO: 99.

124. The recombinant oncolytic virus of any one of embodiments 48-123, wherein the at least one heterologous nucleic acid encoding one or more heterologous gene product comprises one or more heterologous nucleic acid each encoding an apoptosis-inducible protein.

125. The recombinant oncolytic virus of any one of embodiments 48, 50-52, and 54-124, wherein the inactivating mutation of one or more of the at least one viral gene is by insertion of one or more heterologous nucleic acid each encoding an apoptosis-inducible protein.

126. The recombinant oncolytic virus of embodiment 124 or embodiment 125, wherein the apoptosis-inducible protein comprises a proapoptotic molecule fused with an FKBP variant that is able to bind a chemical inducer of dimerization (CID).

127. The recombinant oncolytic virus of embodiment 126, wherein the FKBP variant is FKBP-F36V, optionally wherein the FKBP-F36V comprises the amino acid sequence set forth in SEQ ID NO: 56 or an amino acid sequence that has at least 95%, 96%, 97%, 98%, or 99% sequence identity to the amino acid sequence set forth in SEQ ID NO: 56.

128. The recombinant oncolytic virus of embodiment 126 or embodiment 127, wherein the chemical inducer of dimerization is AP1903 (Rimiducid).

129. The recombinant oncolytic virus of any of embodiments 126-128, wherein the proapoptotic molecule is or comprises Fas, the death effector domain (DED) of the Fas-associated death domain-containing protein (FADD), or a caspase, optionally wherein the caspase is caspase 9.

130. The recombinant oncolytic virus of any one of embodiments 124-129, wherein the apoptosis-inducible protein is an inducible DED (iDED).

131. The recombinant oncolytic virus of embodiment 130, wherein the iDED comprises the amino acid sequence set forth in SEQ ID NO: 27 or an amino acid sequence that has at least 95%, 96%, 97%, 98%, or 99% sequence identity to the amino acid sequence set forth in SEQ ID NO: 27.

132. The recombinant oncolytic virus of any one of embodiments 124-131, wherein the nucleic acid genome of the recombinant oncolytic virus comprises the nucleic acid sequence set forth in SEQ ID NO: 8 or 86, or a nucleic acid sequence that has at least 95%, 96%, 97%, 98%, or 99% sequence identity to the nucleic acid sequence set forth in SEQ ID NO: 8 or 86.

133. The recombinant oncolytic virus of any of embodiments 124-129, wherein the apoptosis-inducible protein is an inducible Fas (iFas).

134. The recombinant oncolytic virus of embodiment 133, wherein the iFas comprises the amino acid sequence set forth in SEQ ID NO: 28 or an amino acid sequence that has at least 95%, 96%, 97%, 98%, or 99% sequence identity to the amino acid sequence set forth in SEQ ID NO: 28.

135. The recombinant oncolytic virus of any one of embodiments 124-129, 133, and 135, wherein the nucleic acid genome of the recombinant oncolytic virus comprises the nucleic acid sequence set forth in SEQ ID NO: 9, or a nucleic acid sequence that has at least 95%, 96%, 97%, 98%, or 99% sequence identity to the nucleic acid sequence set forth in SEQ ID NO: 9.

136. The recombinant oncolytic virus of any of embodiments 124-129, wherein the apoptosis-inducible protein is an inducible caspase 9 (iCas9).

137. The recombinant oncolytic virus of embodiment 136, wherein the iCas9 comprises the amino acid sequence set forth in SEQ ID NO: 26 or an amino acid sequence that has at least 95%, 96%, 97%, 98%, or 99% sequence identity to the amino acid sequence set forth in SEQ ID NO: 26.

138. The recombinant oncolytic virus of any one of embodiments 124-129, 136, and 137, wherein the nucleic acid genome of the recombinant oncolytic virus comprises the nucleic acid sequence set forth in SEQ ID NO: 7, or a nucleic acid sequence that has at least 95%, 96%, 97%, 98%, or 99% sequence identity to the nucleic acid sequence set forth in SEQ ID NO: 7.

139. The recombinant oncolytic virus of any of embodiments 48-138, wherein the at least one heterologous nucleic acid encoding one or more heterologous gene product comprises one or more heterologous nucleic acid each encoding one or more T cell or NK cell evader proteins.

140. The recombinant oncolytic virus of any one of embodiments 48, 50-52, and 54-139, wherein the inactivating mutation of one or more of the at least one viral gene is by insertion of one or more heterologous nucleic acid each encoding one or more T cell or NK cell evader proteins.

141. The recombinant oncolytic vaccinia virus of embodiment 139 or embodiment 140, wherein the one or more T cell or NK cell evader proteins comprises a set of proteins encoded by Cowpox virus ORFs 012, 203 and 018 (CPXV012-203-018).

142. The recombinant oncolytic vaccinia virus of any one of embodiments 139-141, wherein the one or more T cell or NK cell evader proteins comprises a set of proteins that is or comprises the CPXV012, CPXV203, and CPXV018 proteins.

143. The recombinant oncolytic vaccinia virus of embodiment 141 or embodiment 142, wherein the set of proteins encoded by CPXV012-203-018 comprises:
(i) the amino acid sequence set forth in SEQ ID NO: 20 (CPXV012) or an amino acid sequence that has at least 85%, 90%, 91%, 92%, 93%, 94%, 95%, 96%, 97%, 98%, or 99% sequence identity to the amino acid sequence set forth in SEQ ID NO: 20,
(ii) the amino acid sequence set forth in SEQ ID NO: 21 (CPXV0203) or an amino acid sequence that has at least 85%, 90%, 91%, 92%, 93%, 94%, 95%, 96%, 97%, 98%, or 99% sequence identity to the amino acid sequence set forth in SEQ ID NO: 21, and
(iii) the amino acid sequence set forth in SEQ ID NO: 22 (CPXV018) or an amino acid sequence that has at least 85%, 90%, 91%, 92%, 93%, 94%, 95%, 96%, 97%, 98%, or 99% sequence identity to the amino acid sequence set forth in SEQ ID NO: 22.

144. The recombinant oncolytic vaccinia virus of any one of embodiments 141-143, wherein the set of proteins encoded by CPXV012-203-018 comprises the amino acid sequences set forth in SEQ ID NO: 20, SEQ ID NO: 21, and SEQ ID NO: 22.

145. The recombinant oncolytic vaccinia virus of any one of embodiments 139-144, wherein the nucleic acid genome of the recombinant oncolytic virus comprises the nucleic acid sequence set forth in any one of SEQ ID NOs: 10, 89, and 90, or a nucleic acid sequence that has at least 95%, 96%, 97%, 98%, or 99% sequence identity to the nucleic acid sequence set forth in any one of SEQ ID NOs: 10, 89, and 90.

146. The recombinant oncolytic virus of any of embodiments 48-145, wherein the at least one heterologous nucleic acid encoding one or more heterologous gene product comprises one or more heterologous nucleic acid each encoding one or more complement inhibitor.

147. The recombinant oncolytic virus of any one of embodiments 48, 50-52, and 54-146, wherein the inactivating mutation of one or more of the at least one viral gene is by insertion of one or more heterologous nucleic acid each encoding one or more complement inhibitor.

148. The recombinant oncolytic virus of embodiment 146 or embodiment 147, wherein the one or more complement inhibitor is Borrelia burgdorferi complement regulatory-acquiring surface protein-2 (CRASP-2) and/or minimized complement regulator factor H (miniFH).

149. The recombinant oncolytic virus of embodiment 148, wherein the one or more complement inhibitor is or comprises CRASP-2.

150. The recombinant oncolytic virus of embodiment 149, wherein the CRASP-2 comprises the amino acid sequence set forth in SEQ ID NO: 18 or has an amino acid sequence that has at least 95%, 96%, 97%, 98%, or 99% sequence identity to the amino acid sequence set forth in SEQ ID NO: 18.

151. The recombinant oncolytic virus of any one of embodiments 148-150, wherein the one or more complement inhibitor is or comprises miniFH.

152. The recombinant oncolytic virus of embodiment 151, wherein the miniFH comprises the amino acid sequence set forth in SEQ ID NO: 19 or has an amino acid sequence that has at least 95%, 96%, 97%, 98%, or 99% sequence identity to the amino acid sequence set forth in SEQ ID NO: 19.

153. The recombinant oncolytic virus of any one of embodiments 146-152, wherein the one or more heterologous nucleic acid encoding the one or more complement inhibitor is introduced into a viral membrane gene, optionally F14.5L, to produce a fusion gene encoding a fusion protein.

154. The recombinant oncolytic virus of embodiment 153, wherein the fusion protein comprises the complement inhibitor fused to a viral membrane protein encoded by the viral membrane gene.

155. The recombinant oncolytic virus of embodiment 153 or embodiment 154, wherein the viral membrane gene is F14.5L, optionally wherein the fusion is at the C-terminus of the F14.5L protein.

156. The recombinant oncolytic virus of any one of embodiments 153-155, wherein the fusion protein is incorporated into the outer membrane of the intracellular mature virus (IMV).

157. The recombinant oncolytic vaccinia virus of any one of embodiments 153-156, wherein:
the nucleic acid genome of the recombinant oncolytic virus comprises the nucleic acid sequence set forth in SEQ ID NO: 5, or a nucleic acid sequence that has at least 95%, 96%, 97%, 98%, or 99% sequence identity to the nucleic acid sequence set forth in SEQ ID NO: 5; or
the nucleic acid genome of the recombinant oncolytic virus comprises the nucleic acid sequence set forth in SEQ ID NO: 6, or a nucleic acid sequence that has at least 95%, 96%, 97%, 98%, or 99% sequence identity to the nucleic acid sequence set forth in SEQ ID NO: 6; or
the nucleic acid genome of the recombinant oncolytic virus comprises the nucleic acid sequence set forth in SEQ ID NO: 89, or a nucleic acid sequence that has at least 95%, 96%, 97%, 98%, or 99% sequence identity to the nucleic acid sequence set forth in SEQ ID NO: 89; or
the nucleic acid genome of the recombinant oncolytic virus comprises the nucleic acid sequence set forth in SEQ ID NO: 90, or a nucleic acid sequence that has at least 95%, 96%, 97%, 98%, or 99% sequence identity to the nucleic acid sequence set forth in SEQ ID NO: 90.

158. The recombinant oncolytic virus of any one of embodiments 48-157, wherein the at least one heterologous nucleic acid encoding one or more heterologous gene product comprises one or more heterologous nucleic acid each encoding one or more anti-angiogenic protein.

159. The recombinant oncolytic virus of any one of embodiments 48, 50-52, and 54-157, wherein the inactivating mutation of one or more of the at least one viral gene is by insertion of one or more heterologous nucleic acid each encoding one or more anti-angiogenic protein.

160. The recombinant oncolytic virus of embodiment 158 or embodiment 159, wherein the one or more anti-angiogenic protein is a VEGF inhibitor, an angiopoietin inhibitor, versikine, or a fusion protein of any two or more of the foregoing.

161. The recombinant oncolytic virus of any one of embodiments 158-160, wherein the one or more anti-angiogenic protein comprises a VEGF inhibitor and/or an angiopoietin inhibitor, optionally an inhibitor of Ang2.

162. The recombinant oncolytic virus of any one of embodiments 158-161, wherein the one or more anti-angiogenic protein comprises an anti-VEGF antibody and/or an anti-Ang2 antibody.

163. The recombinant oncolytic virus of any one of embodiments 160-162, wherein the VEGF inhibitor is an anti-VEGF antibody, optionally an anti-VEGF-single chain antibody (scAb).

164. The recombinant oncolytic virus of any one of embodiments 160-163, wherein the angiopoietin inhibitor is an anti-Angriopoietin-2 (Ang2) antibody, optionally an anti-Ang2 single chain antibody (scAb).

165. The recombinant oncolytic virus of any one of embodiments 158-164, wherein the one or more anti-angiogenic protein is a bispecific anti-VEGF/anti-Ang2 antibody.

166. The recombinant oncolytic virus of embodiment 165, wherein the bispecific anti-VEGF/anti-Ang2 antibody comprises the amino acid sequence set forth in SEQ ID NO: 23, or an amino acid sequence that has at least 85%, 90%, 91%, 92%, 93%, 94%, 95%, 96%, 97%, 98%, or 99% sequence identity to the sequence set forth in SEQ ID NO: 23.

167. The recombinant oncolytic virus of any one of embodiments 158-166, wherein the one or more anti-angiogenic protein comprises versikine.

168. The recombinant oncolytic virus of embodiment 167, wherein the versikine comprises the amino acid sequence set forth in SEQ ID NO: 24, or comprises an amino acid sequence that has at least 85%, 90%, 91%, 92%, 93%, 94%, 95%, 96%, 97%, 98%, or 99% sequence identity to the sequence set forth in SEQ ID NO: 24.

169. The recombinant oncolytic virus of any one of embodiments 158-168, wherein the nucleic acid genome of the recombinant oncolytic virus comprises the nucleic acid sequence set forth in any one of SEQ ID NOs: 13, 47, 82, 87, and 88, or a nucleic acid sequence that has at least 95%, 96%, 97%, 98%, or 99% sequence identity to the nucleic acid sequence set forth in any one of SEQ ID NOS: 13, 47, 82, 87, and 88.

170. The recombinant oncolytic virus of any of embodiments 48-169, wherein the at least one heterologous nucleic acid encoding one or more heterologous gene product comprises one or more heterologous nucleic acid each encoding one or more therapeutic agent or diagnostic agent.

171. The recombinant oncolytic virus of any one of embodiments 48, 50-52, and 54-170, wherein the inactivating mutation of one or more of the at least one viral gene is by insertion of one or more heterologous nucleic acid each encoding one or more one or more therapeutic agent or diagnostic agent.

172. The recombinant oncolytic virus of embodiment 170 or embodiment 171, wherein the one or more therapeutic agent or diagnostic agent are selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging.

173. The recombinant oncolytic virus of any of embodiments 170-172, wherein the one or more therapeutic agent or diagnostic agent comprises a therapeutic agent selected from among a hormone, a growth factor, cytokine, a chemokine, a costimulatory molecule, ribozymes, a transporter protein, a single chain antibody, an antisense RNA, a prodrug converting enzyme, an siRNA, a microRNA, a toxin, an antitumor oligopeptide, a mitosis inhibitor protein, an antimitotic oligopeptide, an anti-cancer polypeptide antibiotic, an angiogenesis inhibitor, a tumor suppressor, a cytotoxic protein, a cytostatic protein and a tissue factor.

174. The recombinant oncolytic virus of any one of embodiments 48, 50-52, and 54-173, wherein:
(i) the at least one viral gene is or comprises A35R, optionally wherein the nucleic acid genome of the recombinant oncolytic virus comprises the nucleic acid sequence of SEQ ID NO: 3, or a nucleic acid sequence that has at least 95%, 96%, 97%, 98%, or 99% sequence identity to the nucleic acid sequence set forth in SEQ ID NO: 3; or
(ii) the at least one viral gene is or comprises A35R and J2R, optionally wherein the nucleic acid genome of the recombinant oncolytic virus comprises the nucleic acid sequence of SEQ ID NO: 12, or a nucleic acid sequence that has at least 95%, 96%, 97%, 98%, or 99% sequence identity to the nucleic acid sequence set forth in SEQ ID NO: 12; or
(iii) the at least one viral gene is or comprises J2R, and the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018), and
wherein the at least one heterologous nucleic acid encoding one or more heterologous gene product comprises one or more heterologous nucleic acid each encoding one or more complement inhibitor that is introduced into a viral membrane gene to produce a fusion gene encoding a fusion protein, optionally wherein the viral membrane gene is F14.5L, optionally wherein the fusion is at the C-terminus of the F14.5L protein, and optionally wherein the nucleic acid genome of the recombinant oncolytic virus comprises the nucleic acid sequence of SEQ ID NO: 10, or a nucleic acid sequence that has at least 95%, 96%, 97%, 98%, or 99% sequence identity to the nucleic acid sequence set forth in SEQ ID NO: 10; or
(iv) the at least one viral gene is or comprises J2R, optionally wherein the nucleic acid genome of the recombinant oncolytic virus comprises the nucleic acid sequence of SEQ ID NO: 4, or a nucleic acid sequence that has at least 95%, 96%, 97%, 98%, or 99% sequence identity to the nucleic acid sequence set forth in SEQ ID NO: 4; or
(v) the at least one viral gene is or comprises J2R and A35R, and the inactivating mutation of A35R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and
optionally wherein the nucleic acid genome of the recombinant oncolytic virus comprises the nucleic acid sequence of SEQ ID NO: 11, or a nucleic acid sequence that has at least 95%, 96%, 97%, 98%, or 99% sequence identity to the nucleic acid sequence set forth in SEQ ID NO: 11.

175. The recombinant oncolytic virus of any one of embodiments 48, 50-52, and 54-173, wherein:
(i) the at least one viral gene is or comprises J2R and A35R, and the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; and
optionally wherein the nucleic acid genome of the recombinant oncolytic virus comprises the nucleic acid sequence of SEQ ID NO: 13, or a nucleic acid sequence that has at least 95%, 96%, 97%, 98%, or 99% sequence identity to the nucleic acid sequence set forth in SEQ ID NO: 13; or
(ii) the at least one viral gene is or comprises J2R and A35R, and the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and
the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody; and
optionally wherein the nucleic acid genome of the recombinant oncolytic virus comprises the nucleic acid sequence of SEQ ID NO: 47, or a nucleic acid sequence that has at least 95%, 96%, 97%, 98%, or 99% sequence identity to the nucleic acid sequence set forth in SEQ ID NO: 47; or
(iii) the at least one viral gene is or comprises J2R, and the inactivating mutation of J2R is by insertion of one or more heterologous nucleic acid each encoding an apoptosis-inducible protein, optionally wherein the apoptosis-inducible protein is an inducible DED (IDED), an inducible Fas (iFas), or an inducible Cas9 (iCas9), optionally wherein the nucleic acid genome of the recombinant oncolytic virus comprises the nucleic acid sequence of SEQ ID NO: 7, 8, or 9, or a nucleic acid sequence that has at least 95%, 96%, 97%, 98%, or 99% sequence identity to the nucleic acid sequence set forth in SEQ ID NO: 7, 8, or 9; or
(iv) the at least one viral gene is or comprises J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; optionally wherein the nucleic acid genome of the recombinant oncolytic virus comprises the nucleic acid sequence of SEQ ID NO: 49, 50, or 93, or a nucleic acid sequence that has at least 95%, 96%, 97%, 98%, or 99% sequence identity to the nucleic acid sequence set forth in SEQ ID NO: 49, 50, or 93; or
(v) the at least one viral gene is or comprises J2R and B2R, optionally wherein the nucleic acid genome of the recombinant oncolytic virus comprises the nucleic acid sequence of SEQ ID NO: 48, or a nucleic acid sequence that has at least 95%, 96%, 97%, 98%, or 99% sequence identity to the nucleic acid sequence set forth in SEQ ID NO: 48.

176. The recombinant oncolytic virus of any one of embodiments 48, 50-52, and 54-173, wherein the at least one viral gene is or comprises J2R and B2R.

177. The recombinant oncolytic virus of any one of embodiments 48, 50-52, and 54-173, wherein:
(i) the at least one viral gene is or comprises J2R and B2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; optionally wherein the nucleic acid genome of the recombinant oncolytic virus comprises the nucleic acid sequence of SEQ ID NO: 80, or a nucleic acid sequence that has at least 95%, 96%, 97%, 98%, or 99% sequence identity to the nucleic acid sequence set forth in SEQ ID NO: 80; or
(ii) the at least one viral gene is or comprises J2R, B2R, and A35R; wherein:
the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody;
the inactivating mutation of B2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and
the inactivating mutation of A35R is by insertion of one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT; and
optionally wherein the nucleic acid genome of the recombinant oncolytic virus comprises the nucleic acid sequence of SEQ ID NO: 82, or a nucleic acid sequence that has at least 95%, 96%, 97%, 98%, or 99% sequence identity to the nucleic acid sequence set forth in SEQ ID NO: 82; or
(iii) the at least one viral gene is or comprises J2R, B2R, and A56R; wherein:
the inactivating mutation of J2R is by insertion of one or more heterologous nucleic acid encoding one or more immune modulating proteins, optionally wherein the one or more immune modulating proteins is IRF3;
the inactivating mutation of A56R is by insertion of one or more heterologous nucleic acid encoding one or more immune modulating proteins, optionally wherein the one or more immune modulating proteins is IL-2, optionally wherein the IL-2 is an IL-2 superkine, optionally MDNA11; and
optionally wherein the nucleic acid genome of the recombinant oncolytic virus comprises the nucleic acid sequence of SEQ ID NO: 84, or a nucleic acid sequence that has at least 95%, 96%, 97%, 98%, or 99% sequence identity to the nucleic acid sequence set forth in SEQ ID NO: 84; or
(iv) the at least one viral gene is or comprises J2R, B2R, and A56R; wherein:
the inactivating mutation of J2R is by insertion of one or more heterologous nucleic acid encoding one or more immune modulating proteins, optionally wherein the one or more immune modulating proteins is IRF3;
the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; and
optionally wherein the nucleic acid genome of the recombinant oncolytic virus comprises the nucleic acid sequence of SEQ ID NO: 85, or a nucleic acid sequence that has at least 95%, 96%, 97%, 98%, or 99% sequence identity to the nucleic acid sequence set forth in SEQ ID NO: 85; or
(v) the at least one viral gene is or comprises J2R, B2R, and A56R; wherein:
the inactivating mutation of B2R is by insertion of one or more heterologous nucleic acid encoding one or more immune modulating proteins, optionally wherein the one or more immune modulating proteins is IRF3;
the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9;
the inactivating mutation of J2R is by insertion of one or more heterologous nucleic acid each encoding an apoptosis-inducible protein, optionally wherein the apoptosis-inducible protein is an inducible DED (iDED); and
optionally wherein the nucleic acid genome of the recombinant oncolytic virus comprises the nucleic acid sequence of SEQ ID NO: 86, or a nucleic acid sequence that has at least 95%, 96%, 97%, 98%, or 99% sequence identity to the nucleic acid sequence set forth in SEQ ID NO: 86; or
(vi) the at least one viral gene is or comprises J2R, B2R, A35R, and A56R; wherein:
the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody;
the inactivating mutation of B2R is by insertion of one or more heterologous nucleic acid encoding one or more immune modulating proteins, optionally wherein the one or more immune modulating proteins is IRF3;
the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT;
the inactivating mutation of A56R is by insertion of one or more heterologous nucleic acid encoding one or more immune modulating proteins, wherein the one or more immune modulating proteins is IL-2 superkine MDNA11; and
optionally wherein the nucleic acid genome of the recombinant oncolytic virus comprises the nucleic acid sequence of SEQ ID NO: 87, or a nucleic acid sequence that has at least 95%, 96%, 97%, 98%, or 99% sequence identity to the nucleic acid sequence set forth in SEQ ID NO: 87; or
(vii) the at least one viral gene is or comprises J2R, B2R, A35R, and A56R; wherein:
the inactivating mutation of J2R is by insertion of the one or more heterologous nucleic acid encoding one or more anti-angiogenic protein, optionally wherein the one or more anti-angiogenic protein comprises an inhibitor or VEGF and/or an inhibitor of Ang2, optionally wherein the one or more anti-angiogenic protein is a bispecific anti-VEGF/anti-Ang2 antibody;
the inactivating mutation of B2R is by insertion of one or more heterologous nucleic acid encoding one or more immune modulating proteins, optionally wherein the one or more immune modulating proteins is IRF3;
the inactivating mutation of A35R is by insertion of the one or more heterologous nucleic acid encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is LIGHT;
the inactivating mutation of A56R is by insertion of one or more heterologous nucleic acid encoding one or more immune modulating proteins, wherein the one or more immune modulating proteins is IL-2 superkine MDNA11T, optionally wherein the MDNA11T comprises the amino acid sequence set forth in SEQ ID NO: 98; and
optionally wherein the nucleic acid genome of the recombinant oncolytic virus comprises the nucleic acid sequence of SEQ ID NO: 88, or a nucleic acid sequence that has at least 95%, 96%, 97%, 98%, or 99% sequence identity to the nucleic acid sequence set forth in SEQ ID NO: 88; or
(viii) the at least one viral gene is or comprises J2R, B2R, and A56R; wherein:
the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018);
the inactivating mutation of B2R is by insertion of one or more heterologous nucleic acid encoding one or more immune modulating proteins, optionally wherein the one or more immune modulating proteins is IRF3;
the inactivating mutation of A56R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is an IL-2 superkine, optionally MDNA11 or MDNA11T;
the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein, optionally wherein the fusion is at the C-terminus of the F14.5L protein; and
optionally wherein the nucleic acid genome of the recombinant oncolytic virus comprises the nucleic acid sequence of SEQ ID NO: 89, or a nucleic acid sequence that has at least 95%, 96%, 97%, 98%, or 99% sequence identity to the nucleic acid sequence set forth in SEQ ID NO: 89; or
(ix) the at least one viral gene is or comprises J2R, B2R, and A56R; wherein:
the inactivating mutation of J2R is by insertion of one or more heterologous nucleic acid each encoding one or more T cell or NK cell evader proteins, optionally wherein the one or more T cell or NK cell evader proteins comprises a set of proteins encoded by Cowpox virus ORFs 012, 203 and 018 (CPXV012-203-018);
the inactivating mutation of B2R is by insertion of one or more heterologous nucleic acid encoding one or more immune modulating proteins, optionally wherein the one or more immune modulating proteins is IRF3;
the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9;
the at least one heterologous nucleic acid encoding one or more heterologous gene product comprises one or more heterologous nucleic acid each encoding one or more complement inhibitor, optionally CRASP-2, that is introduced into a viral membrane gene, optionally F14.5L, to produce a fusion gene encoding a fusion protein, optionally wherein the fusion is at the C-terminus of the F14.5L protein; and
optionally wherein the nucleic acid genome of the recombinant oncolytic virus comprises the nucleic acid sequence of SEQ ID NO: 90, or a nucleic acid sequence that has at least 95%, 96%, 97%, 98%, or 99% sequence identity to the nucleic acid sequence set forth in SEQ ID NO: 90; or
(x) the at least one viral gene is or comprises B2R and J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and
optionally wherein the nucleic acid genome of the recombinant oncolytic virus comprises the nucleic acid sequence of SEQ ID NO: 91, or a nucleic acid sequence that has at least 95%, 96%, 97%, 98%, or 99% sequence identity to the nucleic acid sequence set forth in SEQ ID NO: 91; or
(xi) the at least one viral gene is or comprises B2R, J2R, and A56R, and
the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and
the inactivating mutation of A56R is by insertion of one or more heterologous nucleic acid encoding one or more immune modulating proteins, optionally wherein the one or more immune modulating proteins comprises two or more immune modulating proteins selected from the group consisting of LIGHT, IRF3, IL-2, IL-12, and CXCL9, optionally wherein the two or more immune modulating proteins comprises IL-12 and CXCL9; and
optionally wherein the nucleic acid genome of the recombinant oncolytic virus comprises the nucleic acid sequence of SEQ ID NO: 92, or a nucleic acid sequence that has at least 95%, 96%, 97%, 98%, or 99% sequence identity to the nucleic acid sequence set forth in SEQ ID NO: 92; or
(xii) the at least one viral gene is or comprises J2R, and the inactivating mutation of J2R is by insertion of one or more heterologous nucleic acid each encoding one or more immune modulating proteins, optionally wherein the one or more immune modulating proteins is selected from the group consisting of LIGHT, IRF3, IL-2, IL-12, and CXCL9, optionally wherein the one or more immune modulating proteins is IRF3; and
optionally wherein the nucleic acid genome of the recombinant oncolytic virus comprises the nucleic acid sequence of SEQ ID NO: 93, or a nucleic acid sequence that has at least 95%, 96%, 97%, 98%, or 99% sequence identity to the nucleic acid sequence set forth in SEQ ID NO: 93.

178. The recombinant oncolytic vaccinia virus of any one of embodiments 48-173, wherein the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

179. The recombinant oncolytic vaccinia virus of any one of embodiments 48-173, wherein the nucleic acid genome of the recombinant oncolytic virus comprises a nucleic acid sequence set forth in any one of SEQ ID NOs: 85, 86, 88, and 90, or a nucleic acid sequence that has at least 95%, 96%, 97%, 98%, or 99% sequence identity to a nucleic acid sequence set forth in any one of SEQ ID NOs 85, 86, 88, and 90.

180. The recombinant oncolytic vaccinia virus of any one of embodiments 48-173, wherein the nucleic acid genome of the recombinant oncolytic virus comprises a nucleic acid sequence set forth in SEQ ID NO: 85, or a nucleic acid sequence that has at least 95%, 96%, 97%, 98%, or 99% sequence identity to a nucleic acid sequence set forth in SEQ ID NO 85.

181. The recombinant oncolytic virus of any of embodiments 48-180, wherein one or more of the heterologous nucleic acid encoding a heterologous gene product is operably linked to a promoter.

182. The recombinant oncolytic virus of embodiment 181, wherein each of the one or more heterologous nucleic acid encoding a heterologous gene product that is operably linked to a promoter is selected from the group consisting of 7.5E, 7.5E/L, SSE, 11KL, SSL, SSEL, mH5, and LEO.

183. The recombinant oncolytic virus of any of embodiments 48-182, wherein each heterologous nucleic acid encoding a heterologous gene product is independently operably linked to a promoter, optionally wherein each heterologous nucleic acid encoding a heterologous gene product is independently operably linked to a promoter selected from the group consisting of 7.5E, 7.5E/L, SSE, 11KL, SSL, SSEL, mH5, and LEO.

184. The recombinant oncolytic virus of embodiment 182 or embodiment 183, wherein the promoter is a poxviral promoter or is a variant or derivative thereof.

185. The recombinant oncolytic virus of embodiment 182 or embodiment 183, wherein the promoter is a vaccinia virus promoter.

186. The recombinant oncolytic virus of any of embodiments 182-185, wherein the promoter is selected from the group consisting of 7.5E, 7.5E/L, SSE, 11KL, SSL, SSEL, mH5, and LEO.

187. The recombinant oncolytic virus of any of embodiments 182-186, wherein the promoter has the sequence of amino acids set forth in any one of SEQ ID NOS: 29, 53, 55, 68, 69, 70, 71, or 72.

188. The recombinant oncolytic virus of any of embodiments 182-187, wherein the promoter is synthetic strong early promoter (SSE).

189. The recombinant VACV strain of embodiment 188, wherein the SSE promoter comprises the sequence set forth in SEQ ID NO: 29.

190. The recombinant oncolytic virus of any of embodiments 182-189, wherein the promoter is a strong early/late promoter (SEL).

191. The recombinant oncolytic virus of embodiment 190, wherein the SEL promoter comprises the sequence set forth in SEQ ID NO: 55.

192. The recombinant oncolytic virus of any of embodiments 182-191, wherein the promoter is mH5.

193. The recombinant oncolytic virus of embodiment 192, wherein the mH5 promoter comprises the sequence set forth in SEQ ID NO: 53.

194. An isolated clonal vaccinia virus (VACV) strain comprising a nucleic acid genome that has at least 95% sequence identity with the nucleic acid sequence set forth in SEQ ID NO: 1 and wherein the nucleic acid genome is characterized by one or more of:
(i) a variant 017 open reading frame (ORF) encoding an amino acid sequence that has at least 95% sequence identity to SEQ ID NO: 57 and comprises a polar uncharged amino acid at position 66, optionally a threonine (T) at position 66;
(ii) a variant 038 (K5L) ORF comprising a nucleotide insertion to effect a frameshift mutation, wherein the 038 (K5L) gene product is altered;
(iii) a variant 059 (E2L) ORF encoding an amino acid sequence that is at least 95% sequence identity to SEQ ID NO: 60 and comprises a hydrophobic amino acid other than leucine at position 419, optionally a phenylalanine (F) at position 419;
(iv) a variant 104 (H4L) ORF encoding an amino acid sequence having at least 95% sequence identity to SEQ ID NO: 61 and comprises a negatively charged amino acid at position 591, optionally aspartic acid (D) at position 591; and
(v) a variant 182 (A56R) ORF comprising deletion of two nucleotides to effect a frameshift mutation, wherein the 182 (A56R) ORF gene product is altered.

195. The isolated clonal VACV strain of embodiment 194, wherein the nucleic acid genome is characterized by (i) and the variant 017 ORF encodes an amino acid sequence that has at least 96%, at least 97%, at least 98%, at least 99% or 100% sequence identity to SEQ ID NO: 57.

196. The isolated clonal VACV strain of embodiment 194 or embodiment 195, wherein the nucleic acid genome is characterized by (i) and the variant 017 ORF encodes the amino acid sequence set forth in SEQ ID NO: 57.

197. The isolated clonal VACV strain of any of embodiments 194-196, wherein the nucleic acid genome is characterized by (ii) and the nucleotide insertion is guanine (G) corresponding to insertion after nucleotide position 32135 of SEQ ID NO: 1, optionally wherein the variant 038 (K5L) ORF is set forth in SEQ ID NO: 58.

198. The isolated clonal VACV strain of any of embodiments 194-197, wherein the nucleic acid genome is characterized by (ii) and the 038 (K5L) gene product is set forth in SEQ ID NO: 59.

199. The isolated clonal VACV strain of any of embodiments 194-198, wherein the nucleic acid genome is characterized by (iii) and the variant 059 (E2L) ORF encodes an amino acid sequence that has at least 96%, at least 97%, at least 98%, at least 99% or 100% sequence identity to SEQ ID NO: 60.

200. The isolated clonal VACV strain of any of embodiments 194-199, wherein the nucleic acid genome is characterized by (iii) and the variant 059 (E2L) ORF encodes the amino acid sequence set forth in SEQ ID NO: 60.

201. The isolated clonal VACV strain of any of embodiments 194-200, wherein the nucleic acid genome is characterized by (iv) and the 104 (H4L) ORF encodes an amino acid sequence that has at least 96%, at least 97%, at least 98%, at least 99% or 100% sequence identity to SEQ ID NO: 61.

202. The isolated clonal VACV strain of any of embodiments 194-201, wherein the nucleic acid genome is characterized by (iv) and wherein the variant 104 (H4L) ORF encodes the amino acid sequence set forth in SEQ ID NO: 61.

203. The isolated clonal VACV strain of any of embodiments 194-202, wherein the nucleic acid genome is characterized by (v) and the deletion of two nucleotides is deletion of two contiguous nucleotides corresponding to nucleotides after nucleotide position 165972 of SEQ ID NO: 2, optionally wherein the variant 182 (A56R) is set forth in SEQ ID NO: 62.

204. The isolated clonal VACV strain of any of embodiments 194-203, wherein the nucleic acid genome is characterized by (v) and the VACV protein is set forth in SEQ ID NO: 63.

205. The isolated clonal VACV strain of any of embodiments 194-204, wherein the nucleic acid genome is characterized by any two of (i)-(v).

206. The the isolated clonal VACV strain of any of embodiments 194-204, wherein the nucleic acid genome is characterized by any three of (i)-(v).

207. The the isolated clonal VACV strain of any of embodiments 194-204, wherein the nucleic acid genome is characterized by any four of (i)-(v).

208. The the isolated clonal VACV strain of any of embodiments 194-204, wherein the nucleic acid genome is characterized by each of (i)-(v).

209. An isolated clonal vaccinia virus (VACV) strain comprising a nucleic acid genome that has at least 95% sequence identity with the nucleic acid sequence set forth in SEQ ID NO: 1, and wherein the nucleic acid genome is characterized by one or more of:
(i) a guanine (G) at the position corresponding to position 7770 of SEQ ID NO: 1;
(ii) a thymine (T) at the position corresponding to position 15261 of SEQ ID NO: 1;
(iii) a G at the position corresponding to position 32136 of SEQ ID NO: 1;
(iv) a G at the position corresponding to position 49455 of SEQ ID NO: 1;
(v) a cytosine (C) at the position corresponding to position 92969 of SEQ ID NO: 1;
(vi) the nucleic acid sequence CACTTATATAT at the positions corresponding to positions 106870 to 106880 of SEQ ID NO: 1;
(vii) the nucleic acid sequence GTTTTCATTA at the positions corresponding to positions 111267 to 111276 of SEQ ID NO: 1;
(viii) an adenine (A) at the position corresponding to position 162715 of SEQ ID NO: 1;
(ix) the nucleic acid sequence TACAGACACC at the positions corresponding to positions 165844 to 185853 of SEQ ID NO: 1; and
(x) a C at the position corresponding to position 187805 of SEQ ID NO: 1.

210. The isolated clonal VACV strain of embodiment 209, wherein the nucleic acid genome is characterized by any two of (i)-(x).

211. The isolated clonal VACV strain of embodiment 209, wherein the nucleic acid genome is characterized by any three of (i)-(x).

212. The isolated clonal VACV strain of embodiment 209, wherein the nucleic acid genome is characterized by any four of (i)-(x).

213. The isolated clonal VACV strain of embodiment 209, wherein the nucleic acid genome is characterized by any five of (i)-(x).

214. The isolated clonal VACV strain of embodiment 209, wherein the nucleic acid genome is characterized by any six of (i)-(x).

215. The isolated clonal VACV strain of embodiment 209, wherein the nucleic acid genome is characterized by any seven of (i)-(x).

216. The isolated clonal VACV strain of embodiment 209, wherein the nucleic acid genome is characterized by any eight of (i)-(x).

217. The isolated clonal VACV strain of embodiment 209, wherein the nucleic acid genome is characterized by any nine of (i)-(x).

218. The isolated clonal VACV strain of embodiment 209, wherein the nucleic acid genome is characterized by each of (i)-(x).

219. The isolated clonal VACV strain of any of embodiments 209-218, wherein the nucleic acid genome has at least 96% sequence identity with the sequence of nucleotides set forth in SEQ ID NO: 1.

220. The isolated clonal VACV strain of any of embodiments 209-219, wherein the nucleic acid genome has at least 97% sequence identity with the sequence of nucleotides set forth in SEQ ID NO: 1.

221. The isolated clonal VACV strain of any of embodiments 209-220, wherein the nucleic acid genome has at least 98% sequence identity with the sequence of nucleotides set forth in SEQ ID NO: 1.

222. The isolated clonal VACV strain of any of embodiments 209-221, wherein the nucleic acid genome has at a least 99% sequence identity with the sequence of nucleotides set forth in SEQ ID NO: 1.

223. An isolated clonal vaccinia virus (VACV) strain comprising a nucleic acid genome that has at least 99% sequence identity with the sequence of nucleotides set forth in SEQ ID NO: 1.

224. The recombinant oncolytic virus of any one of embodiments 1-193, or the isolated clonal VACV strain of any of embodiments 194-223, wherein the nucleic acid genome has at least 99.5% sequence identity with the sequence of nucleotides set forth in SEQ ID NO: 1.

225. The recombinant oncolytic virus of any one of embodiments 1-193, or the isolated clonal VACV strain of any of embodiments 194-224, wherein the nucleic acid genome has at least 99.9% sequence identity with the sequence of nucleotides set forth in SEQ ID NO: 1.

226. The recombinant oncolytic virus of any one of embodiments 1-193, or the isolated clonal VACV strain of any of embodiments 194-225, wherein the nucleic acid genome has at least 99.95% sequence identity with the sequence of nucleotides set forth in SEQ ID NO: 1.

227. The recombinant oncolytic virus of any one of embodiments 1-193, or the isolated clonal VACV strain of any of embodiments 194-226, wherein the nucleic acid genome does not comprise the sequence of nucleotides set forth in SEQ ID NO: 2.

228. The isolated clonal VACV strain of any of embodiments 194-227, wherein the nucleic acid genome is not modified to contain non-viral heterologous nucleic acid containing an open reading frame encoding a non-viral heterologous protein.

229. The isolated clonal VACV strain of any of embodiments 194-228, wherein the nucleic acid genome is set forth in SEQ ID NO: 1.

230. The recombinant oncolytic virus of any one of embodiments 1-193, or the isolated clonal VACV strain of any of embodiments 194-229, wherein the recombinant oncolytic virus or the clonal VACV strain exhibits enhanced production of extracellular enveloped virions (EEV) after cell infection, optionally as determined by percentage of EEV, wherein the percentage of EEV is determined by the formula: viral titer in supernatant/(viral titer in supernatant+viral titer in cell lysate)*100.

231. The recombinant oncolytic virus of embodiment 230, or the isolated clonal VACV strain of embodiment 244, wherein greater than 5% of infectious particles after cell infection are EEV.

232. The recombinant oncolytic virus of embodiment 230, or the isolated clonal VACV strain of embodiment 244, wherein greater than 10% of infectious particles after cell infection are EEV.

233. The recombinant oncolytic virus of embodiment 230, or the isolated clonal VACV strain of embodiment 244, wherein greater than 15% of infectious particles after cell infection are EEV.

234. The recombinant oncolytic virus of any one of embodiments 230-233, or the isolated clonal VACV strain of any one of embodiments 230-233, wherein the recombinant oncolytic virus or the clonal VACV strain exhibits enhanced production of extracellular enveloped virions (EEV) after cell infection, as determined by having a percentage of at least 5%, 10%, or 15% of infectious particles being EEV.

235. The recombinant oncolytic virus of any one of embodiments 1-193, 224-227, and 230-234, or the isolated clonal VACV strain of any of embodiments 194-234, that exhibits oncolytic activity to kill tumor cells.

236. A VACV preparation comprising the isolated clonal VACV strain of any of embodiments 194-235.

237. A VACV preparation comprising the recombinant oncolytic virus of any one of embodiments 1-193, 224-227, and 230-235, wherein the recombinant oncolytic virus is a recombinant oncolytic vaccinia virus.

238. A recombinant oncolytic virus preparation comprising the recombinant oncolytic virus of any one of embodiments 1-193, 224-227, and 230-235, optionally wherein at least 70%, 80%, 90%, 95%, or 98% of the virus particles in the preparation have the genomic sequence of the clonal oncolytic virus strain.

239. The VACV preparation of embodiment 236 or embodiment 237, that is substantially homogenous wherein a plurality of the virus particles in the preparation has the genomic sequence of the clonal VACV strain.

240. The VACV preparation of any one of embodiments 236, 237, and 239, wherein at least 70% of the virus particles in the preparation have the genomic sequence of the clonal VACV strain.

241. The VACV preparation of any one of embodiments 236, 237, and 239, wherein at least 80% of the virus particles in the preparation have the genomic sequence of the clonal VACV strain.

242. The VACV preparation of any one of embodiments 236, 237, and 239, wherein at least 90% of the virus particles in the preparation have the genomic sequence of the clonal VACV strain.

243. The VACV preparation of any one of embodiments 236, 237, and 239, wherein at least 95% of the virus particles in the preparation have the genomic sequence of the clonal VACV strain.

244. The VACV preparation of any one of embodiments 236, 237, and 239, wherein at least 98% of the virus particles in the preparation have the genomic sequence of the clonal VACV strain.

245. A pharmaceutical composition comprising the isolated VACV clonal strain of any of embodiments 208-248.

246. A pharmaceutical composition comprising the VACV preparation of any of embodiments 194-234.

247. A pharmaceutical composition comprising the recombinant oncolytic virus of any one of embodiments 1-193, 224-227, and 230-235.

248. A recombinant vaccinia virus (VACV) strain comprising a nucleic acid genome of the VACV clonal strain of any of embodiments 194-235 that comprises an inactivating mutation in at least one viral gene.

249. The recombinant VACV strain of embodiment 248, wherein the viral gene is selected from the group consisting of hemagglutinin (HA), J2R (thymidine kinase), F14.5L, A56R (hemagglutinin), B2R, vaccinia growth factor (VGF), A35R, A49R, A55R, B14R, C4L, C6L, C16L, NIL/N2L, E2L/E3L, K1L/K2L, K7L, superoxide dismutase locus, 7.5K, C2L-F3L, C4L-F1L, C7-K1L, B13R+B14R, A26L and I4L.

250. The recombinant VACV of embodiment 248 or embodiment 249, wherein the inactivating mutation is a deletion of all or a portion of the at least one viral gene.

251. The recombinant VACV strain of embodiment 250, wherein the deletion of the at least one viral gene is deletion of the entire gene ORF of a viral gene.

252. The recombinant VACV strain of embodiment 250, wherein the deletion of the at least one viral gene is a deletion of a portion of the ORF of a viral gene, and wherein said deletion is sufficient to render the encoded gene product non-functional.

253. The recombinant VACV strain of any of embodiments 248-252, wherein the at least one viral gene is or comprises A35R.

254. The recombinant VACV strain of embodiment 253, wherein the nucleic acid genome of the recombinant VACV strain comprises the nucleic acid sequence set forth in SEQ ID NO: 3, or a nucleic acid sequence that has at least 95%, at least 96%, at least 97%, at least 98% or at least 99% sequence identity to the sequence set forth in SEQ ID NO: 3.

255. The recombinant VACV strain of any of embodiments 248-254, wherein the at least one viral gene is or comprises J2R.

256. The recombinant VACV strain of embodiment 255, wherein the nucleic acid genome of the recombinant VACV strain comprises the nucleic acid sequence set forth in SEQ ID NO: 4, or a nucleic acid sequence that has at least 95%, at least 96%, at least 97%, at least 98% or at least 99% sequence identity to the sequence set forth in SEQ ID NO: 4.

257. The recombinant VACV strain of any of embodiments 248-256, wherein the at least one viral gene is or comprises B2R.

258. The recombinant VACV strain of any of embodiments 248-257, wherein the at least one viral gene is or comprises A35R and J2R.

259. The recombinant VACV strain of embodiment 258, wherein the nucleic acid genome of the recombinant VACV strain comprises the nucleic acid sequence set forth in SEQ ID NO: 12, or a nucleic acid sequence that has at least 95%, at least 96%, at least 97%, at least 98% or at least 99% sequence identity to the sequence set forth in SEQ ID NO: 12.

260. The recombinant VACV strain of any of embodiments 248-259, wherein the at least one viral gene is or comprises B2R and J2R.

261. The recombinant VACV strain of embodiment 260, wherein the nucleic acid genome of the recombinant VACV strain comprises the nucleic acid sequence set forth in SEQ ID NO: 48, or a nucleic acid sequence that has at least 95%, at least 96%, at least 97%, at least 98% or at least 99% sequence identity to the sequence set forth in SEQ ID NO: 48.

262. The recombinant VACV strain of any one of embodiments 248-261, wherein the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

263. The recombinant VACV strain of any one of embodiments 248-261, wherein the nucleic acid genome of the recombinant oncolytic virus comprises a nucleic acid sequence set forth in any one of SEQ ID NOs: 85, 86, 88, and 90, or a nucleic acid sequence that has at least 95%, 96%, 97%, 98%, or 99% sequence identity to a nucleic acid sequence set forth in any one of SEQ ID NOs 85, 86, 88, and 90.

264. The recombinant VACV strain of any one of embodiments 248-261, wherein the nucleic acid genome of the recombinant oncolytic virus comprises a nucleic acid sequence set forth in SEQ ID NO: 85, or a nucleic acid sequence that has at least 95%, 96%, 97%, 98%, or 99% sequence identity to a nucleic acid sequence set forth in SEQ ID NO 85.

265. A nucleic acid comprising a genome of the recombinant oncolytic virus of any one of embodiments 1-193, 224-227, and 230-235, or the isolated VACV clonal strain of any of embodiments 194-234.

266. A recombinant oncolytic virus comprising the nucleic acid of embodiment 265.

267. The nucleic acid of embodiment 265, wherein the recombinant oncolytic virus is a recombinant oncolytic vaccinia virus.

268. The recombinant oncolytic virus of embodiment 266, that is a recombinant oncolytic vaccinia virus.

269. A pharmaceutical composition comprising the recombinant VACV strain of any of embodiments 248-264.

270. A pharmaceutical composition comprising the recombinant oncolytic virus of any one of embodiments 1-193, 224-227, and 230-235, optionally wherein the recombinant oncolytic virus is a recombinant oncolytic vaccinia virus.

271. The pharmaceutical composition of any one of embodiments 245-247, 269, and 270, further comprising a pharmaceutically acceptable carrier.

272. The pharmaceutical composition of any one of embodiments 245-247 and 269-271, that is formulated for intravenous administration, intratumoral administration, intraperitoneal administration or intrapleural administration.

273. The pharmaceutical composition of any one of embodiments 245-247 and 269-272, that is formulated for intravenous administration.

274. The pharmaceutical composition of any one of embodiments 245-247 and 269-273, wherein the pharmaceutical composition is a liquid composition.

275. The pharmaceutical composition of any one of embodiments 245-247 and 269-273, wherein the pharmaceutical composition is lyophilized.

276. A method of treating a proliferative disorder in a subject comprising administering to the subject the recombinant oncolytic virus of any one of embodiments 1-193, 224-227, and 230-235, the isolated clonal VACV strain of any one of embodiments 194-235, the recombinant VACV strain of any one of embodiments 248-264, or the pharmaceutical composition of any one of embodiments 245-247 and 269-275.

277. The method of embodiment 276, wherein the proliferative disorder is a tumor or a metastasis.

278. The method of embodiment 276 or embodiment 277, wherein the proliferative disorder is a cancer.

279. The method of embodiment 278, wherein the cancer is a pancreatic cancer, ovarian cancer, lung cancer, colon cancer, prostate cancer, cervical cancer, breast cancer, rectal cancer, renal (kidney) cancer, gastric cancer, esophageal cancer, hepatic (liver) cancer, endometrial cancer, bladder cancer, brain cancer, head and neck cancer, oral cancer (e.g., oral cavity cancer), cervical cancer, uterine cancer, thyroid cancer, testicular cancer, prostate cancer, skin cancers, such as melanoma, e.g., malignant melanoma, cholangiocarcinoma (bile duct cancer), thymic epithelial cancer, e.g., thymoma, leukemia, lymphoma, or multiple myeloma.

280. The method of embodiment 278 or embodiment 279, wherein the cancer is Microsatellite Stable (MSS) colorectal cancer.

281. The method of embodiment 280, wherein the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

282. The method of any of embodiments 276-281, wherein the recombinant oncolytic virus or the isolated oncolytic virus is administered in an amount from 1×10⁵ pfu to 1×10¹⁴ pfu.

283. The method of any of embodiments 276-282, further comprising administering a second therapeutic agent for the treatment of the proliferative disorder.

284. The method of any of embodiments 276-283, further comprising another treatment selected from among surgery, radiation therapy, immunosuppressive therapy and administration of an anticancer agent.

285. The method of embodiments 284, wherein the another treatment is administration of an anticancer agent selected from among a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anticancer antibody, an anti-cancer antibiotic, an immunotherapeutic agent and a combination of any of the preceding thereof.

286. The method of any of embodiments 276-285, wherein the recombinant oncolytic virus or the isolated oncolytic virus is administered intravenously.

287. The method of any of embodiments 276-286, further comprising administering AP1903 (Rimiducid) to the subject.

288. The method of any of embodiments 276-287, wherein the recombinant oncolytic virus administered to the subject comprises a heterologous nucleic acid encoding an apoptosis inducible protein.

289. The method of embodiment 287 or embodiment 288, wherein the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

290. The method of any of embodiments 276-289, wherein the subject exhibits severe immune deficiency and is sensitive to virus infection.

291. The method of any one of embodiments 276-290, wherein the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

292. A method of inhibiting virus replication, the method comprising contacting cells infected with a recombinant oncolytic virus with AP1903 (Rimiducid), wherein the recombinant oncolytic virus comprises a heterologous nucleic acid encoding an apoptosis inducible protein.

293. A method of inhibiting virus replication, the method comprising contacting cells with AP1903 (Rimiducid), wherein the cells are infected with the recombinant oncolytic virus of any one of embodiments 1-193, 224-227, and 230-235, the isolated clonal VACV strain of any one of embodiments 194-235, or the recombinant VACV strain of any one of embodiments 248-264.

294. The method of embodiment 292 or embodiment 293, wherein the contacting occurs in vivo in a subject.

295. The method of embodiment 292 or embodiment 294, wherein the AP1903 (Rimiducid) has been administered to a subject previously administered with a recombinant oncolytic virus comprising the heterologous nucleic acid encoding an apoptosis inducible protein.

296. The method of embodiment 293 or embodiment 294, wherein the AP1903 (Rimiducid) has been administered to a subject previously administered with the recombinant oncolytic virus of any one of embodiments 1-193, 224-227, and 230-235, the isolated clonal VACV strain of any one of embodiments 194-235, or the recombinant VACV strain of any one of embodiments 248-264.

297. A method of inhibiting virus replication in a subject, the method comprising administering to a subject AP1903 (Rimiducid), wherein the subject has been previously administered a recombinant oncolytic virus comprising a heterologous nucleic acid encoding an apoptosis inducible protein.

298. A method of inhibiting virus replication in a subject, the method comprising administering to a subject AP1903 (Rimiducid), wherein the subject has been previously administered the recombinant oncolytic virus of any one of embodiments 1-193, 224-227, and 230-235, the isolated clonal VACV strain of any one of embodiments 194-235, or the recombinant VACV strain of any one of embodiments 248-264.

299. The method of any of embodiments 287-298, wherein the method inhibits virus replication preferentially in non-cancer cells.

300. The method of any of embodiments 287-299, wherein the apoptosis-inducible protein is an inducible DED (iDED).

301. The method of embodiment 300, wherein the iDED comprises the amino acid sequence set forth in SEQ ID NO: 27, or an amino acid sequence that has at least 85%, 90% or 95% sequence identity to SEQ ID NO: 27.

302. The method of embodiment 300 and embodiment 301, wherein the nucleic acid genome of the recombinant oncolytic virus comprises the nucleic acid sequence set forth in SEQ ID NO: 8, or a nucleic acid sequence that has at least 95%, at least 96%, at least 97%, at least 98% or at least 99% sequence identity to the sequence set forth in SEQ ID NO: 8.

303. The method of any one of embodiments 292-302, wherein the nucleic acid genome of the recombinant oncolytic virus comprises a nucleic acid sequence set forth in any one of SEQ ID NOs: 48, 80, 82, and 84-93, or a nucleic acid sequence that has at least 95%, 96%, 97%, 98%, or 99% sequence identity to a nucleic acid sequence set forth in any one of SEQ ID NOs 48, 80, 82, and 84-93.

### IX. Examples

### Example 1: Isolation and Characterization of Oncolytic Clonal Isolates of Vaccinia Virus

Clonal isolates of vaccinia viruses were isolated and assessed for oncolytic activity of cancer cells.

A. Isolation of Vaccinia Virus Clones with Oncolytic Activity

Vaccinia Virus (VACV) clones were isolated from mix populations of VACV IHD-J (obtained from ATCC-VR-156) and Copenhagen strains. The VACV parental strains were used to infect African green monkey kidney fibroblast cells (CV-1; obtained from ATCC). Confluent CV-1 cell cultures growing in 6-well plates were infected with a series of dilutions of the VACV strains. Two days after infection, six well-isolated plaques were picked for each VACV strain. These plaques were subjected to two more rounds of plaque purification in CV-1 cells. The VACV IHD-J isolated clones were designated as VIP01, VIP02, VIP03, VIP04, VIP05, VIP06 and the Copenhagen clones were designated as VCP01, VCP02, VCP03, VCP04, VCP05, and VCP06 respectively (data not shown).

Following clone isolation, the survival of tumor cells infected with each of the VIP01-06 clone isolates was evaluated in order to select clones with the highest cell killing activity. The human cancer cell line A549 was purchased from ATCC, and cancer cell lines BT-549, HCC-2998, LOX-IMVI and COLO205 were obtained from National Cancer Institute. VIP01, VIP02, VIP03, VIP04, VIP05 and VIP06 clone isolates were incubated with BT-549, A549, HCC-2998 cells (at MOI=0.01) and with LOX-IMVI and COLO-205 (MOI=0.1). Cell viability was measured after 96 hours of incubation and expressed as percentage of survival.

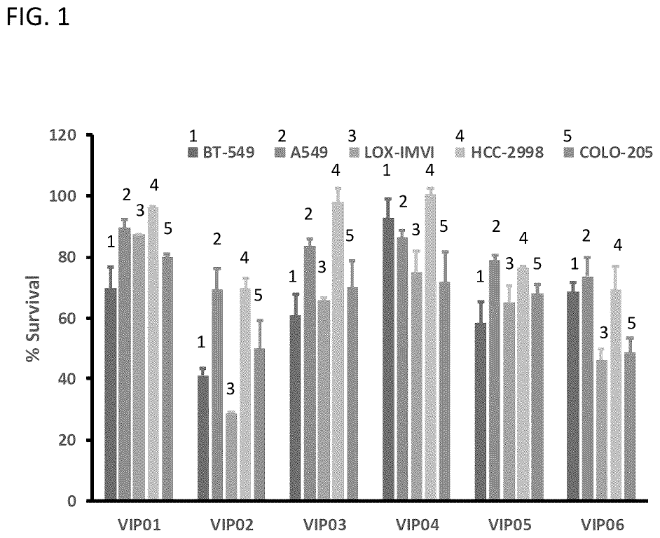

As depicted in FIG. 1, VIP02 showed the lowest percentage of cell survival and the highest in vitro cell killing activity when compared to the other five clone isolates.

### B. Sequencing of VIP02 Clonal Isolate

The VIP02 clone isolate was selected for further analysis as a parental isolate. Genomic DNA of the VIP02 clone isolate was extracted from purified virions using Wizard Genomic DNA Purification kit (Promega, Madison, WI). Libraries were prepared using TruSeq® DNA PCR-Free Library kit (Illumina, San Diego, CA). Sequencing was done using Illumina Miseq system. Library preparation, DNA sequencing and sequence assembly were done by Bio Applied Technologies Joint, Inc. (BATJ, San Diego, CA). The genomic DNA of the VIP02 clone isolate is set forth in SEQ. ID NO: 1. The sequence of VIP02 was compared with the genomic DNA of the IHD-W1 (SEQ. ID NO: 2) (accession number KJ125439.1), the closest match obtained after a database search. Results of this comparison are set forth in Table E1 below.

TABLE E1

| | Nucleotide Change | | | |
|---|---|---|---|---|
| Genomes | Position | Change | AA Change | ORF |
| VIP02 | nt 7770 in VIP02; 7891 in IHDW1 | “T” −> “G” | | |
| vs | nt 15261 in VIP02; 15382 in IHDW1 | “C” −> “T” | “Ala66” −> “Thr” | IHDW1_017 |
| IHDW1 | nt 32135\hat{ }32137 in VIP02; | “G” | ORF shift | IHDW1_038 |
| | 32256 . . . 32257 in IHDW1 | insertion | | (K5L) |
| | nt 49455 in VIP02; 49575 in IHDW1 | “T” −> “G” | “Leu419” −> “Phe” | IHDW1_059 |
| | | | | (E2L) |
| | nt 92969 in VIP02; 93089 in IHDW1 | “T” −> “C” | “Asn591” −> “Asp” | IHDW1_104 |
| | | | | (H4L) |
| | nt 106873 . . . 106874 in VIP02; | “TA” | | |
| | 106993\hat{ }106996 in IHDW1 | deletion | | |
| | nt 111272 . . . 111273 in VIP02; | “ATT” | | |
| | 111394\hat{ }111398 in IHDW1 | deletion | | |
| | nt 162714\hat{ }162716 in VIP02; | “A” | | |
| | 162839 . . . 162840 in IHDW1 | insertion | | |
| | nt 165848 . . . 165849 in VIP02; | “AC” | ORF shift | IHDW1_182 |
| | 165972\hat{ }165975 in IHDW1 | deletion | | (A56R) |
| | nt 187805 in VIP02; | “A” −> “C” | | |
| | 187931 in IHDW1 | | | |

C. Characterization of Extracellular Enveloped Virus (EEV) from Clonal Isolates

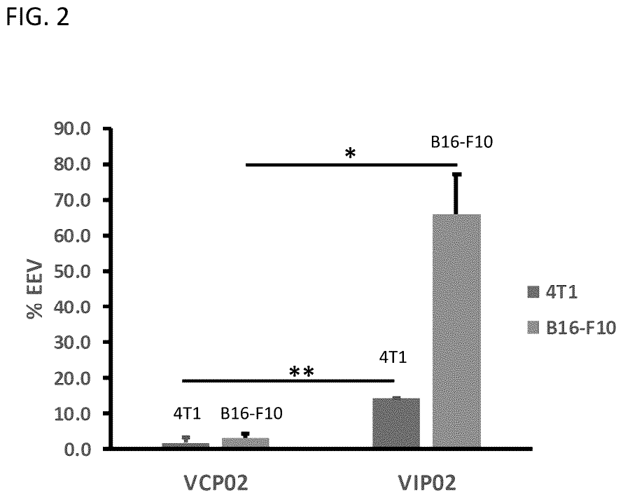

The production of extracellular enveloped virus (EEV) was characterized in the IHD-J derived VIP02 and the Copenhagen derived VCP02 clone isolates. 4T1 and B16-F10 cell cultures were infected with either VIP02 or VCP02 at an MOI of 0.01. The supernatants and the infected cells were harvested at 48 hours post infection (hpi) and were titrated in CV-1 cells. The percentage of EEV was determined by the formula: viral titer in supernatant/(viral titer in supernatant+viral titer in cell lysate)*100. As shown in FIG. 2, the VIP02 clone isolate derived from the IHD-J parental strain produced a significantly larger amount of EEV than the VCP02 clone isolate, in both infected 4T1 and B16-F10 cells.

Without wishing to be bound by theory, the ability to produce large amounts of EEV compared to other clonal results indicate that VIP02 may have a higher ability to evade host immune systems and can spread better within, and among, tumors in the body and, thus, VIP02 is particularly suitable as an oncolytic virus therapy.

### Example 2: Assessment of Tumor Growth Inhibition by VACV Clonal Isolates

The oncolytic activity of VACV clonal isolates, including VIP02, on tumor growth inhibition was tested in vivo and in vitro.

### A. Tumor Growth Inhibition in a Syngeneic Mouse Model

The tumor inhibitory activity of clonal isolates of the Copenhagen and the IHD-J VACV clone isolates was evaluated in vivo using the 4T1 mouse mammary gland carcinoma model.

4T1 cells (ATCC) that stably express red fluorescent protein (RFP), designated 4T1-RFP cells, were used to generate the 4T1 mouse mammary gland carcinoma model. Female BALB/c mice, 4-6 weeks old purchased from Taconic Biosciences Inc. (Rensselaer, NY) were injected subcutaneously (s.c.) into the flank with 2×106 4T1-RFP cells in 100 μL of PBS to generate tumor stocks. The resulting tumor stock tissues were harvested, inspected, and any suspected or grossly necrotic tissue or tumor tissues were removed. Healthy tumor tissue was subsequently cut into small fragments of approximately 1 mm³ and used for orthotopic tumor implantation. All surgical procedures were performed under an 8× magnification microscope under a HEPA-filtered laminar flow hood. Animals were anesthetized by intramuscular injection of a ketamine mixture. The surgical area was sterilized using iodine and alcohol. An incision of approximately 1 cm long was made in the right second mammary gland of the mouse using surgical scissors. The mammary gland was exposed, the capsule at the transplantation site was stripped, and one 4T1-RFP tumor fragment (1 mm³) was transplanted and secured with 8-0 surgical sutures (nylon). The incision was closed with 5-0 surgical sutures.

Virus treatment was initiated when tumor volumes were approximately 100 mm³. Mice were treated (10 mice per group) once with an intravenous dose of 1×10⁷ PFU of VCP02, VIP01 and VIP02. Tumor volume and body weight were measured twice per week using a vernier caliper and an electronic scale, respectively. Tumor volume was estimated by measuring the perpendicular minor dimension (W) and major dimension (L). Approximate tumor volume was calculated with the formula (W²×L)/2. Mice were euthanized when tumors reached termination criteria (tumor volume >=3000 mm³ and/or body weight loss >=20%).

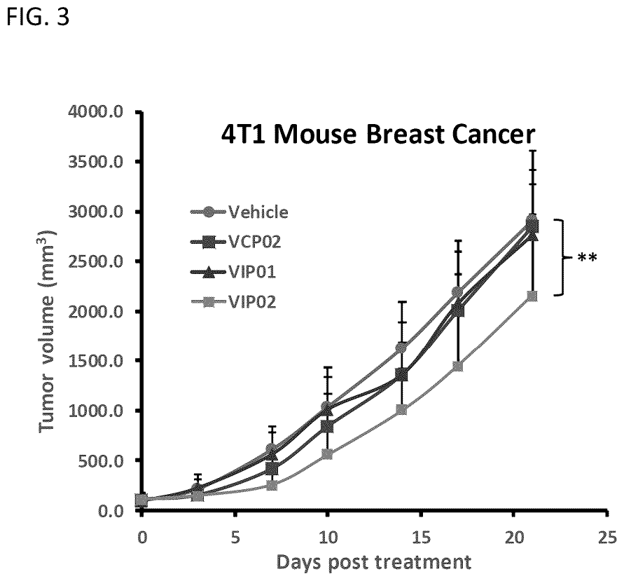

As shown in FIG. 3, a single intravenous delivery of VIP02 at a low dose significantly inhibited tumor growth in the 4T1 mouse mammary gland carcinoma model compared to vehicle only control. In contrast, neither VIP01 nor VCP02 clonal isolates were effective in inhibiting tumor growth.

### B. Tumor Cell Killing In Vitro

To further confirm oncolytic activity, tumor cell killing was assessed in vitro in 2-D and 3-D cultures of different cancer cell lines. The assays were carried out with a variety of different human cancer cell lines: BT-549, NCI-H226, SW1990, M14, LOX-IMVI, COLO 205, U-251, 786-0 or PC-3 cancer cell for 2-D culture assays and MDA-MB-468, A549, M14, HCC-2998, COLO 205, U-251, 786-O, PC-3, 4T1 and CT26.WT for 3-D cell culture assays. The human cancer cell lines were purchased from ATCC or were obtained from National Cancer Institute.

For the 2-D cell culture, cells were seeded into 96-well microplates (Corning, Kennebunk, ME) at a concentration of 3,000 cells per well and incubated overnight at 37° C. under 5% (v/v) CO₂. Cells were then infected with VIP02 at MOI=0.1 (darker bars) and MOI=0.01 (lighter bars). Cell viability was measured at 96 hours post infection using CellTiter 96® AQueous One Solution Cell Proliferation Assay (Promega, Madison, WI) according to the manufacturer's instructions. The percent of viable cells (% survival) was determined.

For the 3-D cell cultures, cells were seeded into 96-well spheroid microplates (Corning, Kennebunk, ME) at a concentration of 30,000 cells per well and centrifuged at 500 g for 5 minutes. After being incubated for 4 days at 37° C. under 5% (v/v) CO₂, cells were infected with VIP02 at MOI=0.1 (darker bars) and MOI=0.01 (lighter bars). Five days after infection (120 hours), cell viability was measured using the CellTiter-Glo 3D Viability Assay (Promega, Madison, WI) according to the manufacturer's instructions. The percentage of viable cells (% survival) was determined.

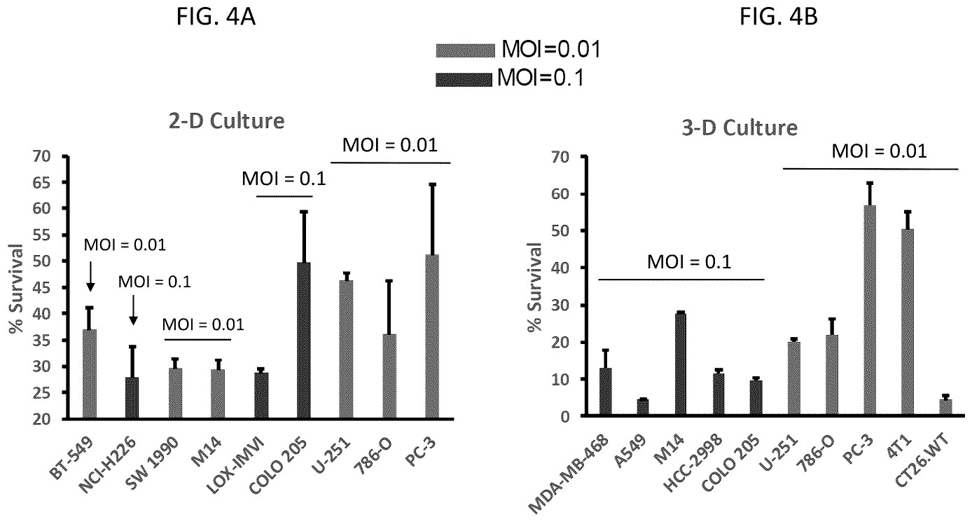

As shown in FIG. 4A (2-D culture) and FIG. 4B (3-D culture), VIP02 had a potent in vitro tumor cell killing activity in both 2-D and 3-D cultures.

Example 3: Generation and Assessment of Recombinant VIP02-Based Viral Clones Engineered with a Gene Encoding a Heterologous Stealth Protein

The VIP02 clonal isolate was chosen as a parent virus for subsequent genetic engineering. A gene encoding a stealth protein was engineered into the VIP02. The exemplary stealth proteins that were engineered into recombinant strains as described in this example include Borrelia burgdorferi complement regulatory-acquiring surface protein-2 (CRASP-2), minimized complement regulator factor H (miniFH) and Cowpox virus ORFs 012, 203 and 018 (CPXV012-203-018).

### A. Generation of Stealth Recombinant Vaccinia Viruses

Recombinant VIP02 VACV strains were generated by homologous recombination using standard techniques. In this method, a gene encoding the heterologous protein (or heterologous gene product) was cloned by standard recombinant DNA techniques into a transfer vector plasmid containing a desired segment of vaccinia DNA to target a particular locus in the VIP02 clonal isolate. Typically, the DNA is inserted into a non-essential gene, for example, the F14.5 gene, and is linked to a vaccinia promoter. The heterologous gene was then inserted into a locus of the genome of the clonal isolate by double reciprocal crossover.

### 1. Transfer Vector Plasmid Construction

Transfer vector plasmids generated to contain a stealth gene were generated as follows:

pIA35R: The left and right flanking sequences of the A35R gene of VACV IHD-J were PCR-amplified from VACV IHD-J genomic DNA using Q5 High-Fidelity 2× Master Mix (New England Biolabs Inc., Ipswich, MA) and the primers: 5′-GCTGAATTCGTAGGTTTAAGAAGTTGTCGGTGC-3′ (SEQ ID NO: 43) and 5′-TTTTTGTTGTCACTTGTAGAATTTTTTAACACATAGTAC-3′ (left flank) (SEQ ID NO: 44), 5′-TACTATGTGTTAAAAAATTCTACAAGTGACAACAAAAACTGCAGCGGCCGCTCGAGAGCTC AGACGGCAATGGATGGATCATAATTGATG-3′ (SEQ ID NO: 45) and 5′-GCGAAGCTTATCTTCATTAAGTACTTTAACAGTC-3′ (right flank) (SEQ ID NO: 46). The two fragments were joined together using the method of gene splicing by overlapping extension (Horton R M, Ho S N, Pullen J K, Hunt H D, Cai Z, Pease L R. Gene splicing by overlap extension. Methods Enzymol. 1993; 217:270-9. Epub 1993 Jan. 1. doi: 10.1016/0076-6879 (93) 17067-f. PubMed PMID: 8474334). The resulting fragment was digested with EcoRI and HindIII and cloned into the same-cut plasmid pVM-1 to yield pIA35R. The flanking sequences of A35R were confirmed by DNA sequencing.

pITK: To generate pITK, the left and right flanking sequences of the J2R (thymidine kinase; TK) gene of VACV IHD-J were PCR-amplified from VACV IHD-J genomic DNA using the Q5 High-Fidelity 2× Master Mix (New England Biolabs Inc., Ipswich, MA) and the primers: 5′-GCTGAATTCTTTTGCGATCAATAAATGGATCACAACCAG-3′ (SEQ ID NO: 35) and 5′-AACTCGTCTAATTAATTCTGTACTTTTACCTG-3′ (left flank) (SEQ ID NO: 36), 5′-CCATGTTTTCAGGTAAAAGTACAGAATTAATTAGACGAGTTGGTACCTGCAGCGGCCGCTCG AGAGCTCGGAGGTAATGATATGTATCAATCGGTGTGTAG-3′ (SEQ ID NO: 37) and 5′-GCGAAGCTTCGTAATTACTTAGTAAATCCGCCGTACTAGG-3′ (right flank) (SEQ ID NO: 38). The two fragments were joined together using the method of gene splicing by overlapping extension (Horton et al., 1993). The resulting fragment was digested with EcoRI and HindIII and cloned into the same-cut plasmid pVM-1 to yield pITK. The flanking sequences of TK were confirmed by DNA sequencing.

Plasmid pIF14.5L-CRASP-2. To construct the pIF14.5L-CRASP-2 plasmid, the left flanking sequence of the F14.5L gene of VACV IHD-J was amplified by PCR from the VACV IHD-J genomic DNA using the Q5 High-Fidelity 2× Master Mix (New England Biolabs Inc., Ipswich, MA) and the primers: 5′-GCTCAATTGCTAGCTCGATGCTTTGTTAAAATAGATACTCCTAG-3′ (SEQ. NO ID: 31) and 5′-CAACAGTAGTTCTTGCTCCTCCTTGATTC-3′ (SEQ. NO ID: 32). The Borrelia burgdorferi complement regulator-acquiring surface protein-2 (CRASP-2, SEQ ID NO: 18) cDNA fused with the right flanking sequence of F14.5L was synthesized by IDT (Coralville, IA). The two fragments were joined together using the method of gene splicing by overlapping extension (Horton et al., 1993) and the primers 5′-GCTCAATTGCTAGCTCGATGCTTTGTTAAAATAGATACTCCTAG-3′ (SEQ. NO ID: 33) and 5′-GCGAAGCTTGACTTTGTAGCTCTCCCAGATTTCTTTTC-3′ (SEQ. NO ID: 34).

The fragment from above was then cloned into plasmid VM-1 (pVM-1). To generate pVM-1, human codon-optimized Escherichia coli guanine phosphoribosyltransferase (gpt) gene driven by the VACV early promoter p7.5E was synthesized by Integrated DNA Technologies (IDT, Coralville, IA) and digested with EcoRI and NdeI. The digested fragment was cloned into the same-cut plasmid pUC19 to yield the plasmid pVM-1. The sequence of the gpt expression cassette was confirmed by DNA sequencing. The fragment with CRASP-2 fused to the left and right flanking sequences of F14.5L from above was digested with MfeI and HindIII, then cloned into the plasmid pVM-1 that was cut with EcoRI and HindIII to yield pIF14.5L-CRASP-2. The DNA sequence of F14.5L-CRASP-2 was confirmed by DNA sequencing.

Plasmid pIF14.5L-miniFH. To construct plasmid pIF14.5L-miniFH, the left flanking sequence of the F14.5L gene of VACV IHD-J was PCR-amplified from the VACV IHD-J genomic DNA using Q5 High-Fidelity 2× Master Mix (New England Biolabs Inc., Ipswich, MA) and the primers: 5′-GCTCAATTGCTAGCTCGATGCTTTGTTAAAATAGATACTCCTAG-3′ (SEQ. NO ID: 31) and 5′-CAACAGTAGTTCTTGCTCCTCCTTGATTC-3′ (SEQ NO ID: 32). The minimized complement regulator factor H (miniFH, SEQ ID NO: 19) cDNA fused with the right flanking sequence of F14.5L was synthesized by IDT (Coralville, IA). The two fragments were joined together using the method of gene splicing by overlapping extension (Horton et al., 1993) and the primers 5′-GCTCAATTGCTAGCTCGATGCTTTGTTAAAATAGATACTCCTAG-3′ (SEQ. NO ID: 33) and 5′-GCGAAGCTTGACTTTGTAGCTCTCCCAGATTTCTTTTC-3′ (SEQ. NO ID: 34). The resulting fragment was digested with MfeI and HindIII, then cloned into the plasmid pVM-1 and cut with EcoRI and HindIII to yield pIF14.5L-miniFH. The DNA sequence of F14.5L-miniFH was confirmed by DNA sequencing.

Plasmid pITK-CPXV012-203-018. The DNA fragment containing cowpox virus (CPXV) open-reading frames 012 (SEQ ID NO: 20), 203 (SEQ ID NO: 21) and 018 (SEQ ID NO: 22) with their own original promoters was synthesized by IDT (Coralville, IA).

The fragment from above was then cloned into plasmid ITK (pITK). The synthesized fragment containing CPXV open-reading frames 012, 203 and 018 from above was digested with KpnI and SacI and cloned into the same-cut plasmid pITK to yield pITK-CPXV012-203-018. The DNA sequence of CPXV012-203-018 was confirmed by DNA sequencing.

### 2. Homologous Recombination and Selection of Recombinant Viruses

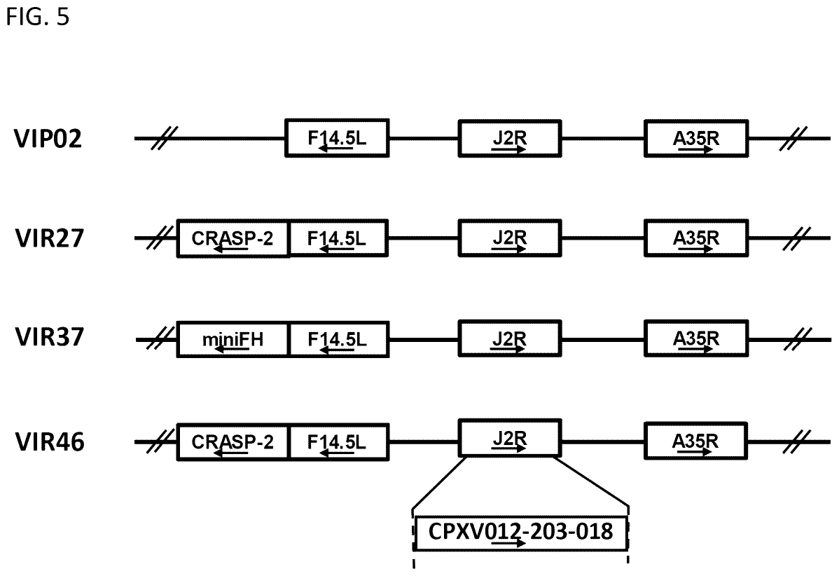

To generate the recombinant vaccinia viruses, CV-1 cells were infected with parental viruses as indicated in Table E2 at a multiplicity of infection (MOI) of 0.1 for 1 hour and then transfected with transfer vectors, as indicted in Table E2 below and FIG. 5, using jetPRIME in vitro DNA & siRNA transfection reagent (Polyplus-transfection Inc., New York, NY). Two days post infection, infected/transfected cells were harvested and recombinant viruses were selected and plaque purified as described previously ((Falkner F G, Moss B. Transient dominant selection of recombinant vaccinia viruses. J Virol. 1990; 64(6): 3108-11. Epub 1990 Jun. 1. PubMed PMID: 2159565).

Table E2 and FIG. 5 summarize the generated recombinant VACV strains, including the parental virus and transfer vector used in their generation.

TABLE E2

| Recombinant | | | |
|---|---|---|---|
| VACVs | Parent Virus | Transfer Vector | Genotype |
| VIR11 | VIP02 | pIA35R | A35R is |
| (SEQ ID NO: 3) | (SEQ ID NO: 1) | | disrupted |
| VIR52 | VIR11 | pITK | A35R and J2R |
| (SEQ ID NO: 12) | (SEQ ID NO: 3) | | are disrupted |
| VIR27 | VIP02 | pIF14.5L- | F14.5L fused |
| (SEQ ID NO: 5 ) | (SEQ ID NO: 1) | CRASP-2 | with CRASP-2 |
| VIR37 | VIP02 | pIF14.5L- | F14.5L fused |
| (SEQ ID NO: 6 ) | (SEQ ID NO: 1) | miniFH | with miniFH |
| VIR46 | VIR27 | pITK-CPXV012- | F14.5L fused |
| (SEQ ID NO: 10) | (SEQ ID NO: 5) | 203-018 | with CRASP- |
| | | | 2; CPXV012, |
| | | | 018 and 203 |
| | | | inserted |
| | | | at J2R |

### B. Assessment of Immune Evasion and Oncolytic Activity

### 1. Stealth Virus In Vitro Avoidance of Host Immune Response

Recombinant virus strains VIR27, VIR37 and VIP02 were assessed for their ability to escape inhibition by complement. For the complement inhibition assay, 2 μL of virus (10⁷ pfu/mL) were mixed with 100 μL of normal human serum (Biochemed Services, Winchester, VA), BALB/c mouse serum (Biochemed Services, Winchester, VA), or DMEM-2.5 in duplicate, respectively, and then the mix was incubated at 37° C. for 1 hour. Virus concentration after incubation was calculated by titration in CV-1 cells and the percent inhibition of the virus titer was determined by comparing the virus titer after incubation with serum with the virus titer after incubation with DMEM-2.5 (FIG. 6).

As shown in FIG. 6, VIP02, and VIP02-based stealth oncolytic viruses VIR27 and VIR37, can escape complement inhibition when assessed in human and mice serum. Moreover, the percentage of inhibition was significantly lower in the VIP02-based stealth oncolytic viruses VIR27 and VIR37 when compared to VIP02, indicating that the introduction of CRASP-2 and miniFH into VIP02 significantly increases the ability of the virus to inhibit the host complement.

### 2. Stealth Virus In Vivo Induced Tumor Size Reduction in Mice

The tumor inhibitory activity of VIP02 and the engineered stealth clone VIR27 expressing CRASP-2 was evaluated in vivo using the 4T1 mouse mammary gland carcinoma model, as described above in Example 2. In this experiment, virus treatment was initiated when tumor volumes were approximately 80 mm³. Mice were treated (8 mice per group) once with an intravenous dose of 1×10⁷ PFU of VIP02 or VIR27. As shown in FIG. 7, mice injected with the VIP02 and VIR27 strains showed a reduction in tumor volume at 9 days after treatment when compared to vehicle treated controls. Furthermore, the stealth strain VIR27 was more efficient than VIP02 at reducing tumor growth, as shown by a statistically significant reduction in tumor volume at 7 days post treatment. These results are consistent with a finding that introduction of the exogenous CRASP-2 protein potentiates the ability of the virus to inhibit complement.

In another similar study, the tumor inhibitory activity of stealth VIR46 (cowpox virus, CPXV, open-reading frames 012, 203 and 018 with their own original promoters) and VIR52 (A35R and J2R are disrupted) was evaluated in vivo using the 4T1 mouse mammary gland carcinoma model, as described above in Example 2. In this experiment, virus treatment was initiated when tumor volumes were approximately 90 mm³. Mice were treated (6 mice per group) once with an intravenous dose of 1×10⁷ PFU of VIP46 or VIR52. As shown in FIG. 8, mice injected with the VIR46 strain showed a statistically significant reduction in tumor volume at 20 days after treatment when compared to vehicle treated mice. In addition, VIR46 showed a significantly improved ability to reduce tumor volume when compared with VIR52.

Example 4: Generation and Assessment of Recombinant VIP02-Based Viral Clones Engineered with a Gene Encoding a Heterologous Immune Stimulating Protein

The VIP02 clonal isolate was chosen as a parent virus for subsequent genetic engineering with an exemplary immune stimulating gene encoding a human LIGHT mutant (hmLIGHT). The generated recombinant virus was evaluated for the ability to inhibit tumor growth.

### A. Generation of Immune Stimulating Recombinant Vaccinia Viruses

A recombinant VACV strain with immune stimulating activity, VIR49, was generated by insertion of the human LIGHT mutant (hmLIGHT) cDNA under the control of the VACV SSE promoter into the endogenous viral J2R gene. The recombinant VIR49 was generated by homologous recombination of a transfer vector and the parental genome.

The transfer vector was generated as follows:

Plasmid pIA35R-SSE-hmLIGHT. The human LIGHT mutant (hmLIGHT, SEQ ID NO: 25) cDNA under the control of the VACV SSE promoter was synthesized by IDT (Coralville, IA). The synthesized fragment was digested with PstI and SacI and cloned into the same-cut plasmid pIA35R described in Example 3 to yield pIA35R-SSE-hmLIGHT. The DNA sequence of SSE-hmLIGHT was confirmed by DNA sequencing.

Parental virus VIR13 was generated by infecting CV-1 cells with VIP02 at an MOI of 0.1 for 1 hour and then transfected with pITK transfer vector described in Example 3, to yield a parental recombinant virus in which the J2R (TK) gene was disrupted. To generate the recombinant VIR49, CV-1 cells were infected with the parental virus VIR13 at an MOI of 0.1 for 1 hour and then transfected with the pIA35R-SSE-hmLIGHT transfer vector. For comparison, recombinant vaccinia strain VIR52 also was generated by infecting CV-1 cells with the parental strain VIR11 at an MOI of 0.1 for 1 hour and then transfected with the pITK transfer vector substantially as described in Example 3.

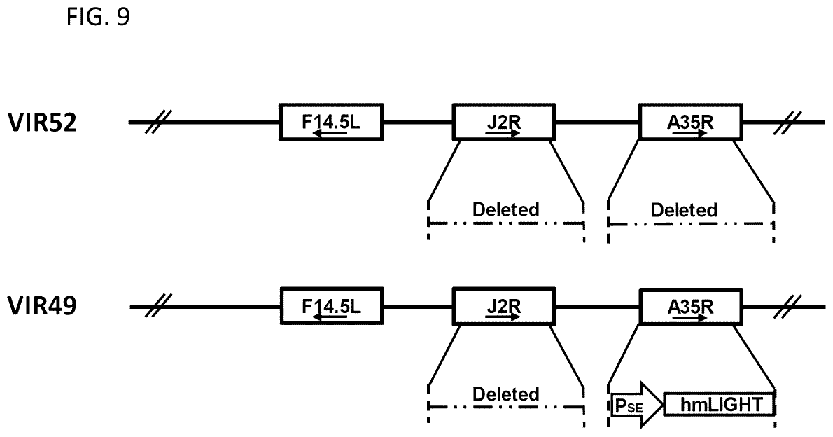

Table E3 and FIG. 9 summarize the generated recombinant VACV strains, including the parental virus and transfer vectors used in their generation.

TABLE E3

| Recombinant | | Transfer | |
|---|---|---|---|
| VACVs | Parent Virus | Vector | Genotype |
| VIR11 | VIP02 | pIA35R | A35R is disrupted |
| VIR52 | VIR11 | pITK | A35R and J2R are |
| (SEQ ID NO: 12) | (SEQ ID NO: 3) | | disrupted |
| VIR13 | VIP02 | pITK | J2R(TK) is disrupted |
| (SEQ ID NO: 4) | | | |
| VIR49 | VIR13 | pIA35R- | J2R(TK) is disrupted, |
| (SEQ ID NO: 11) | (SEQ ID NO: 4) | SSE- | SSE-hmLIGHT |
| | | hmLIGHT | inserted |
| | | | at A35R |

### B. Assessment of Oncolytic Activity

The tumor inhibitory activity of VIR49 (mutant hLIGHT with immune system stimulating activity) was evaluated in vivo using the 4T1 mouse mammary gland carcinoma model as described above in Example 2. In this experiment, virus treatment was initiated when tumor volumes were approximately 90 mm³. Mice were treated (6 mice per group) once with an intravenous dose of 1×10⁷ PFU of VIR49 or VIR52.

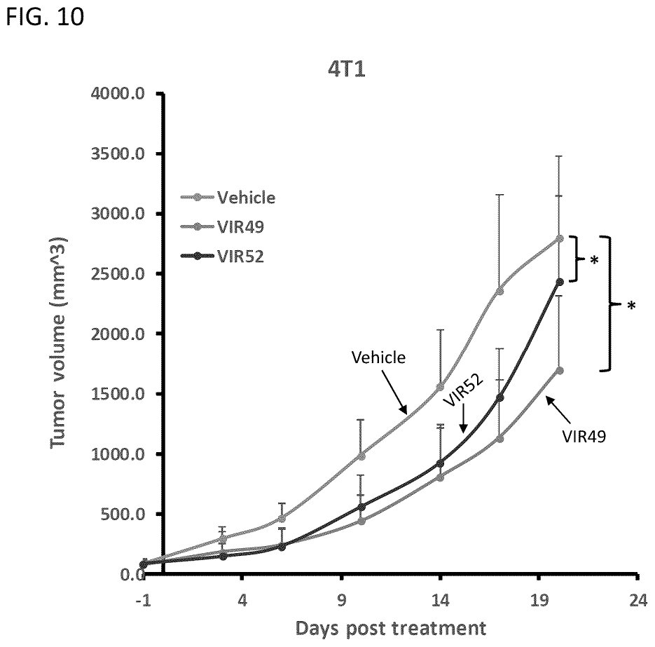

As shown in FIG. 10, mice treated with VIR49 and VIR52 showed a statistically significant reduction in tumor volume when compared to the vehicle treated controls. Moreover, mice treated with VIR49 showed a significantly improved efficacy at reducing tumor volume 20 days after treatment when compared to VIR52 and the vehicle treated controls, indicating that the introduction of the hmLIGHT sequence increases the ability of the virus to stimulate the immune system.

Example 5: Generation and Assessment of Recombinant VIP02-Based Viral Clones Engineered with a Gene Encoding an Anti-Angiogenic Protein

The VIP02 clonal isolate was chosen as a parent virus for subsequent genetic engineering with exemplary anti-angiogenic proteins, namely, anti-VEGF single chain antibody (scab) fused with anti-angiopoietin-2 scAb (anti-VEGF-anti-Ang2) (SEQ ID NO: 23) and human versikine (VK) (SEQ ID NO: 24) (anti-VEGF-anti-Ang2-SEL-VK). The generated recombinant virus was evaluated for the ability to inhibit tumor growth.

### A. Generation of Anti-Angiogenic Recombinant Vaccinia Viruses

A recombinant vaccinia virus clone with anti-angiogenic activity, VIR71, was generated by inserting the mH5-anti-VEGF-anti-Ang2-SEL-VK construct into the endogenous viral J2R gene. The recombinant VIR71 was generated by homologous recombination of a transfer vector and the parental genome.

The transfer vectors used in the recombinant virus generation were generated as follows:

pITK-mH5-hFGL1: The human fibrinogen like 1 (hFGL1) cDNA under the control of the VACV modified H5 early/late promoter (mH5) was synthesized by IDT (Coralville, IA). The synthesized fragment was digested with KpnI and SacI and cloned into the same-cut plasmid pITK described in Example 3 to yield pITK-mH5-hFGL. The DNA sequence of mH5-hFGL was confirmed by DNA sequencing.

pITK-mH5-Versikine: pITK-mH5-hFGL1 was digested with PstI and SacI. The fragment containing the backbone plasmid and the mH5 promoter (pITK-mH5) was gel-purified. The human versikine was synthesized by IDT (Coralville, IA) and digested with PstI and SacI, then cloned into the same-cut plasmid pITK-mH5 to yield pITK-mH5-Versikine. The DNA sequence of mH5-Versikine was confirmed by DNA sequencing.

pITK-mH5-anti-VEGF-anti-Ang2: pITK-mH5-hFGL1 was digested with PstI and SacI. The fragment containing the backbone plasmid and the mH5 promoter (pITK-mH5) was gel-purified. The anti-VEGF single chain antibody (scAb) fused with anti-angiopoietin-2 scAb was synthesized by IDT (Coralville, IA) and digested with PstI and SacI, then cloned into the same-cut plasmid pITK-mH5 to yield pITK-mH5-anti-VEGF-anti-Ang2. The DNA sequence of mH5-anti-VEGF-anti-Ang2 was confirmed by DNA sequencing.

pITK-mH5-anti-VEGF-anti-Ang2-SEL-VK: pITK-mH5-hFGL1 was digested with PstI and SacI. The fragment containing the backbone plasmid and the mH5 promoter (pITK-mH5) was gel-purified. The fragment containing anti-VEGF-anti-Ang2 was PCR-amplified from pITK-mH5-anti-VEGF-anti-Ang2 using Q5 High-Fidelity 2× Master Mix (New England Biolabs Inc., Ipswich, MA) and the primers: 5′-CGACTGCAGACCATGGAAACCGACACAC-3′ (SEQ ID NO: 39) and 5′-CAATTTTTATAAAAATTAATTAATCATTGTCC-3′ (SEQ ID NO: 40). The fragment containing human versikine driven by the VACV synthetic strong early/late promoter (SEL) was PCR-amplified from pITK-mH5-Versikine using Q5 High-Fidelity 2× Master Mix (New England Biolabs Inc., Ipswich, MA) and the primers: 5′-GGACAATGATTAATTAATTTTTATAAAAATTGAAATTTTATTTTTTTTTTTTGGAATATAAAT ACTCGAGACCATGTTCATAAACATAAAATCC-3′ (SEQ ID NO: 41) and 5′-GCTGAGCTCATAAAAATTAATTAATCATTCAGC-3′ (SEQ ID NO: 42). The two fragments were joined together using the method of gene splicing by overlapping extension (Horton et al., 1993). The resulting fragment was digested with PstI and SacI, then cloned into the same-cut plasmid pITK-mH5 to yield pITK-mH5-anti-VEGF-Ang2-SEL-VK. The DNA sequence of mH5-anti-VEGF-Ang2-SEL-VK was confirmed by DNA sequencing.

To generate the recombinant vaccinia virus VIR71, CV-1 cells were infected with parental virus VIR11 at a multiplicity of infection (MOI) of 0.1 for 1 hour and then transfected with pITK-mH5-anti-VEGF-anti-Ang2-SEL-VK transfer vector. For comparison, recombinant vaccinia strain VIR52 also was generated by infecting CV-1 cells with the parental strain VIR11 at a MOI of 0.1 for 1 hour and then transfected with the pITK transfer vector substantially as described in Example 3.

To generate the recombinant vaccinia virus VIR86, CV-1 cells were infected with parental virus VIR49 at a multiplicity of infection (MOI) of 0.1 for 1 hour and then transfected with pITK-mH5-anti-VEGF-anti-Ang2-SEL-VK transfer vector. For comparison, recombinant vaccinia strain VIR13 also was generated by infecting CV-1 cells with the parental strain VIR02 at a MOI of 0.1 for 1 hour and then transfected with the pITK transfer vector substantially as described in Example 4.

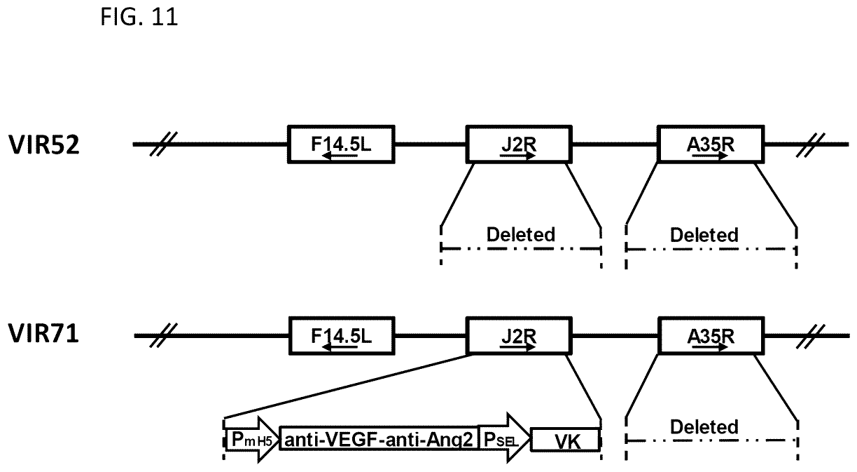

Table E4 and FIG. 11 summarize the generated recombinant VACV strains, including the parental virus and transfer vectors used in their generation.

TABLE E4

| Recombinant | | Transfer | |
|---|---|---|---|
| VACVs | Parent Virus | Vector | Genotype |
| VIR11 | VIP02 | pIA35R | A35R is disrupted |
| VIR52 | VIR11 | pITK | A35R and J2R |
| (SEQ ID NO: 12) | (SEQ ID NO: 3) | | are disrupted |
| VIR71 | VIR11 | pITK-mH5- | A35R is disrupted; |
| (SEQ ID NO: 13) | (SEQ ID NO: 3) | anti-VEGF- | mH5-anti- |
| | | anti-Ang2- | VEGF-anti- |
| | | SEL-VK | Ang2-SEL-VK |
| | | | inserted at J2R |
| VIR86 | VIR49 | pITK-mH5- | SSE-hmLIGHT |
| (SEQ ID NO: 47) | (SEQ ID NO: 11) | anti-VEGF- | inserted at |
| | | anti-Ang2- | A35R; mH5- |
| | | SEL-VK | anti-VEGF-anti- |
| | | | Ang2-SEL-VK |
| | | | inserted at J2R |

### B. Assessment of Oncolytic Activity

The tumor inhibitory activity of VIR71 was evaluated in vivo using the 4T1 mouse mammary gland carcinoma model described above in Example 2. In this experiment, virus treatment was initiated when tumor volumes were approximately 90 mm³. Mice were treated (6 mice per group) once with an intravenous dose of 1×10⁷ PFU of VIR71 or VIR52.

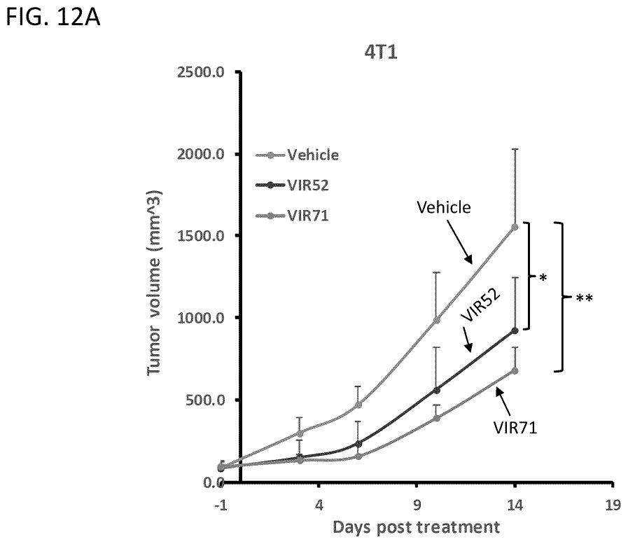

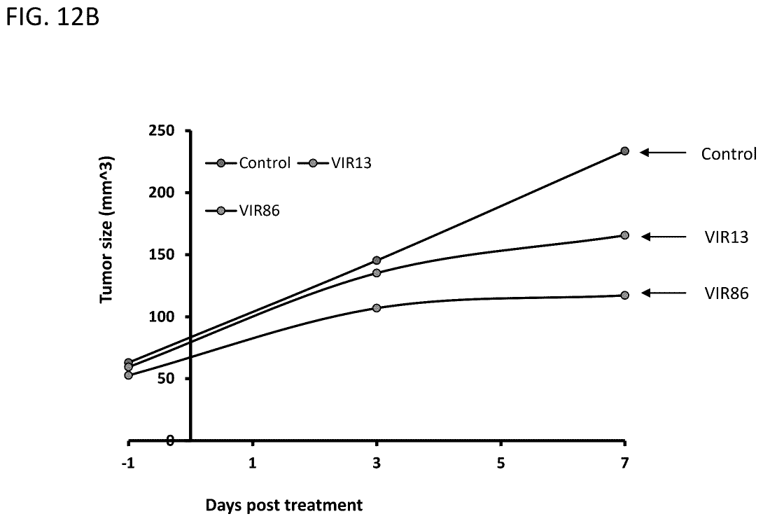

As shown in FIG. 12A, mice injected with the anti-angiogenesis VIR71 strain showed a statistically significant reduction in tumor volume 14 days after treatment when compared with VIR52 and the vehicle treated controls. Also as shown in FIG. 12B, mice injected with the anti-angiogenesis VIR86 showed a reduction in tumor volume across 7 days post treatment when compared to both vehicle treated control and VIR13. The data demonstrates that the anti-angiogenesis VIR71 and VIR 86 strains each showed a statistically significant reduction in tumor volume when compared with the vehicle treated controls and while VIR52 and VIR13 each reduced tumor size, the VIR71 and VIR 86 strains showed a greater reduction in tumor volume than the control VIR52 and VIR13 strains. Versikine, a molecule that can act as an immune modulator, did not significantly reduced tumor size when assessed alone (data not shown). However, when fused to anti-VEGF-anti-Ang2, Versikine, significantly enhanced the tumor cell killing ability of VEGF-anti-Ang2.

Example 6: Generation and Assessment of Recombinant VIP02-Based Viral Clones Engineered with a Gene Encoding an Apoptosis Inducing Protein

The VIP02 clonal isolate was chosen as a parent virus for subsequent genetic engineering with exemplary apoptosis inducing proteins in order to generate a viral inducible system that can inhibit viral replication in healthy cells but not in cancer cells upon induction with Rimiducid, an FDA approved drug for use in humans. The exemplary apoptosis inducing proteins that were engineered into recombinant strains as described in this example include Rimiducid inducible iCasp9, iDED or iFAS constructs containing F36V-FKBP, a full length version of the human FK506 binding protein 12 (FKBP12, also known as FKBP) including a Phe to Val substitution (See Clackson et al. 1998 PNAS, 95:10437-10442) fused to either Casp9, DED or FAS. Rimiducid treatment induces FKBP12 dimerization that leads to activation of inducible Casp9, DED and FAS. The ability of these recombinant vaccinia virus strains to inhibit plaque formation, inhibit viral replication, and inhibit virus-mediated cytotoxicity was evaluated in primary healthy cells and cancer cells.

### A. Generation of Apoptosis Inducing Vaccinia Viruses

The recombinant vaccinia virus clones with apoptosis-inducing activity were generated by replacing the J2R gene in the VIP02 with DNA encoding iCasp9, iDED and iFAS protein. The recombinant strains VIR40, VIR41 and VIR42 were generated by homologous recombination of a transfer vector and the VIP02 parental genome, and VIR13 was used as a control virus.

The transfer vectors used in the recombinant virus generation were generated as follows:

pITK-SSE-hFGL1tm: The human fibrinogen like 1 (hFGL1) cDNA fused with the transmembrane domain of human CD19 under the control of the VACV synthetic strong early promoter (SSE) was synthesized by IDT (Coralville, IA). The synthesized fragment was digested with KpnI and SacI and cloned into the same-cut plasmid pITK described in Example 3 to yield pITK-SSE-hFGLtm. The DNA sequence of SSE-hFGLtm was confirmed by DNA sequencing.

Plasmid pITK-SSE-iCasp9. pITK-SSE-hFGL1tm was digested with PstI and SacI. The fragment containing the backbone plasmid and the SSE promoter (pITK-SSE) was gel-purified. The inducible caspase 9 (iCasp9, SEQ ID NO: 26) was synthesized by IDT (Coralville, IA) and digested with PstI and SacI, then cloned into the same-cut plasmid pITK-SSE to yield pITK-SSE-iCasp9. The DNA sequence of SSE-iCasp9 was confirmed by DNA sequencing.

Plasmid pITK-SSE-iDED. pITK-SSE-hFGL1tm was digested with PstI and SacI. The fragment containing the backbone plasmid and the SSE promoter (pITK-SSE) was gel-purified. The inducible death effector domain (DED, SEQ ID NO: 27) of the Fas-associated death domain-containing protein (FADD) was synthesized by IDT (Coralville, IA) and digested with PstI and SacI, then cloned into the same-cut plasmid pITK-SSE to yield pITK-SSE-iDED. The DNA sequence of SSE-iDED was confirmed by DNA sequencing.

pITK-SSE-iFAS. pITK-SSE-hFGL1tm was digested with PstI and SacI. The fragment containing the backbone plasmid and the SSE promoter (pITK-SSE) was gel-purified. The inducible Fas (SEQ ID NO: 28) was synthesized by IDT (Coralville, IA) and digested with PstI and SacI, then cloned into the same-cut plasmid pITK-SSE to yield pITK-SSE-iFAS. The DNA sequence of SSE-iFAS was confirmed by DNA sequencing.

To generate the recombinant vaccinia viruses, CV-1 cells were infected with parental virus VIP02 at a multiplicity of infection of 0.1 for 1 hour and then transfected with transfer vectors as indicated in Table E5 substantially as described in Example 3.

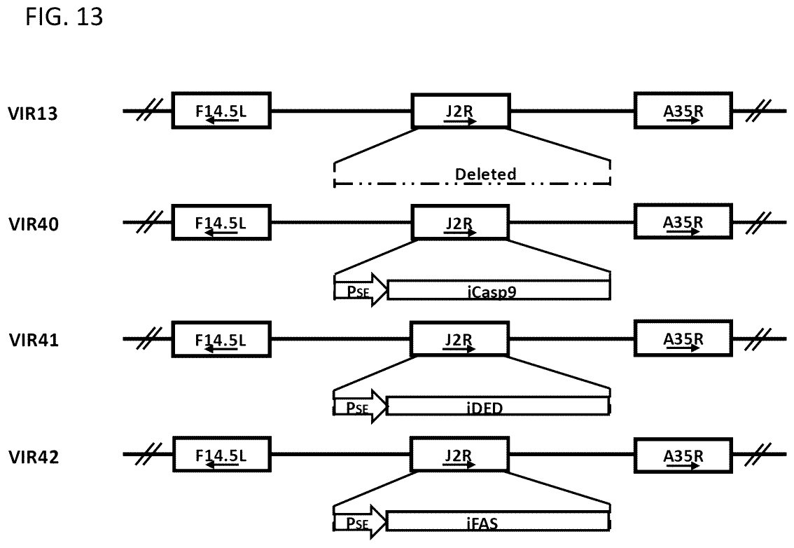

Table E5 and FIG. 13 summarize the generated recombinant VACV strains, including the parental virus and transfer vectors used in their generation.

TABLE E5

| Recombinant VACVs | Parent Virus | Transfer Vector | Genotype |
|---|---|---|---|
| VIR13 (SEQ ID NO: 4) | VIP02 (SEQ | pITK | J2R(TK) |
| | ID NO: 1) | | is disrupted |
| VIR40 (SEQ ID NO: 7) | VIP02 (SEQ | pITK-SSE- | SSE-iCasp9 |
| | ID NO: 1) | iCasp9 | inserted at J2R |
| VIR41 (SEQ ID NO: 8) | VIP02 (SEQ | pITK-SSE- | SSE-iDED |
| | ID NO: 1) | iDED | inserted at J2R |
| VIR42 (SEQ ID NO: 9) | VIP02 (SEQ | pITK-SSE- | SSE-iFAS |
| | ID NO: 1) | iFAS | inserted at J2R |

### B. Assessment of Apoptosis and Oncolytic Activity

d. iCasp9, iDED or iFAS Protein Expression

Expression of iCasp9, iDED and iFAS was assessed in infected cancer cells by monitoring the expression of F36V-FKBP.using an anti-FKBP12 antibody.

HCT-116 Human Colon Carcinoma cells were infected with VIR13, VIR40, VIR41 and VIR42 at an MOI of 10 in the presence of 10 nM Rimiducid or vehicle control. One day after infection, cells were harvested by low-speed centrifugation and lysed using RIPA lysis buffer (VWR, Solon, OH). A protease inhibitor cocktail (Promega, Madison, WI) was added to the lysis buffer. Protein amounts were determined using the RC DC Protein Quantification Kit (Bio-Rad Laboratories, Hercules, CA). Normalized protein amounts were subjected to an SDS-PAGE (Bio-Rad Laboratories, Hercules, CA) and transferred onto a PVDF membrane for Western blotting, which was blotted with primary antibodies followed by horseradish peroxidase (HRP)-conjugated secondary antibodies. Proteins were visualized with a chemiluminescent detection kit (ClarityMax Western ECL Substrate, Bio-Rad Laboratories, Hercules, CA) and Azure Biosystems c300 (Azure Biosystems, Dublin, CA). Antibodies against FKBP12 (1:1,000) and beta-actin (1:2,000) were from Novus Biologicals (Centennial, CO).

Western blot analysis of VIR13, VIR40, VIR41 and VIR42 extracts showed that iCasp9, iDED, and iFAS were expressed in VIR40, VIR41, and VIR42 infected cells, respectively (data now shown). In the same assay, no detectable signal for FKB12 was observed in cells infected with VIR13 consistent with the lack of engineering of the apoptosis inducing systems in this recombinant virus.

e. PARP1 protein expression in infected cells treated with Rimiducid

Activation of the inducible system with Rimiducid can be assessed by Western blot using an antibody that binds to PARP1 protein cleavage products, a marker of apoptosis. The ability of VIR40, VIR41 and VIR42 to induce apoptosis after FKBP12 dimerization induced by Rimiducid was assessed by Western blot of protein from infected human primary mammary epithelial cells and human primary colonic epithelial cells using an anti-PARP1 antibody. Anti-PARP1 (1:2,000) was from OriGene (Rockville, MD). As a control, the levels of PARP1 were also monitored in cells infected with VIR13.

Western blot results showed that Rimiducid induced cleavage of PARP1 in VIR40 and VIR41 but not in VIR13 or VIR42, indicating that iCasp9 and iDED induced apoptosis in infected human primary mammary epithelial cells and colonic epithelial cells in the presence of Rimiducid (data not shown). However, PARP1 cleavage was only detected in Hs578T human mammary gland duct carcinoma cells infected with VIR40 but not when infected with VIR13, VIR41 and VIR42, demonstrating that for cancer cells iCasp9, but not iDED and iFAS, induced apoptosis (data not shown).

f. Plaque Formation in the Presence of Rimiducid

As an alternative method for assessing apoptosis of infected cells, plaque formation after viral infection and treatment with Rimiducid was evaluated to investigate the ability of the VIR13, VIR40, VIR41 and VIR42 viral strains to inhibit viral replication in healthy and cancer cells (data not shown).

Cells were infected with VIR13, VIR40, VIR41 and VIR42 in the presence of each of 10 nM Rimiducid and vehicle control. Two or three days after infection, cells were stained with crystal violet. Pictures were taken using Azure Biosystems c300 (Azure Biosystems, Dublin, CA).

Table E6 summarizes the presence of plaque formation when healthy cells were infected with the recombinant virus strains. As expected, plaque formation was observed in all cells that were infected with the VIR13 control recombinant virus. However, Rimiducid treatment inhibited plaque formation in healthy cells infected with VIR40 and VIR41 but not when infected with VIR42 indicating that iCasp9 and iDED, but not iFAS, inhibit plaque formation in healthy cells.

TABLE E6

| Cell type | | Plaque formation | | | |
|---|---|---|---|---|---|
| Viral clone | Rimiducid | VIR13 | VIR40 | VIR41 | VIR42 |
| Human primary | − | + | + | + | + |
| mammary | + | + | − | − | + |
| epithelial cells | | | | | |
| Human primary | − | + | + | + | + |
| colonic | + | + | − | − | + |
| epithelial cells | | | | | |
| Human primary | − | + | + | + | + |
| bronchial/tracheal | + | + | − | − | + |
| epithelial cells | | | | | |
| Murine primary | − | + | + | + | + |
| mammary | + | + | − | − | + |
| epithelial cells | | | | | |

Table E7 summarizes the presence of plaque formation when various cancer cells were infected with the recombinant virus strains. As expected, plaque formation was observed in all cancer cells that were infected with the VIR13 control recombinant virus. Rimiducid treatment inhibited plaque formation in cancer cells infected with VIR40 but not in cancer cells infected with VIR41 and VIR42 indicating that iCasp9, but not iDED and iFAS, inhibit plaque formation in cancer cells.

Together, these results demonstrate that the iDED (VIR41) inducible system inhibits plaque formation in healthy cells but not in cancer cells after treatment with Rimiducid.

TABLE E7

| Cell type | | Plaque formation | | | |
|---|---|---|---|---|---|
| Viral clone | Rimiducid | VIR13 | VIR40 | VIR41 | VIR42 |
| 4T1 Mouse mammary | − | + | + | + | + |
| Gland Carcinoma | + | + | − | + | + |
| DU-145 Human | − | + | + | + | + |
| Prostate Carcinoma | + | + | − | + | + |
| PC-3 Human | − | + | + | + | + |
| Prostate Carcinoma | + | + | − | + | + |
| M14 Human | − | + | + | + | + |
| Amelanotic Melanoma | + | + | − | + | + |
| HCT-116 Human | − | + | + | + | + |
| Colon Carcinoma | + | + | − | + | + |
| HCT-15 Human | − | + | + | + | + |
| Colon | + | + | − | + | + |
| Adenocarcinoma | | | | | |
| MC-38 Murine | − | + | + | + | + |
| Colon | + | + | − | + | + |
| Adenocarcinoma | | | | | |
| CT26.WT Mouse | − | + | + | + | + |
| Colon Carcinoma | + | + | − | + | + |

### C. Apoptosis Mediated Inhibition of Virus Replication

The ability of the VIR13, VIR40, VIR41 and VIR42 recombinant viruses to inhibit virus replication after treatment with Rimiducid was evaluated in various healthy primary cells and cancer cells. Cells were infected with VIR13, VIR40, VIR41 and VIR42 at an MOI of 0.01 or 10 in duplicate in the presence of each of 10 nM Rimiducid and vehicle control. One day after infection, infected cells were harvested, and virus concentration was titrated in CV-1 cells.

When assessed in cultures of human primary bronchial/tracheal epithelial cells, human primary mammary epithelial cells, and murine primary mammary epithelial cells, Rimiducid treatment significantly inhibited virus replication in cells infected with VIR40 and VIR41, but not VIR13 and VIR42, indicating that iCasp9 and iDED, but not iFAS, inhibit viral replication in healthy cells (FIG. 14).

Rimiducid treatment significantly inhibited virus replication in breast, lung, melanoma and microsatellite instable (MSI) colorectal cancer cells infected with VIR40, but not with VIR41, VIR13 and VIR42, at both MOIs of 0.01 and 10, as demonstrated by BT-549 breast cancer cells (FIG. 15A), Hs578T breast cancer cells (FIG. 15B), MCF-7 and 4T1breast cancer cells (FIG. 15C), A549 and M14 lung and melanoma cancer cells (FIG. 15D), HCT-15 MSI colon cancer cells (FIG. 15E), HCT-116 MSI colon cancer cells (FIG. 15F), and KM12 MSI colon cancer cells (FIG. 15G). These results indicate that iCasp9 engineered into VIR40, but not iDED and iFAS, inhibits virus replication in breast, lung, melanoma and MSI colon cancer cells.

The ability of Rimiducid treatment to inhibit virus replication after infection of cells with VIR13, VIR40, VIR41 and VIR42 was also evaluated in MSS (Microsatellite Stable) colorectal cancer cells, a type of difficult to treat cancer that does not respond to checkpoint inhibitors. MSS colorectal cancer cell cultures were infected with VIR13, VIR40, VIR41 and VIR42 at MOIs of 0.01 or 10 in duplicate in the presence of each of 10 nM Rimiducid and vehicle control. One day after infection, infected cells were harvested and titrated in CV-1 cells. Rimiducid treatment significantly inhibited virus replication of MSS colorectal cells infected with VIR41 and VIR40, but not VIR13 and VIR42, as demonstrated by COLO205 cancer cells (FIG. 16A), HCC-2998 cancer cells (FIG. 16B), and HT-29 cancer cells (FIG. 16C). These results indicate that iCasp9 and iDED, but not iFAS, inhibits virus replication in MSS colorectal cancer cells after treatment with Rimiducid.

These results are consistent with a finding that MSS colon cancer cells respond differently than MSI colon cancer cells to infection. iDED induction with Rimiducid inhibits viral replication in MSS cancer cells but not in MSI cancer cells.

### D. Inhibition of Virus-Mediated Cytotoxicity by Apoptosis Inducing VACV Clones

The ability of Rimiducid treatment to inhibit virus-mediated cytotoxicity of different types of primary healthy and cancer cells after infection with VIR13, VIR40, VIR41 and VIR42 recombinant virus was evaluated.

Different types of cells were seeded into 96-well microplates (Corning, Kennebunk, ME) at a concentration of 3,000 cells per well and incubated overnight at 37° C. under 5% (v/v) CO₂. After incubation, cells were infected with VIR13, VIR40, VIR41 and VIR42 at MOIs of 0.01 and 0.1 in duplicate in the presence of 10 nM rimiducid and DMSO as a control. Cell viability was measured using the CellTiter 96® AQueous One Solution Cell Proliferation Assay (Promega, Madison, WI) according to manufacturer's instructions.

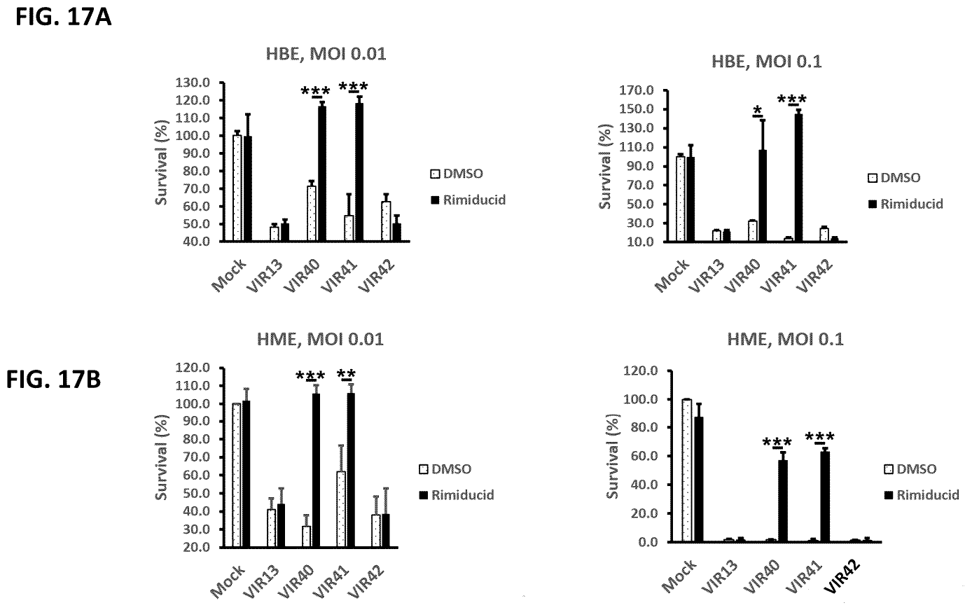

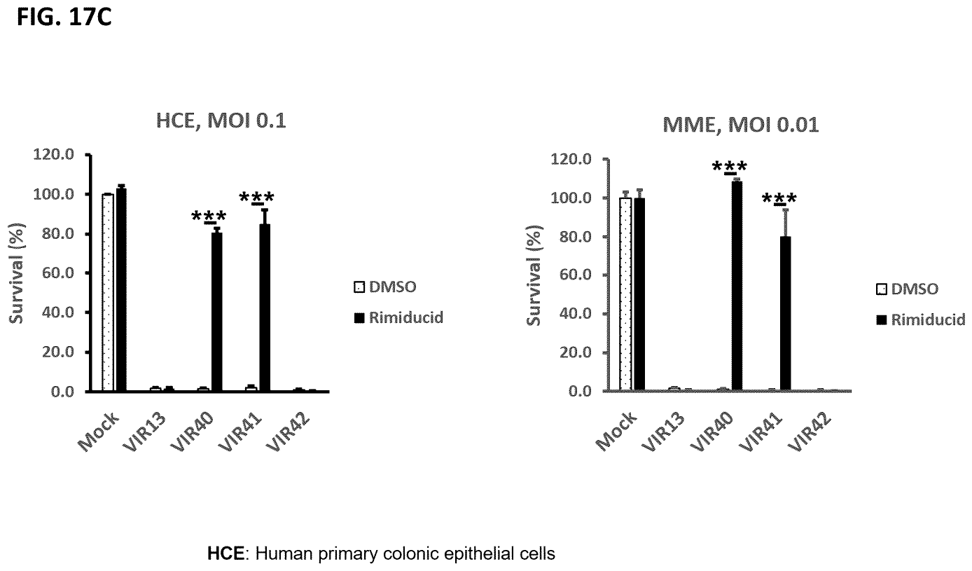

Rimiducid treatment significantly inhibited virus-mediated cytotoxicity after infection with VIR40 and VIR41, but not with VIR13 and VIR42, in human primary bronchial/tracheal epithelial cells (HBE, FIG. 17A), human primary mammary epithelial cells (HME, FIG. 17B), murine primary mammary epithelial cells (MME) and human primary colonic epithelial (HCE, FIG. 17C). These results indicate that iCasp9 and iDED, but not iFAS, inhibit virus-mediated cytotoxicity in healthy cells.

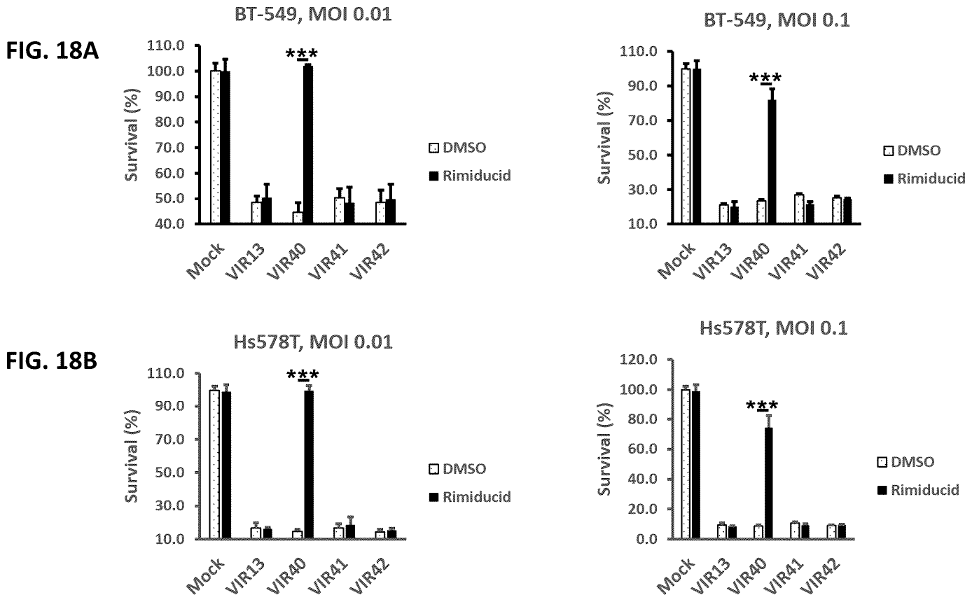

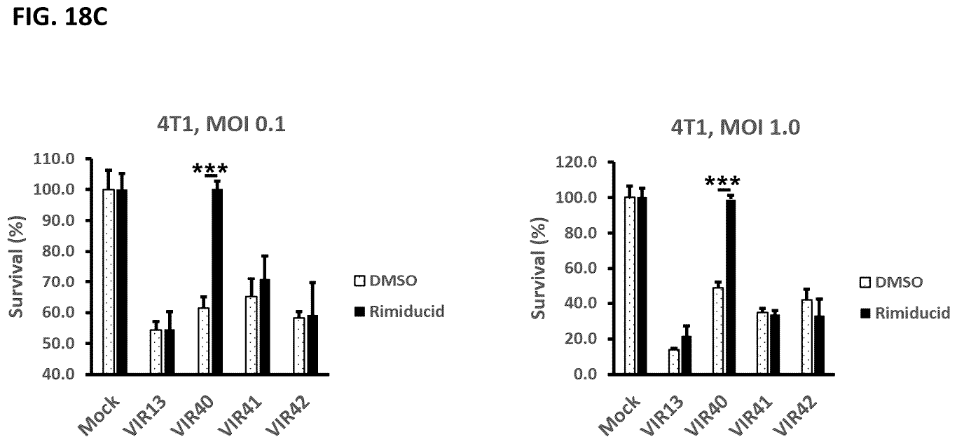

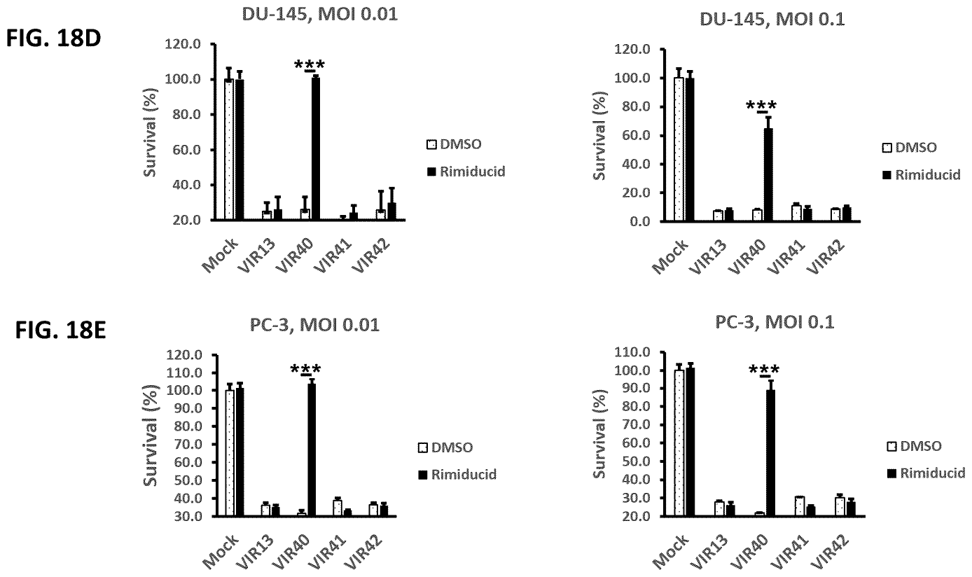

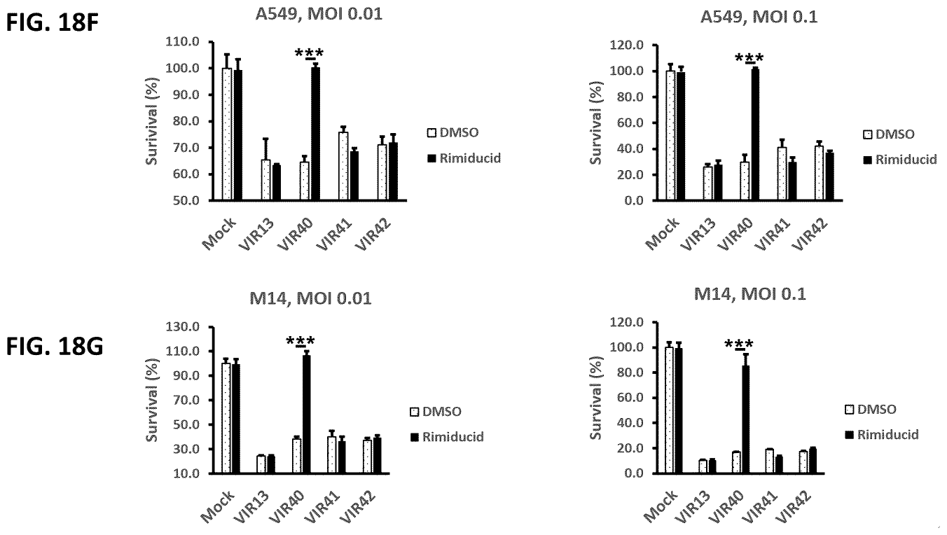

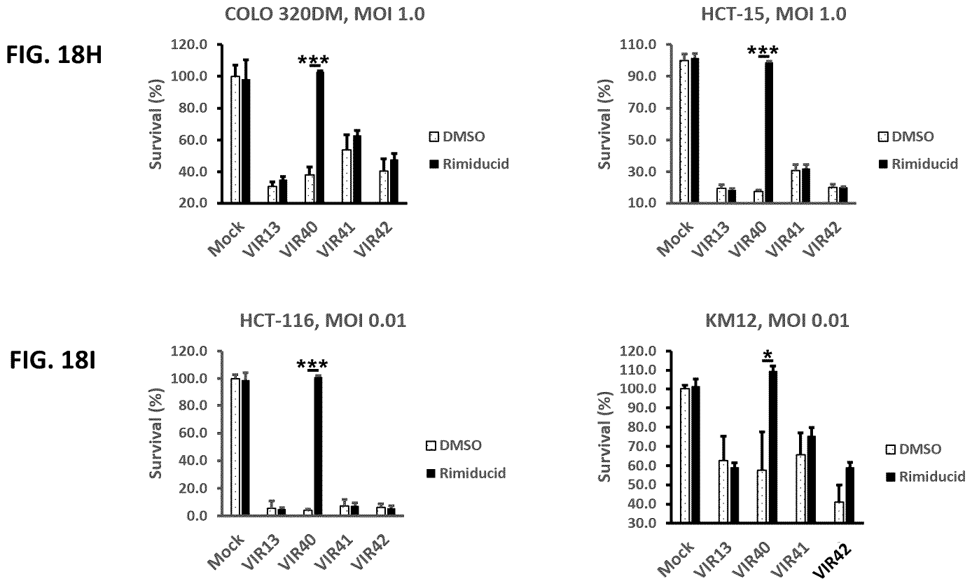

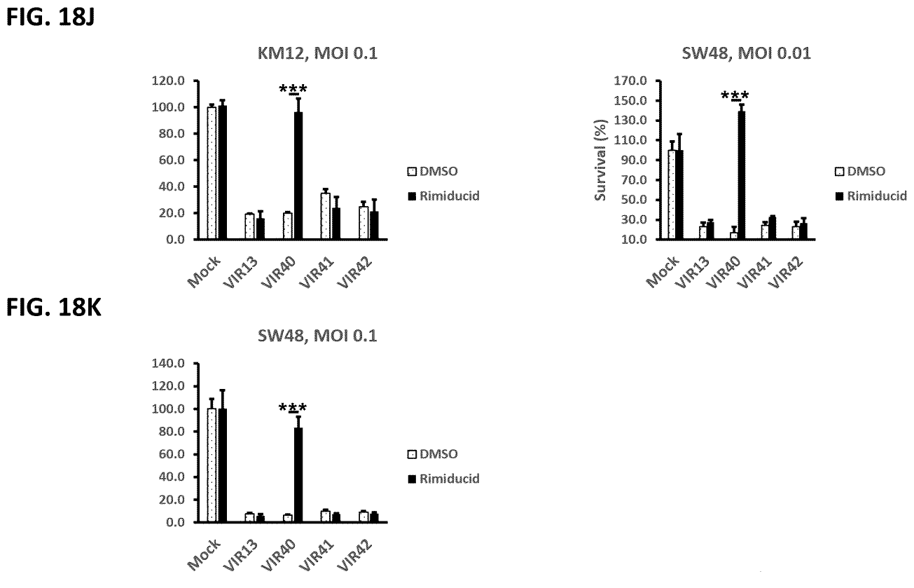

Rimiducid treatment significantly inhibited virus-mediated cytotoxicity in breast, prostate, lung cancer and melanoma, and Microsatellite Instable (MSI) colon cancer cells infected with VIR40, but not with VIR41, VIR13 and VIR42, as demonstrated by BT-549 breast cancer cells (FIG. 18A), Hs578T breast cancer cells (FIG. 18B), 4T1 breast cancer cells (FIG. 18C), DU-145 prostate cancer cells (FIG. 18D), PC-3 prostate cancer cells (FIG. 18E), A549 lung and melanoma cancer cells (FIG. 18F), M14 lung and melanoma cancer cells (FIG. 18G), COLO 320 DM and HCT-15 MSI colon cancer cells (FIG. 18H), HCT-116 and KM12 MSI colon cancer cells (FIG. 18I), KM12 MSI colon cancer cells (FIG. 18J) and SW48 MSI colon cancer cells (FIG. 18K). These results indicate that iCasp9, but not iDED and iFAS, inhibits virus-mediated cytotoxicity in the infected cancer cells.

The ability of Rimiducid treatment to inhibit virus-mediated cytotoxicity after infection of MSS colorectal cancer cells with VIR13, VIR40, VIR41 and VIR42 was also evaluated. MSS colorectal cancer cell lines infected with VIR41 and VIR42 at MOIs of 0.01 and 0.1 showed a significantly lower percentage of survival when compared to cultures infected with VIR13 and VIR42 or the mock control, as demonstrated in COLO205 MSS colon cancer cells (FIG. 19A), HCC-2998 colon cancer cells (FIG. 19B), HT-29 cells (FIG. 19C), LS123 cells (FIG. 19D), LS174T cells (FIG. 19E), SW620 cells (FIG. 19F) and WiDR cells (FIG. 19G). These results indicate that iDED and iFAS, but not iCasp9, enhance virus-mediated cytotoxicity in MSS colon cancer cells.

### Example 7: Assessment of VIR13 Tumor Growth Inhibition in Mice

The oncolytic activity of the exemplary recombinant strain VIR13 was assessed by monitoring in vivo tumor growth following a single dose of VIR13 injected intravenously in an SL-4 mice model of colon adenocarcinoma. As shown in Table E5 and FIG. 13, VIR13 is derived from parental VIP02 but is deleted in the TK (J2R) gene and is not engineered with any further heterologous protein (i.e., heterologous gene product).

Approximately 5×10⁵ SL-4 cells in 100 μL of PBS were implanted into the right flank of female C57BL/6 mice, 4-6 weeks old. SL-4 cells are a C57BL6 murine colon adenocarcinoma cell line and were obtained from AntiCancer, Inc. (San Diego, CA). For all tumor models, virus treatment was initiated when tumor volumes were about 60 mm³. Five mice per group were treated once with an intravenous dose of 1×10⁸ PFU of VIR13 or vehicle as control. Tumor volume and body weight were measured twice per week using a Vernier caliper and an electronic scale, respectively. Tumor volume was estimated by measuring the perpendicular minor dimension (W) and major dimension (L). Approximate tumor volume was calculated with the formula (W²×L)/2. Mice were euthanized when tumors reached termination criteria (tumor volume >=3000 mm³ and/or body weight loss >=20%).

As shown in FIG. 20, treatment of mice with VIR13 significantly reduced tumor progression. Data showed that three out of five (60%) of mice infected with VIR13 were cured 19 days post injection while none of the five mice treated with a control solution showed signs tumor growth inhibition 19 days post treatment.

### Example 8: Generation and Assessment of Recombinant VIP02-Based Viral Clones Compared to VIR13

### A. Generation of Recombinant Vaccinia Viruses

Recombinant VACV strains with immune modulating activity, were generated by insertion of mouse interferon regulatory factor 3 (mIRF3) under the control of the VACV SSE promoter into the endogenous viral J2R gene or by the disruption of J2R and B2R in the viral genome. The recombinant strains were generated by homologous recombination of a transfer vector and the parental genome.

The transfer vectors were generated as follows:

Plasmid pITK-SSE-mIRF3. The mouse IRF3 cDNA under the control of the VACV SSE promoter was synthesized by IDT (Coralville, IA). The synthesized fragment was digested with PstI and SacI and cloned into the same-cut plasmid pITK-SSE described in Example 3 to yield pITK-SSE-mIRF3. The DNA sequence of SSE-mIRF3 was confirmed by DNA sequencing.

Plasmid pITK-mH5-mIRF3. The mouse IRF3 cDNA under the control of the mH5 promoter was synthesized by IDT (Coralville, IA). The synthesized fragment was digested with PstI and SacI and cloned into the same-cut plasmid pITK-mH5 described in Example 3 to yield pITK-mH5-mIRF3. The DNA sequence of mH5-mIRF3 was confirmed by DNA sequencing.

Plasmid pIB2R. The left and right flanking sequences of the B2R gene of VACV IHD-J were PCR-amplified from VACV IHD-J genomic DNA using the primers: 5′-GCTGAATTCGAGATATTAAAGCGAGTAATATAG-3′ (SEQ ID NO: 94) and 5′-AGGAGCTCTCGAGCGGCCGCTGCAGGTACCAGGAAAGGCATGAAGATTCTCGTCG-3 (SEQ ID NO: 95) (left flank), 5′-GGTACCTGCAGCGGCCGCTCGAGAGCTCCTGGTAAAAGTTAAACTTGGGGAGAATG-3′ (SEQ ID NO: 96) and 5′-ACGAAGCTTGGAAGCTGCTGCCGTATACGTGTTCTTAG-3′ (SEQ ID NO: 97) (right flank). The two fragments were joined together using the method of gene splicing by overlapping extension as described in Horton et al., Methods Enzymol., 1993; 217:270-9 hereby incorporated in its entirety. The resulting fragment was digested with EcoRI and HindIII and cloned into the same-cut plasmid pVM-1 to yield pIB2R. The flanking sequences of B2R were confirmed by DNA sequencing.

For VIR94, parental virus VIR13 was generated by infecting CV-1 cells with VIP02 at an MOI of 0.1 for 1 hour and then transfected with pITK transfer vector described in Example 3, to yield a parental recombinant virus in which the J2R (TK) gene was disrupted. To generate the recombinant VIR94, CV-1 cells were infected with the parental virus VIR13 at an MOI of 0.1 for 1 hour and then transfected with the pIB2R transfer vector. Recombinant vaccinia strain VIR93 and VIR 96 were generated by infecting CV-1 cells with the parental strain VIP02 at an MOI of 0.1 for 1 hour and then transfected with the pITK-SSE-mIRF3 or pITK-mH5-mIRF3 transfer vector respectively, as described in Example 3.

Table E8 summarizes the generated recombinant VACV strains, including the parental virus and transfer vectors used in their generation.

TABLE E8

| Recombinant | | | |
|---|---|---|---|
| VACVs | Parent Virus | Transfer Vector | Genotype |
| VIR13 | VIP02 | pITK | J2R(TK) is |
| (SEQ ID NO: 4) | (SEQ ID NO: 1) | | disrupted |
| VIR93 | VIP02 | pITK-SSE-mIRF3 | SSE-mIRF3 |
| (SEQ ID NO: 49) | (SEQ ID NO: 1) | | inserted at |
| | | | J2R |
| VIR94 | VIR13 | pIB2R | J2R and B2R |
| (SEQ ID NO: 48) | (SEQ ID NO: 4) | | are disrupted |
| VIR96 | VIP02 | pITK-mH5-mIRF3 | mH5-mIRF3 |
| (SEQ ID NO: 50) | (SEQ ID NO: 1) | | inserted at |
| | | | J2R |

### B. Assessment of Modified Recombinant Vaccinia Virus Compared to VIR13

The tumor inhibitory activity of VIR41, VIR86, VIR93, VIR94, and VIR96 were evaluated in vivo using the SL-4 GFP mouse colon adenocarcinoma model. Female C57BL/6 mice that were 5-6 weeks old were implanted with 5×10⁵ SL-4 GFP cells in 100 μL of PBS in the right flank of the mice. In this experiment, mice were treated once with an intravenous dose of 1×10⁸ PFU of each indicated virus. Tumor volume and body weight were measured twice per week using vernier calipers and an electronic scale, respectively. Tumor volume was estimated by measuring the perpendicular minor dimension (W) and major dimension (L). Approximate tumor volume was calculated with the formula (W²×L)/2.

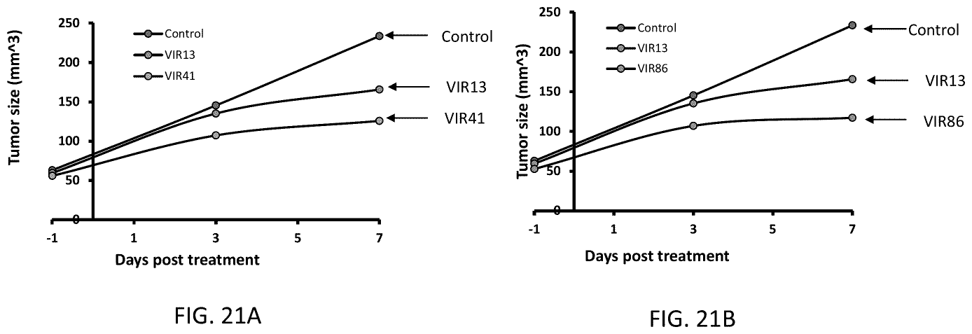

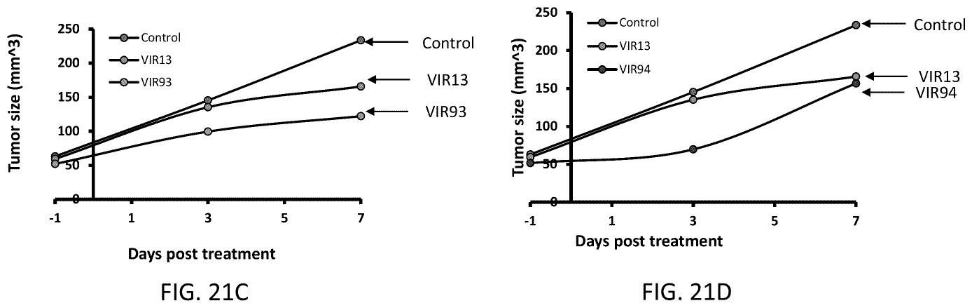

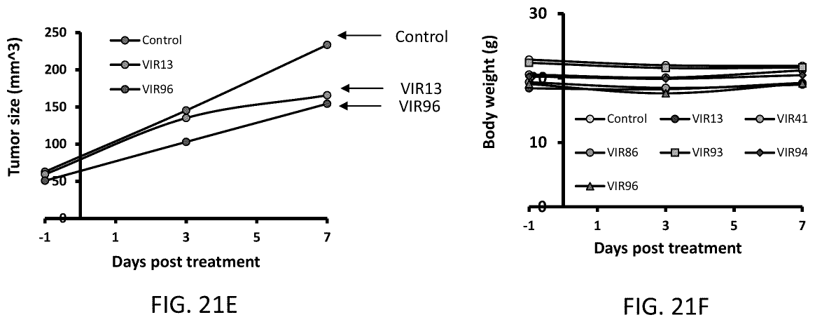

As shown in FIG. 21A-21E, mice treated with VIR13 showed a reduction in tumor volume when compared to the vehicle treated controls. Moreover, mice treated with VIR41, VIR86, VIR93, VIR94, or VIR 96 showed improved efficacy at reducing tumor volume by 3 to 7 days after treatment when compared to VIR13 and the vehicle treated controls, indicating that the introduction IRF3 and the disruption of B2R lead to increased anti-tumor activity of the recombinant virus. Further, as shown in FIG. 21F. there was no drastic loss in body weight between any of the viral treated mice in comparison to the vehicle treated control, indicating the enhanced antitumor activity of the recombinant vaccinia viruses do not induce toxicity.

Example 9: Generation and Assessment of Recombinant VIP02 Based Viral Clones Engineered with Further Immune Modulating Genes
A. Generation of Recombinant Vaccinia Viruses Engineered with Immune Modulating Genes

Recombinant VACV strains with immune genes, were generated by insertion of immune genes under various promoters. These include mouse interferon regulatory factor 3 (mIRF3) under the control of the VACV SSE promoter into the endogenous viral J2R gene, insertion of MDNA11 under the control of 11K promoter inserted into A56R gene, insertion of mCXCL9 under the mH5 promoter inserted at A56R, and/or insertion of mscIL-12 under the 11K promoter inserted at A56R. The recombinant strains were generated by homologous recombination of a transfer vector and the parental genome.

The transfer vectors were generated as follows:

Plasmid pIB2R. The left and right flanking sequences of the B2R gene of VACV IHD-J were PCR-amplified from VACV IHD-J genomic DNA using the primers: 5′-GCTGAATTCGAGATATTAAAGCGAGTAATATAG-3 (SEQ ID NO: 94) and 5′-AGGAGCTCTCGAGCGGCCGCTGCAGGTACCAGGAAAGGCATGAAGATTCTCGTCG-3′ (SEQ ID NO: 95) (left flank), 5′-GGTACCTGCAGCGGCCGCTCGAGAGCTCCTGGTAAAAGTTAAACTTGGGGAGAATG-3′ (SEQ ID NO: 96) and 5′-ACGAAGCTTGGAAGCTGCTGCCGTATACGTGTTCTTAG-3′ (SEQ ID NO: 97) (right flank). The two fragments were joined together using the method of gene splicing by overlapping extension as described in Horton et al., Methods Enzymol., 1993; 217:270-9 hereby incorporated in its entirety. The resulting fragment was digested with EcoRI and HindIII and cloned into the same-cut plasmid pVM-1 to yield pIB2R. The flanking sequences of B2R were confirmed by DNA sequencing.

pIB2R-SSE-mIRF3. pITK-SSE-mIRF3 was digested with KpnI and SacI. The fragment containing SSE-mIRF3 was gel-purified and cloned into the same-cut plasmid pIB2R to yield pIB2R-SSE-mIRF3. The DNA sequence of SSE-mIRF3 was confirmed by DNA sequencing.

pIA56R-11K-MDNA11. A long-acting IL-2 superkine (MDNA11) (Merchant R, et. al., Jounral for immunotherapy of cancer. 2022, 10 (1), hereby incorporated in its entirety) under the control of the VACV 11K promoter was synthesized by IDT (Coralville, IA). The synthesized fragment was digested with KpnI and SacI and cloned into the same-cut plasmid pIA56R to yield pIA56R-11K-MDNA11. The DNA sequence of the synthesized 11K-MDNA11 was confirmed by DNA sequencing.

pIA56R-mH5-mCXCL9-11K-mscIL-12. pIA56R-mH5-hFGL1 was digested with PstI and SacI. The fragment containing the backbone plasmid and the mH5 promoter (pIA56R-mH5) was gel-purified. The DNA fragment mCXCL9-11K-mscIL-12 containing mouse CXCL9 (mCXCL9) and mouse single chain IL-12 (mscIL-12) (Lieschke G J, et. al., Nature biotechnology. 1997, 15 (1): 35-40, hereby incorporated in its entirety) under the control of the VACV 11K promoter was synthesized by IDT (Coralville, IA) and digested with PstI and SacI, then cloned into the same-cut plasmid pIA56R-mH5 to yield pIA56R-mH5-mCXCL9-11K-mscIL-12. The DNA sequence of mCXCL9-11K-mscIL-12 was confirmed by DNA sequencing.

pIA56R-11K-MDNA11T. The synthesized 11K-MDNA11 fragment was mutated using the method of gene splicing by overlapping extension to introduce a “glutamine-126 to threonine” mutation as described in Mo F, et. al,. Nature 2021, 597 (7877): 544-8, hereby incorporated in its entirety. The resulting fragment is called 11K-MDNA11T, which was then digested with KpnI and SacI and cloned into the same-cut plasmid pIA56R to yield pIA56R-11K-MDNA11T. The DNA sequence of the 11K-MDNA11T was confirmed by DNA sequencing.

Plasmid pITK-SSE-mIRF3. The mouse IRF3 cDNA under the control of the VACV SSE promoter was synthesized by IDT (Coralville, IA). The synthesized fragment was digested with PstI and SacI and cloned into the same-cut plasmid pITK-SSE described in Example 3 to yield pITK-SSE-mIRF3. The DNA sequence of SSE-mIRF3 was confirmed by DNA sequencing.

pITK-SSE-hIRF3. pITK-SSE-hFGL1tm was digested with PstI and SacI. The fragment containing the backbone plasmid and the SSE promoter (pITK-SSE) was gel-purified. The human IRF3 (Interferon Regulatory Factor 3) cDNA was synthesized by IDT (Coralville, IA) and digested with PstI and SacI, then cloned into the same-cut plasmid pITK-SSE to yield pITK-SSE-hIRF3. The DNA sequence of SSE-hIRF3 was confirmed by DNA sequencing.

Plasmid pIA56R-mH5-hCXCL9-11k-hscIL-12. pIA56R-mH5-hFGL1 was digested with PstI and SacI. The fragment containing the backbone plasmid and the mH5 promoter (pIA56R-mH5) was gel-purified. The DNA fragment hCXCL9-11K-hscIL-12 containing human CXCL9 (hCXCL9) and human single chain IL-12 (hscIL-12) (Lieschke G J, et. al., Nature biotechnology. 1997, 15 (1): 35-40, hereby incorporated in its entirety) under the control of the VACV 11K promoter was synthesized by IDT (Coralville, IA) and digested with PstI and SacI, then cloned into the same-cut plasmid pIA56R-mH5 to yield pIA56R-mH5-hCXCL9-11K-hscIL-12. The DNA sequence of hCXCL9-11K-hscIL-12 was confirmed by DNA sequencing.

For generation of the recombinant vaccinia viruses listed in Table E9, CV-1 cells were infected with parental viruses at an MOI of 0.1 for 1 hour and then transfected with their respective transfer vectors (see Table E9) by use of jetPRIME in vitro DNA and siRNA transfection reagent (Polyplys-transfection Inc, New Your, NY). Two days post infection, infected/transfected cells were harvested and recombinant viruses were selected and plaque purification was carried out as described in Falkner F G, et. Al., J. Virol. 1990, 64 (6): 3108-11, hereby incorporated in its entirety.

Table E9 summarizes the generated recombinant VACV strains, including the parental virus and transfer vectors used in their generation.

TABLE E9

| Recombinant | Parent | | |
|---|---|---|---|
| VACVs | Virus | Transfer Vector | Genotype |
| VIR94 | VIR13 | pIB2R | J2R and B2R are |
| (SEQ ID | (SEQ ID | | disrupted |
| NO: 48) | NO: 4) | | |
| VIR100 | VIR93 | pIB2R | B2R is disrupted; SSE- |
| (SEQ ID | (SEQ ID | | mIRF3 inserted at J2R |
| NO: 80) | NO: 49) | | |
| VIR103 | VIR86 | pIB2R- | SSE-hmLIGHT inserted |
| (SEQ ID | (SEQ ID | SSE-mIRF3 | at A35R; mH5-anti- |
| NO: 82) | NO: 47) | | VEGF-anti-Ang2-SEL- |
| | | | VK inserted at J2R; |
| | | | SSE-mIRF3 inserted at |
| | | | B2R |
| VIR105 | VIR100 | pIA56R-11K- | B2R is disrupted; SSE- |
| (SEQ ID | (SEQ ID | MDNA11 | mIRF3 inserted at J2R; |
| NO: 84) | NO: 80) | | 11K-MDNA11 inserted |
| | | | at A56R |
| VIR106 | VIR100 | pIA56R-mH5- | B2R is disrupted; SSE- |
| (SEQ ID | (SEQ ID | mCXCL9-11K- | mIRF3 inserted at J2R; |
| NO: 85) | NO: 80) | mscIL-12 | mH5-mCXCL9-11K- |
| | | | mscIL-12 inserted at |
| | | | A56R |
| VIR109 | VIR102 | pIA56R-mH5- | SSE-iDED inserted at |
| (SEQ ID | (SEQ ID | mCXCL9-11K- | J2R; SSE-mIRF3 |
| NO: 86) | NO: 81) | mscIL-12 | inserted at B2R; mH5- |
| | | | mCXCL9-11K-mscIL- |
| | | | 12 inserted at A56R |
| VIR111 | VIR103 | pIA56R-11K- | SSE-hmLIGHT inserted |
| (SEQ ID | (SEQ ID | | at A35R; mH5-anti- |
| NO: 87) | NO: 82) | MDNA11 | VEGF-anti-Ang2-SEL- |
| | | | VK inserted at J2R; |
| | | | SSE-mIRF3 inserted at |
| | | | B2R; 11K-MDNA11 |
| | | | inserted at A56R |
| VIR113 | VIR103 | pIA56R-11K- | SSE-hmLIGHT inserted |
| (SEQ ID | (SEQ ID | MDNA11T | at A35R; mH5-anti- |
| NO: 88) | NO: 82) | | VEGF-anti-Ang2-SEL- |
| | | | VK inserted at J2R; |
| | | | SSE-mIRF3 inserted at |
| | | | B2R; 11K-MDNA11T |
| | | | inserted at A56R |
| VIR114 | VIR104 | pIA56R-11K- | F14.5L fused with |
| (SEQ ID | (SEQ ID | MDNA11 | CRASP-2; CPXV012, |
| NO: 89) | NO: 83) | | 018 and 203 inserted at |
| | | | J2R; SSE-mIRF3 |
| | | | inserted at B2R; 11K- |
| | | | MDNA11 inserted at |
| | | | A56R |
| VIR115 | VIR104 | pIA56R-mH5- | F14.5L fused with |
| (SEQ ID | (SEQ ID | mCXCL9-11K- | CRASP-2; CPXV012, |
| NO: 90) | NO: 83) | mscIL-12 | 018 and 203 inserted at |
| | | | J2R; SSE-mIRF3 |
| | | | inserted at B2R; mH5- |
| | | | mCXCL9-11K-mscIL- |
| | | | 12 inserted at A56R |
| VIR123 | VIR94 | pITK-SSE-hIRF3 | B2R are disrupted; SSE- |
| (SEQ ID | (SEQ ID | | hIRF3 inserted at J2R |
| NO: 91) | NO: 48) | | |
| VIR127 | VIR123 | pIA56R-mH5- | B2R is disrupted; SSE- |
| (SEQ ID | (SEQ ID | hCXCL9-11K- | hIRF3 inserted at J2R; |
| NO: 92) | NO: 91) | hscIL-12 | mH5-hCXCL9-11K- |
| | | | hscIL-12 inserted at |
| | | | A56R |
| VIR128 | VIP02 | pITK-SSE-hIRF3 | SSE-hIRF3 inserted at |
| (SEQ ID | (SEQ ID | | J2R |
| NO: 93) | NO: 1) | | |

B. Assessment of Recombinant Vaccinia Viruses Engineered with Immune Genes with SL-4 Mouse Colon Adenocarcinoma Model

The tumor inhibitory activity of VIR 100, VIR103, VIR105, VIR106, VIR109, VIR113, VIR114, and VIR115 were evaluated in vivo using the SL-4 GFP mouse colon adenocarcinoma model. Female C57BL/6 mice that were 5-6 weeks old were implanted with 5×10⁵ SL-4 GFP cells in 100 μL of PBS in the right flank of the mice. In this experiment, mice were treated once with an intravenous dose of 1×10⁸ PFU of each indicated virus. Tumor volume and body weight were measured twice per week using vernier calipers and an electronic scale, respectively. Tumor volume was estimated by measuring the perpendicular minor dimension (W) and major dimension (L). Approximate tumor volume was calculated with the formula (W²×L)/2.

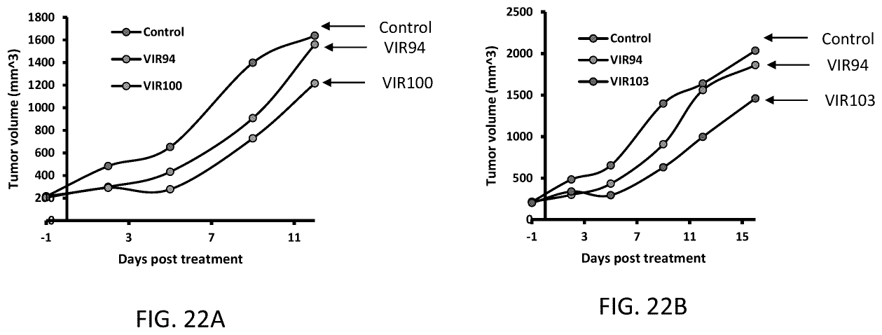

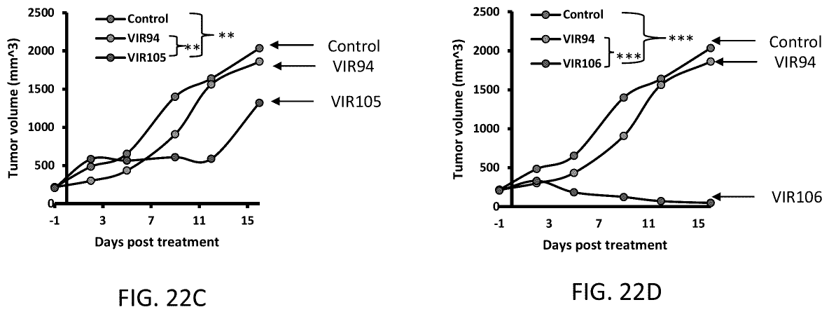

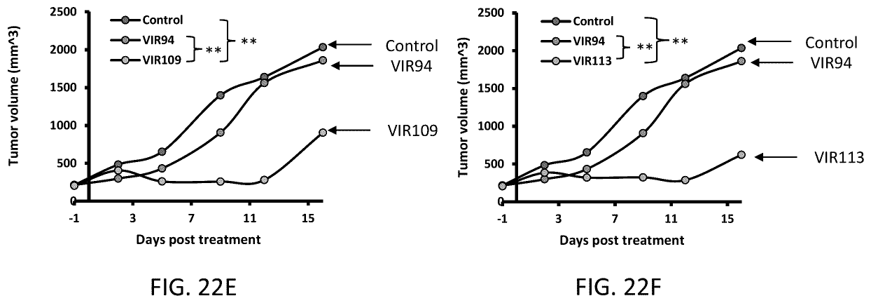

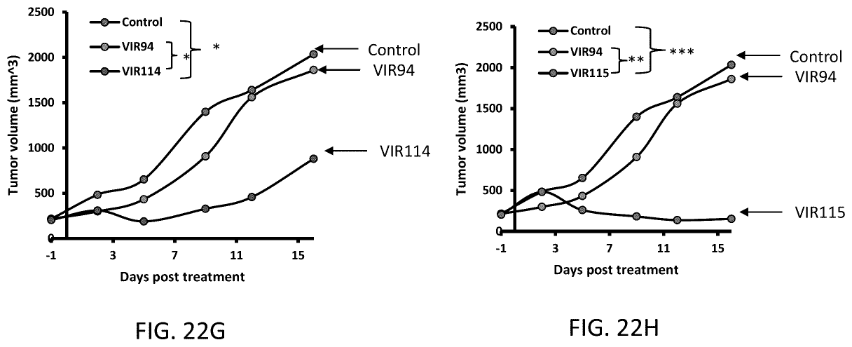

As shown in FIG. 22A-22H, mice treated with VIR94 appeared to have mild reduction in tumor volume when compared to the vehicle treated controls. VIR100 and VIR103, as shown in FIG. 22A and FIG. 22B respectively, had a mild reduction in tumor volume compared to VIR94 and vehicle control. As shown in FIG. 22C-22H for VIR105, VIR106, VIR109, VIR113, VIR114, and VIR115, respectively, all had statistically significant reduction in tumor size when compared to both vehicle treated control mice and VIR94 treated mice. (*=p≤0.05; **=p≤0.01; ***=p≤0.001). In addition, VIR106, and VIR115 were close to eradicating the tumor. These results indicate that recombinant vaccinia viruses engineered to contain immune proteins, such as IL-12, CXCL9, MDNA11 and MDNA11T, have enhanced and potent anti-tumor properties.

C. Comparison of Recombinant Vaccinia Viruses Engineered with MDNA11 or Mutant MDNA11T

MDNA11T (SEQ ID NO: 98) is a mutant MDNA11, which is a long-acting IL-2 superkine, to increase the immune stimulating properties of MDNA11. To test whether the MDNA11T mutant has increased efficacy at decreasing tumor mass compared to parental virus or recombinant virus with MDNA11, VIR113 was evaluated in vivo using the SL-4 GFP mouse colon adenocarcinoma model compared to VIR111, which contained MDNA11, and VIR103, the parental strain to both VIR111 and VIR113. Female C57BL/6 mice that were 5-6 weeks old were implanted with 5×10⁵ SL-4 GFP cells in 100 μL of PBS in the right flank of the mice. In this experiment, mice were treated once with an intravenous dose of 1×10⁸ PFU of each indicated virus. Tumor volume and body weight were measured twice per week using vernier calipers and an electronic scale, respectively. Tumor volume was estimated by measuring the perpendicular minor dimension (W) and major dimension (L). Approximate tumor volume was calculated with the formula (W²×L)/2.

As shown in FIG. 23A, VIR111 did not decrease tumor volume compared to parental VIR103. VIR113 had a significant decrease in tumor volume compared to both VIR 111 and VIR103, indicating that viruses encoding MDNA11T have increased anti-tumor potency compared to viruses containing the unmutated MDNA11 and to recombinant viruses that do not contain either MDNA11 or MDNA11T. FIG. 23B, shows a schematic for how both MDNA11 and MDNA11T are created from wild-type human IL-2 (wt hIL-2).

D. Assessment of Recombinant Vaccinia Viruses Engineered with Immune Genes with Lewis Lung Carcinoma Mouse Model

The three strains with the largest reduction in tumor volume in the SL-4 mouse model (VIR106, VIR113, and VIR115) were evaluated in vivo using a Lewis lung carcinoma mouse cancer model to evaluate their efficacy against other cancers. Female C57BL/6 mice that were 6-7 weeks old were implanted with 1×10⁶ LLCI cells in 100 μL of PBS in the right flank of the mice. In this experiment, mice were treated once with an intravenous dose of 1×10⁸ PFU of each indicated virus. Tumor volume and body weight were measured twice per week using vernier calipers and an electronic scale, respectively. Tumor volume was estimated by measuring the perpendicular minor dimension (W) and major dimension (L). Approximate tumor volume was calculated with the formula (W²×L)/2.

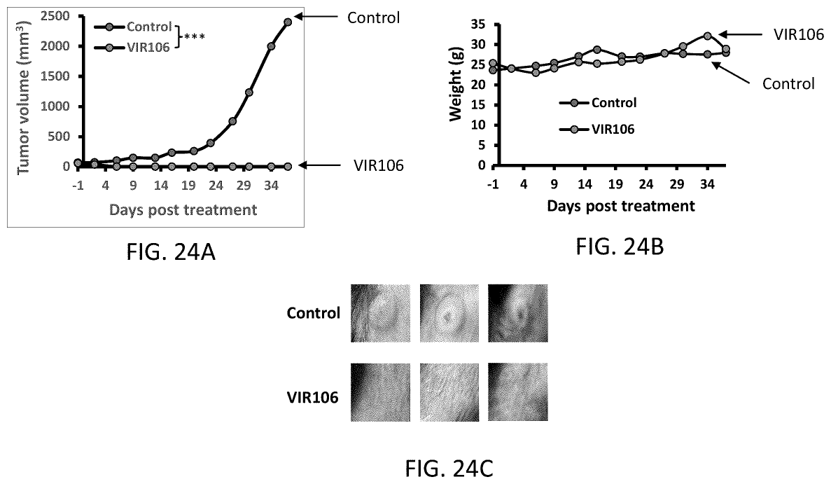

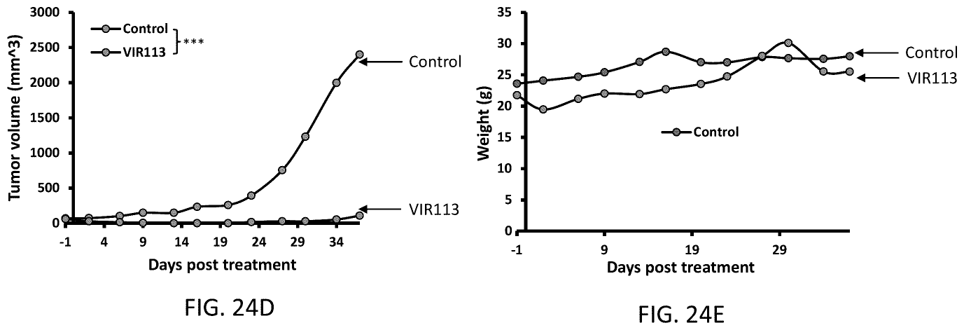

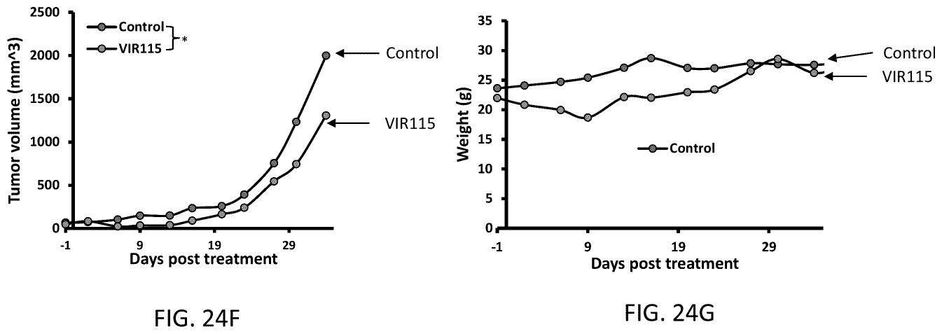

As shown in FIG. 24A, VIR106 cured 100 percent of the tumors, within 6 days after treatment, with no detectable tumor volume compared to the vehicle treated control where the tumor continued to grow over the entire test period. There was no detectable difference in weight between VIR 106 and vehicle treated control indicating the recombinant vaccinia virus was not toxic (FIG. 24B). FIG. 24C shows the results where VIR106 treated mice did not have detectable tumors 8 days after treatment. As shown in FIG. 24D, VIR113 showed similar results with mice being cured of Lewis lung carcinoma, except one mouse which relapsed. VIR113 also showed no toxicity in body weight (FIG. 24E). VIR115 resulted in a statistically significant reduction in tumor volume, as shown in FIG. 23F, while not causing toxicity seen in body weight (FIG. 24G). VIR115 also cured 50% of Lewis lung carcinoma mice in the study (data not shown). These results demonstrate that the recombinant vaccinia viruses engineered with Immune genes are effective against multiple cancer models.

E. Assessment of Recombinant Vaccinia Viruses Engineered with Immune Genes with Melanoma Mouse Model

The three strains, VIR106, VIR113, and VIR115, were further evaluated in vivo using a melanoma mouse cancer model to evaluate their efficacy against other cancers. Male C57BL/6 mice that were 5-6 weeks old were implanted with 1×10⁶ B16-F10 RFP cells in 100 μL of PBS in the right flank of the mice. In this experiment, mice were treated once with an intravenous dose of 1× 108 PFU of each indicated virus. Tumor volume and body weight were measured twice per week using vernier calipers and an electronic scale, respectively. Tumor volume was estimated by measuring the perpendicular minor dimension (W) and major dimension (L). Approximate tumor volume was calculated with the formula (W²×L)/2.

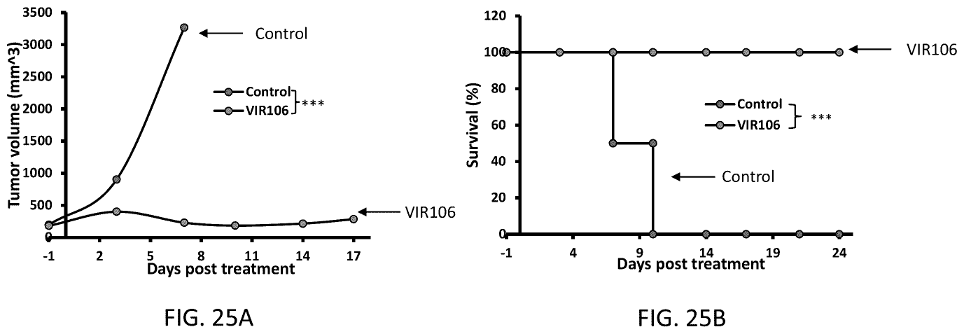

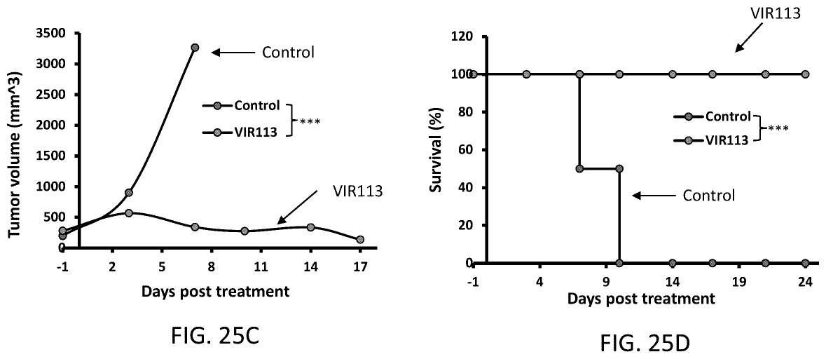

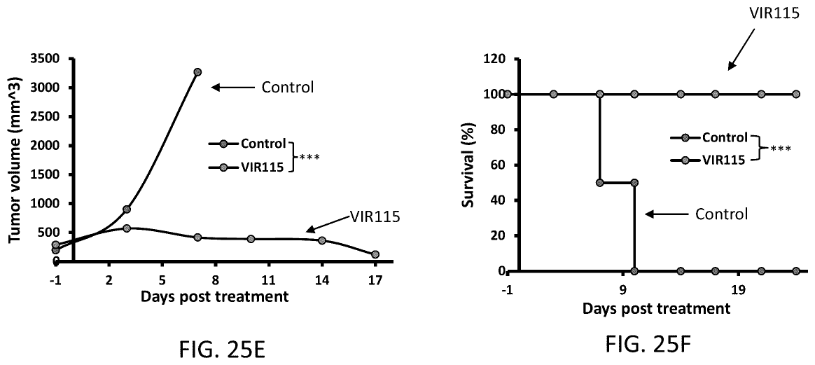

FIG. 25A shows that VIR 106 significantly reduced tumor volume compared to vehicle treated control mice. VIR106 protected mice and lead to a 100 percent survival rate over the test period (24 days) compared to vehicle treated mice which had all died by 10 days post treatment as shown in FIG. 25B. Similar results were obtained for VIR113, with reduced tumor volume compared to vehicle treated control as shown in FIG. 25C, and leading to 100% survival over the test period (24 days post treatment) compared to the vehicle treated mice which all died by 10 days post treatment as shown in FIG. 25D. VIR115 was also effective at statistically significantly reducing tumor volume compared to vehicle treated control mice as shown in FIG. 25E, and leading to 100% survival over the test period (24 days post treatment) compared to the vehicle treated mice which all died by 10 days post treatment as shown in FIG. 25F.

F. Quantification of Immune Proteins after Infection with Recombinant Vaccinia Virus

B16-F10 and Hela S3 cancer cells were infected with various recombinant vaccinia viruses at an MOI of 10 each. Four hours after infection, cells were harvested by low-speed centrifugation and lysed using RIPA lysis buffer (VWR, Solon, OH). A Halt protease and phosphatase inhibitor cocktail (Thermo Scientific, Rockford, IL) was added freshly to the lysis buffer. Protein amounts were determined using RC DC Protein Quantification Kit (Bio-Rad Laboratories, Hercules, CA). Normalized protein amounts were subjected to an SDS-PAGE (Bio-Rad Laboratories, Hercules, CA) and transferred onto a PVDF membrane for Western blotting, which was blotted with primary antibodies followed by horseradish peroxidase (HRP)-conjugated secondary antibodies. Proteins were visualized with a chemiluminescent detection kit (ClarityMax Western ECL Substrate, Bio-Rad Laboratories, Hercules, CA) and Azure Biosystems c300 (Azure Biosystems, Dublin, CA). Antibodies against phospho-IRF3 (1:1,000) and total IRF3 as well as HRP-linked anti-rabbit IgG (1:1000) were from Cell Signaling Technology (Danvers, MA). Anti-beta-actin antibody (1:1,000) was from Novus Biologicals (Centennial, CO). Heat inactivated VIR13 (iVIR13) was used as a positive control for the stimulation of phosphor-IRF3 and beta actin was used as a loading control.

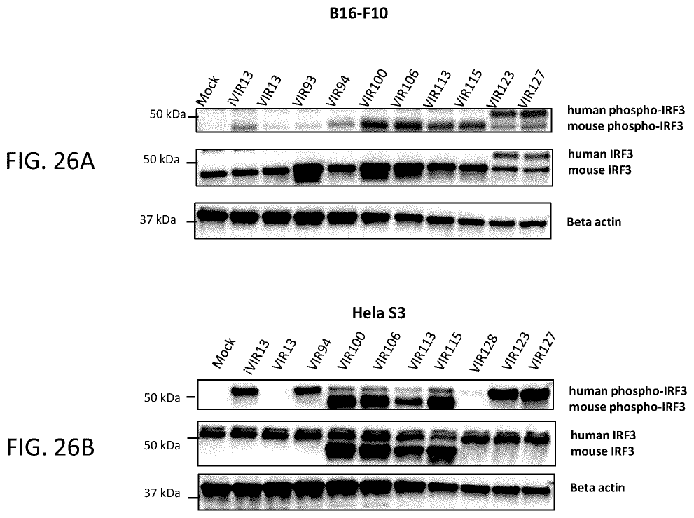

As shown in FIG. 26A, expression of exogeneous mIRF3 (VIR93) resulted in a slight increase in the level of phospho-IRF3 while disruption of the B2R gene (VIR94) led to a significant increase in the level of phospho-IRF3 compared to VIR13, indicating activation of the IRF3 pathway. Combination of expression of exogeneous mIRF3 and disruption of B2R gene (VIR100, VIR106, VIR113 and VIR115) resulted in the highest level of mouse phospho-IRF3, even higher than the positive control iVIR13. Interestingly, hIRF3 (VIR123, and VIR127, both of which had the hIRF3 gene) can be phosphorated in mouse cancer cells. As shown in FIG. 26B, similar results are obtained in human Hela S3 cells. Similarly, combination of expression of exogeneous hIRF3 and disruption of the B2R gene (VIR123 and VIR127) resulted in the highest level of human phospho-IRF3, even higher than the positive control iVIR13. Disruption of B2R (VIR94) resulted in higher level of human phospho-IRF3 than the expression of exogeneous human IRF3 (VIR128). Further, mIRF3 (VIR100, VIR106, VIR113 and VIR115) can also be phosphorated in human cancer cells.

To further quantify induction of the interferon pathways by recombinant vaccinia viruses, B16-F10 and Hela S3 cells were infected with each virus at an MOI of 10. Twenty-four hours after infection, supernatants were harvested. Mouse and human interferon-beta was quantified using Verikine-HS Mouse Interferon Beta Serum ELISA Kit and Verikine-HS Human Interferon Beta Serum ELISA Kit (PBL Assay Science, Piscataway NJ), respectively. Mouse and human CXCL9 was measured using Mouse CXCL9/MIG Duoset ELISA and Human CXCL9/MIG Duoset ELISA (Biotechne/R & D Systems, Minneapolis, MN), respectively. HEK-Blue IL-12 cells (InvivoGen, San Diego, CA) were used to quantify both human and mouse bioactive IL-12.

Table E10 shows the results for interferon-beta (IFN-beta) production. VIR100, VIR106, VIR113, VIR115, VIR128, VIR123, and VIR127 all had increased levels of human IFN-beta and all of those viruses had increased mouse IFN-beta, except VIR128 when compared to mock treated cells, consistent with activation of the IRF3 pathway as shown in FIG. 26. VIR123 and VIR127, which encode human IRF3, had increased human IFN-beta production compared to recombinant viruses encoding mouse IRF3.

TABLE E10

| | B16-F10 | Hela S3 |
|---|---|---|
| Infection | mouse IFN-beta (pg/ml) | human IFN-beta (pg/ml) |
| Mock | BLOD* | BLOD |
| iVIR13 | 5881.81 | 1428.25 |
| VIR13 | BLOD | BLOD |
| VIR93 | BLOD | BLOD |
| VIR94 | BLOD | BLOD |
| VIR100 | 1.40 | 2.23 |
| VIR106 | 1.28 | 1.97 |
| VIR113 | 1.51 | 5.44 |
| VIR115 | 2.39 | 3.17 |
| VIR128 | BLOD | 6.19 |
| VIR123 | 0.88 | 85.97 |
| VIR127 | 0.68 | 51.56 |

*BLOD: Below the limit of detection (0.94 pg/ml for mIFN-beta; 1.2 pg/ml for hIFN-beta).

Table E11 shows the results for CXCL9 ELISAs and IL-12 bioassay quantifying protein production in both B16-F10 and Hela S3-12 cells. The viruses that encoded the mouse CXCL9 gene (VIR106 and VIR115) had detectable levels of mouse CXCL9, indicating that the virus was effective at delivering the gene and driving expression. Similar results were obtained for VIR127, the only recombinant virus to express human CXCL9 and the only recombinant virus to have detectable levels of human CXCL9. IL-12 production was detected in VIR106, VIR115, and VIR 127 for both human and mouse cells, which were the recombinant viruses that encode either human or mouse IL-12. iVIR 13 was able to induce low levels of IL-12 in human Hela S3 cells, but not in mouse B16-F10 cells. Interestingly, VIR113, which does not carry the gene for CXCL9 or IL-12, but does encode MDNA11T and hmLIGHT, was able to induce the expression of mCXCL9 in B16-F10 cells and IL-12 in both B16-F10 and Hela S3 cells, indicating MDNA11T and/or hmLIGHT is sufficient to drive activation of these pathways.

TABLE E11

| | CXCL9 (ng/ml) | | | |
|---|---|---|---|---|
| | B16-F10 | Hela S3 | IL-12 (ng/ml) | |
| Infection | mouse CXCL9 | human CXCL9 | B16-F10 | Hela S3 |
| Mock | BLOD* | BLOD | BLOD | BLOD |
| iVIR13 | BLOD | BLOD | BLOD | 3.64 |
| VIR13 | BLOD | BLOD | BLOD | BLOD |
| VIR93 | BLOD | BLOD | BLOD | BLOD |
| VIR94 | BLOD | BLOD | BLOD | BLOD |
| VIR100 | BLOD | BLOD | BLOD | BLOD |
| VIR106 | 664.42 | BLOD | 2790.41 | 3329.36 |
| VIR113 | 381.39 | BLOD | 2545.02 | 1811.06 |
| VIR115 | 500.09 | BLOD | 3069.89 | 964.81 |
| VIR128 | BLOD | BLOD | BLOD | BLOD |
| VIR123 | BLOD | BLOD | BLOD | BLOD |
| VIR127 | BLOD | 7058.41 | 4519.37 | 1442.08 |

*BLOD: Below the limit of detection (15.6 pg/ml for mCXCL9; 62.5 pg/ml for hCXCL9; 1.56 ng/ml for IL-12).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed is:

1. A recombinant vaccinia virus, comprising a heterologous nucleic acid encoding an inducible apoptosis protein, wherein the apoptosis protein is a death effector domain of the Fas-associated death domain-containing protein (DED), a Fas, or a caspase.

2. The recombinant vaccinia virus of claim 1, wherein the apoptosis protein is a DED.

3. The recombinant vaccinia virus of claim 1, wherein the apoptosis protein is a caspase that is caspase 9 (Cas9).

4. The recombinant vaccinia virus of claim 1, wherein the apoptosis protein is a Fas.

5. The recombinant vaccinia virus of claim 1, wherein the heterologous nucleic acid encoding the inducible apoptosis protein is inserted into the genome of the vaccinia virus at the B2R, J2R, A35R, A56R or F14.5L gene loci.

6. The recombinant vaccinia virus of claim 1, wherein the heterologous nucleic acid encoding the inducible apoptosis protein is inserted into the genome of the vaccinia virus at the J2R gene locus.

7. The recombinant vaccinia virus of claim 1, wherein the inducible apoptosis protein comprises the apoptosis protein fused with an FKBP variant that is able to bind a chemical inducer of dimerization (CID).

8. The recombinant vaccinia virus of claim 7, wherein the FKBP variant is FKBP comprising the mutation F36V.

9. The recombinant vaccinia virus of claim 8, wherein the FKBP variant comprises an amino acid sequence that has at least 95% sequence identity to the amino acid sequence set forth in SEQ ID NO: 56.

10. The recombinant vaccinia virus of claim 7, wherein the chemical inducer of dimerization is AP1903 (Rimiducid).

11. The recombinant vaccinia virus of claim 1, wherein the inducible apoptosis protein:
(i) is an inducible DED (iDED) comprising an amino acid sequence that has at least 95% sequence identity to the amino acid sequence set forth in SEQ ID NO: 27:
(ii) is an inducible Fas (iFas) comprising an amino acid sequence that has at least 95% sequence identity to the amino acid sequence set forth in SEQ ID NO: 28: or
(iii) is an inducible Cas9 (iCas9) comprising an amino acid sequence that has at least 95% sequence identity to the amino acid sequence set forth in SEQ ID NO: 26.

12. The recombinant vaccinia virus of claim 1, wherein the heterologous nucleic acid is an expression cassette comprising the inducible apoptosis protein operably linked to a vaccinia synthetic early promoter PSE.

13. The recombinant vaccinia virus of claim 1, wherein the nucleic acid genome of the recombinant oncolytic vaccinia virus is derived from Vaccinia IHD-J strain genome.

14. The recombinant vaccinia virus of claim 1, wherein the nucleic acid genome of the recombinant oncolytic vaccinia virus has at least 95% sequence identity to the nucleic acid sequence set forth in SEQ ID NO: 1.

15. The recombinant vaccinia virus of claim 1, wherein:
(i) the nucleic acid genome of the recombinant oncolytic virus comprises a nucleic acid sequence that has at least 95% sequence identity to the nucleic acid sequence set forth in SEQ ID NO: 8:
(ii) the nucleic acid genome of the recombinant oncolytic virus comprises a nucleic acid sequence that has at least 95% sequence identity to the nucleic acid sequence set forth in SEQ ID NO: 9; or
(iii) the nucleic acid genome of the recombinant oncolytic virus comprises a nucleic acid sequence that has at least 95% sequence identity to the nucleic acid sequence set forth in SEQ ID NO: 7.

16. A recombinant vaccinia virus, comprising a heterologous nucleic acid encoding an inducible death effector domain (iDED), wherein the iDED comprises a death effector domain (DED) of the Fas-associated death domain-containing protein (FADD) fused with an FKBP variant that is able to bind AP1903 (Rimiducid).

17. The recombinant vaccinia virus of claim 16, wherein the FKBP variant is FKBP comprising the mutationF36V (FKBP-F36V).

18. The recombinant vaccinia virus of claim 17, wherein the FKBP-F36V comprises an amino acid sequence that has at least 95% sequence identity to the amino acid sequence set forth in SEQ ID NO: 56.

19. The recombinant vaccinia virus of claim 16, wherein the iDED comprises an amino acid sequence that has at least 95% sequence identity to the amino acid sequence set forth in SEQ ID NO: 27.

20. The recombinant vaccinia virus of claim 16, wherein the heterologous nucleic acid encoding iDED is inserted into the genome of the vaccinia virus at the B2R, J2R, A35R, A56R or F14.5L gene loci.

21. The recombinant vaccinia virus of claim 16, wherein the heterologous nucleic acid encoding iDED is inserted into the genome of the vaccinia virus at the J2R gene locus.

22. The recombinant vaccinia virus of claim 16, wherein the nucleic acid genome of the recombinant oncolytic vaccinia virus is derived from Vaccinia IHD-J strain genome.

23. The recombinant vaccinia virus of claim 16, wherein the nucleic acid genome of the recombinant oncolytic vaccinia virus is modified from a parental vaccinia virus that has a nucleic acid genome that has at least 99% sequence identity to the nucleic acid sequence set forth in SEQ ID NO: 1.

24. The recombinant vaccinia virus of claim 16, wherein the nucleic acid genome of the recombinant oncolytic vaccinia virus has at least 95% sequence identity to the nucleic acid sequence set forth in SEQ ID NO: 1.

25. A recombinant vaccinia virus derived from Vaccinia IHD-J strain genome, comprising a heterologous nucleic acid encoding an inducible death effector domain (iDED), wherein the iDED comprises a death effector domain (DED) of the Fas-associated death domain-containing protein (FADD) fused with an FKBP variant that is able to bind AP1903 (Rimiducid), the heterologous nucleic acid is inserted into the genome of the vaccinia virus at the J2R gene locus, and the nucleic acid genome of the recombinant oncolytic vaccinia virus has at least 95% sequence identity to the nucleic acid sequence set forth in SEQ ID NO: 1.

26. A recombinant vaccinia virus of claim 25, wherein the iDED comprises an amino acid sequence that has at least 95% sequence identity to the amino acid sequence set forth in SEQ ID NO: 27.

27. A recombinant vaccinia virus comprising a nucleic acid genome that has at least 95% sequence identity to the nucleic acid sequence set forth in SEQ ID NO: 8.

28. The recombinant vaccinia virus of claim 27, wherein the nucleic acid genome of the recombinant oncolytic virus comprises the nucleic acid sequence set forth in SEQ ID NO: 8.

29. A pharmaceutical composition comprising the recombinant vaccinia virus of claim 1.

30. A pharmaceutical composition comprising the recombinant vaccinia virus of claim 27.
